(19) United States
(12) Patent Application Publication
(10) Pub. No.: US 2024/0002473 A1
(43) Pub. Date: Jan. 4, 2024

(54) MEMBRANE PROTEIN TARGETING ENGINEERED DEUBIQUITINASES AND METHODS OF USE THEREOF

(71) Applicant: FLUX THERAPEUTICS, INC., New York, NY (US)

(72) Inventors: Andreas LOEW, MATTAPOISETT, MA (US); Samuel W. HALL, Brooklyn, NY (US)

(21) Appl. No.: 18/251,848

(22) PCT Filed: Nov. 5, 2021
(86) PCT No.: PCT/US2021/058291

§ 371 Date: May 4, 2023

Related U.S. Application Data
(60) Provisional application No. 63/110,619, filed on Nov. 6, 2020.

(51) Int. Cl. C07K 14/81; C07K 16/44

(52) U.S. Cl. CPC C07K 14/8139; C07K 14/8146; C07K 16/44; A61K 38/00

(57) ABSTRACT

Provided herein are fusion protein comprising: an effector domain comprising a catalytic domain of a deubiquitinase, or a functional fragment or functional variant thereof; and a targeting domain comprising a moiety that specifically binds a membrane protein. Also provided herein are methods of using the fusion proteins to treat a disease, including genetic diseases.

### CROSS-REFERENCE TO RELATED APPLICATIONS

This application claims the benefit under 35 U.S.C. § 119(e) of U.S. Provisional Patent Application No. 63/110,619, filed Nov. 6, 2020, the entire disclosure of which is incorporated herein by reference.

### 1. FIELD

This disclosure relates to fusion proteins comprising an effector domain comprising a catalytic domain of a deubiquitinase, or a functional fragment or functional variant thereof; and a targeting domain comprising a moiety that specifically binds a target membrane protein. The disclosure further relates to therapeutic methods of using the same.

### 2. BACKGROUND

A subset of genetic diseases are associated with a decrease in the level of expression of a functional membrane protein or a decrease in the stability of a membrane protein. For example, haploinsufficiency genetic diseases are caused by the presence a single copy of a wild-type allele in heterozygous combination with a loss of function variant allele, wherein the level of functional protein expressed is insufficient to produce the standard phenotype. Haploinsufficiency can arise from a de novo or inherited loss-of-function mutation in the variant allele, such that it produces little or no functional protein. Despite recent developments in gene therapy, there are still no curative treatments for these diseases, and treatment typically centers on the management of symptoms. Therefore, new treatments are needed for diseases, e.g., genetic diseases, that are associated with decreased functional membrane protein expression or stability.

### 3. SUMMARY

Provided herein are, inter alia, engineered deubiquitinases (enDubs) that comprise a targeting moiety that specifically binds a membrane target protein and a catalytic domain of a deubiquitinase. The targeting moiety directs that deubiquitinase catalytic domain to the specific target membrane protein for deubiquitination. The fusion proteins described herein are particularly useful in methods of treating genetic diseases, particularly those associated with or caused by decreased expression or stability of a specific membrane protein.

In one aspect, provided herein are fusion proteins comprising: an effector domain comprising a catalytic domain of a deubiquitinase, or a functional fragment or functional variant thereof; and a targeting domain comprising a targeting moiety that specifically binds a membrane protein that is not an ion channel.

In some embodiments, the deubiquitinase is a cysteine protease or a metalloprotease.

In some embodiments, the deubiquitinase is a cysteine protease. In some embodiments, the cysteine protease is a ubiquitin-specific protease (USP), a ubiquitin C-terminal hydrolase (UCH), a Machado-Josephin domain protease (MJD), an ovarian tumour protease (OTU), a MINDY protease, or a ZUFSP protease.

In some embodiments, the cysteine protease is a USP. In some embodiments, the USP is USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, or USP46.

In some embodiments, the cysteine protease is a UCH. In some embodiments, the UCH is BAP1, UCHL1, UCHL3, or UCHL5.

In some embodiments, the cysteine protease is a MJD. In some embodiments, the MJD is ATXN3 or ATXN3L.

In some embodiments, the cysteine protease is a OTU. In some embodiments, the OTU is OTUB1 or OTUB2.

In some embodiments, the cysteine protease is a MINDY. In some embodiments, the MINDY is MINDY1, MINDY2, MINDY3, or MINDY4.

In some embodiments, the cysteine protease is a ZUFSP. In some embodiments, the ZUFSP is ZUP1.

In some embodiments, the deubiquitinase is a metalloprotease. In some embodiments, the metalloprotease is a Jab1/Mov34/Mpr1 Pad1 N-terminal+(MPN+) (JAMM) domain protease.

In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-112.

In some embodiments, the catalytic domain comprises a catalytic domain derived from a deubiquitinase at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-112.

In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 113-220 or 293.

In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 293.

In some embodiments, the moiety that specifically binds a membrane protein comprises an antibody, or functional fragment or functional variant thereof. In some embodiments, the antibody, or functional fragment or functional variant thereof, comprises a full-length antibody, a single chain variable fragment (scFv), a scFv2, a scFv-Fc, a Fab, a Fab′, a F(ab′)2, a F(v), a VHH, or a (VHH)₂. In some embodiments, the antibody, or functional fragment or functional variant thereof, comprises a VHH or a (VHH)₂.

In some embodiments, the membrane protein is selected from the group consisting of solute carrier family 2, facilitated glucose transporter member 1 (SLC2A1), proline-rich transmembrane protein 2 (PRRT2), usherin (USH2A), protocadherin-19 (PCDH19), tuberin (TSC2), hamartin (TSC1), dystrophin (DMD), Rhodopsin (RHO), protein jagged-1 (JAG1), inositol 1,4,5-trisphosphate receptor type 1 (ITPR1), sugar transporter SWEET1 (SLC50A1), transmembrane protein 258 (TMEM258), or follicle stimulating hormone receptor (FSHR).

In some embodiments, the membrane protein comprises an amino acid sequence at least 90%, 91%, 92%, 93%, 94%, 95%, 96%, 97%, 98%, 99%, or 100% identical to the amino acid sequence of any one of SEQ ID NOS: 221-227 or 243-245.

In some embodiments, the effector domain is directly operably connected to the targeting domain. In some embodiments, the effector domain is indirectly operably connected to the targeting domain. In some embodiments, the effector domain is indirectly operably connected to the targeting domain via a peptide linker. In some embodiments, the effector domain is indirectly operably connected to the targeting domain via a peptide linker of sufficient length such that the effector domain and the targeting domain can simultaneous bind the respective target proteins. In some embodiments, the peptide linker comprises the amino acid sequence of any one of SEQ ID NOS: 297-424, or the amino acid sequence of any one of SEQ ID NOS: 297-424 comprising 1, 2, or 3 amino acid modifications. In some embodiments, the peptide linker comprises the amino acid sequence of any one of SEQ ID NOS: 297-306, or the amino acid sequence of any one of SEQ ID NOS: 297-306 comprising 1, 2, or 3 amino acid modifications.

In some embodiments, the effector domain is operably connected either directly or indirectly to the C terminus of the targeting domain. In some embodiments, the effector moiety is operably connected either directly or indirectly to the N terminus of the targeting domain.

In one aspect, provided herein are fusion proteins comprising: an effector domain comprising a catalytic domain of a deubiquitinase, or a functional fragment or functional variant thereof; and a targeting domain comprising a targeting moiety that specifically binds a membrane protein selected from the group consisting of glutamate receptor ionotropic NMDA 2B (GRIN2B), cystic fibrosis transmembrane conductance regulator (CFTR), sodium channel protein type 1 subunit alpha (SCN1A), copper-transporting ATPase 2 (ATP7B), potassium voltage-gated channel subfamily KQT member 2 (KCNQ2), sodium channel protein type 2 subunit alpha (SCN2A), voltage-dependent P/Q-type calcium channel subunit alpha-1A (CACNA1A), sodium channel protein type 8 subunit alpha (SCN8A), glutamate receptor ionotropic, NMDA 2A (GRIN2A), sodium- and chloride-dependent GABA transporter 1 (SLC6A1), sodium/potassium-transporting ATPase subunit alpha-2 (ATP1A2), sodium/potassium-transporting ATPase subunit alpha-3 (ATP1A3), sodium channel protein type 9 subunit alpha (SCN9A), gamma-aminobutyric acid receptor subunit beta-3 (GABRB3), and potassium voltage-gated channel subfamily KQT member 3 (KCNQ3).

In some embodiments, the moiety that specifically binds a membrane protein comprises an antibody, or functional fragment or functional variant thereof. In some embodiments, the antibody, or functional fragment or functional variant thereof, comprises a full-length antibody, a single chain variable fragment (scFv), a scFv2, a scFv-Fc, a Fab, a Fab′, a F(ab′)2, a F(v), a VHH, or a (VHH)₂. In some embodiments, the antibody, or functional fragment or functional variant thereof, comprises a VHH or a (VHH)₂.

In some embodiments, the membrane protein comprises an amino acid sequence at least 90%, 91%, 92%, 93%, 94%, 95%, 96%, 97%, 98%, 99%, or 100% identical to the amino acid sequence of any one of SEQ ID NOS: 228-245.

In some embodiments, the deubiquitinase is a cysteine protease or a metalloprotease. In some embodiments, the deubiquitinase is a cysteine protease.

In some embodiments, the cysteine protease is a ubiquitin-specific protease (USP), a ubiquitin C-terminal hydrolase (UCH), a Machado-Josephin domain protease (MJD), an ovarian tumour protease (OTU), a MINDY protease, or a ZUFSP protease.

In some embodiments, the cysteine protease is a USP. In some embodiments, the USP is USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, or USP46.

In some embodiments, the cysteine protease is a UCH. In some embodiments, the UCH is BAP1, UCHL1, UCHL3, or UCHL5.

In some embodiments, the cysteine protease is a MJD. In some embodiments, the MJD is ATXN3 or ATXN3L.

In some embodiments, the cysteine protease is a OTU. In some embodiments, the OTU is OTUB1 or OTUB2.

In some embodiments, the cysteine protease is a MINDY. In some embodiments, the MINDY is MINDY1, MINDY2, MINDY3, or MINDY4.

In some embodiments, the cysteine protease is a ZUFSP. In some embodiments, the ZUFSP is ZUP1.

In some embodiments, the deubiquitinase is a metalloprotease. In some embodiments, the metalloprotease is a Jab1/Mov34/Mpr1 Pad1 N-terminal+(MPN+) (JAMM) domain protease.

In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-112.

In some embodiments, the catalytic domain comprises a catalytic domain derived from a deubiquitinase at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-112.

In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 113-220 or 293.

In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 293.

In some embodiments, the effector domain is directly operably connected to the targeting domain. In some embodiments, the effector domain is indirectly operably connected to the targeting domain. In some embodiments, the effector domain is indirectly operably connected to the targeting domain via a peptide linker. In some embodiments, the effector domain is indirectly operably connected to the targeting domain via a peptide linker of sufficient length such that the effector domain and the targeting domain can simultaneous bind the respective target proteins. In some embodiments, the peptide linker comprises the amino acid sequence of any one of SEQ ID NOS: 297-424, or the amino acid sequence of any one of SEQ ID NOS: 297-424 comprising 1, 2, or 3 amino acid modifications. In some embodiments, the peptide linker comprises the amino acid sequence of any one of SEQ ID NOS: 297-306, or the amino acid sequence of any one of SEQ ID NOS: 297-306 comprising 1, 2, or 3 amino acid modifications.

In some embodiments, the effector domain is operably connected either directly or indirectly to the C terminus of the targeting domain. In some embodiments, the effector moiety is operably connected either directly or indirectly to the N terminus of the targeting domain.

In one aspect, provided herein are nucleic acid molecules encoding a fusion protein described herein. In some embodiments, the nucleic acid molecule is a DNA molecule. In some embodiments, the nucleic acid molecule is an RNA molecule.

In one aspect, provided herein are vectors comprising a nucleic acid molecule described herein (e.g., a nucleic acid molecule encoding a fusion protein described herein). In some embodiments, the vector is a plasmid or a viral vector.

In one aspect, provided herein are viral particles comprising a nucleic acid molecule described herein (e.g., a nucleic acid molecule encoding a fusion protein described herein).

In one aspect, provided herein are in vitro cell or population of cells comprising a fusion protein described herein, a nucleic acid molecule described herein, or a vector described herein.

In one aspect, provided herein are pharmaceutical compositions comprising a fusion protein described herein, a nucleic acid described herein, a vector described herein, or a viral particle described herein, and an excipient.

In one aspect, provided herein are methods of making a fusion protein described herein, comprising introducing into an in vitro cell or population of cells a nucleic acid molecule described herein, a vector described herein, or a viral particle described herein; culturing the cell or population of cells in a culture medium under conditions suitable for expression of the fusion protein, isolating the fusion protein from the culture medium, and optionally purifying the fusion protein.

In one aspect, provided herein are methods of treating or preventing a disease in a subject comprising administering a fusion protein described herein, a nucleic acid molecule described herein, a vector described herein, a viral particle described herein, or a pharmaceutical composition described herein, to a subject in need thereof. In some embodiments, the subject is human.

In some embodiments, the disease is associated with decreased expression of a functional version of the mitochondrial protein relative to a non-diseased control. In some embodiments, the disease is associated with decreased stability of a functional version of the mitochondrial protein relative to a non-diseased control. In some embodiments, the disease is associated with increased ubiquitination of the nuclear protein relative to a non-diseased control. In some embodiments, the disease is associated with increased ubiquitination and degradation of the mitochondrial protein relative to a non-diseased control. In some embodiments, the disease is a genetic disease. In some embodiments, the disease is a genetic disease. In some embodiments, the genetic disease is a haploinsufficiency disease.

In some embodiments, the disease is a GRIN2B-Related Disorder, a SCN2A-Related Disorder, a SCN8A-Related Disorder, SLC6A1-Related Disorder, a PRRT2 Dyskinesia & Epilepsy, a GRIN2A-Related Disorder, a CACNA1A-Related Disorder, a SCN9A Epilepsy, a PCDH19 Encephalopathy, GLUT1 deficiency syndrome, episodic kinesigenic dyskinesia 1, Usher syndrome type 2A, early infantile epileptic encephalopathy type 9, tuberous sclerosis type 2; tuberous sclerosis type 1, a KCNQ2-Related Disorder (e.g., epileptic encephalopathy), Becker Muscular Dystrophy, autosomal Dominant RP, Alagille syndrome 1, Gillespie Syndrome.

In some embodiments, the disease is early infantile epileptic encephalopathy type 11, early infantile epileptic encephalopathy type 13, early infantile epileptic encephalopathy type 27, cystic fibrosis, Dravet syndrome, Wilson disease, episodic ataxia type 2; epilepsy (e.g., focal, with speech disorder and with or without mental retardation), myoclonic-atonic epilepsy, alternating hemiplegia of childhood, alternating hemiplegia of childhood type 2, epilepsy type 7, GABRB3 associated epilepsy, or a KCNQ2-Related Disorder (e.g., epileptic encephalopathy).

In some embodiments, the disease is a GRIN2B-Related Disorder, a SCN2A-Related Disorder, a SCN8A-Related Disorder, SLC6A1-Related Disorder, a PRRT2 Dyskinesia & Epilepsy, a GRIN2A-Related Disorder, a CACNA1A-Related Disorder, a SCN9A Epilepsy, a PCDH19 Encephalopathy, early infantile epileptic encephalopathy type 9, early infantile epileptic encephalopathy type 11, early infantile epileptic encephalopathy type 13, early infantile epileptic encephalopathy type 27, cystic fibrosis, Dravet syndrome, Wilson disease, episodic ataxia type 2; GLUT1 deficiency syndrome, episodic kinesigenic dyskinesia 1, epilepsy (e.g., focal, with speech disorder and with or without mental retardation), KCNQ2 encephalopathy, myoclonic-atonic epilepsy, Usher syndrome type 2A, alternating hemiplegia of childhood, alternating hemiplegia of childhood type 2, epilepsy type 7, GABRB3 associated epilepsy; tuberous sclerosis type 2; tuberous sclerosis type 1, Becker Muscular Dystrophy, autosomal Dominant RP, Alagille syndrome 1, or Gillespie Syndrome.

In some embodiments, the target membrane protein is GRIN2B, and the disease is a GRIN2B related disorder (e.g., an epileptic encephalopathy); the target membrane protein is GRIN2B, and the disease is an early infantile epileptic encephalopathy; the target membrane protein is GRIN2B, and the disease is early infantile epileptic encephalopathy type 27; the target membrane protein is CFTR, and the disease is cystic fibrosis; the target membrane protein is SCN1A, and the disease is Dravet syndrome; the target membrane protein is ATP7B, and the disease is Wilson disease; the target membrane protein is CACNA1A, and the disease is a CACA1A related disorder; the target membrane protein is CACNA1A, and the disease is episodic ataxia type 2; the target membrane protein is KCNQ2, and the disease is an KCNQ2 encephalopathy; the target membrane protein is KCNQ2, and the disease is an epileptic encephalopathy; the target membrane protein is SCN2A, and the disease is a SCN2A related disorder (e.g., an epileptic encephalopathy); the target membrane protein is SCN2A, and the disease is early infantile epileptic encephalopathy type 11; the target membrane protein is SLC2A1, and the disease is GLUT1 deficiency syndrome; the target membrane protein is SCN8A, and the disease is a SCN8A related disorder (e.g., an epileptic encephalopathy); the target membrane protein is SCN8A, and the disease is an epileptic encephalopathy; the target membrane protein is SCN8A, and the disease is early infantile epileptic encephalopathy type 13; the target membrane protein is PRRT2, and the disease is a PRRPT2 dyskinesia and/or epilepsy; the target membrane protein is PRRT2, and the disease is an episodic kinesigenic dyskinesia type; the target membrane protein is PRRT2, and the disease is episodic kinesigenic dyskinesia type 1; the target membrane protein is GRIN2A, and the disease is a GRIN2A related disorder; the target membrane protein is GRIN2A, and the disease is epilepsy; the target membrane protein is GRIN2A, and the disease is focal epilepsy; the target membrane protein is GRIN2A, and the disease is focal epilepsy with speech disorder and with or without mental retardation; the target membrane protein is SLC6A1, and the disease is a SLC6A1 related disorder; the target membrane protein is SLC6A1, and the disease is epilepsy; the target membrane protein is SLC6A1, and the disease is myoclonic-atonic epilepsy; the target membrane protein is USH2A, and the disease is Usher syndrome; the target membrane protein is USH2A, and the disease is Usher syndrome type 2A; the target membrane protein is ATP1A2, and the disease is alternating hemiplegia of childhood; the target membrane protein is ATP1A2, and the disease is alternating hemiplegia of childhood type 1; the target membrane protein is ATP1A3, and the disease is alternating hemiplegia of childhood; the target membrane protein is ATP1A3, and the disease is alternating hemiplegia of childhood type 2; the target membrane protein is SCN9A, and the disease an SCN9A epilepsy; the target membrane protein is SCN9A1, and the disease an SCN9A epilepsy; the target membrane protein is SCN9A1, and the disease is epilepsy; the target membrane protein is SCN9A1, and the disease is epilepsy type 7; the target membrane protein is PCDH19, and the disease is PCDH19 encephalopathy; the target membrane protein is PCDH19, and the disease is an early infantile epileptic encephalopathy; the target membrane protein is PCDH19, and the disease is early infantile epileptic encephalopathy type 9; the target membrane protein is GABRB3, and the disease is epilepsy; the target membrane protein is GABRB3, and the disease is GABRB3 associated epilepsy; the target membrane protein is TSC2, and the disease is tuberous sclerosis; the target membrane protein is TSC2, and the disease is tuberous sclerosis type 2; the target membrane protein is TSC2, and the disease is tuberous sclerosis type 1; the target membrane protein is TSC1, and the disease is tuberous sclerosis; the target membrane protein is TSC1, and the disease is tuberous sclerosis type 1; the target membrane protein is TSC1, and the disease is tuberous sclerosis type 2; the target membrane protein is KCNQ3, and the disease is KCNQ2-Related Disorders (Epileptic Encephalopathy); the target membrane protein is DMD, and the disease is Becker Muscular Dystrophy; the target membrane protein is RHO, and the disease is Autosomal Dominant RP; the target membrane protein is JAG1, and the disease is Alagille syndrome 1; the target membrane protein is ITPR1, and the disease is Gillespie Syndrome; or the target membrane protein is FSHR, and the disease is ovarian dysgenesis 1 (ODG1).

In some embodiments, the fusion protein is administered at a therapeutically effective dose. In some embodiments, the fusion protein is administered systematically or locally. In some embodiments, the fusion protein is administered intravenously, subcutaneously, or intramuscularly.

In one aspect, provided herein are fusion proteins described herein, polynucleotides described herein, DNA described herein, RNA described herein, vectors described herein, viral particles described herein, and pharmaceutical compositions described herein for use as a medicament.

In one aspect, provided herein are fusion proteins described herein, polynucleotides described herein, DNA described herein, RNA described herein, vectors described herein, viral particles described herein, and pharmaceutical compositions described herein for use in treating or inhibiting a genetic disorder.

In one aspect, provided herein are fusion proteins comprising: (a) an effector domain comprising a catalytic domain of a deubiquitinase, or a functional fragment or functional variant thereof; and (b) a targeting domain comprising a targeting moiety that specifically binds a membrane protein that is not an ion channel.

In some embodiments, the deubiquitinase is a cysteine protease or a metalloprotease.

In some embodiments, the deubiquitinase is a cysteine protease. In some embodiments, the cysteine protease is a ubiquitin-specific protease (USP), a ubiquitin C-terminal hydrolase (UCH), a Machado-Josephin domain protease (MJD), an ovarian tumour protease (OTU), a MINDY protease, or a ZUFSP protease.

In some embodiments, the cysteine protease is a USP. In some embodiments, the USP is selected from the group consisting of USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, and USP46.

In some embodiments, the cysteine protease is a UCH. In some embodiments, the UCH is selected from the group consisting of BAP1, UCHL1, UCHL3, and UCHL5.

In some embodiments, the cysteine protease is a MJD. In some embodiments, the MJD is selected from the group consisting of ATXN3 and ATXN3L.

In some embodiments, the cysteine protease is a OTU. In some embodiments, the OTU is selected from the group consisting of OTUB1 and OTUB2.

In some embodiments, the cysteine protease is a MINDY. In some embodiments, the MINDY is selected from the group consisting of MINDY1, MINDY2, MINDY3, and MINDY4.

In some embodiments, the cysteine protease is a ZUFSP. In some embodiments, the ZUFSP is ZUP1.

In some embodiments, the deubiquitinase is a metalloprotease. In some embodiments, the metalloprotease is a Jab1/Mov34/Mpr1 Pad1 N-terminal+(MPN+) (JAMM) domain protease.

In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 1-112.

In some embodiments, the catalytic domain comprises a catalytic domain derived from a deubiquitinase at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 1-112.

In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 113-220.

In some embodiments, the moiety that specifically binds a membrane protein comprises an antibody, or functional fragment or functional variant thereof. In some embodiments, the antibody, or functional fragment or functional variant thereof, comprises a full-length antibody, a single chain variable fragment (scFv), a scFv2, a scFv-Fc, a Fab, a Fab′, a F(ab′)2, a F(v), or a VHH. In some embodiments, the antibody, or functional fragment or functional variant thereof, comprises a VHH.

In some embodiments, the membrane protein is selected from the group consisting of solute carrier family 2, facilitated glucose transporter member 1 (SLC2A1), proline-rich transmembrane protein 2 (PRRT2), usherin (USH2A), protocadherin-19 (PCDH19), tuberin (TSC2), hamartin (TSC1), and dystrophin (DMD).

In some embodiments, the membrane protein comprises an amino acid sequence at least 90%, 91%, 92%, 93%, 94%, 95%, 96%, 97%, 98%, 99%, or 100% identical to any one of SEQ ID NOS: 221-227 or 243-245.

In some embodiments, the effector domain is directly fused to the targeting domain.

In some embodiments, the effector domain is indirectly fused to the targeting domain.

In some embodiments, the effector domain is indirectly fused to the targeting domain via a peptide linker. In some embodiments, the effector domain is indirectly fused to the targeting domain via a peptide linker of sufficient length such that the effector domain and the targeting domain can simultaneous bind the respective target proteins.

In some embodiments, the effector domain is fused to the C terminus of the targeting domain. In some embodiments, the effector moiety is fused to the N terminus of the targeting domain.

In one aspect, provided herein are fusion proteins comprising: (a) an effector domain comprising a catalytic domain of a deubiquitinase, or a functional fragment or functional variant thereof; and (b) a targeting domain comprising a targeting moiety that specifically binds a membrane protein selected from the group consisting of glutamate receptor ionotropic NMDA 2B (GRIN2B), cystic fibrosis transmembrane conductance regulator (CFTR), sodium channel protein type 1 subunit alpha (SCN1A), copper-transporting ATPase 2 (ATP7B), potassium voltage-gated channel subfamily KQT member 2 (KCNQ2), sodium channel protein type 2 subunit alpha (SCN2A), voltage-dependent P/Q-type calcium channel subunit alpha-1A (CACNA1A), sodium channel protein type 8 subunit alpha (SCN8A), glutamate receptor ionotropic, NMDA 2A (GRIN2A), sodium- and chloride-dependent GABA transporter 1 (SLC6A1), sodium/potassium-transporting ATPase subunit alpha-2 (ATP1A2), sodium/potassium-transporting ATPase subunit alpha-3 (ATP1A3), sodium channel protein type 9 subunit alpha (SCN9A), gamma-aminobutyric acid receptor subunit beta-3 (GABRB3), and potassium voltage-gated channel subfamily KQT member 3 (KCNQ3).

In some embodiments, the moiety that specifically binds a membrane protein comprises an antibody, or functional fragment or functional variant thereof.

In some embodiments, the antibody, or functional fragment or functional variant thereof, comprises a full-length antibody, a single chain variable fragment (scFv), a scFv2, a scFv-Fc, a Fab, a Fab′, a F(ab′)2, a F(v), or a VHH. In some embodiments, the antibody, or functional fragment or functional variant thereof, comprises a VHH.

In some embodiments, the membrane protein comprises an amino acid sequence at least 90%, 91%, 92%, 93%, 94%, 95%, 96%, 97%, 98%, 99%, or 100% identical to any one of SEQ ID NOS: 228-245.

In some embodiments, the deubiquitinase is a cysteine protease or a metalloprotease.

In some embodiments, the deubiquitinase is a cysteine protease. In some embodiments, the cysteine protease is a ubiquitin-specific protease (USP), a ubiquitin C-terminal hydrolase (UCH), a Machado-Josephin domain protease (MJD), an ovarian tumour protease (OTU), a MINDY protease, or a ZUFSP protease.

In some embodiments, the cysteine protease is a USP. In some embodiments, the USP is selected from the group consisting of USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, and USP46.

In some embodiments, the cysteine protease is a UCH. In some embodiments, the UCH is selected from the group consisting of BAP1, UCHL1, UCHL3, and UCHL5.

In some embodiments, the cysteine protease is a MJD. In some embodiments, the MJD is selected from the group consisting of ATXN3 and ATXN3L.

In some embodiments, the cysteine protease is a OTU. In some embodiments, the OTU is selected from the group consisting of OTUB1 and OTUB2.

In some embodiments, the cysteine protease is a MINDY. In some embodiments, the MINDY is selected from the group consisting of MINDY1, MINDY2, MINDY3, and MINDY4.

In some embodiments, the cysteine protease is a ZUFSP. In some embodiments, the ZUFSP is ZUP1.

In some embodiments, the deubiquitinase is a metalloprotease. In some embodiments, the metalloprotease is a Jab1/Mov34/Mpr1 Pad1 N-terminal+(MPN+) (JAMM) domain protease.

In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 1-112.

In some embodiments, the catalytic domain comprises a catalytic domain derived from a deubiquitinase at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 1-112.

In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 113-220.

In some embodiments, the effector domain is directly fused to the targeting domain.

In some embodiments, the effector domain is indirectly fused to the targeting domain.

In some embodiments, the effector domain is indirectly fused to the targeting domain via a peptide linker. In some embodiments, the effector domain is indirectly fused to the targeting domain via a peptide linker of sufficient length such that the effector domain and the targeting domain can simultaneous bind the respective target proteins.

In some embodiments, the effector domain is fused to the C terminus of the targeting domain. In some embodiments, the effector moiety is fused to the N terminus of the targeting domain.

In one aspect, provided herein are nucleic acid molecules encoding the fusion protein described herein. In some embodiments, the nucleic acid molecule is a DNA molecule. In some embodiments, the nucleic acid molecule is an RNA molecule.

In one aspect, provided herein are vectors comprising a nucleic acid molecule described herein. In some embodiments, the vector is a plasmid or a viral vector.

In one aspect, provided herein are viral particles comprising a nucleic acid described herein.

In one aspect, described herein is an in vitro cell or population of cells comprising a fusion protein described herein, a nucleic acid molecule described herein, or a vector described herein.

In one aspect, provided herein are pharmaceutical compositions comprising a fusion protein described herein, a nucleic acid molecule described herein, a vector described herein, or a viral particle described herein, and an excipient.

In one aspect, provided herein are methods of making a fusion protein described herein, comprising (a) introducing into an in vitro cell or population of cells a nucleic acid described herein, a vector described herein, or a viral particle described herein; (b) culturing the cell or population of cells in a culture medium under conditions suitable for expression of the fusion protein, (c) isolating the fusion protein from the culture medium, and (d) optionally purifying the fusion protein.

In one aspect, provided herein are methods of treating a disease in a subject comprising administering a fusion protein described herein, a nucleic acid described herein, a vector described herein, or a viral particle described herein, or a pharmaceutical composition described herein, to a subject in need thereof.

In some embodiments, the subject is human.

In some embodiments, the disease is associated with decreased expression of a functional version of the membrane protein relative to a non-diseased control.

In some embodiments, the disease is associated with decreased stability of a functional version of the membrane protein relative to a non-diseased control.

In some embodiments, the disease is associated with increased ubiquitination and degradation of the membrane protein relative to a non-diseased control.

In some embodiments, the disease is a genetic disease.

In some embodiments, the disease is GLUT1 deficiency syndrome, episodic kinesigenic dyskinesia 1, Usher syndrome type 2A, early infantile epileptic encephalopathy type 9, tuberous sclerosis type 2; tuberous sclerosis type 1, a KCNQ2-Related Disorder (e.g., epileptic encephalopathy), Becker Muscular Dystrophy, autosomal Dominant RP, Alagille syndrome 1, and Gillespie Syndrome.

In some embodiments, the disease is selected from the group consisting of early infantile epileptic encephalopathy type 11, early infantile epileptic encephalopathy type 13, early infantile epileptic encephalopathy type 27, cystic fibrosis, Dravet syndrome, Wilson disease, episodic ataxia type 2; epilepsy (e.g., focal, with speech disorder and with or without mental retardation), myoclonic-atonic epilepsy, alternating hemiplegia of childhood, alternating hemiplegia of childhood type 2, epilepsy type 7, GABRB3 associated epilepsy, and a KCNQ2-Related Disorder (e.g., epileptic encephalopathy).

In some embodiments, the disease is selected from the group consisting of GRIN2B-Related Disorder, early infantile epileptic encephalopathy type 9, early infantile epileptic encephalopathy type 11, early infantile epileptic encephalopathy type 13, early infantile epileptic encephalopathy type 27, cystic fibrosis, Dravet syndrome, Wilson disease, episodic ataxia type 2; GLUT1 deficiency syndrome, episodic kinesigenic dyskinesia 1, epilepsy (e.g., focal, with speech disorder and with or without mental retardation), myoclonic-atonic epilepsy, Usher syndrome type 2A, alternating hemiplegia of childhood, alternating hemiplegia of childhood type 2, epilepsy type 7, GABRB3 associated epilepsy; tuberous sclerosis type 2; tuberous sclerosis type 1, Becker Muscular Dystrophy, autosomal Dominant RP, Alagille syndrome 1, and Gillespie Syndrome.

In some embodiments, the disease is a haploinsufficiency disease.

In some embodiments, the fusion protein is administered at a therapeutically effective dose.

In some embodiments, the fusion protein is administered systematically or locally.

In some embodiments, the fusion protein is administered intravenously, subcutaneously, or intramuscularly.

In one aspect, provided herein are nucleic acid molecules encoding the fusion protein described herein. In some embodiments, the nucleic acid molecule is a DNA molecule. In some embodiments, the nucleic acid molecule is an RNA molecule.

In one aspect, provided herein are vectors comprising a nucleic acid molecule described herein. In some embodiments, the vector is a plasmid or a viral vector.

In one aspect, provided herein are viral particles comprising a nucleic acid described herein.

In one aspect, described herein is an in vitro cell or population of cells comprising a fusion protein described herein, a nucleic acid molecule described herein, or a vector described herein.

In one aspect, provided herein are pharmaceutical compositions comprising a fusion protein described herein, a nucleic acid molecule described herein, a vector described herein, or a viral particle described herein, and an excipient.

In one aspect, provided herein are methods of making a fusion protein described herein, comprising (a) introducing into an in vitro cell or population of cells a nucleic acid described herein, a vector described herein, or a viral particle described herein; (b) culturing the cell or population of cells in a culture medium under conditions suitable for expression of the fusion protein, (c) isolating the fusion protein from the culture medium, and (d) optionally purifying the fusion protein.

In one aspect, provided herein are methods of treating a disease in a subject comprising administering a fusion protein described herein, a nucleic acid described herein, a vector described herein, or a viral particle described herein, or a pharmaceutical composition described herein, to a subject in need thereof.

In some embodiments, the subject is human.

In some embodiments, the disease is associated with decreased expression of a functional version of the membrane protein relative to a non-diseased control.

In some embodiments, the disease is associated with decreased stability of a functional version of the membrane protein relative to a non-diseased control.

In some embodiments, the disease is associated with increased ubiquitination and degradation of the membrane protein relative to a non-diseased control.

In some embodiments, the disease is a genetic disease.

In some embodiments, the disease is early infantile epileptic encephalopathy type 2, Wilson disease, early infantile epileptic encephalopathy type 4, mental retardation autosomal dominant 5, aphasia, alagille syndrome 1, epilepsy, tuberous sclerosis-2, tuberous sclerosis-1, KIF1A-associated neurological disorder, encephalopathy, Phelan-McDermid syndrome, Becker Muscular Dystrophy, RP1, retinitis pigmentosa 1, dilated cardiomyopathy 1G, DYNC1H1 Syndrome, TRIO-Related intellectual disability (ID), and USP9X Development Disorder.

The method of any one of claims 43-48, wherein the disease is early infantile epileptic encephalopathy type 2, Wilson disease, early infantile epileptic encephalopathy type 4, mental retardation autosomal dominant 5, aphasia primary progressive & FTD (frontotemporal degeneration), alagille syndrome 1, epilepsy familial focal with variable foci 1, tuberous sclerosis-2, tuberous sclerosis-1, KIF1A-associated neurological disorder, encephalopathy, Phelan-McDermid syndrome, Becker Muscular Dystrophy, RP1, retinitis pigmentosa 1, dilated cardiomyopathy 1G, DYNC1H1 Syndrome, TRIO-Related intellectual disability (ID), and USP9X Development Disorder.

In some embodiments, the disease is a haploinsufficiency disease.

In some embodiments, the fusion protein is administered at a therapeutically effective dose.

In some embodiments, the fusion protein is administered systematically or locally.

In some embodiments, the fusion protein is administered intravenously, subcutaneously, or intramuscularly.

### 4. BRIEF DESCRIPTION OF THE FIGURES

FIGS. 1A-1D provides a schematic representation of exemplary fusion proteins described herein. FIG. 1A is a schematic of an engineered deubiquitinase comprising from N′ to C′ terminus a VHH that specifically binds a membrane target protein and the catalytic domain of a deubiquitinase. In this specific embodiment, the C-terminus of the VHH is directly connected to the N-terminus of the catalytic domain of the deubiquitinase. FIG. 1B is a schematic of an engineered deubiquitinase comprising from N′ to C′ terminus the catalytic domain of a deubiquitinase that specifically binds a membrane target protein and a VHH that specifically binds a membrane target protein. In this specific embodiment, the C-terminus of the catalytic domain of the deubiquitinase is directly connected to the N-terminus of the VHH. FIG. 1C is a schematic of an engineered deubiquitinase comprising from N′ to C′ terminus a VHH that specifically binds a membrane target protein and the catalytic domain of a deubiquitinase. In this specific embodiment, the C-terminus of the VHH is indirectly connected to the N-terminus of the catalytic domain of the deubiquitinase through a peptide linker. FIG. 1D is a schematic of an engineered deubiquitinase comprising from N′ to C′ terminus the catalytic domain of a deubiquitinase that specifically binds a membrane target protein and a VHH that specifically binds a membrane target protein. In this specific embodiment, the C-terminus of the catalytic domain of the deubiquitinase is indirectly connected to the N-terminus of the VHH through a peptide linker.

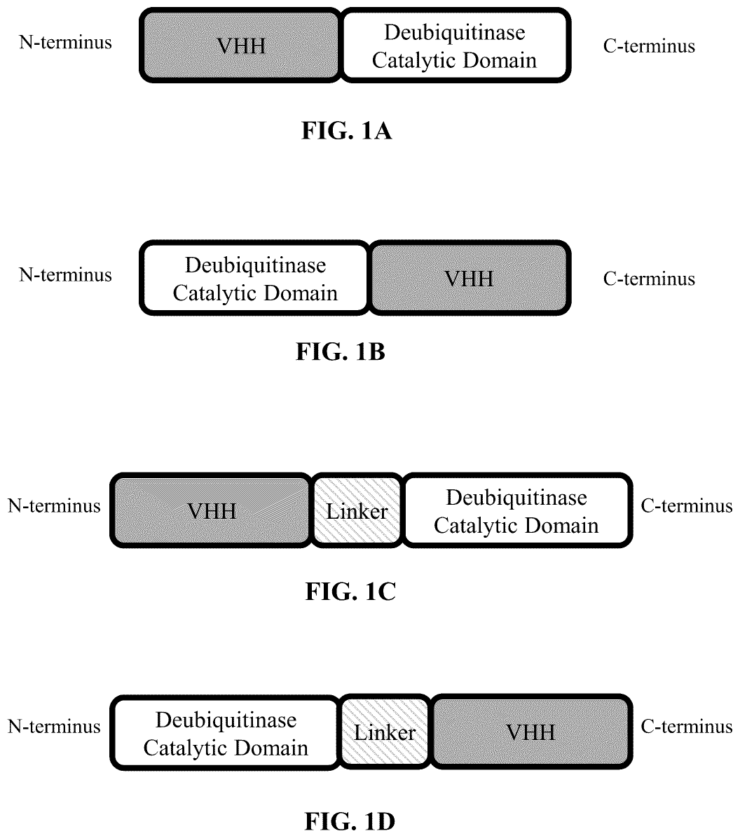

FIG. 2 is a schematic representation of the assay utilized in Example 3, to screen the effect of targeted deubiquitination of different membrane proteins on target protein expression.

FIG. 3 is a bar graph depicting the fold change in KCNQ1 protein expression relative to control (as indicated).

FIG. 4 is a bar graph depicting the fold change in SCN1A protein expression relative to control (as indicated).

FIG. 5 is a bar graph depicting the fold change in GRIN2B protein expression relative to control (as indicated).

FIG. 6 is a bar graph depicting the fold change in SLC50A1 protein expression relative to control (deubiquitinase without the nanobody targeting the alfa-tag).

FIG. 7 is a bar graph depicting the fold change in TREM258 protein expression relative to control (deubiquitinase without the nanobody targeting the alfa-tag).

FIG. 8 is a bar graph depicting the fold change in FSHR protein expression relative to control (deubiquitinase without the nanobody targeting the alfa-tag).

### 5. DETAILED DESCRIPTION

### 5.1 Overview

Ubiquitination is the process by which ubiquitin ligases mediate the addition of ubiquitin, a 76 amino acid regulatory protein, to a substrate protein. Ubiquitination generally starts by the attachment of a single ubiquitin molecule to a lysine amino acid residue of the substrate protein. Mevissen T. et al. Mechanisms of Deubiquitinase Specificity and Regulation Annual Review of Biochemistry 86:1, 159-192 (2017), the entire contents of which is incorporated by reference herein. These monoubiquitination events are abundant and serve various functions. Ubiquitin itself contains seven lysine residues, all of which can be ubiquitinated resulting in polyubiquitinated proteins. Komander, D. et al. Breaking the chains: structure and function of the deubiquitinases. Nat Rev Mol Cell Biol 10, 550-563 (2009), the entire contents of which is incorporated by reference herein. Mono and polyubiquitination can have multiple effects on the substrate protein, including marking the substrate protein for degradation via the proteasome, altering the protein's cellular location, altering the protein's activity, and/or promoting or preventing normal protein interactions. See e.g., Hershko A. et al. The ubiquitin system. Annu Rev Biochem. 67:425-79 (1998); Nandi D, et al. The ubiquitin-proteasome system. J Biosci. March; 31(1):137-55 (2006), the entire contents of each of which is incorporated by reference herein. The effects of ubiquitination can be reversed or prevented by removing the ubiquitin protein(s) from the substrate protein. The removal of ubiquitin from a substrate protein is mediated by deubiquitinase (DUB) proteins. Id.

Numerous genetic diseases are associated with or caused by a decrease in the level of expression of a functional membrane protein or the stability of the membrane protein. For example, haploinsufficiency genetic diseases are caused by the presence a single copy of a wild-type allele in heterozygous combination with a loss of function variant allele, wherein the level of functional protein expressed is insufficient to produce the standard phenotype. See e.g., Johnson, A. et al, Causes and effects of haploinsufficiency. Biol Rev, 94: 1774-1785 (2019), the entire contents of which is incorporated by reference herein. Haploinsufficiency can arise from a de novo or inherited loss-of-function mutation in the variant allele, such that it produces little or no functional protein. Other genetic disorders result from the ubiquitination and subsequent degradation of variant but functional proteins, resulting in a decrease in expression of the functional protein.

The present disclosure provides, inter alia, novel fusion proteins that comprise the catalytic domain (or functional fragment thereof) of a deubiquitinase and a targeting moiety, such as a VHH, that specifically binds to a target membrane protein. In some embodiments, decreased expression of a functional version of the target membrane protein or decreased stability of a functional version of the target membrane protein is associated with a disease phenotype. As such, the fusion proteins described herein are particularly useful in the treatment of genetic diseases characterized by a decrease in the level of expression of a functional target membrane protein or the stability of the target membrane protein. Upon expression of the fusion protein by host cells, the catalytic domain of the deubiquitinase will be specifically targeted to the target membrane protein and deubiquitinated, resulting in increased expression of the target membrane protein, e.g., to a level sufficient to alleviate the disease phenotype.

### 5.2 Definitions

The section headings used herein are for organizational purposes only and are not to be construed as limiting the subject matter described.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

It is to be understood that the foregoing general description and the following detailed description are exemplary and explanatory only and are not restrictive of any subject matter claimed. In this application, the use of the singular includes the plural unless specifically stated otherwise.

It must be noted that, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and/or “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As used herein, the term “catalytic domain” in reference to a deubiquitinase refers to an amino acid sequence, or a variant thereof, of a deubiquitinase that is capable of mediating deubiquitination of a target protein. The catalytic domain may comprise a naturally occurring amino acid sequence of a deubiquitinase or it may comprise a variant amino acid sequence of a naturally occurring deubiquitinase. The catalytic domain may comprise the minimum amino acid sequence of a deubiquitinase to mediate deubiquitination of a target protein. The catalytic domain may comprise more than the minimum amino acid sequence of a deubiquitinase to mediate deubiquitination of a target protein.

The terms “polynucleotide” and “nucleic acid sequence” are used interchangeably herein and refer to a polymer of DNA or RNA. The polynucleotide sequence can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified polynucleotide sequence. Polynucleotide sequences include, but are not limited to, all polynucleotide sequences which are obtained by any means available in the art, including, without limitation, recombinant means, e.g., the cloning of polynucleotide sequences from a recombinant library or a cell genome, using ordinary cloning technology and polymerase chain reaction, and the like, and by synthetic means.

The terms “amino acid sequence” and “polypeptide” are used interchangeably herein and refer to a polymer of amino acids connected by one or more peptide bonds.

The term “functional variant” as used herein in reference to a protein or polypeptide refers to a protein that comprises at least one amino acid modification (e.g., a substitution, deletion, addition) compared to the amino acid sequence of a reference protein, that retains at least one particular function. In some embodiments, the reference protein is a wild type protein. For example, a functional variant of an IL-2 protein can refer to an IL-2 protein comprising an amino acid substitution as compared to a wild type IL-2 protein that retains the ability to bind the intermediate affinity IL-2 receptor but abrogates the ability of the protein to bind the high affinity IL-2 receptor. Not all functions of the reference wild type protein need be retained by the functional variant of the protein. In some instances, one or more functions are selectively reduced or eliminated.

The term “functional fragment” as used herein in reference to a protein or polypeptide refers to a fragment of a reference protein that retains at least one particular function. For example, a functional fragment of an anti-HER2 antibody can refer to a fragment of the anti-HER2 antibody that retains the ability to specifically bind the HER2 antigen. Not all functions of the reference protein need be retained by a functional fragment of the protein. In some instances, one or more functions are selectively reduced or eliminated.

As used herein, the term “modification,” with reference to a polynucleotide sequence, refers to a polynucleotide sequence that comprises at least one substitution, alteration, inversion, addition, or deletion of nucleotide compared to a reference polynucleotide sequence. Modifications can include non-naturally nucleotides. As used herein, the term “modification,” with reference to an amino acid sequence refers to an amino acid sequence that comprises at least one substitution, alteration, inversion, addition, or deletion of an amino acid residue compared to a reference amino acid sequence. Modifications can include the inclusion of non-naturally occurring amino acid residues.

As used herein, the term “derived from” with reference to an amino acid sequence refers to an amino acid sequence that has at least 80% sequence identity to a reference naturally occurring amino acid sequence. For example, a catalytic domain derived from a naturally occurring deubiquitinase means that the catalytic domain has an amino acid sequence with at least 80% sequence identity to the sequence of the deubiquitinase catalytic domain from which it is derived. The term “derived from” as used herein does not denote any specific process or method for obtaining the amino acid sequence. For example, the amino acid sequence can be chemically or recombinantly synthesized.

The term “fusion protein” and grammatical equivalents as used herein refers to a protein that comprises an amino acid sequence derived from at least two separate proteins. The amino acid sequence of the at least two separate proteins can be directly connected through a peptide bond; or can be operably connected through an amino acid linker. Therefore, the term fusion protein encompasses embodiments, wherein the amino acid sequence of e.g., Protein A is directly connected to the amino acid sequence of Protein B through a peptide bond (Protein A-Protein B), and embodiments, wherein the amino acid sequence of e.g., Protein A is operably connected to the amino acid sequence of Protein B through an amino acid linker (Protein A-linker-Protein B).

The term “fuse” and grammatical equivalents thereof as used herein refers to the operable connection of an amino acid sequence derived from one protein to the amino acid sequence derived from different protein. The term fuse encompasses both a direct connection of the two amino acid sequences through a peptide bond, and the indirect connection through an amino acid linker.

An “isolated antibody” refers to an antibody that is substantially free of other antibodies having different antigenic specificities (e.g., an isolated antibody that binds specifically to HER2 is substantially free of antibodies that bind specifically to antigens other than HER2). An isolated antibody that binds specifically to HER2 may, however, cross-react with other antigens, such as HER2 molecules from different species. Moreover, an isolated antibody may be substantially free of other cellular material and/or chemicals. By comparison, an “isolated” nucleic acid refers to a nucleic acid composition of matter that is markedly different, i.e., has a distinctive chemical identity, nature and utility, from nucleic acids as they exist in nature. For example, an isolated DNA, unlike native DNA, is a freestanding portion of a native DNA and not an integral part of a larger structural complex, the chromosome, found in nature. Further, an isolated DNA, unlike native DNA, can be used as a PCR primer or a hybridization probe for, among other things, measuring gene expression and detecting biomarker genes or mutations for diagnosing disease or predicting the efficacy of a therapeutic. An isolated nucleic acid may also be purified so as to be substantially free of other cellular components or other contaminants, e.g., other cellular nucleic acids or proteins, using standard techniques well known in the art.

As used herein, the term “antibody” or “antibodies” are used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity (i.e. antigen binding fragments as defined herein). The term antibody thus includes, for example, include full-length antibodies, antigen-binding fragments of full-length antibodies, molecules comprising antibody CDRs, VH regions, and/or VL regions; and antibody-like scaffolds (e.g., fibronectins). Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies (e.g., VHH, (VHH)₂), monovalent antibodies, single chain antibodies, single-chain Fvs (scFv; (scFv)₂), camelized antibodies, affybodies, Fab fragments (e.g., Fab, single chain Fab (scFab), F(ab′)₂ fragments, disulfide-linked Fvs (sdFv), anti-idiotypic (anti-Id) antibodies (including, e.g., anti-anti-Id antibodies), diabodies, tribodies, and antibody-like scaffolds (e.g., fibronectins), Fc fusions (e.g., Fab-Fc, scFv-Fc, VHH-Fc, (scFv)₂-Fc, (VHH)₂—Fc, and antigen-binding fragments of any of the above, and conjugates or fusion proteins comprising any of the above. In certain embodiments, antibodies described herein refer to polyclonal antibody populations. In certain embodiments, antibodies described herein refer to monoclonal antibody populations. Antibodies can be of any type (e.g., IgG, IgE, IgM, IgD, IgA or IgY), any class (e.g., IgG₁, IgG₂, IgG₃, IgG₄, IgA₁ or IgA₂), or any subclass (e.g., IgG₂ₐ or IgG_{2b}) of immunoglobulin (Ig) molecule. In certain embodiments, antibodies described herein are IgG antibodies, or a class (e.g., human IgG₁ or IgG₄) or subclass thereof. In a specific embodiment, the antibody is a humanized monoclonal antibody. In another specific embodiment, the antibody is a human monoclonal antibody.

The term “full-length antibody,” as used herein refers to an antibody having a structure substantially similar to a native antibody structure comprising two heavy chains and two light chains interconnected by disulfide bonds. In some embodiments, the two heavy chains comprise a substantially identical amino acid sequence; and the two light chains comprise a substantially identical amino acid sequence. Antibody chains may be substantially identical but not entirely identical if they differ due to post-translational modifications, such as C-terminal cleavage of lysine residues, alternative glycosylation patterns, etc.

The terms “antigen binding fragment” and “antigen binding domain” are used interchangeably herein and refer to one or more polypeptides, other than a full-length antibody, that is capable of specifically binding to antigen and comprises a portion of a full-length antibody (e.g., a VH, a VL). Exemplary antigen binding fragments include, but are not limited to, single domain antibodies (e.g., VHH, (VHH)₂), single chain antibodies, single-chain Fvs (scFv; (scFv)₂), camelized antibodies, affybodies, Fab fragments (e.g., Fab, single chain Fab (scFab), F(ab′)₂ fragments, and disulfide-linked Fvs (sdFv). The antigen binding domain can be part of a larger protein, e.g., a full-length antibody.

The term “(scFv)₂” as used herein refers to an antibody that comprises a first and a second scFv operably connected (e.g., via a linker). The first and second scFv can specifically bind the same or different antigens. In some embodiments, the first and second scFv are operably connected by an amino via an amino acid linker.

The term “(VHH)₂” as used herein refers to an antibody that comprises a first and a second VHH operably connected (e.g., via a linker). The first and the second VHH can specifically bind the same or different antigens. In some embodiments, the first and second VHH are operably connected by an amino via an amino acid linker.

The term “Fab-Fc” as used herein refers to an antibody that comprises a Fab operably linked to an Fc domain or a subunit of an Fc domain. A full-length antibody described herein comprises two Fabs, one Fab operably connected to one Fc domain and the other Fab operably connected to a second Fc domain.

The term “scFv-Fc” as used herein refers to an antibody that comprises a scFv operably linked to an Fc domain or subunit of an Fc domain.

The term “VHH-Fc” as used herein refers to an antibody that comprises a VHH operably linked to an Fc domain or a subunit of an Fc domain.

The term “(scFv)₂-Fc” as used herein refers to a (scFv)₂ operably linked to an Fc domain or a subunit of an Fc domain.

The term “(VHH)₂—Fc” as used herein refers to (VHH)₂ operably linked to an Fc domain or a subunit of an Fc domain.

“Antibody-like scaffolds” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). Exemplary antibody-like scaffold proteins include, but are not limited to, lipocalins (Anticalin), Protein A-derived molecules such as Z-domains of Protein A (Affibody), an A-domain (Avimer/Maxibody), a serum transferrin (trans-body); a designed ankyrin repeat protein (DARPin), VNAR fragments, a fibronectin (AdNectin), a C-type lectin domain (Tetranectin); a variable domain of a new antigen receptor beta-lactamase (VNAR fragments), a human gamma-crystallin or ubiquitin (Affilin molecules); a kunitz type domain of human protease inhibitors, microbodies such as the proteins from the knottin family, peptide aptamers and fibronectin (adnectin).

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), all of which are herein incorporated by reference in their entireties. Unless otherwise specified, the term “CDR” is a CDR as defined by Kabat et al., J. Biol. Chem. 252, 6609-6616 (1977) and Kabat et al., Sequences of protein of immunological interest. (1991).

As used herein, the term “framework (FR) amino acid residues” refers to those amino acids in the framework region of an antibody variable region. The term “framework region” or “FR region” as used herein, includes the amino acid residues that are part of the variable region, but are not part of the CDRs (e.g., using the Kabat definition of CDRs).

As used herein, the term “heavy chain” when used in reference to an antibody can refer to any distinct type, e.g., alpha (α), delta (δ), epsilon (γ), gamma (γ), and mu (μ), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

As used herein, the terms “variable region” refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

As used herein, the terms “constant region” and “constant domain” are interchangeable and are common in the art. The constant region is an antibody portion, e.g., a carboxyl terminal portion of a light and/or heavy chain which is not directly involved in binding of an antibody to antigen but which can exhibit various effector functions, such as interaction with an Fc receptor (e.g., Fc gamma receptor). The constant region of an immunoglobulin (Ig) molecule generally has a more conserved amino acid sequence relative to an immunoglobulin (Ig) variable domain.

The term “Fc region” as used herein refers to the C-terminal region of an immunoglobulin (Ig) heavy chain that comprises from N- to C-terminus at least a CH2 domain operably connected to a CH3 domain. In some embodiments, the Fc region comprises an immunoglobulin (Ig) hinge region operably connected to the N-terminus of the CH2 domain. Examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, which is incorporated by reference herein).

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, each of which is herein incorporated by reference in its entirety.

As used herein, the term “Kabat numbering system” refers to the Kabat numbering convention for variable regions of an antibody, see e.g., Kabat et al, Sequences of Proteins of Immunological Interest, U.S. Dept. Health and Human Services, 5th edition, 1991. Unless otherwise noted, numbering of the variable regions of an antibody are denoted according to the Kabat numbering system.

As used herein, the terms “specifically binds,” refers to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In a specific embodiment, molecules that specifically bind to an antigen bind to the antigen with a K_{A} that is at least 2 logs (e.g., factors of 10), 2.5 logs, 3 logs, 4 logs or greater than the K_{A} when the molecules bind non-specifically to another antigen. The skilled worker will appreciate that an antibody, as described herein, can specifically bind to more than one antigen (e.g., via different regions of the antibody molecule). The term specifically binds includes molecules that are cross reactive with the same antigen of a different species. For example, an antigen binding domain that specifically binds human CD20 may be cross reactive with CD20 of another species (e.g., cynomolgus monkey, or murine), and still be considered herein to specifically bind human CD20.

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity, which reflects a 1:1 interaction between members of a binding pair (e.g., an antigen binding moiety and an antigen, or a receptor and its ligand). The affinity of a molecule X for its partner Y can generally be represented by the dissociation constant (KD), which is the ratio of dissociation and association rate constants (koff and kon, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

The determination of “percent identity” between two sequences (e.g., amino acid sequences or nucleic acid sequences) can be accomplished using a mathematical algorithm. Identity measures the percent of identical matches between the smaller of two or more sequences with gap alignments (if any) addressed by a particular mathematical model or computer program (i.e., “algorithms”). A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the BLASTN, BLASTP, BLASTX programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the BLASTP program parameters set, e.g., default settings; to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of BLASTP and BLASTN) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted. As described above, the percent identity is based on the amino acid matches between the smaller of two proteins. Therefore, for example, using NCBI Basic Local Alignment Tool-BLASTP program on the default settings (Search Parameters: word size 3, expect value 0.05, hitlist 100, Gapcosts 11,1; Matrix BLOSUM62, Filter string: F; Genetic Code: 1; Window Size: 40; Threshold: 11; Composition Based Stats: 2; Karlin-Altschul Statistics: Lambda: 0.31293; 0.267; K: 0.132922; 0.041; H: 0.401809; 0.14; and Relative Statistics: Effective search space: 288906); the percent identity between SEQ ID NO: 80 and SEQ ID NO: 293 is 100% identity.

As used herein, the term “operably connected” refers to a linkage of polynucleotide sequence elements or amino acid sequence elements in a functional relationship. For example, a polynucleotide sequence is operably connected when it is placed into a functional relationship with another polynucleotide sequence. In some embodiments, a transcription regulatory polynucleotide sequence e.g., a promoter, enhancer, or other expression control element is operably-linked to a polynucleotide sequence that encodes a protein if it affects the transcription of the polynucleotide sequence that encodes the protein.

The terms “subject” and “patient” are used interchangeably herein and include any human or nonhuman animal. The term “nonhuman animal” includes, but is not limited to, vertebrates such as nonhuman primates, sheep, dogs, and rodents such as mice, rats and guinea pigs. In some embodiments, the subject is a human.

As used herein, the term “administering” refers to the physical introduction of a therapeutic agent (or a precursor of the therapeutic agent that is metabolized or altered within the body of the subject to produce the therapeutic agent in vivo) to a subject, using any of the various methods and delivery systems known to those skilled in the art. Exemplary routes of include intravenous, intramuscular, subcutaneous, intraperitoneal, spinal or other parenteral routes of administration, for example by injection or infusion. The term “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent may be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

A “therapeutically effective amount” or “therapeutically effective dose” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “disease,” “disorder,” and “syndrome” are used interchangeably herein.

As used herein, the terms “treat,” treating,” “treatment,” and the like refer to reducing or ameliorating a disease and/or symptom(s) associated therewith or obtaining a desired pharmacologic and/or physiologic effect. It will be appreciated that, although not precluded, treating a disease does not require that the disease or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease. To this end, the presently disclosed methods comprise administering a therapeutically effective amount of a compositions as described herein.

### 5.3 Fusion Proteins

In certain aspects, provided herein are fusion proteins that comprise an effector domain comprising a catalytic domain of a deubiquitinase, or a functional fragment or functional variant thereof; and a targeting domain comprising a moiety that specifically binds a target cytosolic protein.

### 5.3.1 Effector Domain

In some embodiments, the effector domain comprises a catalytic domain of a deubiquitinase, or a functional fragment or functional variant thereof. In some embodiments, the deubiquitinase is human. In some embodiments, the catalytic domain is derived from a naturally occurring deubiquitinase (e.g., a naturally occurring human deubiquitinase).

In some embodiments, the amino acid sequence of the effector domain comprises the amino acid sequence of a full length deubiquitinase. In some embodiments, the amino acid sequence of the effector domain comprises the amino acid sequence of a catalytic domain of a deubiquitinase and an additional amino acid sequence at the N-terminal, C-terminal, or N-terminal and C-terminal end of the catalytic domain.

In some embodiments, the catalytic domain comprises a naturally occurring amino acid sequence of a deubiquitinase. In some embodiments, the catalytic domain comprises a variant of a naturally occurring deubiquitinase. In some embodiments, the amino acid sequence of the catalytic domain of the fusion protein is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of a naturally occurring deubiquitinase. In some embodiments, the amino acid sequence of the catalytic domain of the fusion protein comprises 1, 2, 3, 4, 5, 6, 7, 8, 9, 10, 15, or 20 amino acid modifications compared to the amino acid sequence of the catalytic domain of a naturally occurring deubiquitinase.

In some embodiments, the catalytic domain comprises the minimum amino acid sequence of a naturally occurring deubiquitinase sufficient to mediate deubiquitination of a target protein. In some embodiments, the catalytic domain comprises more than the minimum amino acid sequence of a naturally occurring deubiquitinase sufficient to mediate deubiquitination of a target protein.

In some embodiments, the deubiquitinase is a cysteine protease or a metalloprotease. In some embodiments, the deubiquitinase is a cysteine protease. In some embodiments, the deubiquitinase is a metalloprotease. In some embodiments, the deubiquitinase is a ubiquitin-specific protease (USP), a ubiquitin C-terminal hydrolase (UCH), a Machado-Josephin domain protease (MJD), an ovarian tumor protease (OTU), a MINDY protease, or a ZUFSP protease.

Exemplary deubiquitinases include, but are not limited to, USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, BAP1, UCHL1, UCHL3, UCHL5, ATXN3, ATXN3L, OTUB1, OTUB2, MINDY1, MINDY2, MINDY3, MINDY4, and ZUP1. Exemplary deubiquitinases for use in the present disclosure are also disclosed in Komander, D. et al. Breaking the chains: structure and function of the deubiquitinases. Nat Rev Mol Cell Biol 10, 550-563 (2009), the entire contents of which is incorporated by reference herein.

In some embodiments, the deubiquitinase is selected from the group consisting of USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, and USP46.

In some embodiments, the deubiquitinase is BAP1, UCHL1, UCHL3, or UCHL5. In some embodiments, the deubiquitinase is ATXN3 or ATXN3L. In some embodiments, the deubiquitinase is OTUB1 or OTUB2. In some embodiments, the deubiquitinase is MINDY1, MINDY2, MINDY3, or MINDY4. In some embodiments, the deubiquitinase is ZUP1. In some embodiments, the deubiquitinase is a Jab1/Mov34/Mpr1 Pad1 N-terminal+(MPN+) (JAMM) domain protease.

In some embodiments, the deubiquitinase is a deubiquitinase described in Table 1. In some embodiments, the amino acid sequence of the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of a deubiquitinase in Table 1. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a catalytic domain of a deubiquitinase in Table 1. In some embodiments, the effector domain comprises a functional fragment of a deubiquitinase in Table 1. In some embodiments, the effector domain deubiquitinase comprises a functional variant of deubiquitinase in Table 1. In some embodiments, the catalytic domain comprises a functional fragment of a catalytic domain of a deubiquitinase in Table 1. In some embodiments, the catalytic domain comprises a functional variant of a catalytic domain of a deubiquitinase in Table 1.

In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical any one of SEQ ID NOS: 1-112. In some embodiments, the deubiquitinase consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical any one of SEQ ID NOS: 1-112.

In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 2. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 4. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 5. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 6. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 7. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 8. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 9. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 10. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 11. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 12. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 13. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 14. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 15. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 16. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 17. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 18. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 22. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 23. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 24. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 25. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 26. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 27. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 28. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 30. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 31. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 32. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 33. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 34. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 35. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 36. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 37. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 38. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 39. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 40. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 42. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 43. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 44. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 45. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 46. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 47. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 48. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 49. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 50. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 51. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 52. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 53. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 54. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 55. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 56. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 57. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 58. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 59. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 60. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 61. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 62. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 63. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 64. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 65. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 66. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 67. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 68. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 69. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 70. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 71. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 72. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 73. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 74. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 75. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 76. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 77. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 78. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 79. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 80. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 81. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 82. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 83. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 84. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 85. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 86. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 87. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 88. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 89. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 90. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 91. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 92. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 93. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 94. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 95. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 96. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 97. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 98. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 99. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 100. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 101. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 102. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 103. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 104. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 105. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 106. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 107. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 108. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 109. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 110. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 111. In some embodiments, the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 112.

In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of any one of SEQ ID NOS: 1-112. In some embodiments, the amino acid sequence of the effector domain consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of any one of SEQ ID NOS: 1-112.

In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 1. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 2. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 3. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 4. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 5. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 6. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 7. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 8. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 9. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 10. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 11. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 12. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 13. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 14. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 15. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 16. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 17. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 18. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 19. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 20. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 21. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 22. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 23. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 24. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 25. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 26. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 27. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 28. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 29. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 30. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 31. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 32. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 33. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 34. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 35. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 36. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 37. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 38. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 39. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 40. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 41. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 42. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 43. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 44. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 45. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 46. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 47. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 48. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 49. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 50. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 51. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 52. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 53. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 54. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 55. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 56. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 57. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 58. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 59. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 60. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 61. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 62. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 63. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 64. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 65. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 66. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 67. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 68. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 69. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 70. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 71. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 72. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 73. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 74. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 75. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 76. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 77. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 78. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 79. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 80. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 81. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 82. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 83. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 84. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 85. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 86. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 87. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 88. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 89. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 90. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 91. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 92. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 93. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 94. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 95. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 96. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 97. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 98. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 99. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 100. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 101. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 102. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 103. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 104. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 105. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 106. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 107. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 108. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 109. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 110. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 111. In some embodiments, the amino acid sequence of the effector domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the catalytic domain of SEQ ID NO: 112.

In some embodiments, the catalytic domain is derived from a deubiquitinase that comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-112. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-112.

In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 1. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 2. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 3. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 4. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 5. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 6. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 7. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 8. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 9. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 10. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 11. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 12. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 13. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 14. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 15. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 16. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 17. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 18. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 19. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 20. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 21. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 22. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 23. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 24. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 25. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 26. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 27. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 28. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 29. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 30. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 31. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 32. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 33. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 34. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 35. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 36. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 37. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 38. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 39. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 40. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 41. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 42. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 43. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 44. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 45. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 46. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 47. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 48. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 49. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 50. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 51. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 52. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 53. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 54. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 55. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 56. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 57. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 58. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 59. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 60. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 61. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 62. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 63. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 64. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 65. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 66. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 67. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 68. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 69. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 70. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 71. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 72. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 73. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 74. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 75. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 76. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 77. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 78. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 79. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 80. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 81. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 82. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 83. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 84. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 85. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 86. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 87. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 88. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 89. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 90. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 91. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 92. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 93. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 94. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 95. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 96. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 97. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 98. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 99. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 100. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 101. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 102. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 102. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 104. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 105. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 106. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 107. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 108. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 109. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 110. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 111. In some embodiments, the catalytic domain is derived from a deubiquitinase that consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 112.

In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 113-220 or 293. In some embodiments, the catalytic domain consists of an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 113-220.

In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 113. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 114. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 115. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 116. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 117. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 118. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 119. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 120. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 121. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 122. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 123. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 124. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 125. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 126. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 127. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 128. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 129. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 130. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 131. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 132. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 133. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 134. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 135. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 136. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 137. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 138. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 139. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 140. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 141. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 142. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 143. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 144. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 145. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 146. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 147. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 148. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 149. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 150. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 151. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 152. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 153. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 154. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 155. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 156. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 157. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 158. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 159. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 160. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 161. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 162. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 163. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 164. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 165. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 166. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 167. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 168. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 169. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 170. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 171. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 172. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 173. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 174. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 175. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 176. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 177. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 178. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 179. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 180. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 181. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 182. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 183. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 184. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 185. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 186. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 187. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 188. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 189. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 190. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 191. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 192. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 193. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 194. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 195. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 196. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 197. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 198. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 199. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 200. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 201. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 202. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 203. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 204. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 205. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 206. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 207. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 208. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 209. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 210. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 211. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 212. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 213. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 214. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 215. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 216. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 217. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 218. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 219. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 220. In some embodiments, the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 293.

Table 1 below describes, the amino acid sequence of exemplary human deubiquitinases and exemplary catalytic domains of the exemplary human deubiquitinases. The catalytic domains are exemplary. A person of ordinary skill in the art could readily determine a sufficient amino acid sequence of a human deubiquitinase to mediate deubiquitination (e.g., a catalytic domain). Any of the human deubiquitinases (functional fragment or variants thereof) may be used to derive a catalytic domain for use in a fusion protein described herein.

TABLE 1
The amino acid sequence of exemplary human deubiquitinases
and exemplary catalytic domains of the same

| | SEQ | | SEQ | Exemplary Catalytic Domains |
|---|---|---|---|---|
| Description | ID NO | Amino Acid Sequence | ID NO | (Amino Acid Sequence) |
| UBP27_HUMAN | 1 | MCKDYVYDKDIEQIAKEEQGEA | 113 | SSFTIGLRGLINLGNTCEMN |
| Ubiquitin | | LKLQASTSTEVSHQQCSVPGLG | | CIVQALTHTPILRDFFLSDR |
| carboxyl- | | EKFPTWETTKPELELLGHNPRR | | HRCEMPSPELCLVCEMSSLE |
| terminal | | RRITSSFTIGLRGLINLGNTCF | | RELYSGNPSPHVPYKLLHLV |
| hydrolase 27 | | MNCIVQALTHTPILRDFFLSDR | | WIHARHLAGYRQQDAHEFLI |
| | | HRCEMPSPELCLVCEMSSLFRE | | AALDVLHRHCKGDDVGKAAN |
| | | LYSGNPSPHVPYKLLHLVWIHA | | NPNHCNCIIDQIFTGGLQSD |
| | | RHLAGYRQQDAHEFLIAALDVL | | VTCQACHGVSTTIDPCWDIS |
| | | HRHCKGDDVGKAANNPNHCNCI | | LDLPGSCTSFWPMSPGRESS |
| | | IDQIFTGGLQSDVTCQACHGVS | | VNGESHIPGITTLTDCLRRE |
| | | TTIDPCWDISLDLPGSCTSFWP | | TRPEHLGSSAKIKCGSCQSY |
| | | MSPGRESSVNGESHIPGITTLT | | QESTKQLTMNKLPVVACFHF |
| | | DCLRRFTRPEHLGSSAKIKCGS | | KRFEHSAKQRRKITTYISEP |
| | | CQSYQESTKQLTMNKLPVVACF | | LELDMTPFMASSKESRMNGQ |
| | | HFKRFEHSAKQRRKITTYISEP | | LQLPTNSGNNENKYSLFAVV |
| | | LELDMTPEMASSKESRMNGQLQ | | NHQGTLESGHYTSFIRHHKD |
| | | LPTNSGNNENKYSLFAVVNHQG | | QWFKCDDAVITKASIKDVLD |
| | | TLESGHYTSFIRHHKDQWEKCD | | SEGYLLFYHKQVLEHESEKV |
| | | DAVITKASIKDVLDSEGYLLFY | | KEMNTQAY |
| | | HKQVLEHESEKVKEMNTQAY | | |
| UBP48_HUMAN | 2 | MAPRLQLEKAAWRWAETVRPEE | 114 | NSFHNIDDPNCERRKKNSFV |
| Ubiquitin | | VSQEHIETAYRIWLEPCIRGVC | | GLTNLGATCYVNTFLQVWEL |
| carboxyl- | | RRNCKGNPNCLVGIGEHIWLGE | | NLELRQALYLCPSTCSDYML |
| terminal | | IDENSFHNIDDPNCERRKKNSF | | GDGIQEEKDYEPQTICEHLQ |
| hydrolase 48 | | VGLTNLGATCYVNTFLQVWFLN | | YLFALLQNSNRRYIDPSGFV |
| | | LELRQALYLCPSTCSDYMLGDG | | KALGLDTGQQQDAQEFSKLE |
| | | IQEEKDYEPQTICEHLQYLFAL | | MSLLEDTLSKQKNPDVRNIV |
| | | LQNSNRRYIDPSGFVKALGLDT | | QQQFCGEYAYVTVCNQCGRE |
| | | GQQQDAQEFSKLFMSLLEDTLS | | SKLLSKFYELELNIQGHKQL |
| | | KQKNPDVRNIVQQQFCGEYAYV | | TDCISEFLKEEKLEGDNRYF |
| | | TVCNQCGRESKLLSKFYELELN | | CENCQSKQNATRKIRLLSLP |
| | | IQGHKQLTDCISEFLKEEKLEG | | CTLNLQLMRFVEDRQTGHKK |
| | | DNRYFCENCQSKQNATRKIRLL | | KLNTYIGFSEILDMEPYVEH |
| | | SLPCTLNLQLMRFVEDRQTGHK | | KGGSYVYELSAVLIHRGVSA |
| | | KKLNTYIGFSEILDMEPYVEHK | | YSGHYIAHVKDPQSGEWYKE |
| | | GGSYVYELSAVLIHRGVSAYSG | | NDEDIEKMEGKKLQLGIEED |
| | | HYIAHVKDPQSGEWYKENDEDI | | LAEPSKSQTRKPKCGKGTHC |
| | | EKMEGKKLQLGIEEDLAEPSKS | | SRNAYMLVYRLQT |
| | | QTRKPKCGKGTHCSRNAYMLVY | | |
| | | RLQTQEKPNTTVQVPAFLQELV | | |
| | | DRDNSKFEEWCIEMAEMRKQSV | | |
| | | DKGKAKHEEVKELYQRLPAGAE | | |
| | | PYEFVSLEWLQKWLDESTPTKP | | |
| | | IDNHACLCSHDKLHPDKISIMK | | |
| | | RISEYAADIFYSRYGGGPRLTV | | |
| | | KALCKECVVERCRILRLKNQLN | | |
| | | EDYKTVNNLLKAAVKGSDGFWV | | |
| | | GKSSLRSWRQLALEQLDEQDGD | | |
| | | AEQSNGKMNGSTLNKDESKEER | | |
| | | KEEEELNENEDILCPHGELCIS | | |
| | | ENERRLVSKEAWSKLQQYFPKA | | |
| | | PEFPSYKECCSQCKILEREGEE | | |
| | | NEALHKMIANEQKTSLPNLFQD | | |
| | | KNRPCLSNWPEDTDVLYIVSQF | | |
| | | FVEEWRKFVRKPTRCSPVSSVG | | |
| | | NSALLCPHGGLMFTFASMTKED | | |
| | | SKLIALIWPSEWQMIQKLFVVD | | |
| | | HVIKITRIEVGDVNPSETQYIS | | |
| | | EPKLCPECREGLLCQQQRDLRE | | |
| | | YTQATIYVHKVVDNKKVMKDSA | | |
| | | PELNVSSSETEEDKEEAKPDGE | | |
| | | KDPDFNQSNGGTKRQKISHQNY | | |
| | | IAYQKQVIRRSMRHRKVRGEKA | | |
| | | LLVSANQTLKELKIQIMHAFSV | | |
| | | APFDQNLSIDGKILSDDCATLG | | |
| | | TLGVIPESVILLKADEPIADYA | | |
| | | AMDDVMQVCMPEEGFKGTGLLG | | |
| | | H | | |
| UBP3_HUMAN | 3 | MECPHLSSSVCIAPDSAKEPNG | 115 | TAICATGLRNLGNTCEMNAI |
| Ubiquitin | | SPSSWCCSVCRSNKSPWVCLTC | | LQSLSNIEQFCCYFKELPAV |
| carboxyl- | | SSVHCGRYVNGHAKKHYEDAQV | | ELRNGKTAGRRTYHTRSQGD |
| terminal | | PLTNHKKSEKQDKVQHTVCMDC | | NNVSLVEEFRKTLCALWQGS |
| hydrolase 3 | | SSYSTYCYRCDDFVVNDTKLGL | | QTAFSPESLFYVVWKIMPNF |
| | | VQKVREHLQNLENSAFTADRHK | | RGYQQQDAHEFMRYLLDHLH |
| | | KRKLLENSTLNSKLLKVNGSTT | | LELQGGENGVSRSAILQENS |
| | | AICATGLRNLGNTCEMNAILQS | | TLSASNKCCINGASTVVTAI |
| | | LSNIEQFCCYFKELPAVELRNG | | FGGILQNEVNCLICGTESRK |
| | | KTAGRRTYHTRSQGDNNVSLVE | | FDPELDLSLDIPSQFRSKRS |
| | | EFRKTLCALWQGSQTAFSPESL | | KNQENGPVCSLRDCLRSFTD |
| | | FYVVWKIMPNERGYQQQDAHEF | | LEELDETELYMCHKCKKKQK |
| | | MRYLLDHLHLELQGGENGVSRS | | STKKFWIQKLPKVLCLHLKR |
| | | AILQENSTLSASNKCCINGAST | | FHWTAYLRNKVDTYVEFPLR |
| | | VVTAIFGGILQNEVNCLICGTE | | GLDMKCYLLEPENSGPESCL |
| | | SRKFDPFLDLSLDIPSQFRSKR | | YDLAAVVVHHGSGVGSGHYT |
| | | SKNQENGPVCSLRDCLRSFTDL | | AYATHEGRWFHENDSTVTLT |
| | | EELDETELYMCHKCKKKQKSTK | | DEETVVKAKAYILFYVEHQ |
| | | KFWIQKLPKVLCLHLKRFHWTA | | |
| | | YLRNKVDTYVEFPLRGLDMKCY | | |
| | | LLEPENSGPESCLYDLAAVVVH | | |
| | | HGSGVGSGHYTAYATHEGRWFH | | |
| | | FNDSTVTLTDEETVVKAKAYIL | | |
| | | FYVEHQAKAGSDKL | | |
| U17LB_HUMAN | 4 | QLAPREKLPLSSRRPAAVGAGL | 116 | AVGAGLQNMGNTCYVNASLQ |
| Ubiquitin | | QNMGNTCYVNASLQCLTYTPPL | | CLTYTPPLANYMLSREHSQT |
| carboxyl- | | ANYMLSREHSQTCHRHKGCMLC | | CHRHKGCMLCTMQAHITRAL |
| terminal | | TMQAHITRALHNPGHVIQPSQA | | HNPGHVIQPSQALAAGFHRG |
| hydrolase 17- | | LAAGFHRGKQEDAHEFLMFTVD | | KQEDAHEFLMFTVDAMKKAC |
| like protein 11 | | AMKKACLPGHKQVDHHSKDTTL | | LPGHKQVDHHSKDTTLIHQI |
| | | IHQIFGGYWRSQIKCLHCHGIS | | FGGYWRSQIKCLHCHGISDT |
| | | DTFDPYLDIALDIQAAQSVQQA | | FDPYLDIALDIQAAQSVQQA |
| | | LEQLVKPEELNGENAYHCGVCL | | LEQLVKPEELNGENAYHCGV |
| | | QRAPASKTLTLHTSAKVLILVL | | CLQRAPASKTLTLHTSAKVL |
| | | KRFSDVTGNKIAKNVQYPECLD | | ILVLKRFSDVTGNKIAKNVQ |
| | | MQPYMSQTNTGPLVYVLYAVLV | | YPECLDMQPYMSQTNTGPLV |
| | | HAGWSCHNGHYFSYVKAQEGQW | | YVLYAVLVHAGWSCHNGHYF |
| | | YKMDDAEVTASSITSVLSQQAY | | SYVKAQEGQWYKMDDAEVTA |
| | | VLFYIQKSEWERHSESVSRGRE | | SSITSVLSQQAYVLFYIQKS |
| | | PRALGAEDTDRRATQGELKRDH | | |
| | | PCLQAPELDEHLVERATQESTL | | |
| | | DHWKFLQEQNKTKPEENVRKVE | | |
| | | GTLPPDVLVIHQSKYKCGMKNH | | |
| | | HPEQQSSLLNLSSTTPTHQESM | | |
| | | NTGTLASLRGRARRSKGKNKHS | | |
| | | KRALLVCQ | | |
| UBP1_HUMAN | 5 | MPGVIPSESNGLSRGSPSKKNR | 117 | LPFVGLNNLGNTCYLNSILQ |
| Ubiquitin | | LSLKFFQKKETKRALDETDSQE | | VLYFCPGFKSGVKHLENIIS |
| carboxyl- | | NEEKASEYRASEIDQVVPAAQS | | RKKEALKDEANQKDKGNCKE |
| terminal | | SPINCEKRENLLPFVGLNNLGN | | DSLASYELICSLQSLIISVE |
| hydrolase 1 | | TCYLNSILQVLYFCPGFKSGVK | | QLQASFLLNPEKYTDELATQ |
| | | HLENIISRKKEALKDEANQKDK | | PRRLLNTLRELNPMYEGYLQ |
| | | GNCKEDSLASYELICSLQSLII | | HDAQEVLQCILGNIQETCQL |
| | | SVEQLQASFLLNPEKYTDELAT | | LKKEEVKNVAELPTKVEEIP |
| | | QPRRLLNTLRELNPMYEGYLQH | | HPKEEMNGINSIEMDSMRHS |
| | | DAQEVLQCILGNIQETCQLLKK | | EDFKEKLPKGNGKRKSDTEF |
| | | EEVKNVAELPTKVEEIPHPKEE | | GNMKKKVKLSKEHQSLEENQ |
| | | MNGINSIEMDSMRHSEDEKEKL | | RQTRSKRKATSDTLESPPKI |
| | | PKGNGKRKSDTEFGNMKKKVKL | | IPKYISENESPRPSQKKSRV |
| | | SKEHQSLEENQRQTRSKRKATS | | KINWLKSATKQPSILSKFCS |
| | | DTLESPPKIIPKYISENESPRP | | LGKITTNQGVKGQSKENECD |
| | | SQKKSRVKINWLKSATKQPSIL | | PEEDLGKCESDNTTNGCGLE |
| | | SKFCSLGKITTNQGVKGQSKEN | | SPGNTVTPVNVNEVKPINKG |
| | | ECDPEEDLGKCESDNTTNGCGL | | EEQIGFELVEKLFQGQLVLR |
| | | ESPGNTVTPVNVNEVKPINKGE | | TRCLECESLTERREDFQDIS |
| | | EQIGFELVEKLFQGQLVLRTRC | | VPVQEDELSKVEESSEISPE |
| | | LECESLTERREDFQDISVPVQE | | PKTEMKTLRWAISQFASVER |
| | | DELSKVEESSEISPEPKTEMKT | | IVGEDKYFCENCHHYTEAER |
| | | LRWAISQFASVERIVGEDKYFC | | SLLEDKMPEVITIHLKCFAA |
| | | ENCHHYTEAERSLLEDKMPEVI | | SGLEFDCYGGGLSKINTPLL |
| | | TIHLKCFAASGLEFDCYGGGLS | | TPLKLSLEEWSTKPTNDSYG |
| | | KINTPLLTPLKLSLEEWSTKPT | | LFAVVMHSGITISSGHYTAS |
| | | NDSYGLFAVVMHSGITISSGHY | | VKVTDLNSLELDKGNFVVDQ |
| | | TASVKVTDLNSLELDKGNFVVD | | MCEIGKPEPLNEEEARGVVE |
| | | QMCEIGKPEPLNEEEARGVVEN | | NYNDEEVSIRVGGNTQPSKV |
| | | YNDEEVSIRVGGNTQPSKVLNK | | LNKKNVEAIGLLGGQKSKAD |
| | | KNVEAIGLLGGQKSKADYELYN | | YELYNKASNPDKVASTAFAE |
| | | KASNPDKVASTAFAENRNSETS | | NRNSETSDTTGTHESDRNKE |
| | | DTTGTHESDRNKESSDQTGINI | | SSDQTGINISGFENKISYVV |
| | | SGFENKISYVVQSLKEYEGKWL | | QSLKEYEGKWLLFDDSEVKV |
| | | LEDDSEVKVTEEKDELNSLSPS | | TEEKDELNSLSPSTSPTSTP |
| | | TSPTSTPYLLFYKKL | | YLLFYKKL |
| UBP40_HUMAN | 6 | MFGDLFEEEYSTVSNNQYGKGK | 118 | FTNLSGIRNQGGTCYLNSLL |
| Ubiquitin | | KLKTKALEPPAPREFTNLSGIR | | QTLHFTPEFREALFSLGPEE |
| carboxyl- | | NQGGTCYLNSLLQTLHFTPEER | | LGLFEDKDKPDAKVRIIPLQ |
| terminal | | EALFSLGPEELGLFEDKDKPDA | | LQRLFAQLLLLDQEAASTAD |
| hydrolase 40 | | KVRIIPLQLQRLFAQLLLLDQE | | LTDSFGWTSNEEMRQHDVQE |
| | | AASTADLTDSFGWTSNEEMRQH | | LNRILFSALETSLVGTSGHD |
| | | DVQELNRILFSALETSLVGTSG | | LIYRLYHGTIVNQIVCKECK |
| | | HDLIYRLYHGTIVNQIVCKECK | | NVSERQEDFLDLTVAVKNVS |
| | | NVSERQEDFLDLTVAVKNVSGL | | GLEDALWNMYVEEEVEDCDN |
| | | EDALWNMYVEEEVEDCDNLYHC | | LYHCGTCDRLVKAAKSAKLR |
| | | GTCDRLVKAAKSAKLRKLPPEL | | KLPPELTVSLLRENFDFVKC |
| | | TVSLLRENFDFVKCERYKETSC | | ERYKETSCYTFPLRINLKPF |
| | | YTFPLRINLKPFCEQSELDDLE | | CEQSELDDLEYIYDLESVII |
| | | YIYDLFSVIIHKGGCYGGHYHV | | HKGG |
| | | YIKDVDHLGNWQFQEEKSKPDV | | CYGGHYHVYIKDVDHLGNWQ |
| | | NLKDLQSEEEIDHPLMILKAIL | | FQEEKSKPDVNLKDLQSEEE |
| | | LEENNLIPVDQLGQKLLKKIGI | | IDHPLMILKAILLEENNLIP |
| | | SWNKKYRKQHGPLRKFLQLHSQ | | VDQLGQKLLKKIGISWNKKY |
| | | IFLLSSDESTVRLLKNSSLQAE | | RKQHGPLRKFLQLHSQIFLL |
| | | SDFQRNDQQIFKMLPPESPGLN | | SSDESTVRLLKNSSLQAESD |
| | | NSISCPHWFDINDSKVQPIREK | | FQRNDQQIFKMLPPESPGLN |
| | | DIEQQFQGKESAYMLFYRKSQL | | NSISCPHWFDINDSKVQPIR |
| | | QRPPEARANPRYGVPCHLLNEM | | EKDIEQQFQGKESAYMLFYR |
| | | DAANIELQTKRAECDSANNTFE | | KSQLQRPPEARANPRYGVPC |
| | | LHLHLGPQYHFENGALHPVVSQ | | HLLNEMDAANIELQTKRAEC |
| | | TESVWDLTEDKRKTLGDLRQSI | | DSANNTFELHLHLGPQYHFF |
| | | FQLLEFWEGDMVLSVAKLVPAG | | NGALHPVVSQTESVWDLTED |
| | | LHIYQSLGGDELTLCETEIADG | | KRKTLGDLRQSIFQLLEFWE |
| | | EDIFVWNGVEVGGVHIQTGIDC | | GDMVLSVAKLVPAGLHIYQS |
| | | EPLLLNVLHLDTSSDGEKCCQV | | LGGDELTLCETEIADGEDIF |
| | | IESPHVFPANAEVGTVLTALAI | | VWNGVEVGGVHIQTGIDCEP |
| | | PAGVIFINSAGCPGGEGWTAIP | | LLLNVLHLDTSSDGEKCCQV |
| | | KEDMRKTFREQGLRNGSSILIQ | | IESPHVFPANAEVGTVLTAL |
| | | DSHDDNSLLTKEEKWVTSMNEI | | AIPAGVIFINSAGCPGGEGW |
| | | DWLHVKNLCQLESEEKQVKISA | | TAIPKEDMRKTFREQGLRNG |
| | | TVNTMVEDIRIKAIKELKLMKE | | SSILIQDSHDDNSLLTKEEK |
| | | LADNSCLRPIDRNGKLLCPVPD | | WVTSMNEIDWLHVKNLCQLE |
| | | SYTLKEAELKMGSSLGLCLGKA | | SEEKQVKISATVNTMVEDIR |
| | | PSSSQLFLFFAMGSDVQPGTEM | | IKAIKELKLMKELADNSCLR |
| | | EIVVEETISVRDCLKLMLKKSG | | PIDRNGKLLCPVPDSYTLKE |
| | | LQGDAWHLRKMDWCYEAGEPLC | | AELKMGSSLGLCLGKAPSSS |
| | | EEDATLKELLICSGDTLLLIEG | | QLFLFFAMGSDVQPGTEMEI |
| | | QLPPLGFLKVPIWWYQLQGPSG | | VVEETISVRDCLKLMLKKSG |
| | | HWESHQDQTNCTSSWGRVWRAT | | LQGDAWHLRKMDWCYEAGEP |
| | | SSQGASGNEPAQVSLLYLGDIE | | LCEEDATLKELLICSGDTLL |
| | | ISEDATLAELKSQAMTLPPFLE | | LIEGQLPPLGELKVPIWWYQ |
| | | FGVPSPAHLRAWTVERKRPGRL | | LQGPSGHWESHQDQTNCTSS |
| | | LRTDRQPLREYKLGRRIEICLE | | WGRVWRATSSQGASGNEPAQ |
| | | PLQKGENLGPQDVLLRTQVRIP | | VSLLYLGDIEISEDATLAEL |
| | | GERTYAPALDLVWNAAQGGTAG | | KSQAMTLPPFLEFGVPSPAH |
| | | SLRQRVADFYRLPVEKIEIAKY | | LRAWTVERKRPGRLLRTDRQ |
| | | FPEKFEWLPISSWNQQITKRKK | | PLREYKLGRRIEICLEPLQK |
| | | KKKQDYLQGAPYYLKDGDTIGV | | GENLGPQDVLLRTQVRIPGE |
| | | KNLLIDDDDDESTIRDDTGKEK | | RTYAPALDLVWNAAQGGTAG |
| | | QKQRALGRRKSQEALHEQSSYI | | SLRQRVADFYRLPVEKIEIA |
| | | LSSAETPARPRAPETSLSIHVG | | KYFPEKFEWLPISSWNQQIT |
| | | SFR | | KRKKKKKQDYLQGAPYYLKD |
| | | | | GDTIGVKNLLIDDDDDESTI |
| | | | | RDDTGKEKQKQRALGRRKSQ |
| UBP7_HUMAN | 7 | MNHQQQQQQQKAGEQQLSEPED | 119 | TGYVGLKNQGATCYMNSLLQ |
| Ubiquitin | | MEMEAGDTDDPPRITQNPVING | | TLFFTNQLRKAVYMMPTEGD |
| carboxyl- | | NVALSDGHNTAEEDMEDDTSWR | | DSSKSVPLALQRVFYELQHS |
| terminal | | SEATFQFTVERFSRLSESVLSP | | DKPVGTKKLTKSFGWETLDS |
| hydrolase 7 | | PCFVRNLPWKIMVMPRFYPDRP | | FMQHDVQELCRVLLDNVENK |
| | | HQKSVGFFLQCNAESDSTSWSC | | MKGTCVEGTIPKLFRGKMVS |
| | | HAQAVLKIINYRDDEKSFSRRI | | YIQCKEVDYRSDRREDYYDI |
| | | SHLFFHKENDWGESNEMAWSEV | | QLSIKGKKNIFESFVDYVAV |
| | | TDPEKGFIDDDKVTFEVFVQAD | | EQLDGDNKYDAGEHGLQEAE |
| | | APHGVAWDSKKHTGYVGLKNQG | | KGVKFLTLPPVLHLQLMREM |
| | | ATCYMNSLLQTLFFTNQLRKAV | | YDPQTDQNIKINDRFEFPEQ |
| | | YMMPTEGDDSSKSVPLALQRVE | | LPLDEFLQKTDPKDPANYIL |
| | | YELQHSDKPVGTKKLTKSFGWE | | HAVLVHSGDNHGGHYVVYLN |
| | | TLDSFMQHDVQELCRVLLDNVE | | PKGDGKWCKFDDDVVSRCTK |
| | | NKMKGTCVEGTIPKLERGKMVS | | EEAIEHNYGGHDDDLSVRHC |
| | | YIQCKEVDYRSDRREDYYDIQL | | TNAYMLVYIRE |
| | | SIKGKKNIFESFVDYVAVEQLD | | |
| | | GDNKYDAGEHGLQEAEKGVKFL | | |
| | | TLPPVLHLQLMREMYDPQTDQN | | |
| | | IKINDRFEFPEQLPLDEFLQKT | | |
| | | DPKDPANYILHAVLVHSGDNHG | | |
| | | GHYVVYLNPKGDGKWCKEDDDV | | |
| | | VSRCTKEEAIEHNYGGHDDDLS | | |
| | | VRHCTNAYMLVYIRESKLSEVL | | |
| | | QAVTDHDIPQQLVERLQEEKRI | | |
| | | EAQKRKERQEAHLYMQVQIVAE | | |
| | | DQFCGHQGNDMYDEEKVKYTVE | | |
| | | KVLKNSSLAEFVQSLSQTMGFP | | |
| | | QDQIRLWPMQARSNGTKRPAML | | |
| | | DNEADGNKTMIELSDNENPWTI | | |
| | | FLETVDPELAASGATLPKEDKD | | |
| | | HDVMLFLKMYDPKTRSLNYCGH | | |
| | | IYTPISCKIRDLLPVMCDRAGE | | |
| | | IQDTSLILYEEVKPNLTERIQD | | |
| | | YDVSLDKALDELMDGDIIVFQK | | |
| | | DDPENDNSELPTAKEYERDLYH | | |
| | | RVDVIFCDKTIPNDPGFVVTLS | | |
| | | NRMNYFQVAKTVAQRLNTDPML | | |
| | | LQFFKSQGYRDGPGNPLRHNYE | | |
| | | GTLRDLLQFFKPRQPKKLYYQQ | | |
| | | LKMKITDFENRRSEKCIWLNSQ | | |
| | | FREEEITLYPDKHGCVRDLLEE | | |
| | | CKKAVELGEKASGKLRLLEIVS | | |
| | | YKIIGVHQEDELLECLSPATSR | | |
| | | TFRIEEIPLDQVDIDKENEMLV | | |
| | | TVAHFHKEVEGTFGIPFLLRIH | | |
| | | QGEHFREVMKRIQSLLDIQEKE | | |
| | | FEKFKFAIVMMGRHQYINEDEY | | |
| | | EVNLKDFEPQPGNMSHPRPWLG | | |
| | | LDHENKAPKRSRYTYLEKAIKI | | |
| | | HN | | |
| U17L5_HUMAN | 8 | MEDDSLYLRGEWQFNHESKLTS | 120 | AVGAGLQNMGNTCYVNASLQ |
| Ubiquitin | | SRPDAAFAEIQRTSLPEKSPLS | | CLTYTPPLANYMLSREHSQT |
| carboxyl- | | CETRVDLCDDLAPVARQLAPRE | | CHRHKGCMLCTMQAHITRAL |
| terminal | | KLPLSSRRPAAVGAGLQNMGNT | | HNPGHVIQPSQALAAGFHRG |
| hydrolase 17- | | CYVNASLQCLTYTPPLANYMLS | | KQEDAHEFLMFTVDAMKKAC |
| like protein 5 | | REHSQTCHRHKGCMLCTMQAHI | | LPGHKQVDHHSKDTTLIHQI |
| | | TRALHNPGHVIQPSQALAAGFH | | FGGYWRSQIKCLHCHGISDT |
| | | RGKQEDAHEFLMFTVDAMKKAC | | FDPYLDIALDIQAAQSVQQA |
| | | LPGHKQVDHHSKDTTLIHQIFG | | LEQLAKPEELNGENAYHCGV |
| | | GYWRSQIKCLHCHGISDTEDPY | | CLQRAPASKTLTLHTSAKVL |
| | | LDIALDIQAAQSVQQALEQLAK | | ILVLKRFSDVTGNKIAKNVQ |
| | | PEELNGENAYHCGVCLQRAPAS | | YPECLDMQPYMSQPNTGPLV |
| | | KTLTLHTSAKVLILVLKRESDV | | YVLYAVLVHAGWSCHNGHYF |
| | | TGNKIAKNVQYPECLDMQPYMS | | SYVKAQEGQWYKMDDAEVTA |
| | | QPNTGPLVYVLYAVLVHAGWSC | | SSITSVLSQQAYVLFYIQKS |
| | | HNGHYFSYVKAQEGQWYKMDDA | | EWERHSESVSRGREPRALGA |
| | | EVTASSITSVLSQQAYVLFYIQ | | EDTDRRATQGELKRDHPCLQ |
| | | KSEWERHSESVSRGREPRALGA | | APEL |
| | | EDTDRRATQGELKRDHPCLQAP | | |
| | | ELDEHLVERATQESTLDHWKEL | | |
| | | QEQNKTKPEFNVRKVEGTLPPD | | |
| | | VLVIHQSKYKCGMKNHHPEQQS | | |
| | | SLLNLSSSTPTHQESMNTGTLA | | |
| | | SLRGRARRSKGKNKHSKRALLV | | |
| | | CQ | | |
| U17LL_HUMAN | 9 | MEEDSLYLGGEWQFNHESKLTS | 121 | AVGAGLQNMGNTCYVNASLQ |
| Ubiquitin | | SRPDAAFAEIQRTSLPEKSPLS | | CLTYTPPLANYMLSREHSQT |
| carboxyl- | | CETRVDLCDDLAPVARQLAPRE | | CHRHKGCMLCTMQAHITRAL |
| terminal | | KLPLSNRRPAAVGAGLQNMGNT | | HNPGHVIQPSQALAAGFHRG |
| hydrolase 17- | | CYVNASLQCLTYTPPLANYMLS | | KQEDAHEFLMFTVDAMKKAC |
| like protein 21 | | REHSQTCHRHKGCMLCTMQAHI | | LPGHKQVDHHSKDTTLIHQI |
| | | TRALHNPGHVIQPSQALAAGEH | | FGGYWRSQIKCLHCHGISDT |
| | | RGKQEDAHEFLMFTVDAMKKAC | | FDPYLDIALDIQAAQSVQQA |
| | | LPGHKQVDHHSKDTTLIHQIFG | | LEQLVKPEELNGENAYHCGV |
| | | GYWRSQIKCLHCHGISDTFDPY | | CLQRAPASKMLTLLTSAKVL |
| | | LDIALDIQAAQSVQQALEQLVK | | ILVLKRFSDVTGNKIAKNVQ |
| | | PEELNGENAYHCGVCLQRAPAS | | YPECLDMQPYMSQPNTGPLV |
| | | KMLTLLTSAKVLILVLKRESDV | | YVLYAVLVHAGWSCHNGHYF |
| | | TGNKIAKNVQYPECLDMQPYMS | | SYVKAQEGQWYKMDDAEVTA |
| | | QPNTGPLVYVLYAVLVHAGWSC | | SSITSVLSQQAYVLFYIQKS |
| | | HNGHYFSYVKAQEGQWYKMDDA | | EWERHSESVSRGREPRALGA |
| | | EVTASSITSVLSQQAYVLFYIQ | | EDTDRRATQGELKRDHPCLQ |
| | | KSEWERHSESVSRGREPRALGA | | APEL |
| | | EDTDRRATQGELKRDHPCLQAP | | |
| | | ELDEHLVERATQESTLDHWKEL | | |
| | | QEQNKTKPEFNVRKVEGTLPPD | | |
| | | VLVIHQSKYKCGMKNHHPEQQS | | |
| | | SLLNLSSSTPTHQESMNTGTLA | | |
| | | SLRGRARRSKGKNKHSKRALLV | | |
| | | CQ | | |
| U17LA_HUMAN | 10 | MEDDSLYLGGEWQFNHESKLTS | 122 | AVGAGLQNMGNTCYVNASLQ |
| Ubiquitin | | SRPDAAFAEIQRTSLPEKSPLS | | CLTYKPPLANYMLFREHSQT |
| carboxyl- | | CETRVDLCDDLAPVARQLAPRE | | CHRHKGCMLCTMQAHITRAL |
| terminal | | KPPLSSRRPAAVGAGLQNMGNT | | HIPGHVIQPSQALAAGFHRG |
| hydrolase 17- | | CYVNASLQCLTYKPPLANYMLF | | KQEDAHEFLMFTVDAMRKAC |
| like protein 10 | | REHSQTCHRHKGCMLCTMQAHI | | LPGHKQVDRHSKDTTLIHQI |
| | | TRALHIPGHVIQPSQALAAGFH | | FGGYWRSQIKCLHCHGISDT |
| | | RGKQEDAHEFLMFTVDAMRKAC | | FDPYLDIALDIQAAQSVQQA |
| | | LPGHKQVDRHSKDTTLIHQIFG | | LEQLVKPEELNGENAYHCGV |
| | | GYWRSQIKCLHCHGISDTFDPY | | CLQRAPASKTLTLHNSAKVL |
| | | LDIALDIQAAQSVQQALEQLVK | | ILVLKRFPDVTGNKIAKNVQ |
| | | PEELNGENAYHCGVCLQRAPAS | | YPECLDMQPYMSQQNTGPLV |
| | | KTLTLHNSAKVLILVLKRFPDV | | YVLYAVLVHAGWSCHNGHYS |
| | | TGNKIAKNVQYPECLDMQPYMS | | SYVKAQEGQWYKMDDAEVTA |
| | | QQNTGPLVYVLYAVLVHAGWSC | | SSITSVLSQQAYVLFYIQKS |
| | | HNGHYSSYVKAQEGQWYKMDDA | | EWERHSESVSRGREPRALGV |
| | | EVTASSITSVLSQQAYVLFYIQ | | EDTDRRATQGELKRDHPCLQ |
| | | KSEWERHSESVSRGREPRALGV | | APEL |
| | | EDTDRRATQGELKRDHPCLQAP | | |
| | | ELDEHLVERATQESTLDHWKEL | | |
| | | QEQNKTKPEFNVRRVEGTVPPD | | |
| | | VLVIHQSKYKCRMKNHHPEQQS | | |
| | | SLLNLSSTTPTDQESMNTGTLA | | |
| | | SLRGRTRRSKGKNKHSKRALLV | | |
| | | CQ | | |
| UBP41_HUMAN | 11 | MDGVLFRAHQCQYVHPCVHVYV | 123 | WGLVGLHNIGQTCCLNSLIQ |
| Putative | | TVGLMDPLCERKEKASKQEREN | | VFVMNVDFARILKRITVPRG |
| ubiquitin | | PLAHLAAWGLVGLHNIGQTCCL | | ADEQRRSVPFQMLLLLEKMQ |
| carboxyl- | | NSLIQVFVMNVDFARILKRITV | | DSRQKAVWPLELAYCLQKYN |
| terminal | | PRGADEQRRSVPFQMLLLLEKM | | VPLFVQHDAAQLYLKLWNLI |
| hydrolase 41 | | QDSRQKAVWPLELAYCLQKYNV | | KDQIADVHLVERLQALYMIR |
| | | PLFVQHDAAQLYLKLWNLIKDQ | | MKDSLICLDCAMESSRNSSM |
| | | IADVHLVERLQALYMIRMKDSL | | LTLRLSFFDVDSKPLKTLED |
| | | ICLDCAMESSRNSSMLTLRLSF | | ALHCFFQPRELSSKSKCFCE |
| | | FDVDSKPLKTLEDALHCFFQPR | | NCGKKTRGKQVLKLTHLPQT |
| | | ELSSKSKCFCENCGKKTRGKQV | | LTIHLMRESIRNSQTRKICH |
| | | LKLTHLPQTLTIHLMRESIRNS | | SLYFPQSLDFSQILPMKRES |
| | | QTRKICHSLYFPQSLDESQILP | | CDAEEQSGGQYELFAVIAHV |
| | | MKRESCDAEEQSGGQYELFAVI | | GMADSGHYCVYIRNAVDGKW |
| | | AHVGMADSGHYCVYIRNAVDGK | | FCFNDSNICLVSWEDIQCTY |
| | | WFCENDSNICLVSWEDIQCTYG | | GNPNYHW |
| | | NPNYHW | | |
| UBP38_HUMAN | 12 | MDKILEGLVSSSHPLPLKRVIV | 124 | SETGKTGLINLGNTCYMNSV |
| Ubiquitin | | RKVVESAEHWLDEAQCEAMEDL | | IQALFMATDERRQVLSLNLN |
| carboxyl- | | TTRLILEGQDPFQRQVGHQVLE | | GCNSLMKKLQHLFAFLAHTQ |
| terminal | | AYARYHRPEFESFENKTFVLGL | | REAYAPRIFFEASRPPWFTP |
| hydrolase 38 | | LHQGYHSLDRKDVAILDYIHNG | | RSQQDCSEYLRELLDRLHEE |
| | | LKLIMSCPSVLDLFSLLQVEVL | | EKILKVQASHKPSEILECSE |
| | | RMVCERPEPQLCARLSDLLTDF | | TSLQEVASKAAVLTETPRTS |
| | | VQCIPKGKLSITFCQQLVRTIG | | DGEKTLIEKMFGGKLRTHIR |
| | | HFQCVSTQERELREYVSQVTKV | | CLNCRSTSQKVEAFTDLSLA |
| | | SNLLQNIWKAEPATLLPSLQEV | | FCPSSSLENMSVQDPASSPS |
| | | FASISSTDASFEPSVALASLVQ | | IQDGGLMQASVPGPSEEPVV |
| | | HIPLQMITVLIRSLTTDPNVKD | | YNPTTAAFICDSLVNEKTIG |
| | | ASMTQALCRMIDWLSWPLAQHV | | SPPNEFYCSENTSVPNESNK |
| | | DTWVIALLKGLAAVQKFTILID | | ILVNKDVPQKPGGETTPSVT |
| | | VTLLKIELVENRLWFPLVRPGA | | DLLNYFLAPEILTGDNQYYC |
| | | LAVLSHMLLSFQHSPEAFHLIV | | ENCASLQNAEKTMQITEEPE |
| | | PHVVNLVHSFKNDGLPSSTAFL | | YLILTLLRFSYDQKYHVRRK |
| | | VQLTELIHCMMYHYSGFPDLYE | | ILDNVSLPLVLELPVKRITS |
| | | PILEAIKDEPKPSEEKIKLILN | | FSSLSESWSVDVDFTDLSEN |
| | | QSAWTSQSNSLASCLSRLSGKS | | LAKKLKPSGTDEASCTKLVP |
| | | ETGKTGLINLGNTCYMNSVIQA | | YLLSSVVVHSGISSESGHYY |
| | | LEMATDERRQVLSLNLNGCNSL | | SYARNITSTDSSYQMYHQSE |
| | | MKKLQHLFAFLAHTQREAYAPR | | ALALASSQSHLLGRDSPSAV |
| | | IFFEASRPPWFTPRSQQDCSEY | | FEQDLENKEMSKEWFLENDS |
| | | LRFLLDRLHEEEKILKVQASHK | | RVTFTSFQSVQKITSREPKD |
| | | PSEILECSETSLQEVASKAAVL | | TAYVLLYKKQH |
| | | TETPRTSDGEKTLIEKMEGGKL | | |
| | | RTHIRCLNCRSTSQKVEAFTDL | | |
| | | SLAFCPSSSLENMSVQDPASSP | | |
| | | SIQDGGLMQASVPGPSEEPVVY | | |
| | | NPTTAAFICDSLVNEKTIGSPP | | |
| | | NEFYCSENTSVPNESNKILVNK | | |
| | | DVPQKPGGETTPSVTDLLNYFL | | |
| | | APEILTGDNQYYCENCASLQNA | | |
| | | EKTMQITEEPEYLILTLLRFSY | | |
| | | DQKYHVRRKILDNVSLPLVLEL | | |
| | | PVKRITSFSSLSESWSVDVDFT | | |
| | | DLSENLAKKLKPSGTDEASCTK | | |
| | | LVPYLLSSVVVHSGISSESGHY | | |
| | | YSYARNITSTDSSYQMYHQSEA | | |
| | | LALASSQSHLLGRDSPSAVFEQ | | |
| | | DLENKEMSKEWFLENDSRVTFT | | |
| | | SFQSVQKITSRFPKDTAYVLLY | | |
| | | KKQHSTNGLSGNNPTSGLWING | | |
| | | DPPLQKELMDAITKDNKLYLQE | | |
| | | QELNARARALQAASASCSERPN | | |
| | | GFDDNDPPGSCGPTGGGGGGGF | | |
| | | NTVGRLVF | | |
| UBP43_HUMAN | 13 | MDLGPGDAAGGGPLAPRPRRRR | 125 | RPPGAQGLKNHGNTCEMNAV |
| Ubiquitin | | SLRRLFSRELLALGSRSRPGDS | | VQCLSNTDLLAEFLALGRYR |
| carboxyl- | | PPRPQPGHCDGDGEGGFACAPG | | AAPGRAEVTEQLAALVRALW |
| terminal | | PVPAAPGSPGEERPPGPQPQLQ | | TREYTPQLSAEFKNAVSKYG |
| hydrolase 43 | | LPAGDGARPPGAQGLKNHGNTC | | SQFQGNSQHDALEFLLWLLD |
| | | FMNAVVQCLSNTDLLAEFLALG | | RVHEDLEGSSRGPVSEKLPP |
| | | RYRAAPGRAEVTEQLAALVRAL | | EATKTSENCLSPSAQLPLGQ |
| | | WTREYTPQLSAEFKNAVSKYGS | | SFVQSHFQAQYRSSLTCPHC |
| | | QFQGNSQHDALEFLLWLLDRVH | | LKQSNTFDPFLCVSLPIPLR |
| | | EDLEGSSRGPVSEKLPPEATKT | | QTRFLSVTLVFPSKSQRFLR |
| | | SENCLSPSAQLPLGQSFVQSHE | | VGLAVPILSTVAALRKMVAE |
| | | QAQYRSSLTCPHCLKQSNTEDP | | EGGVPADEVILVELYPSGFQ |
| | | FLCVSLPIPLRQTRFLSVTLVE | | RSFFDEEDLNTIAEGDNVYA |
| | | PSKSQRFLRVGLAVPILSTVAA | | FQVPPSPSQGTLSAHPLGLS |
| | | LRKMVAEEGGVPADEVILVELY | | ASPRLAAREGQRFSLSLHSE |
| | | PSGFQRSFFDEEDLNTIAEGDN | | SKVLILFCNLVGSGQQASRF |
| | | VYAFQVPPSPSQGTLSAHPLGL | | GPPFLIREDRAVSWAQLQQS |
| | | SASPRLAAREGQRFSLSLHSES | | ILSKVRHLMKSEAPVQNLGS |
| | | KVLILFCNLVGSGQQASRFGPP | | LFSIRVVGLSVACSYLSPKD |
| | | FLIREDRAVSWAQLQQSILSKV | | SRPLCHWAVDRVLHLRRPGG |
| | | RHLMKSEAPVQNLGSLESIRVV | | PPHVKLAVEWDSSVKERLFG |
| | | GLSVACSYLSPKDSRPLCHWAV | | SLQEERAQDADSVWQQQQAH |
| | | DRVLHLRRPGGPPHVKLAVEWD | | QQHSCTLDECFQFYTKEEQL |
| | | SSVKERLFGSLQEERAQDADSV | | AQDDAWKCPHCQVLQQGMVK |
| | | WQQQQAHQQHSCTLDECFQFYT | | LSLWTLPDILIIHLKRFCQV |
| | | KEEQLAQDDAWKCPHCQVLQQG | | GERRNKLSTLVKFPLSGLNM |
| | | MVKLSLWTLPDILIIHLKRFCQ | | APHVAQRSTSPEAGLGPWPS |
| | | VGERRNKLSTLVKFPLSGLNMA | | WKQPDCLPTSYPLDFLYDLY |
| | | PHVAQRSTSPEAGLGPWPSWKQ | | AVCNHHGNLQGGHYTAYCRN |
| | | PDCLPTSYPLDFLYDLYAVCNH | | SLDGQWYSYDDSTVEPLRED |
| | | HGNLQGGHYTAYCRNSLDGQWY | | EVNTRGAYILFYQKRN |
| | | SYDDSTVEPLREDEVNTRGAYI | | |
| | | LFYQKRNSIPPWSASSSMRGST | | |
| | | SSSLSDHWLLRLGSHAGSTRGS | | |
| UBP2_HUMAN | | LLSWSSAPCPSLPQVPDSPIFT | | SAQGLAGLRNLGNTCEMNSI |
| Ubiquitin | | NSLCNQEKGGLEPRRLVRGVKG | | LQCLSNTRELRDYCLQRLYM |
| carboxyl- | | RSISMKAPTTSRAKQGPFKTMP | | RDLHHGSNAHTALVEEFAKL |
| | | LRWSFGSKEKPPGASVELVEYL | | IQTIWTSSPNDVVSPSEFKT |
| | | ESRRRPRSTSQSIVSLLTGTAG | | QIQRYAPRFVGYNQQDAQEF |
| | | EDEKSASPRSNVALPANSEDGG | | LRFLLDGLHNEVNRVTLRPK |
| | | RAIERGPAGVPCPSAQPNHCLA | | SNPENLDHLPDDEKGRQMWR |
| | | PGNSDGPNTARKLKENAGQDIK | | KYLEREDSRIGDLFVGQLKS |
| | | LPRKFDLPLTVMPSVEHEKPAR | | SLTCTDCGYCSTVEDPEWDL |
| | | PEGQKAMNWKESFQMGSKSSPP | | |
| | | SPYMGFSGNSKDSRRGTSELDR | | |
| | | PLQGTLTLLRSVERKKENRRNE | | |
| | | RAEVSPQVPPVSLVSGGLSPAM | | |
| | | DGQAPGSPPALRIPEGLARGLG | | |
| | | SRLERDVWSAPSSLRLPRKASR | | |
| | | APRGSALGMSQRTVPGEQASYG | | |
| | | TFQRVKYHTLSLGRKKTLPESS | | |
| | | F | | |
| | | MSQLSSTLKRYTESARYTDAHY | | |
| | | AKSGYGAYTPSSYGANLAASLL | | |
| | | EKEKLGFKPVPTSSFLTRPRTY | | |
| | | GPSSLLDYDRGRPLLRPDITGG | | |
| | | GKRAESQTRGTERPLGSGLSGG | | |
| terminal | 14 | SGFPYGVTNNCLSYLPINAYDQ | 126 | SLPIAKRGYPEVTLMDCMRL |
| hydrolase 2 | | GVTLTQKLDSQSDLARDESSLR | | FTKEDVLDGDEKPTCCRCRG |
| | | TSDSYRIDPRNLGRSPMLARTR | | RKRCIKKFSIQRFPKILVLH |
| | | KELCTLQGLYQTASCPEYLVDY | | LKRFSESRIRTSKLTTFVNF |
| | | LENYGRKGSASQVPSQAPPSRV | | PLRDLDLREFASENTNHAVY |
| | | PEIISPTYRPIGRYTLWETGKG | | NLYAVSNHSGTTMGGHYTAY |
| | | QAPGPSRSSSPGRDGMNSKSAQ | | CRSPGTGEWHTENDSSVTPM |
| | | GLAGLRNLGNTCEMNSILQCLS | | SSSQVRTSDAYLLFYELAS |
| | | NTRELRDYCLQRLYMRDLHHGS | | |
| | | NAHTALVEEFAKLIQTIWTSSP | | |
| | | NDVVSPSEFKTQIQRYAPRFVG | | |
| | | YNQQDAQEFLRFLLDGLHNEVN | | |
| | | RVTLRPKSNPENLDHLPDDEKG | | |
| | | RQMWRKYLEREDSRIGDLFVGQ | | |
| | | LKSSLTCTDCGYCSTVEDPEWD | | |
| | | LSLPIAKRGYPEVTLMDCMRLE | | |
| | | TKEDVLDGDEKPTCCRCRGRKR | | |
| | | CIKKFSIQRFPKILVLHLKRFS | | |
| | | ESRIRTSKLTTFVNFPLRDLDL | | |
| | | REFASENTNHAVYNLYAVSNHS | | |
| | | GTTMGGHYTAYCRSPGTGEWHT | | |
| | | FNDSSVTPMSSSQVRTSDAYLL | | |
| | | FYELASPPSRM | | |
| UBP45_HUMAN | 15 | MRVKDPTKALPEKAKRSKRPTV | 127 | LSVRGITNLGNTCFFNAVMQ |
| Ubiquitin | | PHDEDSSDDIAVGLTCQHVSHA | | NLAQTYTLTDLMNEIKESST |
| carboxyl- | | ISVNHVKRAIAENLWSVCSECL | | KLKIFPSSDSQLDPLVVELS |
| terminal | | KERRFYDGQLVLTSDIWLCLKC | | RPGPLTSALFLFLHSMKETE |
| hydrolase 45 | | GFQGCGKNSESQHSLKHFKSSR | | KGPLSPKVLFNQLCQKAPRF |
| | | TEPHCIIINLSTWIIWCYECDE | | KDFQQQDSQELLHYLLDAVR |
| | | KLSTHCNKKVLAQIVDELQKHA | | TEETKRIQASILKAFNNPTT |
| | | SKTQTSAFSRIMKLCEEKCETD | | KTADDETRKKVKAYGKEGVK |
| | | EIQKGGKCRNLSVRGITNLGNT | | MNFIDRIFIGELTSTVMCEE |
| | | CFFNAVMQNLAQTYTLTDLMNE | | CANISTVKDPFIDISLPIIE |
| | | IKESSTKLKIFPSSDSQLDPLV | | ERVSKPLLWGRMNKYRSLRE |
| | | VELSRPGPLTSALFLFLHSMKE | | TDHDRYSGNVTIENIHQPRA |
| | | TEKGPLSPKVLENQLCQKAPRE | | AKKHSSSKDKSQLIHDRKCI |
| | | KDFQQQDSQELLHYLLDAVRTE | | RKLSSGETVTYQKNENLEMN |
| | | ETKRIQASILKAFNNPTTKTAD | | GDSLMFASLMNSESRLNESP |
| | | DETRKKVKAYGKEGVKMNFIDR | | TDDSEKEASHSESNVDADSE |
| | | IFIGELTSTVMCEECANISTVK | | PSESESASKQTGLERSSSGS |
| | | DPFIDISLPIIEERVSKPLLWG | | GVQPDGPLYPLSAGKLLYTK |
| | | RMNKYRSLRETDHDRYSGNVTI | | ETDSGDKEMAEAISELRLSS |
| | | ENIHQPRAAKKHSSSKDKSQLI | | TVTGDQDFDRENQPLNISNN |
| | | HDRKCIRKLSSGETVTYQKNEN | | LCFLEGKHLRSYSPQNAFQT |
| | | LEMNGDSLMFASLMNSESRLNE | | LSQSYITTSKECSIQSCLYQ |
| | | SPTDDSEKEASHSESNVDADSE | | FTSMELLMGNNKLLCENCTK |
| | | PSESESASKQTGLERSSSGSGV | | NKQKYQEETSFAEKKVEGVY |
| | | QPDGPLYPLSAGKLLYTKETDS | | TNARKQLLISAVPAVLILHL |
| | | GDKEMAEAISELRLSSTVTGDQ | | KRFHQAGLSLRKVNRHVDFP |
| | | DEDRENQPLNISNNLCFLEGKH | | LMLDLAPFCSATCKNASVGD |
| | | LRSYSPQNAFQTLSQSYITTSK | | KVLYGLYGIVEHSGSMREGH |
| | | ECSIQSCLYQFTSMELLMGNNK | | YTAYVKVRTPSRKLSEHNTK |
| | | LLCENCTKNKQKYQEETSFAEK | | KKNVPGLKAADNESAGQWVH |
| | | KVEGVYTNARKQLLISAVPAVL | | VSDTYLQVVPESRALSAQAY |
| | | ILHLKRFHQAGLSLRKVNRHVD | | LLFYERVL |
| | | FPLMLDLAPFCSATCKNASVGD | | |
| | | KVLYGLYGIVEHSGSMREGHYT | | |
| | | AYVKVRTPSRKLSEHNTKKKNV | | |
| | | PGLKAADNESAGQWVHVSDTYL | | |
| | | QVVPESRALSAQAYLLFYERVL | | |
| UBP32_HUMAN | 16 | MGAKESRIGFLSYEEALRRVTD | 128 | TEKGATGLSNLGNTCEMNSS |
| Ubiquitin | | VELKRLKDAFKRTCGLSYYMGQ | | IQCVSNTQPLTQYFISGRHL |
| carboxyl- | | HCFIREVLGDGVPPKVAEVIYC | | YELNRTNPIGMKGHMAKCYG |
| terminal | | SFGGTSKGLHENNLIVGLVLLT | | DLVQELWSGTQKNVAPLKLR |
| hydrolase 32 | | RGKDEEKAKYIFSLFSSESGNY | | WTIAKYAPRENGFQQQDSQE |
| | | VIREEMERMLHVVDGKVPDTLR | | LLAFLLDGLHEDLNRVHEKP |
| | | KCFSEGEKVNYEKERNWLFLNK | | YVELKDSDGRPDWEVAAEAW |
| | | DAFTFSRWLLSGGVYVTLTDDS | | DNHLRRNRSIVVDLFHGQLR |
| | | DTPTFYQTLAGVTHLEESDIID | | SQVKCKTCGHISVREDPENE |
| | | LEKRYWLLKAQSRTGREDLETF | | LSLPLPMDSYMHLEITVIKL |
| | | GPLVSPPIRPSLSEGLFNAFDE | | DGTTPVRYGLRLNMDEKYTG |
| | | NRDNHIDFKEISCGLSACCRGP | | LKKQLSDLCGLNSEQILLAE |
| | | LAERQKFCFKVFDVDRDGVLSR | | VHGSNIKNFPQDNQKVRLSV |
| | | VELRDMVVALLEVWKDNRTDDI | | SGFLCAFEIPVPVSPISASS |
| | | PELHMDLSDIVEGILNAHDTTK | | PTQTDFSSSPSTNEMFTLTT |
| | | MGHLTLEDYQIWSVKNVLANEF | | NGDLPRPIFIPNGMPNTVVP |
| | | LNLLFQVCHIVLGLRPATPEEE | | CGTEKNFTNGMVNGHMPSLP |
| | | GQIIRGWLERESRYGLQAGHNW | | DSPFTGYIIAVHRKMMRTEL |
| | | FIISMQWWQQWKEYVKYDANPV | | YFLSSQKNRPSLFGMPLIVP |
| | | VIEPSSVLNGGKYSFGTAAHPM | | |
| | | EQVEDRIGSSLSYVNTTEEKES | | |
| | | DNISTASEASETAGSGFLYSAT | | |
| | | PGADVCFARQHNTSDNNNQCLL | | |
| | | GANGNILLHLNPQKPGAIDNQP | | |
| | | LVTQEPVKATSLTLEGGRLKRT | | |
| | | PQLIHGRDYEMVPEPVWRALYH | | |
| | | WYGANLALPRPVIKNSKTDIPE | | |
| | | LELFPRYLLFLRQQPATRTQQS | | |
| | | NIWVNMGNVPSPNAPLKRVLAY | | |
| | | TGCFSRMQTIKEIHEYLSQRLR | | |
| | | IKEEDMRLWLYNSENYLTLLDD | | |
| | | EDHKLEYLKIQDEQHLVIEVRN | | |
| | | KDMSWPEEMSFIANSSKIDRHK | | |
| | | VPTEKGATGLSNLGNTCFMNSS | | |
| | | IQCVSNTQPLTQYFISGRHLYE | | |
| | | LNRTNPIGMKGHMAKCYGDLVQ | | |
| | | ELWSGTQKNVAPLKLRWTIAKY | | |
| | | APRFNGFQQQDSQELLAFLLDG | | |
| | | LHEDLNRVHEKPYVELKDSDGR | | |
| | | PDWEVAAEAWDNHLRRNRSIVV | | |
| | | DLFHGQLRSQVKCKTCGHISVR | | |
| | | FDPFNFLSLPLPMDSYMHLEIT | | |
| | | VIKLDGTTPVRYGLRLNMDEKY | | |
| | | TGLKKQLSDLCGLNSEQILLAE | | |
| | | VHGSNIKNFPQDNQKVRLSVSG | | |
| | | FLCAFEIPVPVSPISASSPTQT | | |
| | | DFSSSPSTNEMFTLTTNGDLPR | | |
| | | PIFIPNGMPNTVVPCGTEKNFT | | |
| | | NGMVNGHMPSLPDSPFTGYIIA | | |
| | | VHRKMMRTELYFLSSQKNRPSL | | |
| | | FGMPLIVPCTVHTRKKDLYDAV | | |
| | | WIQVSRLASPLPPQEASNHAQD | | |
| | | CDDSMGYQYPFTLRVVQKDGNS | | |
| | | CAWCPWYRFCRGCKIDCGEDRA | | |
| | | FIGNAYIAVDWDPTALHLRYQT | | |
| | | SQERVVDEHESVEQSRRAQAEP | | |
| | | INLDSCLRAFTSEEELGENEMY | | |
| | | YCSKCKTHCLATKKLDLWRLPP | | |
| | | ILIIHLKRFQFVNGRWIKSQKI | | |
| | | VKFPRESFDPSAFLVPRDPALC | | |
| | | QHKPLTPQGDELSEPRILAREV | | |
| | | KKVDAQSSAGEEDVLLSKSPSS | | |
| | | LSANIISSPKGSPSSSRKSGTS | | |
| | | CPSSKNSSPNSSPRTLGRSKGR | | |
| | | LRLPQIGSKNKLSSSKENLDAS | | |
| | | KENGAGQICELADALSRGHVLG | | |
| | | GSQPELVTPQDHEVALANGFLY | | |
| | | EHEACGNGYSNGQLGNHSEEDS | | |
| | | TDDQREDTRIKPIYNLYAISCH | | |
| | | SGILGGGHYVTYAKNPNCKWYC | | |
| | | YNDSSCKELHPDEIDTDSAYIL | | |
| | | FYEQQGIDYAQFLPKTDGKKMA | | |
| | | DTSSMDEDFESDYKKYCVLQ | | |
| | | YNDSSCKELHPDEIDTDSAYIL | | |
| | | FYEQQGIDYAQFLPKTDGKKMA | | |
| | | DTSSMDEDFESDYKKYCVLQ | | |
| U17L6_HUMAN | 17 | MEDDSLYLRGEWQENHESKLTS | 129 | AVGAGLQNMGNTCYVNASLQ |
| Ubiquitin | | SRPDAAFAEIQRTSLPEKSPLS | | CLTYTPPLANYMLSREHSQT |
| carboxyl- | | CETRVDLCDDLAPVARQLAPRE | | CHRHKGCMLCTMQAHITRAL |
| terminal | | KLPLSSRRPAAVGAGLQNMGNT | | HNPGHVIQPSQALAAGFHRG |
| hydrolase 17- | | CYVNASLQCLTYTPPLANYMLS | | KQEDAHEFLMFTVDAMKKAC |
| like protein 6 | | REHSQTCHRHKGCMLCTMQAHI | | LPGHKQVDHHSKDTTLIHQI |
| | | TRALHNPGHVIQPSQALAAGFH | | FGGYWRSQIKCLHCHGISDT |
| | | RGKQEDAHEFLMFTVDAMKKAC | | FDPYLDIALDIQAAQSVQQA |
| | | LPGHKQVDHHSKDTTLIHQIFG | | LEQLVKPEELNGENAYHCGV |
| | | GYWRSQIKCLHCHGISDTEDPY | | CLQRAPASKTLTLHTSAKVL |
| | | LDIALDIQAAQSVQQALEQLVK | | ILVLKRFSDVTGNKIAKNVQ |
| | | PEELNGENAYHCGVCLQRAPAS | | YPECLDMQPYMSQQNTGPLV |
| | | KTLTLHTSAKVLILVLKRESDV | | YVLYAVLVHAGWSCHNGHYF |
| | | TGNKIAKNVQYPECLDMQPYMS | | SYVKAQEGQWYKMDDAEVTA |
| | | QQNTGPLVYVLYAVLVHAGWSC | | SSITSVLSQQAYVLFYIQKS |
| | | HNGHYFSYVKAQEGQWYKMDDA | | |
| | | EVTASSITSVLSQQAYVLFYIQ | | |
| | | KSEWERHSESVSRGREPRALGS | | |
| | | ED | | |
| UBP42_HUMAN | 18 | MTIVDKASESSDPSAYQNQPGS | 130 | RVGAGLQNLGNTCFANAALQ |
| Ubiquitin | | SEAVSPGDMDAGSASWGAVSSL | | CLTYTPPLANYMLSHEHSKT |
| carboxyl- | | NDVSNHTLSLGPVPGAVVYSSS | | CHAEGFCMMCTMQAHITQAL |
| terminal | | SVPDKSKPSPQKDQALGDGIAP | | SNPGDVIKPMFVINEMRRIA |
| hydrolase 42 | | PQKVLFPSEKICLKWQQTHRVG | | RHFREGNQEDAHEFLQYTVD |
| | | AGLQNLGNTCFANAALQCLTYT | | AMQKACLNGSNKLDRHTQAT |
| | | PPLANYMLSHEHSKTCHAEGFC | | TLVCQIFGGYLRSRVKCLNC |
| | | MMCTMQAHITQALSNPGDVIKP | | KGVSDTFDPYLDITLEIKAA |
| | | MFVINEMRRIARHEREGNQEDA | | QSVNKALEQFVKPEQLDGEN |
| | | HEFLQYTVDAMQKACLNGSNKL | | SYKCSKCKKMVPASKRFTIH |
| | | DRHTQATTLVCQIFGGYLRSRV | | RSSNVLTLSLKRFANFTGGK |
| | | KCLNCKGVSDTFDPYLDITLEI | | IAKDVKYPEYLDIRPYMSQP |
| | | KAAQSVNKALEQFVKPEQLDGE | | NGEPIVYVLYAVLVHTGENC |
| | | NSYKCSKCKKMVPASKRFTIHR | | HAGHYFCYIKASNGLWYQMN |
| | | SSNVLTLSLKRFANFTGGKIAK | | DSIVSTSDIRSVLSQQAYVL |
| | | DVKYPEYLDIRPYMSQPNGEPI | | FYIRSHDVKNGGE |
| | | VYVLYAVLVHTGENCHAGHYFC | | |
| | | YIKASNGLWYQMNDSIVSTSDI | | |
| | | RSVLSQQAYVLFYIRSHDVKNG | | |
| | | GELTHPTHSPGQSSPRPVISQR | | |
| | | VVTNKQAAPGFIGPQLPSHMIK | | |
| | | NPPHLNGTGPLKDTPSSSMSSP | | |
| | | NGNSSVNRASPVNASASVQNWS | | |
| | | VNRSSVIPEHPKKQKITISIHN | | |
| | | KLPVRQCQSQPNLHSNSLENPT | | |
| | | KPVPSSTITNSAVQSTSNASTM | | |
| | | SVSSKVTKPIPRSESCSQPVMN | | |
| | | GKSKLNSSVLVPYGAESSEDSD | | |
| | | EESKGLGKENGIGTIVSSHSPG | | |
| | | QDAEDEEATPHELQEPMTLNGA | | |
| | | NSADSDSDPKENGLAPDGASCQ | | |
| | | GQPALHSENPFAKANGLPGKLM | | |
| | | PAPLLSLPEDKILETERLSNKL | | |
| | | KGSTDEMSAPGAERGPPEDRDA | | |
| | | EPQPGSPAAESLEEPDAAAGLS | | |
| | | STKKAPPPRDPGTPATKEGAWE | | |
| | | AMAVAPEEPPPSAGEDIVGDTA | | |
| | | PPDLCDPGSLTGDASPLSQDAK | | |
| | | GMIAEGPRDSALAEAPEGLSPA | | |
| | | PPARSEEPCEQPLLVHPSGDHA | | |
| | | RDAQDPSQSLGAPEAAERPPAP | | |
| | | VLDMAPAGHPEGDAEPSPGERV | | |
| | | EDAAAPKAPGPSPAKEKIGSLR | | |
| | | KVDRGHYRSRRERSSSGEPARE | | |
| | | SRSKTEGHRHRRRRTCPRERDR | | |
| | | QDRHAPEHHPGHGDRLSPGERR | | |
| | | SLGRCSHHHSRHRSGVELDWVR | | |
| | | HHYTEGERGWGREKFYPDRPRW | | |
| | | DRCRYYHDRYALYAARDWKPFH | | |
| | | GGREHERAGLHERPHKDHNRGR | | |
| | | RGCEPARERERHRPSSPRAGAP | | |
| | | HALAPHPDRESHDRTALVAGDN | | |
| | | CNLSDRFHEHENGKSRKRRHDS | | |
| | | VENSDSHVEKKARRSEQKDPLE | | |
| | | EPKAKKHKKSKKKKKSKDKHRD | | |
| | | RDSRHQQDSDLSAACSDADLHR | | |
| | | HKKKKKKKKRHSRKSEDFVKDS | | |
| | | ELHLPRVTSLETVAQFRRAQGG | | |
| | | FPLSGGPPLEGVGPFREKTKHL | | |
| | | RMESRDDRCRLFEYGQGKRRYL | | |
| | | ELGR | | |
| U17L7_HUMAN | 19 | MEDDSLYLGGDWQFNHESKLTS | 131 | AVGAGLQKIGNTFYVNVSLQ |
| Inactive | | SRLDAAFAEIQRTSLSEKSPLS | | CLTYTLPLSNYMLSREDSQT |
| ubiquitin | | SETREDLCDDLAPVARQLAPRE | | CHLHKCCMFCTMQAHITWAL |
| carboxyl- | | KLPLSSRRPAAVGAGLQKIGNT | | HSPGHVIQPSQVLAAGFHRG |
| terminal | | FYVNVSLQCLTYTLPLSNYMLS | | EQEDAHEFLMFTVDAMKKAC |
| hydrolase 17- | | REDSQTCHLHKCCMFCTMQAHI | | LPGHKQLDHHSKDTTLIHQI |
| like protein 7 | | TWALHSPGHVIQPSQVLAAGFH | | FGAYWRSQIKYLHCHGVSDT |
| | | RGEQEDAHEFLMFTVDAMKKAC | | FDPYLDIALDIQAAQSVKQA |
| | | LPGHKQLDHHSKDTTLIHQIFG | | LEQLVKPKELNGENAYHCGL |
| | | AYWRSQIKYLHCHGVSDTEDPY | | CLQKAPASKTLTLPTSAKVL |
| | | LDIALDIQAAQSVKQALEQLVK | | ILVLKRFSDVTGNKLAKNVQ |
| | | PKELNGENAYHCGLCLQKAPAS | | YPKCRDMQPYMSQQNTGPLV |
| | | KTLTLPTSAKVLILVLKRESDV | | YVLYAVLVHAGWSCHNGHYF |
| | | TGNKLAKNVQYPKCRDMQPYMS | | SYVKAQEGQWYKMDDAEVTA |
| | | QQNTGPLVYVLYAVLVHAGWSC | | SGITSVLSQQAYVLFYIQKS |
| | | HNGHYFSYVKAQEGQWYKMDDA | | EWERHSESVSRGREPRALGA |
| | | EVTASGITSVLSQQAYVLFYIQ | | EDTDRPATQGELKRDHPCLQ |
| | | KSEWERHSESVSRGREPRALGA | | VPEL |
| | | EDTDRPATQGELKRDHPCLQVP | | |
| | | ELDEHLVERATQESTLDHWKFP | | |
| | | QEQNKTKPEFNVRKVEGTLPPN | | |
| | | VLVIHQSKYKCGMKNHHPEQQS | | |
| | | SLLNLSSTKPTDQESMNTGTLA | | |
| | | SLQGSTRRSKGNNKHSKRSLLV | | |
| | | CQ | | |
| U17LH_HUMAN | 20 | MEDDSLYLGGEWQFNHESKLTS | 132 | AVGAGLQNMGNTCYVNASLQ |
| Ubiquitin | | SRPDAAFAEIQRTSLPEKSPLS | | CLTYTPPLANYMLSREHSQT |
| carboxyl- | | CETRVDLCDDLAPVARQLAPRE | | CHRHKGCMLCTMQAHITRAL |
| terminal | | KLPLSSRRPAAVGAGLQNMGNT | | HNPGHVIQPSQALAAGFHRG |
| hydrolase 17- | | CYVNASLQCLTYTPPLANYMLS | | KQEDAHEFLMFTVDAMKKAC |
| like protein 17 | | REHSQTCHRHKGCMLCTMQAHI | | LPGHKQVDHHSKDTTLIHQI |
| | | TRALHNPGHVIQPSQALAAGFH | | FGGYWRSQIKCLHCHGISDT |
| | | RGKQEDAHEFLMFTVDAMKKAC | | FDPYLDIALDIQAAQSVQQA |
| | | LPGHKQVDHHSKDTTLIHQIFG | | LEQLVKPEELNGENAYHCGV |
| | | GYWRSQIKCLHCHGISDTFDPY | | CLQRAPASKTLTLHTSAKVL |
| | | LDIALDIQAAQSVQQALEQLVK | | ILVLKRFSDVTGNKIAKNVQ |
| | | PEELNGENAYHCGVCLQRAPAS | | YPECLDMQPYMSQQNTGPLV |
| | | KTLTLHTSAKVLILVLKRESDV | | YVLYAVLVHAGWSCHNGHYF |
| | | TGNKIAKNVQYPECLDMQPYMS | | SYVKAQEGQWYKMDDAEVTA |
| | | QQNTGPLVYVLYAVLVHAGWSC | | ASITSVLSQQAYVLFYIQKS |
| | | HNGHYFSYVKAQEGQWYKMDDA | | EWERHSESVSRGREPRALGA |
| | | EVTAASITSVLSQQAYVLFYIQ | | EDTDRRATQGELKRDHPCLQ |
| | | KSEWERHSESVSRGREPRALGA | | APEL |
| | | EDTDRRATQGELKRDHPCLQAP | | |
| | | ELDEHLVERATQESTLDHWKEL | | |
| | | QEQNKTKPEFNVRKVEGTLPPD | | |
| | | VLVIHQSKYKCGMKNHHPEQQS | | |
| | | SLLNLSSSTPTHQESMNTGTLA | | |
| | | SLRGRARRSKGKNKHSKRALLV | | |
| | | CQ | | |
| UBP13_HUMAN | 21 | MQRRGALFGMPGGSGGRKMAAG | 133 | YGPGYTGLKNLGNSCYLSSV |
| Ubiquitin | | DIGELLVPHMPTIRVPRSGDRV | | MQAIFSIPEFQRAYVGNLPR |
| carboxyl- | | YKNECAFSYDSPNSEGGLYVCM | | IFDYSPLDPTQDENTQMTKL |
| terminal | | NTFLAFGREHVERHERKTGQSV | | GHGLLSGQYSKPPVKSELIE |
| hydrolase 13 | | YMHLKRHVREKVRGASGGALPK | | QVMKEEHKPQQNGISPRMEK |
| | | RRNSKIFLDLDTDDDLNSDDYE | | AFVSKSHPEFSSNRQQDAQE |
| | | YEDEAKLVIFPDHYEIALPNIE | | FFLHLVNLVERNRIGSENPS |
| | | ELPALVTIACDAVLSSKSPYRK | | DVERELVEERIQCCQTRKVR |
| | | QDPDTWENELPVSKYANNLTQL | | YTERVDYLMQLPVAMEAATN |
| | | DNGVRIPPSGWKCARCDLRENL | | KDELIAYELTRREAEANRRP |
| | | WLNLTDGSVLCGKWFFDSSGGN | | LPELVRAKIPFSACLQAFSE |
| | | GHALEHYRDMGYPLAVKLGTIT | | PENVDDEWSSALQAKSAGVK |
| | | PDGADVYSFQEEEPVLDPHLAK | | TSRFASFPEYLVVQIKKETE |
| | | HLAHFGIDMLHMHGTENGLQDN | | GLDWVPKKFDVSIDMPDLLD |
| | | DIKLRVSEWEVIQESGTKLKPM | | INHLRARGLQPGEEELPDIS |
| | | YGPGYTGLKNLGNSCYLSSVMQ | | PPIVIPDDSKDRLMNQLIDP |
| | | AIFSIPEFQRAYVGNLPRIFDY | | SDIDESSVMQLAEMGFPLEA |
| | | SPLDPTQDENTQMTKLGHGLLS | | CRKAVYFTGNMGAEVAFNWI |
| | | GQYSKPPVKSELIEQVMKEEHK | | IVHMEEPDFAEPLTMPGYGG |
| | | PQQNGISPRMFKAFVSKSHPEF | | AASAGASVEGASGLDNQPPE |
| | | SSNRQQDAQEFFLHLVNLVERN | | EIVAIITSMGFQRNQAIQAL |
| | | RIGSENPSDVFRELVEERIQCC | | RATNNNLERALDWIFSHPEF |
| | | QTRKVRYTERVDYLMQLPVAME | | EEDSDEVIEMENNANANIIS |
| | | AATNKDELIAYELTRREAEANR | | EAKPEGPRVKDGSGTYELFA |
| | | RPLPELVRAKIPFSACLQAFSE | | FISHMGTSTMSGHYICHIKK |
| | | PENVDDFWSSALQAKSAGVKTS | | EGRWVIYNDHKVCASERPPK |
| | | RFASFPEYLVVQIKKETFGLDW | | DLGYMYFYRRIPS |
| | | VPKKFDVSIDMPDLLDINHLRA | | |
| | | RGLQPGEEELPDISPPIVIPDD | | |
| | | SKDRLMNQLIDPSDIDESSVMQ | | |
| | | LAEMGFPLEACRKAVYFTGNMG | | |
| | | AEVAFNWIIVHMEEPDFAEPLT | | |
| | | MPGYGGAASAGASVEGASGLDN | | |
| | | QPPEEIVAIITSMGFQRNQAIQ | | |
| | | ALRATNNNLERALDWIFSHPEF | | |
| | | EEDSDEVIEMENNANANIISEA | | |
| | | KPEGPRVKDGSGTYELFAFISH | | |
| | | MGTSTMSGHYICHIKKEGRWVI | | |
| | | YNDHKVCASERPPKDLGYMYFY | | |
| | | RRIPS | | |
| UBP11_HUMAN | 22 | MAVAPRLEGGLCFRFRDQNPEV | 134 | KGQPGICGLTNLGNTCEMNS |
| Ubiquitin | | AVEGRLPISHSCVGCRRERTAM | | ALQCLSNVPQLTEYFLNNCY |
| carboxyl- | | ATVAANPAAAAAAVAAAAAVTE | | LEELNERNPLGMKGEIAEAY |
| terminal | | DREPQHEELPGLDSQWRQIENG | | ADLVKQAWSGHHRSIVPHVE |
| hydrolase 11 | | ESGRERPLRAGESWELVEKHWY | | KNKVGHFASQFLGYQQHDSQ |
| | | KQWEAYVQGGDQDSSTFPGCIN | | ELLSFLLDGLHEDLNRVKKK |
| | | NATLFQDEINWRLKEGLVEGED | | EYVELCDAAGRPDQEVAQEA |
| | | YVLLPAAAWHYLVSWYGLEHGQ | | WQNHKRRNDSVIVDTFHGLE |
| | | PPIERKVIELPNIQKVEVYPVE | | KSTLVCPDCGNVSVTFDPFC |
| | | LLLVRHNDLGKSHTVQFSHTDS | | YLSVPLPISHKRVLEVFFIP |
| | | IGLVLRTARERELVEPQEDTRL | | MDPRRKPEQHRLVVPKKGKI |
| | | WAKNSEGSLDRLYDTHITVLDA | | SDLCVALSKHTGISPERMMV |
| | | ALETGQLIIMETRKKDGTWPSA | | ADVESHRFYKLYQLEEPLSS |
| | | QLHVMNNNMSEEDEDEKGQPGI | | ILDRDDIFVYEVSGRIEAIE |
| | | CGLTNLGNTCFMNSALQCLSNV | | GSREDIVVPVYLRERTPARD |
| | | PQLTEYFLNNCYLEELNERNPL | | YNNSYYGLMLFGHPLLVSVP |
| | | GMKGEIAEAYADLVKQAWSGHH | | RDRFTWEGLYNVLMYRLSRY |
| | | RSIVPHVEKNKVGHFASQFLGY | | VTKPNSDDEDDGDEKEDDEE |
| | | QQHDSQELLSFLLDGLHEDLNR | | DKDDVPGPSTGGSLRDPEPE |
| | | VKKKEYVELCDAAGRPDQEVAQ | | QAGPSSGVTNRCPFLLDNCL |
| | | EAWQNHKRRNDSVIVDTFHGLF | | GTSQWPPRRRRKQLFTLQTV |
| | | KSTLVCPDCGNVSVTFDPFCYL | | NSNGTSDRTTSPEEVHAQPY |
| | | SVPLPISHKRVLEVFFIPMDPR | | IAIDWEPEMKKRYYDEVEAE |
| | | RKPEQHRLVVPKKGKISDLCVA | | GYVKHDCVGYVMKKAPVRLQ |
| | | LSKHTGISPERMMVADVESHRE | | ECIELFTTVETLEKENPWYC |
| | | YKLYQLEEPLSSILDRDDIFVY | | PSCKQHQLATKKLDLWMLPE |
| | | EVSGRIEAIEGSREDIVVPVYL | | ILIIHLKRFSYTKESREKLD |
| | | RERTPARDYNNSYYGLMLFGHP | | TLVEFPIRDLDESEFVIQPQ |
| | | LLVSVPRDRETWEGLYNVLMYR | | NESNPELYKYDLIAVSNHYG |
| | | LSRYVTKPNSDDEDDGDEKEDD | | GMRDGHYTTFACNKDSGQWH |
| | | EEDKDDVPGPSTGGSLRDPEPE | | YFDDNSVSPVNENQIESKAA |
| | | QAGPSSGVTNRCPFLLDNCLGT | | YVLFYQRQD |
| | | SQWPPRRRRKQLFTLQTVNSNG | | |
| | | TSDRTTSPEEVHAQPYIAIDWE | | |
| | | PEMKKRYYDEVEAEGYVKHDCV | | |
| | | GYVMKKAPVRLQECIELFTTVE | | |
| | | TLEKENPWYCPSCKQHQLATKK | | |
| | | LDLWMLPEILIIHLKRFSYTKE | | |
| | | SREKLDTLVEFPIRDLDESEFV | | |
| | | IQPQNESNPELYKYDLIAVSNH | | |
| | | YGGMRDGHYTTFACNKDSGQWH | | |
| | | YFDDNSVSPVNENQIESKAAYV | | |
| | | LFYQRQDVARRLLSPAGSSGAP | | |
| | | ASPACSSPPSSEFMDVN | | |
| U17L1_HUMAN | 23 | MGDDSLYLGGEWQFNHFSKLTS | 135 | AVGAGLQNMGNTCYENASLQ |
| Ubiquitin | | SRPDAAFAEIQRTSLPEKSPLS | | CLTYTLPLANYMLSREHSQT |
| carboxyl- | | SETRVDLCDDLAPVARQLAPRE | | CQRPKCCMLCTMQAHITWAL |
| terminal | | KLPLSSRRPAAVGAGLQNMGNT | | HSPGHVIQPSQALAAGFHRG |
| hydrolase 17- | | CYENASLQCLTYTLPLANYMLS | | KQEDVHEFLMFTVDAMKKAC |
| like protein 1 | | REHSQTCQRPKCCMLCTMQAHI | | LPGHKQVDHHCKDTTLIHQI |
| | | TWALHSPGHVIQPSQALAAGFH | | FGGCWRSQIKCLHCHGISDT |
| | | RGKQEDVHEFLMFTVDAMKKAC | | FDPYLDIALDIQAAQSVKQA |
| | | LPGHKQVDHHCKDTTLIHQIFG | | LEQLVKPEELNGENAYHCGL |
| | | GCWRSQIKCLHCHGISDTEDPY | | CLQRAPASNTLTLHTSAKVL |
| | | LDIALDIQAAQSVKQALEQLVK | | ILVLKRFSDVAGNKLAKNVQ |
| | | PEELNGENAYHCGLCLQRAPAS | | YPECLDMQPYMSQQNTGPLV |
| | | NTLTLHTSAKVLILVLKRFSDV | | YVLYAVLVHAGWSCHDGHYF |
| | | AGNKLAKNVQYPECLDMQPYMS | | SYVKAQEVQWYKMDDAEVTV |
| | | QQNTGPLVYVLYAVLVHAGWSC | | CSIISVLSQQAYVLFYIQKS |
| | | HDGHYFSYVKAQEVQWYKMDDA | | |
| | | EVTVCSIISVLSQQAYVLFYIQ | | |
| | | KSEWERHSESVSRGREPRALGA | | |
| | | EDTDRRAKQGELKRDHPCLQAP | | |
| | | ELDEHLVERATQESTLDHWKEL | | |
| | | QEQNKTKPEFNVGKVEGTLPPN | | |
| | | ALVIHQSKYKCGMKNHHPEQQS | | |
| | | SLLNLSSTTRTDQESMNTGTLA | | |
| | | SLQGRTRRAKGKNKHSKRALLV | | |
| | | CQ | | |
| UBP14_HUMAN | 24 | MPLYSVTVKWGKEKFEGVELNT | 136 | ASAMELPCGLTNLGNTCYMN |
| Ubiquitin | | DEPPMVFKAQLFALTGVQPARQ | | ATVQCIRSVPELKDALKRYA |
| carboxyl- | | KVMVKGGTLKDDDWGNIKIKNG | | GALRASGEMASAQYITAALR |
| terminal | | MTLLMMGSADALPEEPSAKTVE | | DLFDSMDKTSSSIPPIILLQ |
| hydrolase 14 | | VEDMTEEQLASAMELPCGLTNL | | FLHMAFPQFAEKGEQGQYLQ |
| | | GNTCYMNATVQCIRSVPELKDA | | QDANECWIQMMRVLQQKLEA |
| | | LKRYAGALRASGEMASAQYITA | | IEDDSVKETDSSSASAATPS |
| | | ALRDLEDSMDKTSSSIPPIILL | | KKKSLIDQFFGVEFETTMKC |
| | | QFLHMAFPQFAEKGEQGQYLQQ | | TESEEEEVTKGKENQLQLSC |
| | | DANECWIQMMRVLQQKLEAIED | | FINQEVKYLFTGLKLRLQEE |
| | | DSVKETDSSSASAATPSKKKSL | | ITKQSPTLQRNALYIKSSKI |
| | | IDQFFGVEFETTMKCTESEEEE | | SRLPAYLTIQMVRFFYKEKE |
| | | VTKGKENQLQLSCFINQEVKYL | | SVNAKVLKDVKFPLMLDMYE |
| | | FTGLKLRLQEEITKQSPTLQRN | | LCTPELQEKMVSERSKEKDL |
| | | ALYIKSSKISRLPAYLTIQMVR | | EDKKVNQQPNTSDKKSSPQK |
| | | FFYKEKESVNAKVLKDVKFPLM | | EVKYEPFSFADDIGSNNCGY |
| | | LDMYELCTPELQEKMVSERSKF | | YDLQAVLTHQGRSSSSGHYV |
| | | KDLEDKKVNQQPNTSDKKSSPQ | | SWVKRKQDEWIKEDDDKVSI |
| | | KEVKYEPFSFADDIGSNNCGYY | | VTPEDILRLSGGGDWHIAYV |
| | | DLQAVLTHQGRSSSSGHYVSWV | | LLYGPRR |
| | | KRKQDEWIKFDDDKVSIVTPED | | |
| | | ILRLSGGGDWHIAYVLLYGPRR | | |
| | | VEIMEEESEQ | | |
| Q13107\|UBP4 | 25 | MAEGGGCRERPDAETQKSELGP | 137 | SHIQPGLCGLGNLGNTCFMN |
| HUMAN | | LMRTTLQRGAQWYLIDSRWFKQ | | SALQCLSNTAPLTDYELKDE |
| Ubiquitin | | WKKYVGFDSWDMYNVGEHNLEP | | YEAEINRDNPLGMKGEIAEA |
| carboxyl- | | GPIDNSGLFSDPESQTLKEHLI | | YAELIKQMWSGRDAHVAPRM |
| terminal | | DELDYVLVPTEAWNKLLNWYGC | | FKTQVGRFAPQFSGYQQQDS |
| hydrolase 4 | | VEGQQPIVRKVVEHGLFVKHCK | | QELLAFLLDGLHEDLNRVKK |
| | | VEVYLLELKLCENSDPTNVLSC | | KPYLELKDANGRPDAVVAKE |
| | | HFSKADTIATIEKEMRKLENIP | | AWENHRLRNDSVIVDTFHGL |
| | | AERETRLWNKYMSNTYEQLSKL | | FKSTLVCPECAKVSVTEDPF |
| | | DNTVQDAGLYQGQVLVIEPQNE | | CYLTLPLPLKKDRVMEVFLV |
| | | DGTWPRQTLQSKSSTAPSRNFT | | PADPHCRPTQYRVTVPLMGA |
| | | TSPKSSASPYSSVSASLIANGD | | VSDLCEALSRLSGIAAENMV |
| | | STSTCGMHSSGVSRGGSGESAS | | VADVYNHRFHKIFQMDEGLN |
| | | YNCQEPPSSHIQPGLCGLGNLG | | HIMPRDDIFVYEVCSTSVDG |
| | | NTCFMNSALQCLSNTAPLTDYF | | SECVTLPVYFRERKSRPSST |
| | | LKDEYEAEINRDNPLGMKGEIA | | SSASALYGQPLLLSVPKHKL |
| | | EAYAELIKQMWSGRDAHVAPRM | | TLESLYQAVCDRISRYVKQP |
| | | FKTQVGRFAPQFSGYQQQDSQE | | LPDEFGSSPLEPGACNGSRN |
| | | LLAFLLDGLHEDLNRVKKKPYL | | SCEGEDEEEMEHQEEGKEQL |
| | | ELKDANGRPDAVVAKEAWENHR | | SETEGSGEDEPGNDPSETTQ |
| | | LRNDSVIVDTFHGLFKSTLVCP | | KKIKGQPCPKRLFTESLVNS |
| | | ECAKVSVTFDPFCYLTLPLPLK | | YGTADINSLAADGKLLKLNS |
| | | KDRVMEVFLVPADPHCRPTQYR | | RSTLAMDWDSETRRLYYDEQ |
| | | VTVPLMGAVSDLCEALSRLSGI | | ESEAYEKHVSMLQPQKKKKT |
| | | AAENMVVADVYNHRFHKIFQMD | | TVALRDCIELFTTMETLGEH |
| | | EGLNHIMPRDDIFVYEVCSTSV | | DPWYCPNCKKHQQATKKEDL |
| | | DGSECVTLPVYFRERKSRPSST | | WSLPKILVVHLKRFSYNRYW |
| | | SSASALYGQPLLLSVPKHKLTL | | RDKLDTVVEFPIRGLNMSEF |
| | | ESLYQAVCDRISRYVKQPLPDE | | VCNLSARPYVYDLIAVSNHY |
| | | FGSSPLEPGACNGSRNSCEGED | | GAMGVGHYTAYAKNKLNGKW |
| | | EEEMEHQEEGKEQLSETEGSGE | | YYFDDSNVSLASEDQIVTKA |
| | | DEPGNDPSETTQKKIKGQPCPK | | AYVLFYQRRD |
| | | RLFTFSLVNSYGTADINSLAAD | | |
| | | GKLLKLNSRSTLAMDWDSETRR | | |
| | | LYYDEQESEAYEKHVSMLQPQK | | |
| | | KKKTTVALRDCIELFTTMETLG | | |
| | | EHDPWYCPNCKKHQQATKKEDL | | |
| | | WSLPKILVVHLKRFSYNRYWRD | | |
| | | KLDTVVEFPIRGLNMSEFVCNL | | |
| | | SARPYVYDLIAVSNHYGAMGVG | | |
| | | HYTAYAKNKLNGKWYYFDDSNV | | |
| | | SLASEDQIVTKAAYVLFYQRRD | | |
| | | DEFYKTPSLSSSGSSDGGTRPS | | |
| | | SSQQGFGDDEACSMDTN | | |
| UBP26_HUMAN | 26 | MAALFLRGFVQIGNCKTGISKS | 138 | KICHGLPNLGNTCYMNAVLQ |
| Ubiquitin | | KEAFIEAVERKKKDRLVLYFKS | | SLLSIPSFADDLLNQSFPWG |
| carboxyl- | | GKYSTFRLSDNIQNVVLKSYRG | | KIPLNALTMCLARLLFFKDT |
| terminal | | NQNHLHLTLQNNNGLFIEGLSS | | YNIEIKEMLLLNLKKAISAA |
| hydrolase 26 | | TDAEQLKIFLDRVHQNEVQPPV | | AEIFHGNAQNDAHEFLAHCL |
| | | RPGKGGSVFSSTTQKEINKTSF | | DQLKDNMEKLNTIWKPKSEF |
| | | HKVDEKSSSKSFEIAKGSGTGV | | GEDNFPKQVFADDPDTSGES |
| | | LQRMPLLTSKLTLTCGELSENQ | | CPVITNFELELLHSIACKAC |
| | | HKKRKRMLSSSSEMNEEFLKEN | | GQVILKTELNNYLSINLPQR |
| | | NSVEYKKSKADCSRCVSYNREK | | IKAHPSSIQSTEDLFFGAEE |
| | | QLKLKELEENKKLECESSCIMN | | LEYKCAKCEHKTSVGVHSES |
| | | ATGNPYLDDIGLLQALTEKMVL | | RLPRILIVHLKRYSLNEFCA |
| | | VFLLQQGYSDGYTKWDKLKLFF | | LKKNDQEVIISKYLKVSSHC |
| | | ELFPEKICHGLPNLGNTCYMNA | | NEGTRPPLPLSEDGEITDFQ |
| | | VLQSLLSIPSFADDLLNQSFPW | | LLKVIRKMTSGNISVSWPAT |
| | | GKIPLNALTMCLARLLFFKDTY | | KESKDILAPHIGSDKESEQK |
| | | NIEIKEMLLLNLKKAISAAAEI | | KGQTVFKGASRRQQQKYLGK |
| | | FHGNAQNDAHEFLAHCLDQLKD | | NSKPNELESVYSGDRAFIEK |
| | | NMEKLNTIWKPKSEFGEDNEPK | | EPLAHLMTYLEDTSLCQFHK |
| | | QVFADDPDTSGFSCPVITNFEL | | AGGKPASSPGTPLSKVDFQT |
| | | ELLHSIACKACGQVILKTELNN | | VPENPKRKKYVKTSKEVAFD |
| | | YLSINLPQRIKAHPSSIQSTED | | RIINPTKDLYEDKNIRIPER |
| | | LFFGAEELEYKCAKCEHKTSVG | | FQKVSEQTQQCDGMRICEQA |
| | | VHSFSRLPRILIVHLKRYSLNE | | PQQALPQSFPKPGTQGHTKN |
| | | FCALKKNDQEVIISKYLKVSSH | | LLRPTKLNLQKSNRNSLLAL |
| | | CNEGTRPPLPLSEDGEITDFQL | | GSNKNPRNKDILDKIKSKAK |
| | | LKVIRKMTSGNISVSWPATKES | | ETKRNDDKGDHTYRLISVVS |
| | | KDILAPHIGSDKESEQKKGQTV | | HLGKTLKSGHYICDAYDFEK |
| | | FKGASRRQQQKYLGKNSKPNEL | | QIWFTYDDMRVLGIQEAQMQ |
| | | ESVYSGDRAFIEKEPLAHLMTY | | EDRRCTGYIFFYMHN |
| | | LEDTSLCQFHKAGGKPASSPGT | | |
| | | PLSKVDFQTVPENPKRKKYVKT | | |
| | | SKFVAFDRIINPTKDLYEDKNI | | |
| | | RIPERFQKVSEQTQQCDGMRIC | | |
| | | EQAPQQALPQSFPKPGTQGHTK | | |
| | | NLLRPTKLNLQKSNRNSLLALG | | |
| | | SNKNPRNKDILDKIKSKAKETK | | |
| | | RNDDKGDHTYRLISVVSHLGKT | | |
| | | LKSGHYICDAYDFEKQIWFTYD | | |
| | | DMRVLGIQEAQMQEDRRCTGYI | | |
| | | FFYMHNEIFEEMLKREENAQLN | | |
| | | SKEVEETLQKE | | |
| UBP19_HUMAN | 27 | MSGGASATGPRRGPPGLEDTTS | 139 | LPGFTGLVNLGNTCEMNSVI |
| Ubiquitin | | KKKQKDRANQESKDGDPRKETG | | QSLSNTRELRDFFHDRSFEA |
| carboxyl- | | SRYVAQAGLEPLASGDPSASAS | | EINYNNPLGTGGRLAIGFAV |
| terminal | | HAAGITGSRHRTRLFFPSSSGS | | LLRALWKGTHHAFQPSKLKA |
| hydrolase 19 | | ASTPQEEQTKEGACEDPHDLLA | | IVASKASQFTGYAQHDAQEF |
| | | TPTPELLLDWRQSAEEVIVKLR | | MAFLLDGLHEDLNRIQNKPY |
| | | VGVGPLQLEDVDAAFTDTDCVV | | TETVDSDGRPDEVVAEEAWQ |
| | | RFAGGQQWGGVFYAEIKSSCAK | | RHKMRNDSFIVDLFQGQYKS |
| | | VQTRKGSLLHLTLPKKVPMLTW | | KLVCPVCAKVSITFDPFLYL |
| | | PSLLVEADEQLCIPPLNSQTCL | | PVPLPQKQKVLPVFYFAREP |
| | | LGSEENLAPLAGEKAVPPGNDP | | HSKPIKFLVSVSKENSTASE |
| | | VSPAMVRSRNPGKDDCAKEEMA | | VLDSLSQSVHVKPENLRLAE |
| | | VAADAATLVDEPESMVNLAFVK | | VIKNRFHRVFLPSHSLDTVS |
| | | NDSYEKGPDSVVVHVYVKEICR | | PSDTLLCFELLSSELAKERV |
| | | DTSRVLFREQDFTLIFQTRDGN | | VVLEVQQRPQVPSVPISKCA |
| | | FLRLHPGCGPHTTFRWQVKLRN | | ACQRKQQSEDEKLKRCTRCY |
| | | LIEPEQCTFCFTASRIDICLRK | | RVGYCNQLCQKTHWPDHKGL |
| | | RQSQRWGGLEAPAARVGGAKVA | | CRPENIGYPFLVSVPASRLT |
| | | VPTGPTPLDSTPPGGAPHPLTG | | YARLAQLLEGYARYSVSVFQ |
| | | QEEARAVEKDKSKARSEDTGLD | | PPFQPGRMALESQSPGCTTL |
| | | SVATRTPMEHVTPKPETHLASP | | LSTGSLEAGDSERDPIQPPE |
| | | KPTCMVPPMPHSPVSGDSVEEE | | LQLVTPMAEGDTGLPRVWAA |
| | | EEEEKKVCLPGFTGLVNLGNTC | | PDRGPVPSTSGISSEMLASG |
| | | FMNSVIQSLSNTRELRDFFHDR | | PIEVGSLPAGERVSRPEAAV |
| | | SFEAEINYNNPLGTGGRLAIGF | | PGYQHPSEAMNAHTPQFFIY |
| | | AVLLRALWKGTHHAFQPSKLKA | | KIDSSNREQRLEDKGDTPLE |
| | | IVASKASQFTGYAQHDAQEFMA | | LGDDCSLA |
| | | FLLDGLHEDLNRIQNKPYTETV | | LVWRNNERLQEFVLVASKEL |
| | | DSDGRPDEVVAEEAWQRHKMRN | | ECAEDPGSAGEAARAGHFTL |
| | | DSFIVDLFQGQYKSKLVCPVCA | | DQCLNLFTRPEVLAPEEAWY |
| | | KVSITFDPFLYLPVPLPQKQKV | | CPQCKQHREASKQLLLWRLP |
| | | LPVFYFAREPHSKPIKFLVSVS | | NVLIVQLKRFSFRSFIWRDK |
| | | KENSTASEVLDSLSQSVHVKPE | | INDLVEFPVRNLDLSKFCIG |
| | | NLRLAEVIKNRFHRVFLPSHSL | | QKEEQLPSYDLYAVINHYGG |
| | | DTVSPSDTLLCFELLSSELAKE | | MIGGHYTACARLPNDRSSQR |
| | | RVVVLEVQQRPQVPSVPISKCA | | SDVGWRLFDDSTVTTVDESQ |
| | | ACQRKQQSEDEKLKRCTRCYRV | | VVTRYAYVLFYRRRN |
| | | GYCNQLCQKTHWPDHKGLCRPE | | |
| | | NIGYPFLVSVPASRLTYARLAQ | | |
| | | LLEGYARYSVSVFQPPFQPGRM | | |
| | | ALESQSPGCTTLLSTGSLEAGD | | |
| | | SERDPIQPPELQLVTPMAEGDT | | |
| | | GLPRVWAAPDRGPVPSTSGISS | | |
| | | EMLASGPIEVGSLPAGERVSRP | | |
| | | EAAVPGYQHPSEAMNAHTPQFF | | |
| | | IYKIDSSNREQRLEDKGDTPLE | | |
| | | LGDDCSLALVWRNNERLQEFVL | | |
| | | VASKELECAEDPGSAGEAARAG | | |
| | | HFTLDQCLNLFTRPEVLAPEEA | | |
| | | WYCPQCKQHREASKQLLLWRLP | | |
| | | NVLIVQLKRFSFRSFIWRDKIN | | |
| | | DLVEFPVRNLDLSKFCIGQKEE | | |
| | | QLPSYDLYAVINHYGGMIGGHY | | |
| | | TACARLPNDRSSQRSDVGWRLF | | |
| | | DDSTVTTVDESQVVTRYAYVLF | | |
| | | YRRRNSPVERPPRAGHSEHHPD | | |
| | | LGPAAEAAASQASRIWQELEAE | | |
| | | EEPVPEGSGPLGPWGPQDWVGP | | |
| | | LPRGPTTPDEGCLRYFVLGTVA | | |
| | | ALVALVLNVFYPLVSQSRWR | | |
| UBP10_HUMAN | 28 | MALHSPQYIFGDESPDEFNQFF | 140 | SLQPRGLINKGNWCYINATL |
| Ubiquitin | | VTPRSSVELPPYSGTVLCGTQA | | QALVACPPMYHLMKFIPLYS |
| carboxyl- | | VDKLPDGQEYQRIEFGVDEVIE | | KVQRPCTSTPMIDSFVRLMN |
| terminal | | PSDTLPRTPSYSISSTLNPQAP | | EFTNMPVPPKPRQALGDKIV |
| hydrolase 10 | | EFILGCTASKITPDGITKEASY | | RDIRPGAAFEPTYIYRLLTV |
| | | GSIDCQYPGSALALDGSSNVEA | | NKSSLSEKGRQEDAEEYLGE |
| | | EVLENDGVSGGLGQRERKKKKK | | ILNGLHEEMLNLKKLLSPSN |
| | | RPPGYYSYLKDGGDDSISTEAL | | EKLTISNGPKNHSVNEEEQE |
| | | VNGHANSAVPNSVSAEDAEFMG | | EQGEGSEDEWEQVGPRNKTS |
| | | DMPPSVTPRTCNSPQNSTDSVS | | VTRQADFVQTPITGIFGGHI |
| | | DIVPDSPFPGALGSDTRTAGQP | | RSVVYQQSSKESATLQPFFT |
| | | EGGPGADFGQSCFPAEAGRDTL | | LQLDIQSDKIRTVQDALESL |
| | | SRTAGAQPCVGTDTTENLGVAN | | VARESVQGYTTKTKQEVEIS |
| | | GQILESSGEGTATN | | RRVTLEKLPPVLVLHLKREV |
| | | GVELHTTESIDLDPTKPESASP | | YEKTGGCQKLIKNIEYPVDL |
| | | PADGTGSASGTLPVSQPKSWAS | | EISKELLSPGVKNKNFKCHR |
| | | LFHDSKPSSSSPVAYVETKYSP | | TYRLFAVVYHHGNSATGGHY |
| | | PAISPLVSEKQVEVKEGLVPVS | | TTDVFQIGLNGWLRIDDQTV |
| | | EDPVAIKIAELLENVTLIHKPV | | KVINQYQVVKPTAERTAYLL |
| | | SLQPRGLINKGNWCYINATLQA | | YYRRVD |
| | | LVACPPMYHLMKFIPLYSKVQR | | |
| | | PCTSTPMIDSFVRLMNEFTNMP | | |
| | | VPPKPRQALGDKIVRDIRPGAA | | |
| | | FEPTYIYRLLTVNKSSLSEKGR | | |
| | | QEDAEEYLGFILNGLHEEMLNL | | |
| | | KKLLSPSNEKLTISNGPKNHSV | | |
| | | NEEEQEEQGEGSEDEWEQVGPR | | |
| | | NKTSVTRQADFVQT | | |
| | | PITGIFGGHIRSVVYQQSSKES | | |
| | | ATLQPFFTLQLDIQSDKIRTVQ | | |
| | | DALESLVARESVQGYTTKTKQE | | |
| | | VEISRRVTLEKLPPVLVLHLKR | | |
| | | FVYEKTGGCQKLIKNIEYPVDL | | |
| | | EISKELLSPGVKNKNFKCHRTY | | |
| | | RLFAVVYHHGNSATGGHYTTDV | | |
| | | FQIGLNGWLRIDDQTVKVINQY | | |
| | | QVVKPTAERTAYLLYYRRVDLL | | |
| UBP49_HUMAN | 29 | MDRCKHVGRLRLAQDHSILNPQ | 141 | MDRCKHVGRLRLAQDHSILN |
| Ubiquitin | | KWCCLECATTESVWACLKCSHV | | PQKWCCLECATTESVWACLK |
| carboxyl- | | ACGRYIEDHALKHFEETGHPLA | | CSHVACGRYIEDHALKHFEE |
| terminal | | MEVRDLYVFCYLCKDYVLNDNP | | TGHPLAMEVRDLYVFCYLCK |
| hydrolase 49 | | EGDLKLLRSSLLAVRGQKQDTP | | DYVLNDNPEGDLKLLRSSLL |
| | | VRRGRTLRSMASGEDVVLPQRA | | AVRGQKQDTPVRRGRTLRSM |
| | | PQGQPQMLTALWYRRQRLLART | | ASGEDVVLPQRAPQGQPQML |
| | | LRLWFEKSSRGQAKLEQRRQEE | | TALWYRRQRLLARTLRLWFE |
| | | ALERKKEEARRRRREVKRRLLE | | KSSRGQAKLEQRRQEEALER |
| | | ELASTPPRKSARLLLHTPRDAG | | KKEEARRRRREVKRRLLEEL |
| | | PAASRPAALPTSRRVPAATLKL | | ASTPPRKSARLLLHTPRDAG |
| | | RRQPAMAPGVTGLRNLGNTCYM | | PAASRPAALPTSRRVPAATL |
| | | NSILQVLSHLQKFRECFLNLDP | | KLRRQPAMAPGVTGLRNLGN |
| | | SKTEHLFPKATNGK | | TCYMNSILQVLSHLQKFREC |
| | | TQLSGKPTNSSATELSLRNDRA | | FLNLDPSKTEHLFPKATNGK |
| | | EACEREGFCWNGRASISRSLEL | | TQLSGKPTNSSATELSLRND |
| | | IQNKEPSSKHISLCRELHTLER | | RAEACEREGFCWNGRASISR |
| | | VMWSGKWALVSPFAMLHSVWSL | | SLELIQNKEPSSKHISLCRE |
| | | IPAFRGYDQQDAQEFLCELLHK | | LHTLFRVMWSGKWALVSPFA |
| | | VQQELESEGTTRRILIPFSQRK | | MLHSVWSLIPAFRGYDQQDA |
| | | LTKQVLKVVNTIFHGQLLSQVT | | QEFLCELLHKVQQELESEGT |
| | | CISCNYKSNTIEPFWDLSLEEP | | TRRILIPFSQRKLTKQVLKV |
| | | ERYHCIEKGFVPLNQTECLLTE | | VNTIFHGQLLSQVTCISCNY |
| | | MLAKFTETEALEGRIYACDQCN | | KSNTIEPFWDLSLEFPERYH |
| | | SKRRKSNPKPLVLSEARKQLMI | | CIEKGFVPLNQTECLLTEML |
| | | YRLPQVLRLHLKRFRWSGRNHR | | AKFTETEALEGRIYACDQCN |
| | | EKIGVHVVEDQVLTMEPYCCRD | | SKRRKSNPKPLVLSEARKQL |
| | | MLSSLDKETFAYDL | | MIYRLPQVLRLHLKRFRWSG |
| | | SAVVMHHGKGFGSGHYTAYCYN | | RNHREKIGVHVVEDQVLTME |
| | | TEGGFWVHCNDSKLNVCSVEEV | | PYCCRDMLSSLDKETFAYDL |
| | | CKTQAYILFYTQRTVQGNARIS | | SAVVMHHGKGFGSGHYTAYC |
| | | ETHLQAQVQSSNNDEGRPQTES | | YNTEGGFWVHCNDSKLNVCS |
| | | | | VEEVCKTQAYILFYTQRT |
| U17L8_HUMAN | 30 | MEDDSLYLGGEWQFNHESKLTS | 142 | AVGAGLQNMGNTCYLNASLQ |
| Inactive | | PRPDAAFAEIQRTSLPEKSPLS | | CLTYTPPLANYMLSREHSQT |
| ubiquitin | | SETRVDLCDDLAPVARQLAPRE | | CQRPKCCMLCTMQAHITWAL |
| carboxyl- | | KLPLSSRRPAAVGAGLQNMGNT | | HSPGHVIQPSQALAAGFHRG |
| terminal | | CYLNASLQCLTYTPPLANYMLS | | KQEDAHEFLMFTVDAMKKAC |
| hydrolase 17- | | REHSQTCQRPKCCMLCTMQAHI | | LPGHKQVDHHSKDTTLIHQI |
| like protein 8 | | TWALHSPGHVIQPSQALAAGFH | | FGGCWRSQIKCLHCHGISDT |
| | | RGKQEDAHEFLMFTVDAMKKAC | | FDPYLDIALDIQAAQSVKQA |
| | | LPGHKQVDHHSKDTTLIHQIFG | | LEQLVKPEELNGENAYPCGL |
| | | GCWRSQIKCLHCHGISDTEDPY | | CLQRAPASNTLTLHTSAKVL |
| | | LDIALDIQAAQSVKQALEQLVK | | ILVLKRFCDVTGNKLAKNVQ |
| | | PEELNGENAYPCGLCLQRAPAS | | YPECLDMQPYMSQQNTGPLV |
| | | NTLTLHTSAKVLILVLKRFCDV | | YVLYAVLVHAGWSCHNGYYF |
| | | TGNKLAKNVQYPEC | | SYVKAQEGQWYKMDDAEVTA |
| | | LDMQPYMSQQNTGPLVYVLYAV | | CSITSVLSQQAYVLFYIQKS |
| | | LVHAGWSCHNGYYFSYVKAQEG | | |
| | | QWYKMDDAEVTACSITSVLSQQ | | |
| | | AYVLFYIQKSEWERHSESVSRG | | |
| | | REPRALGAEDTDRPATQGELKR | | |
| | | DHPCLQVPELDEHLVERATEES | | |
| | | TLDHWKFPQEQNKMKPEFNVRK | | |
| | | VEGTLPPNVLVIHQSKYKCGMK | | |
| | | NHHPEQQSSLLNLSSMNSTDQE | | |
| | | SMNTGTLASLQGRTRRSKGKNK | | |
| | | HSKRSLLVCQ | | |
| 6VN6_1 | 31 | GSKKHTGYVGLKNQGATCYMNS | 143 | TGYVGLKNQGATCYMNSLLQ |
| | | LLQTLFFTNQLRKAVYMMPTEG | | TLFFTNQLRKAVYMMPTEGD |
| | | DDSSKSVPLALQRVFYELQHSD | | DSSKSVPLALQRVFYELQHS |
| | | KPVGTKKLTKSFGWETLDSFMQ | | DKPVGTKKLTKSFGWETLDS |
| | | HDVQELCRVLLDNVENKMKGTC | | FMQHDVQELCRVLLDNVENK |
| | | VEGTIPKLFRGKMVSYIQCKEV | | MKGTCVEGTIPKLFRGKMVS |
| | | DYRSDRREDYYDIQLSIKGKKN | | YIQCKEVDYRSDRREDYYDI |
| | | IFESFVDYVAVEQLDGDNKYDA | | QLSIKGKKNIFESFVDYVAV |
| | | GEHGLQEAEKGVKFLTLPPVLH | | EQLDGDNKYDAGEHGLQEAE |
| | | LQLMRFMYDPQTDQNIKINDRE | | KGVKFLTLPPVLHLQLMREM |
| | | EFPEQLPLDEFLQKTDPKDPAN | | YDPQTDQNIKINDRFEFPEQ |
| | | YILHAVLVHSGDNHGGHYVVYL | | LPLDEFLQKTDPKDPANYIL |
| | | NPKGDGKWCKFDDDVVSRCTKE | | HAVLVHSGDNHGGHYVVYLN |
| | | EAIEHNYGGHDDDLSVRHCTNA | | PKGDGKWCKFDDDVVSRCTK |
| | | YMLVYIRESKLSEVLQAVTDHD | | EEAIEHNYGGHDDDLSVRHC |
| | | IPQQLVERLQEEKRIEAQKR | | TNAYMLVYIRE |
| 6DGF_1 | 32 | AQGLAGLRNLGNTCEMNSILQC | 144 | AQGLAGLRNLGNTCEMNSIL |
| | | LSNTRELRDYCLQRLYMRDLHH | | QCLSNTRELRDYCLQRLYMR |
| | | GSNAHTALVEEFAKLIQTIWTS | | DLHHGSNAHTALVEEFAKLI |
| | | SPNDVVSPSEFKTQIQRYAPRE | | QTIWTSSPNDVVSPSEFKTQ |
| | | VGYNQQDAQEFLRFLLDGLHNE | | IQRYAPRFVGYNQQDAQEFL |
| | | VNRVTLRPKSNPENLDHLPDDE | | RFLLDGLHNEVNRVTLRPKS |
| | | KGRQMWRKYLEREDSRIGDLFV | | NPENLDHLPDDEKGRQMWRK |
| | | GQLKSSLTCTDCGYCSTVEDPF | | YLEREDSRIGDLFVGQLKSS |
| | | WDLSLPIAKRGYPEVTLMDCMR | | LTCTDCGYCSTVEDPEWDLS |
| | | LFTKEDVLDGDEKPTCCRCRGR | | LPIAKRGYPEVTLMDCMRLF |
| | | KRCIKKFSIQRFPKILVLHLKR | | TKEDVLDGDEKPTCCRCRGR |
| | | FSESRIRTSKLTTFVNFPLRDL | | KRCIKKFSIQRFPKILVLHL |
| | | DLREFASENTNHAVYNLYAVSN | | KRFSESRIRTSKLTTFVNFP |
| | | HSGTTMGGHYTAYCRSPGTGEW | | LRDLDLREFASENTNHAVYN |
| | | HTENDSSVTPMSSSQVRTSDAY | | LYAVSNHSGTTMGGHYTAYC |
| | | LLFYELASPPSRM | | RSPGTGEWHTENDSSVTPMS |
| | | | | SSQVRTSDAYLLFYELAS |
| 2VHF_1 | 33 | GLEIMIGKKKGIQGHYNSCYLD | 145 | MIGKKKGIQGHYNSCYLDST |
| | | STLFCLFAFSSVLDTVLLRPKE | | LFCLFAFSSVLDTVLLRPKE |
| | | KNDVEYYSETQELLRTEIVNPL | | KNDVEYYSETQELLRTEIVN |
| | | RIYGYVCATKIMKLRKILEKVE | | PLRIYGYVCATKIMKLRKIL |
| | | AASGFTSEEKDPEEFLNILFHH | | EKVEAASGFTSEEKDPEEFL |
| | | ILRVEPLLKIRSAGQKVQDCYF | | NILFHHILRVEPLLKIRSAG |
| | | YQIFMEKNEKVGVPTIQQLLEW | | QKVQDCYFYQIFMEKNEKVG |
| | | SFINSNLKFAEAPSCLIIQMPR | | VPTIQQLLEWSFINSNLKFA |
| | | FGKDFKLEKKIFPSLELNITDL | | EAPSCLIIQMPREGKDFKLE |
| | | LEDTPRQCRICGGLAMYECREC | | KKIFPSLELNITDLLEDTPR |
| | | YDDPDISAGKIKQFCKTCNTQV | | QCRICGGLAMYECRECYDDP |
| | | HLHPKRLNHKYNPVSLPKDLPD | | DISAGKIKQFCKTCNTQVHL |
| | | WDWRHGCIPCQNMELFAVLCIE | | HPKRLNHKYNPVSLPKDLPD |
| | | TSHYVAFVKYGKDDSAWLFFDS | | WDWRHGCIPCQNMELFAVLC |
| | | MADRDGGQNGFNIPQVTPCPEV | | IETSHYVAFVKYGKDDSAWL |
| | | GEYLKMSLEDLHSLDSRRIQGC | | FFDSMADRDGGQNGFNIPQV |
| | | ARRLLCDAYMCMYQSPTMSLYK | | TPCPEVGEYLKMSLEDLHSL |
| | | | | DSRRIQGCARRLLCDAYMCM |
| | | | | YQS |
| U17LI_HUMAN | 34 | MEDDSLYLGGEWQFNHFSKLTS | 146 | AVGAGLQNMGNTCYVNASLQ |
| Ubiquitin | | SRPDAAFAEIQRTSLPEKSPLS | | CLTYTPPLANYMLSREHSQT |
| carboxyl- | | CETRVDLCDDLAPVARQLAPRE | | CHRHKGCMLCTMQAHITRAL |
| terminal | | KLPLSSRRPAAVGAGLQNMGNT | | HNPGHVIQPSQALAAGFHRG |
| hydrolase 17- | | CYVNASLQCLTYTPPLANYMLS | | KQEDAHEFLMFTVDAMKKAC |
| like protein 18 | | REHSQTCHRHKGCMLCTMQAHI | | LPGHKQVDHHSKDTTLIHQI |
| | | TRALHNPGHVIQPSQALAAGFH | | FGGYWRSQIKCLHCHGISDT |
| | | RGKQEDAHEFLMFTVDAMKKAC | | FDPYLDIALDIQAAQSVQQA |
| | | LPGHKQVDHHSKDTTLIHQIFG | | LEQLVKPEELNGENAYHCGV |
| | | GYWRSQIKCLHCHGISDTEDPY | | CLQRAPASKTLTLHTSAKVL |
| | | LDIALDIQAAQSVQQALEQLVK | | ILVLKRFSDVTGNKIAKNVQ |
| | | PEELNGENAYHCGVCLQRAPAS | | YPECLDMQPYMSQTNTGPLV |
| | | KTLTLHTSAKVLILVLKRESDV | | YVLYAVLVHAGWSCHNGHYF |
| | | TGNKIAKNVQYPEC | | SYVKAQEGQWYKMDDAEVTA |
| | | LDMQPYMSQTNTGPLVYVLYAV | | SSITSVLSQQAYVLFYIQKS |
| | | LVHAGWSCHNGHYFSYVKAQEG | | |
| | | QWYKMDDAEVTASSITSVLSQQ | | |
| | | AYVLFYIQKSEWERHSESVSRG | | |
| | | REPRALGAEDTDRRAKQGELKR | | |
| | | DHPCLQAPELDEHLVERATQES | | |
| | | TLDHWKFLQEQNKTKPEFNVRK | | |
| | | VEGTLPPDVLVIHQSKYKCGMK | | |
| | | NHHPEQQSSLLNLSSTTPTHQE | | |
| | | SMNTGTLASLRGRARRSKGKNK | | |
| | | HSKRALLVCQ | | |
| UBP22_HUMAN | 35 | MVSRPEPEGEAMDAELAVAPPG | 147 | LGNTCFMNCIVQALTHTPLL |
| Ubiquitin | | CSHLGSFKVDNWKQNLRAIYQC | | RDFFLSDRHRCEMQSPSSCL |
| carboxyl- | | FVWSGTAEARKRKAKSCICHVC | | VCEMSSLFQEFYSGHRSPHI |
| terminal | | GVHLNRLHSCLYCVFFGCFTKK | | PYKLLHLVWTHARHLAGYEQ |
| hydrolase 22 | | HIHEHAKAKRHNLAIDLMYGGI | | QDAHEFLIAALDVLHRHCKG |
| | | YCFLCQDYIYDKDMEIIAKEEQ | | DDNGKKANNPNHCNCIIDQI |
| | | RKAWKMQGVGEKESTWEPTKRE | | FTGGLQSDVTCQVCHGVSTT |
| | | LELLKHNPKRRKITSNCTIGLR | | IDPFWDISLDLPGSSTPFWP |
| | | GLINLGNTCEMNCIVQALTHTP | | LSPGSEGNVVNGESHVSGTT |
| | | LLRDFFLSDRHRCEMQSPSSCL | | TLTDCLRRFTRPEHLGSSAK |
| | | VCEMSSLFQEFYSGHRSPHIPY | | IKCSGCHSYQESTKQLTMKK |
| | | KLLHLVWTHARHLAGYEQQDAH | | LPIVACFHLKRFEHSAKLRR |
| | | EFLIAALDVLHRHCKGDDNGKK | | KITTYVSFPLELDMTPEMAS |
| | | ANNPNHCNCIIDQIFTGGLQSD | | SKESRMNGQYQQPTDSLNND |
| | | VTCQVCHGVSTTIDPFWDISLD | | NKYSLFAVVNHQGTLESGHY |
| | | LPGSSTPFWPLSPGSEGNVVNG | | TSFIRQHKDQWFKCDDAIIT |
| | | ESHVSGTTTLTDCLRRFTRPEH | | KASIKDVLDSEGYLLFYHKQ |
| | | LGSSAKIKCSGCHSYQESTKQL | | E |
| | | TMKKLPIVACFHLKRFEHSAKL | | |
| | | RRKITTYVSFPLELDMTPEMAS | | |
| | | SKESRMNGQYQQPTDSLNNDNK | | |
| | | YSLFAVVNHQGTLESGHYTSFI | | |
| | | RQHKDQWFKCDDAIITKASIKD | | |
| | | VLDSEGYLLFYHKQFLEYE | | |
| UBP18_HUMAN | 36 | MSKAFGLLRQICQSILAESSQS | 148 | KGLVPGLVNLGNTCEMNSLL |
| Ubl | | PADLEEKKEEDSNMKREQPRER | | QGLSACPAFIRWLEEFTSQY |
| carboxyl- | | PRAWDYPHGLVGLHNIGQTCCL | | SRDQKEPPSHQYLSLTLLHL |
| terminal | | NSLIQVFVMNVDFTRILKRITV | | LKALSCQEVTDDEVLDASCL |
| hydrolase 18 | | PRGADEQRRSVPFQMLLLLEKM | | LDVLRMYRWQISSFEEQDAH |
| | | QDSRQKAVRPLELAYCLQKCNV | | ELFHVITSSLEDERDRQPRV |
| | | PLFVQHDAAQLYLKLWNLIKDQ | | THLFDVHSLEQQSEITPKQI |
| | | ITDVHLVERLQALYTIRVKDSL | | TCRTRGSPHPTSNHWKSQHP |
| | | ICVDCAMESSRNSSMLTLPLSL | | FHGRLTSNMVCKHCEHQSPV |
| | | FDVDSKPLKTLEDALHCFFQPR | | RFDTFDSLSLSIPAATWGHP |
| | | ELSSKSKCFCENCGKKTRGKQV | | LTLDHCLHHFISSESVRDVV |
| | | LKLTHLPQTLTIHLMRESIRNS | | CDNCTKIEAKGTLNGEKVEH |
| | | QTRKICHSLYFPQSLDESQILP | | QRTTFVKQLKLGKLPQCLCI |
| | | MKRESCDAEEQSGG | | HLQRLSWSSHGTPLKRHEHV |
| | | QYELFAVIAHVGMADSGHYCVY | | QFNEFLMMDIYKYHLLGHKP |
| | | IRNAVDGKWFCENDSNICLVSW | | SQHNPKLNKNPGPTLELQDG |
| | | EDIQCTYGNPNYHWQETAYLLV | | PGAPTPVLNQPGAPKTQIFM |
| | | YMKMEC | | NGACSPSLLPTLSAPMPFPL |
| | | | | PVVPDYSSSTYLERLMAVVV |
| | | | | HHGDMHSGHFVTYRRSPPSA |
| | | | | RNPLSTSNQWLWVSDDTVRK |
| | | | | ASLQEVLSSSAYLLEYERVL |
| UBP28_HUMAN | 37 | MTAELQQDDAAGAADGHGSSCQ | 149 | GWPVGLKNVGNTCWFSAVIQ |
| Ubiquitin | | MLLNQLREITGIQDPSFLHEAL | | SLFQLPEFRRLVLSYSLPQN |
| carboxyl- | | KASNGDITQAVSLLTDERVKEP | | VLENCRSHTEKRNIMEMQEL |
| terminal | | SQDTVATEPSEVEGSAANKEVL | | QYLFALMMGSNRKFVDPSAA |
| hydrolase 28 | | AKVIDLTHDNKDDLQAAIALSL | | LDLLKGAFRSSEEQQQDVSE |
| | | LESPKIQADGRDLNRMHEATSA | | FTHKLLDWLEDAFQLAVNVN |
| | | ETKRSKRKRCEVWGENPNPNDW | | SPRNKSENPMVQLFYGTELT |
| | | RRVDGWPVGLKNVGNTCWFSAV | | EGVREGKPFCNNETFGQYPL |
| | | IQSLFQLPEFRRLVLSYSLPQN | | QVNGYRNLDECLEGAMVEGD |
| | | VLENCRSHTEKRNIMFMQELQY | | VELLPSDHSVKYGQERWFTK |
| | | LFALMMGSNRKFVDPSAALDLL | | LPPVLTFELSRFEFNQSLGQ |
| | | KGAFRSSEEQQQDVSEFTHKLL | | PEKIHNKLEFPQIIYMDRYM |
| | | DWLEDAFQLAVNVNSPRNKSEN | | YRSKELIRNKRECIRKLKEE |
| | | PMVQLFYGTELTEG | | IKILQQKLERYVKYGSGPAR |
| | | VREGKPFCNNETFGQYPLQVNG | | FPLPDMLKYVIEFASTKPAS |
| | | YRNLDECLEGAMVEGDVELLPS | | ESCPPESDTHMTLPLSSVHC |
| | | DHSVKYGQERWFTKLPPVLTFE | | SVSDQTSKESTSTESSSQDV |
| | | LSRFEFNQSLGQPEKIHNKLEF | | ESTESSPEDSLPKSKPLTSS |
| | | PQIIYMDRYMYRSKELIRNKRE | | RSSMEMPSQPAPRTVTDEEI |
| | | CIRKLKEEIKILQQKLERYVKY | | NFVKTCLQRWRSEIEQDIQD |
| | | GSGPARFPLPDMLKYVIEFAST | | LKTCIASTTQTIEQMYCDPL |
| | | KPASESCPPESDTHMTLPLSSV | | LRQVPYRLHAVLVHEGQANA |
| | | HCSVSDQTSKESTSTESSSQDV | | GHYWAYIYNQPRQSWLKYND |
| | | ESTESSPEDSLPKSKPLTSSRS | | ISVTESSWEEVERDSYGGLR |
| | | SMEMPSQPAPRTVTDEEINFVK | | NVSAYCLMYINDKLPY |
| | | TCLQRWRSEIEQDIQDLKTCIA | | |
| | | STTQTIEQMYCDPLLRQVPYRL | | |
| | | HAVLVHEGQANAGHYWAYIYNQ | | |
| | | PRQSWLKYNDISVTESSWEEVE | | |
| | | RDSYGGLRNVSAYCLMYINDKL | | |
| | | PYFNAEAAPTESDQMSEVEALS | | |
| | | VELKHYIQEDNWRFEQEVEEWE | | |
| | | EEQSCKIPQMESSINSSSQDYS | | |
| | | TSQEPSVASSHGVRCLSSEHAV | | |
| | | IVKEQTAQAIANTARAYEKSGV | | |
| | | EAALSEVMLSPAMQGVILAIAK | | |
| | | ARQTFDRDGSEAGLIKAFHEEY | | |
| | | SRLYQLAKETPTSHSDPRLQHV | | |
| | | LVYFFQNEAPKRVVERTLLEQF | | |
| | | ADKNLSYDERSISIMKVAQAKL | | |
| | | KEIGPDDMNMEEYKKWHEDYSL | | |
| | | FRKVSVYLLTGLELYQKGKYQE | | |
| | | ALSYLVYAYQSNAALLMKGPRR | | |
| | | GVKESVIALYRRKCLLELNAKA | | |
| | | ASLFETNDDHSVTEGINVMNEL | | |
| | | IIPCIHLIINNDISKDDLDAIE | | |
| | | VMRNHWCSYLGQDIAENLQLCL | | |
| | | GEFLPRLLDPSAEIIVLKEPPT | | |
| | | IRPNSPYDLCSRFAAVMESIQG | | |
| | | VSTVTVK | | |
| | | MEDDSLYLGGEWQFNHFSKLTS | | |
| | | SRPDAAFAEIQRTSLPEKSPLS | | |
| | | SEARVDLCDDLAPVARQLAPRK | | |
| | | KLPLSSRRPAAVGAGLQNMGNT | | |
| | | CYENASLQCLTYTPPLANYMLS | | |
| U17L2_HUMAN | 38 | REHSQTCQRPKCCMLCTMQAHI | 150 | AVGAGLQNMGNTCYENASLQ |
| Ubiquitin | | TWALHSPGHVIQPSQALAAGFH | | CLTYTPPLANYMLSREHSQT |
| carboxyl- | | RGKQEDAHEFLMFTVDAMKKAC | | CQRPKCCMLCTMQAHITWAL |
| terminal | | LPGHKQVDHHSKDTTLIHQIFG | | HSPGHVIQPSQALAAGFHRG |
| hydrolase 17 | | GCWRSQIKCLHCHGISDTFDPY | | KQEDAHEFLMFTVDAMKKAC |
| | | LDIALDIQAAQSVKQALEQLVK | | LPGHKQVDHHSKDTTLIHQI |
| | | PEELNGENAYHCGLCLQRAPAS | | FGGCWRSQIKCLHCHGISDT |
| | | KTLTLHTSAKVLILVLKRESDV | | FDPYLDIALDIQAAQSVKQA |
| | | TGNKLAKNVQYPEC | | LEQLVKPEELNGENAYHCGL |
| | | LDMQPYMSQQNTGPLVYVLYAV | | CLQRAPASKTLTLHTSAKVL |
| | | LVHAGWSCHDGHYFSYVKAQEG | | ILVLKRFSDVTGNKLAKNVQ |
| | | QWYKMDDAKVTACSITSVLSQQ | | YPECLDMQPYMSQQNTGPLV |
| | | AYVLFYIQKSEWERHSESVSRG | | YVLYAVLVHAGWSCHDGHYF |
| | | REPRALGAEDTDRRATQGELKR | | SYVKAQEGQWYKMDDAKVTA |
| | | DHPCLQAPELDERLVERATQES | | CSITSVLSQQAYVLFYIQKS |
| | | TLDHWKFPQEQNKTKPEFNVRK | | |
| | | VEGTLPPNVLVIHQSKYKCGMK | | |
| | | NHHPEQQSSLLNLSSTTRTDQE | | |
| | | SVNTGTLASLQGRTRRSKGKNK | | |
| | | HSKRALLVCQ | | |
| UBP31_HUMAN | 39 | MSKVTAPGSGPPAAASGKEKRS | 151 | PVPGVAGLRNHGNTCEMNAT |
| Ubiquitin | | FSKRLERSGRAGGGGAGGPGAS | | LQCLSNTELFAEYLALGQYR |
| carboxyl- | | GPAAPSSPSSPSSARSVGSEMS | | AGRPEPSPDPEQPAGRGAQG |
| terminal | | RVLKTLSTLSHLSSEGAAPDRG | | QGEVTEQLAHLVRALWTLEY |
| hydrolase 31 | | GLRSCFPPGPAAAPTPPPCPPP | | TPQHSRDFKTIVSKNALQYR |
| | | PASPAPPACAAEPVPGVAGLRN | | GNSQHDAQEFLLWLLDRVHE |
| | | HGNTCFMNATLQCLSNTELFAE | | DLNHSVKQSGQPPLKPPSET |
| | | YLALGQYRAGRPEPSPDPEQPA | | DMMPEGPSFPVCSTEVQELE |
| | | GRGAQGQGEVTEQLAHLVRALW | | QAQYRSSLTCPHCQKQSNTF |
| | | TLEYTPQHSRDFKTIVSKNALQ | | DPFLCISLPIPLPHTRPLYV |
| | | YRGNSQHDAQEFLLWLLDRVHE | | TVVYQGKCSHCMRIGVAVPL |
| | | DLNHSVKQSGQPPLKPPSETDM | | SGTVARLREAVSMETKIPTD |
| | | MPEGPSFPVCSTFVQELFQAQY | | QIVLTEMYYDGFHRSFCDTD |
| | | RSSLTCPHCQKQSN | | DLETVHESDCIFAFETPEIF |
| | | TFDPFLCISLPIPLPHTRPLYV | | RPEGILSQRGIHLNNNLNHL |
| | | TVVYQGKCSHCMRIGVAVPLSG | | KFGLDYHRLSSPTQTAAKQG |
| | | TVARLREAVSMETKIPTDQIVL | | KMDSPTSRAGSDKIVLLVCN |
| | | TEMYYDGFHRSFCDTDDLETVH | | RACTGQQGKRFGLPFVLHLE |
| | | ESDCIFAFETPEIFRPEGILSQ | | KTIAWDLLQKEILEKMKYFL |
| | | RGIHLNNNLNHLKFGLDYHRLS | | RPTVCIQVCPFSLRVVSVVG |
| | | SPTQTAAKQGKMDSPTSRAGSD | | ITYLLPQEEQPLCHPIVE |
| | | KIVLLVCNRACTGQQGKRFGLP | | RALKSCGPGGTAHVKLVVEW |
| | | FVLHLEKTIAWDLLQKEILEKM | | DKETRDFLFVNTEDEYIPDA |
| | | KYFLRPTVCIQVCPFSLRVVSV | | ESVRLQRERHHQPQTCTLSQ |
| | | VGITYLLPQEEQPLCHPIVERA | | CFQLYTKEERLAPDDAWRCP |
| | | LKSCGPGGTAHVKLVVEWDKET | | HCKQLQQGSITLSLWTLPDV |
| | | RDFLFVNTEDEYIPDAESVRLQ | | LIIHLKRFRQEGDRRMKLQN |
| | | RERHHQPQTCTLSQ | | MVKFPLTGLDMTPHVVKRSQ |
| | | CFQLYTKEERLAPDDAWRCPHC | | SSWSLPSHWSPWRRPYGLGR |
| | | KQLQQGSITLSLWTLPDVLIIH | | DPEDYIYDLYAVCNHHGTMQ |
| | | LKRFRQEGDRRMKLQNMVKFPL | | GGHYTAYCKNSVDGLWYCFD |
| | | TGLDMTPHVVKRSQSSWSLPSH | | DSDVQQLSEDEVCTQTAYIL |
| | | WSPWRRPYGLGRDPEDYIYDLY | | FYQRRT |
| | | AVCNHHGTMQGGHYTAYCKNSV | | |
| | | DGLWYCFDDSDVQQLSEDEVCT | | |
| | | QTAYILFYQRRTAIPSWSANSS | | |
| | | VAGSTSSSLCEHWVSRLPGSKP | | |
| | | ASVTSAASSRRTSLASLSESVE | | |
| | | MTGERSEDDGGFSTRPFVRSVQ | | |
| | | RQSLSSRSSVTSPLAVNENCMR | | |
| | | PSWSLSAKLQMRSNSPSRFSGD | | |
| | | SPIHSSASTLEKIG | | |
| | | EAADDKVSISCFGSLRNLSSSY | | |
| | | QEPSDSHSRREHKAVGRAPLAV | | |
| | | MEGVFKDESDTRRLNSSVVDTQ | | |
| | | SKHSAQGDRLPPLSGPFDNNNQ | | |
| | | IAYVDQSDSVDSSPVKEVKAPS | | |
| | | HPGSLAKKPESTTKRSPSSKGT | | |
| | | SEPEKSLRKGRPALASQESSLS | | |
| | | STSPSSPLPVKVSLKPSRSRSK | | |
| | | ADSSSRGSGRHSSPAPAQPKKE | | |
| | | SSPKSQDSVSSPSPQKQKSASA | | |
| | | LTYTASSTSAKKASGPATRSPF | | |
| | | PPGKSRTSDHSLSREGSRQSLG | | |
| | | SDRASATSTSKPNSPRVSQARA | | |
| | | GEGRGAGKHVRSSS | | |
| | | MASLRSPSTSIKSGLKRDSKSE | | |
| | | DKGLSFFKSALRQKETRRSTDL | | |
| | | GKTALLSKKAGGSSVKSVCKNT | | |
| | | GDDEAERGHQPPASQQPNANTT | | |
| | | GKEQLVTKDPASAKHSLLSARK | | |
| | | SKSSQLDSGVPSSPGGRQSAEK | | |
| | | SSKKLSSSMQTSARPSQKPQ | | |
| U17LJ_HUMAN | 40 | MEEDSLYLGGEWQFNHESKLTS | 152 | AVGAGLQNMGNTCYVNASLQ |
| Ubiquitin | | SRPDAAFAEIQRTSLPEKSPLS | | CLTYTPPLANYMLSREHSQT |
| carboxyl- | | CETRVDLCDDLAPVARQLAPRE | | CHRHKGCMLCTMQAHITRAL |
| terminal | | KLPLSSRRPAAVGAGLQNMGNT | | HNPGHVIQPSQALAAGFHRG |
| hydrolase 17- | | CYVNASLQCLTYTPPLANYMLS | | KQEDAHEFLMFTVDAMKKAC |
| like protein 19 | | REHSQTCHRHKGCMLCTMQAHI | | LPGHKQVDHHSKDTTLIHQI |
| | | TRALHNPGHVIQPSQALAAGFH | | FGGYWRSQIKCLHCHGISDT |
| | | RGKQEDAHEFLMFTVDAMKKAC | | FDPYLDIALDIQAAQSVQQA |
| | | LPGHKQVDHHSKDTTLIHQIFG | | LEQLVKPEELNGENAYHCGV |
| | | GYWRSQIKCLHCHGISDTFDPY | | CLQRAPASKTLTLHTSAKVL |
| | | LDIALDIQAAQSVQQALEQLVK | | ILVLKRFSDVTGNKIAKNVQ |
| | | PEELNGENAYHCGVCLQRAPAS | | YPECLDMQPYMSQTNTGPLV |
| | | KTLTLHTSAKVLILVLKRFSDV | | YVLYAVLVHAGWSCHNGHYF |
| | | TGNKIAKNVQYPEC | | SYVKAQEGQWYKMDDAEVTA |
| | | LDMQPYMSQTNTGPLVYVLYAV | | SSITSVLSQQAYVLFYIQKS |
| | | LVHAGWSCHNGHYFSYVKAQEG | | EWERHSESVSRGREPRALGA |
| | | QWYKMDDAEVTASSITSVLSQQ | | EDTDRRATQGELKRDHPCLQ |
| | | AYVLFYIQKSEWERHSESVSRG | | APEL |
| | | REPRALGAEDTDRRATQGELKR | | |
| | | DHPCLQAPELDEHLVERATQES | | |
| | | TLDHWKFLQEQNKTKPEFNVRK | | |
| | | VEGTLPPDVLVIHQSKYKCGMK | | |
| | | NHHPEQQSSLLKLSSTTPTHQE | | |
| | | SMNTGTLASLRGRARRSKGKNK | | |
| | | HSKRALLVCQ | | |
| U17LF_HUMAN | 41 | MEDDSLYLGGEWQFNHESKLTS | 153 | AVGAGLQNMGNTCYVNASLQ |
| Ubiquitin | | SRPDAAFAEIQRTSLPEKSPLS | | CLTYTPPLANYMLSREHSQT |
| carboxyl- | | CETRVDLCDDLAPVARQLAPRE | | CHRHKGCMLCTMQAHITRAL |
| terminal | | KLPLSSRRPAAVGAGLQNMGNT | | HNPGHVIQPSQALAAGFHRG |
| hydrolase 17- | | CYVNASLQCLTYTPPLANYMLS | | KQEDAHEFLMFTVDAMKKAC |
| like protein 15 | | REHSQTCHRHKGCMLCTMQAHI | | LPGHKQVDHHSKDTTLIHQI |
| | | TRALHNPGHVIQPSQALAAGEH | | FGGYWRSQIKCLHCHGISDT |
| | | RGKQEDAHEFLMFTVDAMKKAC | | FDPYLDIALDIQAAQSVQQA |
| | | LPGHKQVDHHSKDTTLIHQIFG | | LEQLVKPEELNGENAYHCGV |
| | | GYWRSQIKCLHCHGISDTEDPY | | CLQRAPASKTLTLHTSAKVL |
| | | LDIALDIQAAQSVQQALEQLVK | | ILVLKRFSDVTGNKIDKNVQ |
| | | PEELNGENAYHCGVCLQRAPAS | | YPECLDMKLYMSQTNSGPLV |
| | | KTLTLHTSAKVLILVLKRESDV | | YVLYAVLVHAGWSCHNGHYF |
| | | TGNKIDKNVQYPEC | | SYVKAQEGQWYKMDDAEVTA |
| | | LDMKLYMSQTNSGPLVYVLYAV | | SSITSVLSQQAYVLFYIQKS |
| | | LVHAGWSCHNGHYFSYVKAQEG | | |
| | | QWYKMDDAEVTASSITSVLSQQ | | |
| | | AYVLFYIQKSEWERHSESVSRG | | |
| | | REPRALGAEDTDRRATQGELKR | | |
| | | DHPCLQAPELDEHLVERATQES | | |
| | | TLDHWKFLQEQNKTKPEFNVRK | | |
| | | VEGTLPPDVLVIHQSKYKCGMK | | |
| | | NHHPEQQSSLLNLSSTTPTHQE | | |
| | | SMNTGTLASLRGRARRSKGKNK | | |
| | | HSKRALLVCQWSQWKYRPTRRG | | |
| | | AHTHAHTQTHT | | |
| UBP47_HUMAN | 42 | MVPGEENQLVPKEDVEWRCRQN | 154 | ETGYVGLVNQAMTCYLNSLL |
| Ubiquitin | | IFDEMKKKFLQIENAAEEPRVL | | QTLEMTPEFRNALYKWEFEE |
| carboxyl- | | CIIQDTTNSKTVNERITLNLPA | | SEEDPVTSIPYQLQRLFVLL |
| terminal | | STPVRKLFEDVANKVGYINGTF | | QTSKKRAIETTDVTRSFGWD |
| hydrolase 47 | | DLVWGNGINTADMAPLDHTSDK | | SSEAWQQHDVQELCRVMEDA |
| | | SLLDANFEPGKKNFLHLTDKDG | | LEQKWKQTEQADLINELYQG |
| | | EQPQILLEDSSAGEDSVHDRFI | | KLKDYVRCLECGYEGWRIDT |
| | | GPLPREGSGGSTSDYVSQSYSY | | YLDIPLVIRPYGSSQAFASV |
| | | SSILNKSETGYVGLVNQAMTCY | | EEALHAFIQPEILDGPNQYF |
| | | LNSLLQTLFMTPEFRNALYKWE | | CERCKKKCDARKGLRFLHEP |
| | | FEESEEDPVTSIPYQLQRLFVL | | YLLTLQLKRFDFDYTTMHRI |
| | | LQTSKKRAIETTDVTRSFGWDS | | KLNDRMTFPEELDMSTFIDV |
| | | SEAWQQHDVQELCRVMEDALEQ | | EDEKSPQTESCTDSGAENEG |
| | | KWKQTEQADLINEL | | SCHSDQMSNDFSNDDGVDEG |
| | | YQGKLKDYVRCLECGYEGWRID | | ICLETNSGTEKISKSGLEKN |
| | | TYLDIPLVIRPYGSSQAFASVE | | SLIYELFSVMVHSGSAAGGH |
| | | EALHAFIQPEILDGPNQYFCER | | YYACIKSESDEQWYSENDQH |
| | | CKKKCDARKGLRFLHFPYLLTL | | VSRITQEDIKKTHGGSSGSR |
| | | QLKRFDEDYTTMHRIKLNDRMT | | GYYSSAFASSTNAYMLIYRL |
| | | FPEELDMSTFIDVEDEKSPQTE | | KD |
| | | SCTDSGAENEGSCHSDQMSNDE | | |
| | | SNDDGVDEGICLETNSGTEKIS | | |
| | | KSGLEKNSLIYELFSVMVHSGS | | |
| | | AAGGHYYACIKSESDEQWYSEN | | |
| | | DQHVSRITQEDIKKTHGGSSGS | | |
| | | RGYYSSAFASSTNAYMLIYRLK | | |
| | | DPARNAKFLEVDEYPEHIKNLV | | |
| | | QKERELEEQEKRQR | | |
| | | EIERNTCKIKLFCLHPTKQVMM | | |
| | | ENKLEVHKDKTLKEAVEMAYKM | | |
| | | MDLEEVIPLDCCRLVKYDEFHD | | |
| | | YLERSYEGEEDTPMGLLLGGVK | | |
| | | STYMEDLLLETRKPDQVFQSYK | | |
| | | PGEVMVKVHVVDLKAESVAAPI | | |
| | | TVRAYLNQTVTEFKQLISKAIH | | |
| | | LPAETMRIVLERCYNDLRLLSV | | |
| | | SSKTLKAEGFERSNKVFVESSE | | |
| | | TLDYQMAFADSHLWKLLDRHAN | | |
| | | TIRLFVLLPEQSPVSYSKRTAY | | |
| | | QKAGGDSGNVDDDCERVKGPVG | | |
| | | SLKSVEAILEESTEKLKSLSLQ | | |
| | | QQQDGDNGDSSKST | | |
| | | ETSDFENIESPLNERDSSASVD | | |
| | | NRELEQHIQTSDPENFQSEERS | | |
| | | DSDVNNDRSTSSVDSDILSSSH | | |
| | | SSDTLCNADNAQIPLANGLDSH | | |
| | | SITSSRRTKANEGKKETWDTAE | | |
| | | EDSGTDSEYDESGKSRGEMQYM | | |
| | | YFKAEPYAADEGSGEGHKWLMV | | |
| | | HVDKRITLAAFKQHLEPFVGVL | | |
| | | SSHFKVERVYASNQEFESVRLN | | |
| | | ETLSSFSDDNKITIRLGRALKK | | |
| | | GEYRVKVYQLLVNEQEPCKELL | | |
| | | DAVFAKGMTVRQSKEELIPQLR | | |
| | | EQCGLELSIDRERLRKKTWKNP | | |
| | | GTVELDYHIYEEDI | | |
| | | NISSNWEVELEVLDGVEKMKSM | | |
| | | SQLAVLSRRWKPSEMKLDPFQE | | |
| | | VVLESSSVDELREKLSEISGIP | | |
| | | LDDIEFAKGRGTFPCDISVLDI | | |
| | | HQDLDWNPKVSTLNVWPLYICD | | |
| | | DGAVIFYRDKTEELMELTDEQR | | |
| | | NELMKKESSRLQKTGHRVTYSP | | |
| | | RKEKALKIYLDGAPNKDLTQD | | |
| UBP51_HUMAN | 43 | MAQVRETSLPSGSGVRWISGGG | 155 | YTVGLRGLINLGNTCEMNCI |
| Ubiquitin | | GGASPEEAVEKAGKMEEAAAGA | | VQALTHIPLLKDFFLSDKHK |
| carboxyl- | | TKASSRREAEEMKLEPLQEREP | | CIMTSPSLCLVCEMSSLFHA |
| terminal | | APEENLTWSSSGGDEKVLPSIP | | MYSGSRTPHIPYKLLHLIWI |
| hydrolase 51 | | LRCHSSSSPVCPRRKPRPRPQP | | HAEHLAGYRQQDAHEFLIAI |
| | | RARSRSQPGLSAPPPPPARPPP | | LDVLHRHSKDDSGGQEANNP |
| | | PPPPPPPPAPRPRAWRGSRRRS | | NCCNCIIDQIFTGGLQSDVT |
| | | RPGSRPQTRRSCSGDLDGSGDP | | CQACHSVSTTIDPCWDISLD |
| | | GGLGDWLLEVEFGQGPTGCSHV | | LPGSCATFDSQNPERADSTV |
| | | ESFKVGKNWQKNLRLIYQREVW | | SRDDHIPGIPSLTDCLQWFT |
| | | SGTPETRKRKAKSCICHVCSTH | | RPEHLGSSAKIKCNSCQSYQ |
| | | MNRLHSCLSCVFFGCFTEKHIH | | ESTKQLTMKKLPIVACFHLK |
| | | KHAETKQHHLAVDLYHGVIYCF | | RFEHVGKQRRKINTFISFPL |
| | | MCKDYVYDKDIEQI | | ELDMTPFLASTKESRMKEGQ |
| | | AKETKEKILRLLTSTSTDVSHQ | | PPTDCVPNENKYSLFAVINH |
| | | QFMTSGFEDKQSTCETKEQEPK | | HGTLESGHYTSFIRQQKDQW |
| | | LVKPKKKRRKKSVYTVGLRGLI | | FSCDDAIITKATIEDLLYSE |
| | | NLGNTCFMNCIVQALTHIPLLK | | GYLLFYHKQG |
| | | DFFLSDKHKCIMTSPSLCLVCE | | |
| | | MSSLFHAMYSGSRTPHIPYKLL | | |
| | | HLIWIHAEHLAGYRQQDAHEFL | | |
| | | IAILDVLHRHSKDDSGGQEANN | | |
| | | PNCCNCIIDQIFTGGLQSDVTC | | |
| | | QACHSVSTTIDPCWDISLDLPG | | |
| | | SCATFDSQNPERADSTVSRDDH | | |
| | | IPGIPSLTDCLQWFTRPEHLGS | | |
| | | SAKIKCNSCQSYQESTKQLTMK | | |
| | | KLPIVACFHLKRFE | | |
| | | HVGKQRRKINTFISFPLELDMT | | |
| | | PFLASTKESRMKEGQPPTDCVP | | |
| | | NENKYSLFAVINHHGTLESGHY | | |
| | | TSFIRQQKDQWFSCDDAIITKA | | |
| | | TIEDLLYSEGYLLFYHKQGLEK | | |
| | | D | | |
| UBP36_HUMAN | 44 | MPIVDKLKEALKPGRKDSADDG | 156 | RVGAGLHNLGNTCELNATIQ |
| Ubiquitin | | ELGKLLASSAKKVLLQKIEFEP | | CLTYTPPLANYLLSKEHARS |
| carboxyl- | | ASKSFSYQLEALKSKYVLLNPK | | CHQGSFCMLCVMQNHIVQAF |
| terminal | | TEGASRHKSGDDPPARRQGSEH | | ANSGNAIKPVSFIRDLKKIA |
| hydrolase 36 | | TYESCGDGVPAPQKVLFPTERL | | RHFREGNQEDAHEFLRYTID |
| | | SLRWERVERVGAGLHNLGNTCF | | AMQKACLNGCAKLDRQTQAT |
| | | LNATIQCLTYTPPLANYLLSKE | | TLVHQIFGGYLRSRVKCSVC |
| | | HARSCHQGSFCMLCVMQNHIVQ | | KSVSDTYDPYLDVALEIRQA |
| | | AFANSGNAIKPVSFIRDLKKIA | | ANIVRALELFVKADVLSGEN |
| | | RHFREGNQEDAHEFLRYTIDAM | | AYMCAKCKKKVPASKRFTIH |
| | | QKACLNGCAKLDRQTQATTLVH | | RTSNVLTLSLKRFANFSGGK |
| | | QIFGGYLRSRVKCSVCKSVSDT | | ITKDVGYPEFLNIRPYMSQN |
| | | YDPYLDVALEIRQAANIVRALE | | NG |
| | | LFVKADVLSGENAY | | DPVMYGLYAVLVHSGYSCHA |
| | | MCAKCKKKVPASKRFTIHRTSN | | GHYYCYVKASNGQWYQMNDS |
| | | VLTLSLKRFANESGGKITKDVG | | LVHSSNVKVVLNQQAYVLFY |
| | | YPEFLNIRPYMSQNNGDPVMYG | | LRIP |
| | | LYAVLVHSGYSCHAGHYYCYVK | | |
| | | ASNGQWYQMNDSLVHSSNVKVV | | |
| | | LNQQAYVLFYLRIPGSKKSPEG | | |
| | | LISRTGSSSLPGRPSVIPDHSK | | |
| | | KNIGNGIISSPLTGKRQDSGTM | | |
| | | KKPHTTEEIGVPISRNGSTLGL | | |
| | | KSQNGCIPPKLPSGSPSPKLSQ | | |
| | | TPTHMPTILDDPGKKVKKPAPP | | |
| | | QHFSPRTAQGLPGTSNSNSSRS | | |
| | | GSQRQGSWDSRDVVLSTSPKLL | | |
| | | ATATANGHGLKGND | | |
| | | ESAGLDRRGSSSSSPEHSASSD | | |
| | | STKAPQTPRSGAAHLCDSQETN | | |
| | | CSTAGHSKTPPSGADSKTVKLK | | |
| | | SPVLSNTTTEPASTMSPPPAKK | | |
| | | LALSAKKASTLWRATGNDLRPP | | |
| | | PPSPSSDLTHPMKTSHPVVAST | | |
| | | WPVHRARAVSPAPQSSSRLQPP | | |
| | | FSPHPTLLSSTPKPPGTSEPRS | | |
| | | CSSISTALPQVNEDLVSLPHQL | | |
| | | PEASEPPQSPSEKRKKTFVGEP | | |
| | | QRLGSETRLPQHIREATAAPHG | | |
| | | KRKRKKKKRPEDTAASALQEGQ | | |
| | | TQRQPGSPMYRREGQAQLPAVR | | |
| | | RQEDGTQPQVNGQQ | | |
| | | VGCVTDGHHASSRKRRRKGAEG | | |
| | | LGEEGGLHQDPLRHSCSPMGDG | | |
| | | DPEAMEESPRKKKKKKRKQETQ | | |
| | | RAVEEDGHLKCPRSAKPQDAVV | | |
| | | PESSSCAPSANGWCPGDRMGLS | | |
| | | QAPPVSWNGERESDVVQELLKY | | |
| | | SSDKAYGRKVLTWDGKMSAVSQ | | |
| | | DAIEDSRQARTETVVDDWDEEF | | |
| | | DRGKEKKIKKEKREKRRNFNAF | | |
| | | QKLQTRRNEWSVTHPAKAASLS | | |
| | | YRR | | |
| UBP44_HUMAN | 45 | MLAMDTCKHVGQLQLAQDHSSL | 157 | TPGVTGLRNLGNTCYMNSVL |
| Ubiquitin | | NPQKWHCVDCNTTESIWACLSC | | QVLSHLLIFRQCFLKLDLNQ |
| carboxyl- | | SHVACGRYIEEHALKHFQESSH | | WLAMTASEKTRSCKHPPVTD |
| terminal | | PVALEVNEMYVFCYLCDDYVLN | | TVVYQMNECQEKDTGFVCSR |
| hydrolase 44 | | DNTTGDLKLLRRTLSAIKSQNY | | QSSLSSGLSGGASKGRKMEL |
| | | HCTTRSGRFLRSMGTGDDSYEL | | IQPKEPTSQYISLCHELHTL |
| | | HDGAQSLLQSEDQLYTALWHRR | | FQVMWSGKWALVSPFAMLHS |
| | | RILMGKIFRTWFEQSPIGRKKQ | | VWRLIPAFRGYAQQDAQEFL |
| | | EEPFQEKIVVKREVKKRRQELE | | CELLDKIQRELETTGTSLPA |
| | | YQVKAELESMPPRKSLRLQGLA | | LIPTSQRKLIKQVLNVVNNI |
| | | QSTIIEIVSVQVPAQTPASPAK | | FHGQLLSQVTCLACDNKSNT |
| | | DKVLSTSENEISQKVSDSSVKR | | IEPFWDLSLEFPERYQCSGK |
| | | RPIVTPGVTGLRNLGNTCYMNS | | DIASQPCLVTEMLAKFTETE |
| | | VLQVLSHLLIFRQC | | ALEGKIYVCDQCNSKRRRES |
| | | FLKLDLNQWLAMTASEKTRSCK | | SKPVVLTEAQKQLMICHLPQ |
| | | HPPVTDTVVYQMNECQEKDTGF | | VLRLHLKRFRWSGRNNREKI |
| | | VCSRQSSLSSGLSGGASKGRKM | | GVHVGFEEILNMEPYCCRET |
| | | ELIQPKEPTSQYISLCHELHTL | | LKSLRPECFIYDLSAVVMHH |
| | | FQVMWSGKWALVSPFAMLHSVW | | GKGFGSGHYTAYCYNSEGGF |
| | | RLIPAFRGYAQQDAQEFLCELL | | WVHCNDSKLSMCTMDEVCKA |
| | | DKIQRELETTGTSLPALIPTSQ | | QAYILFYTQRV |
| | | RKLIKQVLNVVNNIFHGQLLSQ | | |
| | | VTCLACDNKSNTIEPFWDLSLE | | |
| | | FPERYQCSGKDIASQPCLVTEM | | |
| | | LAKFTETEALEGKIYVCDQCNS | | |
| | | KRRRFSSKPVVLTEAQKQLMIC | | |
| | | HLPQVLRLHLKRFRWSGRNNRE | | |
| | | KIGVHVGFEEILNM | | |
| | | EPYCCRETLKSLRPECFIYDLS | | |
| | | AVVMHHGKGFGSGHYTAYCYNS | | |
| | | EGGFWVHCNDSKLSMCTMDEVC | | |
| | | KAQAYILFYTQRVTENGHSKLL | | |
| | | PPELLLGSQHPNEDADTSSNEI | | |
| | | LS | | |
| UBP8_HUMAN | 46 | MPAVASVPKELYLSSSLKDLNK | 158 | PALTGLRNLGNTCYMNSILQ |
| Ubiquitin | | KTEVKPEKISTKSYVHSALKIF | | CLCNAPHLADYENRNCYQDD |
| carboxyl- | | KTAEECRLDRDEERAYVLYMKY | | INRSNLLGHKGEVAEEFGII |
| terminal | | VTVYNLIKKRPDFKQQQDYFHS | | MKALWTGQYRYISPKDFKIT |
| hydrolase 8 | | ILGPGNIKKAVEEAERLSESLK | | IGKINDQFAGYSQQDSQELL |
| | | LRYEEAEVRKKLEEKDRQEEAQ | | LFLMDGLHEDLNKADNRKRY |
| | | RLQQKRQETGREDGGTLAKGSL | | KEENNDHLDDFKAAEHAWQK |
| | | ENVLDSKDKTQKSNGEKNEKCE | | HKQLNESIIVALFQGQFKST |
| | | TKEKGAITAKELYTMMTDKNIS | | VQCLTCHKKSRTFEAFMYLS |
| | | LIIMDARRMQDYQDSCILHSLS | | LPLASTSKCTLQDCLRLESK |
| | | VPEEAISPGVTASWIEAHLPDD | | EEKLTDNNRFYCSHCRARRD |
| | | SKDTWKKRGNVEYVVLLDWESS | | SLKKIEIWKLPPVLLVHLKR |
| | | AKDLQIGTTLRSLKDALFKWES | | FSYDGRWKQKLQTSVDEPLE |
| | | KTVLRNEPLVLEGG | | NLDLSQYVIGPKNNLKKYNL |
| | | YENWLLCYPQYTTNAKVTPPPR | | FSVSNHYGGLDGGHYTAYCK |
| | | RQNEEVSISLDFTYPSLEESIP | | NAARQRWFKEDDHEVSDISV |
| | | SKPAAQTPPASIEVDENIELIS | | SSVKSSAAYILFYTSLG |
| | | GQNERMGPLNISTPVEPVAASK | | |
| | | SDVSPIIQPVPSIKNVPQIDRT | | |
| | | KKPAVKLPEEHRIKSESTNHEQ | | |
| | | QSPQSGKVIPDRSTKPVVESPT | | |
| | | LMLTDEEKARIHAETALLMEKN | | |
| | | KQEKELRERQQEEQKEKLRKEE | | |
| | | QEQKAKKKQEAEENEITEKQQK | | |
| | | AKEEMEKKESEQAKKEDKETSA | | |
| | | KRGKEITGVKRQSKSEHETSDA | | |
| | | KKSVEDRGKRCPTPEIQKKSTG | | |
| | | DVPHTSVTGDSGSG | | |
| | | KPFKIKGQPESGILRTGTFRED | | |
| | | TDDTERNKAQREPLTRARSEEM | | |
| | | GRIVPGLPSGWAKFLDPITGTF | | |
| | | RYYHSPTNTVHMYPPEMAPSSA | | |
| | | PPSTPPTHKAKPQIPAERDREP | | |
| | | SKLKRSYSSPDITQAIQEEEKR | | |
| | | KPTVTPTVNRENKPTCYPKAEI | | |
| | | SRLSASQIRNLNPVEGGSGPAL | | |
| | | TGLRNLGNTCYMNSILQCLCNA | | |
| | | PHLADYFNRNCYQDDINRSNLL | | |
| | | GHKGEVAEEFGIIMKALWTGQY | | |
| | | RYISPKDFKITIGKINDQFAGY | | |
| | | SQQDSQELLLFLMDGLHEDLNK | | |
| | | ADNRKRYKEENNDH | | |
| | | LDDEKAAEHAWQKHKQLNESII | | |
| | | VALFQGQFKSTVQCLTCHKKSR | | |
| | | TFEAFMYLSLPLASTSKCTLQD | | |
| | | CLRLFSKEEKLTDNNRFYCSHC | | |
| | | RARRDSLKKIEIWKLPPVLLVH | | |
| | | LKRFSYDGRWKQKLQTSVDFPL | | |
| | | ENLDLSQYVIGPKNNLKKYNLF | | |
| | | SVSNHYGGLDGGHYTAYCKNAA | | |
| | | RQRWFKEDDHEVSDISVSSVKS | | |
| | | SAAYILFYTSLGPRVTDVAT | | |
| UBP37_HUMAN | 47 | MSPLKIHGPIRIRSMQTGITKW | 159 | QQLQGFSNLGNTCYMNAILQ |
| Ubiquitin | | KEGSFEIVEKENKVSLVVHYNT | | SLFSLQSFANDLLKQGIPWK |
| carboxyl- | | GGIPRIFQLSHNIKNVVLRPSG | | KIPLNALIRRFAHLLVKKDI |
| terminal | | AKQSRLMLTLQDNSFLSIDKVP | | CNSETKKDLLKKVKNAISAT |
| hydrolase 37 | | SKDAEEMRLFLDAVHQNRLPAA | | AERFSGYMQNDAHEFLSQCL |
| | | MKPSQGSGSFGAILGSRTSQKE | | DQLKEDMEKLNKTWKTEPVS |
| | | TSRQLSYSDNQASAKRGSLETK | | GEENSPDISATRAYTCPVIT |
| | | DDIPFRKVLGNPGRGSIKTVAG | | NLEFEVQHSIICKACGEIIP |
| | | SGIARTIPSLISTSTPLRSGLL | | KREQFNDLSIDLPRRKKPLP |
| | | ENRTEKRKRMISTGSELNEDYP | | PRSIQDSLDLFFRAEELEYS |
| | | KENDSSSNNKAMTDPSRKYLTS | | CEKCGGKCALVRHKENRLPR |
| | | SREKQLSLKQSEENRTSGLLPL | | VLILHLKRYSENVALSLNNK |
| | | QSSSFYGSRAGSKEHSSGGTNL | | IGQQVIIPRYLTLSSHCTEN |
| | | DRTNVSSQTPSAKR | | TKP |
| | | SLGFLPQPVPLSVKKLRCNQDY | | PFTLGWSAHMAISRPLKASQ |
| | | TGWNKPRVPLSSHQQQQLQGES | | MVNSCITSPSTPSKKFTEKS |
| | | NLGNTCYMNAILQSLFSLQSFA | | KSSLALCLDSDSEDELKRSV |
| | | NDLLKQGIPWKKIPLNALIRRE | | ALSQRLCEMLGNEQQQEDLE |
| | | AHLLVKKDICNSETKKDLLKKV | | KDSKLCPIEPDKSELENSGE |
| | | KNAISATAERFSGYMQNDAHEF | | DRMSEEELLAAVLEISKRDA |
| | | LSQCLDQLKEDMEKLNKTWKTE | | SPSLSHEDDDKPTSSPDTGE |
| | | PVSGEENSPDISATRAYTCPVI | | AEDDIQEMPENPDTMETEKP |
| | | TNLEFEVQHSIICKACGEIIPK | | KTITELDPASFTEITKDCDE |
| | | REQENDLSIDLPRRKKPLPPRS | | NKENKTPEGSQGEVDWLQQY |
| | | IQDSLDLFFRAEELEYSCEKCG | | DMEREREEQELQQALAQSLQ |
| | | GKCALVRHKENRLPRVLILHLK | | EQEAWEQKEDDDLKRATELS |
| | | RYSFNVALSLNNKIGQQVIIPR | | LQEENNSFVDALGSDEDSGN |
| | | YLTLSSHCTENTKP | | EDVEDMEYTEAEAEELKRNA |
| | | PFTLGWSAHMAISRPLKASQMV | | ETGNLPHSYRLISVVSHIGS |
| | | NSCITSPSTPSKKFTFKSKSSL | | TSSSGHYISDVYDIKKQAWF |
| | | ALCLDSDSEDELKRSVALSQRL | | TYNDLEVSKIQEAAVQSDRD |
| | | CEMLGNEQQQEDLEKDSKLCPI | | RSGYIFFYMHK |
| | | EPDKSELENSGEDRMSEEELLA | | |
| | | AVLEISKRDASPSLSHEDDDKP | | |
| | | TSSPDTGFAEDDIQEMPENPDT | | |
| | | METEKPKTITELDPASFTEITK | | |
| | | DCDENKENKTPEGSQGEVDWLQ | | |
| | | QYDMEREREEQELQQALAQSLQ | | |
| | | EQEAWEQKEDDDLKRATELSLQ | | |
| | | EFNNSFVDALGSDEDSGNEDVE | | |
| | | DMEYTEAEAEELKRNAETGNLP | | |
| | | HSYRLISVVSHIGS | | |
| | | TSSSGHYISDVYDIKKQAWFTY | | |
| | | NDLEVSKIQEAAVQSDRDRSGY | | |
| | | IFFYMHKEIFDELLETEKNSQS | | |
| | | LSTEVGKTTRQAL | | |
| U17LD_HUMAN | 48 | MEEDSLYLGGEWQFNHESKLTS | 160 | AVGAGLQNMGNTCYVNASLQ |
| Ubiquitin | | SRLDAAFAEIQRTSLPEKSPLS | | CLTYTPPLANYMLSREHSQT |
| carboxyl- | | CETRVDLCDDLVPEARQLAPRE | | CHRHKGCMLCTMQAHITRAL |
| terminal | | KLPLSSRRPAAVGAGLQNMGNT | | HNPGHVIQPSQALAAGFHRG |
| hydrolase 17- | | CYVNASLQCLTYTPPLANYMLS | | KQEDAHEFLMFTVDAMKKAC |
| like protein 13 | | REHSQTCHRHKGCMLCTMQAHI | | LPGHKQVDHPSKDTTLIHQI |
| | | TRALHNPGHVIQPSQALAAGFH | | FGGYWRSQIKCLHCHGISDT |
| | | RGKQEDAHEFLMFTVDAMKKAC | | FDPYLDIALDIQAAQSVQQA |
| | | LPGHKQVDHPSKDTTLIHQIFG | | LEQLVKPEELNGENAYHCGV |
| | | GYWRSQIKCLHCHGISDTFDPY | | CLQRAPASKTLTLHTSAKVL |
| | | LDIALDIQAAQSVQQALEQLVK | | ILVLKRFSDVTGNKIAKNVQ |
| | | PEELNGENAYHCGVCLQRAPAS | | YPECLDMQPYMSQQNTGPLV |
| | | KTLTLHTSAKVLILVLKRFSDV | | YVLYAVLVHAGWSCHNGHYF |
| | | TGNKIAKNVQYPEC | | SYVKAQEGQWYKMDDAEVTA |
| | | LDMQPYMSQQNTGPLVYVLYAV | | ASITSVLSQQAYVLFYIQKS |
| | | LVHAGWSCHNGHYFSYVKAQEG | | |
| | | QWYKMDDAEVTAASITSVLSQQ | | |
| | | AYVLFYIQKSEWERHSESVSRG | | |
| | | REPRALGAEDTDRRATQGELKR | | |
| | | DHPCLQAPELDEHLVERATQES | | |
| | | TLDRWKFLQEQNKTKPEFNVRK | | |
| | | VEGTLPPDVLVIHQSKYKCGMK | | |
| | | NHHPEQQSSLLNLSSSTPTHQE | | |
| | | SMNTGTLASLRGRARRSKGKNK | | |
| | | HSKRALLVCQ | | |
| U17L3_HUMAN | 49 | MGDDSLYLGGEWQFNHFSKLTS | 161 | AVGAGLQNMGNTCYENASLQ |
| Ubiquitin | | SRPDAAFAEIQRTSLPEKSPLS | | CLTYTLPLANYMLSREHSQT |
| carboxyl- | | SETRVDLCDDLAPVARQLAPRE | | CQRPKCCMLCTMQAHITWAL |
| terminal | | KLPLSSRRPAAVGAGLQNMGNT | | HSPGHVIQPSQALASGFHRG |
| hydrolase 17- | | CYENASLQCLTYTLPLANYMLS | | KQEDVHEFLMFTVDAMKKAC |
| like protein 3 | | REHSQTCQRPKCCMLCTMQAHI | | LPGHKQVDHHSKDTTLIHQI |
| | | TWALHSPGHVIQPSQALASGFH | | FGGCWRSQIKCLHCHGISDT |
| | | RGKQEDVHEFLMFTVDAMKKAC | | FDPYLDIALDIQAAQSVKQA |
| | | LPGHKQVDHHSKDTTLIHQIFG | | LEQLVKPEELNGENAYHCGL |
| | | GCWRSQIKCLHCHGISDTEDPY | | CLQRAPASNTLTLHTSAKVL |
| | | LDIALDIQAAQSVKQALEQLVK | | ILVLKRESDVAGNKLAKNVQ |
| | | PEELNGENAYHCGLCLQRAPAS | | YPECLDMQPYMSQQNTGPLV |
| | | NTLTLHTSAKVLILVLKRFSDV | | YVLYAVLVHAGWSCHDGHYF |
| | | AGNKLAKNVQYPEC | | SYVKAQEGQWYKMDDAEVTV |
| | | LDMQPYMSQQNTGPLVYVLYAV | | CSITSVLSQQAYVLFYIQKS |
| | | LVHAGWSCHDGHYFSYVKAQEG | | |
| | | QWYKMDDAEVTVCSITSVLSQQ | | |
| | | AYVLFYIQKSEWERHSESVSRG | | |
| | | REPRALGAEDTDRRAKQGELKR | | |
| | | DHPCLQAPELDEHLVERATQES | | |
| | | TLDHWKFLQEQNKTKPEFNVGK | | |
| | | VEGTLPPNALVIHQSKYKCGMK | | |
| | | NHHPEQQSSLLNLSSTTRTDQE | | |
| | | SMNTGTLASLQGRTRRAKGKNK | | |
| | | HSKRALLVCQ | | |
| UBP54_HUMAN | 50 | MSWKRNYFSGGRGSVQGMFAPR | 162 | APSKGLSNEPGQNSCFLNSA |
| Inactive | | SSTSIAPSKGLSNEPGQNSCEL | | LQVLWHLDIFRRSFRQLTTH |
| ubiquitin | | NSALQVLWHLDIFRRSFRQLTT | | KCMGDSCIFCALKGIFNQFQ |
| carboxyl- | | HKCMGDSCIFCALKGIFNQFQC | | CSSEKVLPSDTLRSALAKTF |
| terminal | | SSEKVLPSDTLRSALAKTFQDE | | QDEQRFQLGIMDDAAECFEN |
| hydrolase 54 | | QRFQLGIMDDAAECFENLLMRI | | LLMRIHFHIADETKEDICTA |
| | | HFHIADETKEDICTAQHCISHQ | | QHCISHQKFAMTLFEQCVCT |
| | | KFAMTLFEQCVCTSCGATSDPL | | SCGATSDPLPFIQ |
| | | PFIQMVHYISTTSLCNQAICML | | MVHYISTTSLCNQAICMLER |
| | | ERREKPSPSMFGELLQNASTMG | | REKPSPSMFGELLQNASTMG |
| | | DLRNCPSNCGERIRIRRVLMNA | | DLRNCPSNCGERIRIRRVLM |
| | | PQIITIGLVWDSDHSDLAEDVI | | NAPQIITIGLVWDSDHSDLA |
| | | HSLGTCLKLGDLFFRVTDDRAK | | EDVIHSLGTCLKLGDLFFRV |
| | | QSELYLVGMICYYG | | TDDRAKQSELYLVGMICYYG |
| | | KHYSTFFFQTKIRKWMYEDDAH | | KHYSTFFFQTKIRKWMYFDD |
| | | VKEIGPKWKDVVTKCIKGHYQP | | AHVKEIGPKWKDVVTKCIKG |
| | | LLLLYADPQGTPVSTQDLPPQA | | HYQPLLLLYADPQGTPVSTQ |
| | | EFQSYSRTCYDSEDSGREPSIS | | DLPPQAEFQSYSRTCYDSED |
| | | SDTRTDSSTESYPYKHSHHESV | | SGREPSISSDTRTDSSTESY |
| | | VSHFSSDSQGTVIYNVENDSMS | | PYKHSHHESVVSHFSSDSQG |
| | | QSSRDTGHLTDSECNQKHTSKK | | TVIYNVEND |
| | | GSLIERKRSSGRVRRKGDEPQA | | |
| | | SGYHSEGETLKEKQAPRNASKP | | |
| | | SSSTNRLRDFKETVSNMIHNRP | | |
| | | SLASQTNVGSHCRGRGGDQPDK | | |
| | | KPPRTLPLHSRDWEIESTSSES | | |
| | | KSSSSSKYRPTWRPKRESLNID | | |
| | | SIFSKDKRKHCGYT | | |
| | | QLSPFSEDSAKEFIPDEPSKPP | | |
| | | SYDIKFGGPSPQYKRWGPARPG | | |
| | | SHLLEQHPRLIQRMESGYESSE | | |
| | | RNSSSPVSLDAALPESSNVYRD | | |
| | | PSAKRSAGLVPSWRHIPKSHSS | | |
| | | SILEVDSTASMGGWTKSQPFSG | | |
| | | EEISSKSELDELQEEVARRAQE | | |
| | | QELRRKREKELEAAKGENPHPS | | |
| | | REMDLDELQNQGRSDGFERSLQ | | |
| | | EAESVFEESLHLEQKGDCAAAL | | |
| | | ALCNEAISKLRLALHGASCSTH | | |
| | | SRALVDKKLQISIRKARSLQDR | | |
| | | MQQQQSPQQPSQPSACLPTQAG | | |
| | | TLSQPTSEQPIPLQ | | |
| | | VLLSQEAQLESGMDTEFGASSE | | |
| | | FHSPASCHESHSSLSPESSAPQ | | |
| | | HSSPSRSALKLLTSVEVDNIEP | | |
| | | SAFHRQGLPKAPGWTEKNSHHS | | |
| | | WEPLDAPEGKLQGSRCDNSSCS | | |
| | | KLPPQEGRGIAQEQLFQEKKDP | | |
| | | ANPSPVMPGIATSERGDEHSLG | | |
| | | CSPSNSSAQPSLPLYRTCHPIM | | |
| | | PVASSFVLHCPDPVQKTNQCLQ | | |
| | | GQSLKTSLTLKVDRGSEETYRP | | |
| | | EFPSTKGLVRSLAEQFQRMQGV | | |
| | | SMRDSTGFKDRSLSGSLRKNSS | | |
| | | PSDSKPPFSQGQEKGHWPWAKQ | | |
| | | QSSLEGGDRPLSWE | | |
| | | ESTEHSSLALNSGLPNGETSSG | | |
| | | GQPRLAEPDIYQEKLSQVRDVR | | |
| | | SKDLGSSTDLGTSLPLDSWVNI | | |
| | | TRFCDSQLKHGAPRPGMKSSPH | | |
| | | DSHTCVTYPERNHILLHPHWNQ | | |
| | | DTEQETSELESLYQASLQASQA | | |
| | | GCSGWGQQDTAWHPLSQTGSAD | | |
| | | GMGRRLHSAHDPGLSKTSTAEM | | |
| | | EHGLHEARTVRTSQATPCRGLS | | |
| | | RECGEDEQYSAENLRRISRSLS | | |
| | | GTVVSEREEAPVSSHSFDSSNV | | |
| | | RKPLETGHRCSSSSSLPVIHDP | | |
| | | SVFLLGPQLYLPQPQFLSPDVL | | |
| | | MPTMAGEPNRLPGT | | |
| | | SRSVQQFLAMCDRGETSQGAKY | | |
| | | TGRTLNYQSLPHRSRTDNSWAP | | |
| | | WSETNQHIGTRELTTPGCNPQL | | |
| | | TYTATLPERSKGLQVPHTQSWS | | |
| | | DLFHSPSHPPIVHPVYPPSSSL | | |
| | | HVPLRSAWNSDPVPGSRTPGPR | | |
| | | RVDMPPDDDWRQSSYASHSGHR | | |
| | | RTVGEGFLFVLSDAPRREQIRA | | |
| | | RVLQHSQW | | |
| SNUT2_HUMAN | 51 | MSGRSKRESRGSTRGKRESESR | 163 | LPGIVGLNNIKANDYANAVL |
| U4/U6.U5 | | GSSGRVKRERDREREPEAASSR | | QALSNVPPLRNYFLEEDNYK |
| tri-snRNP- | | GSPVRVKREFEPASAREAPASV | | NIKRPPGDIMELLVQRFGEL |
| associated | | VPFVRVKREREVDEDSEPEREV | | MRKLWNPRNFKAHVSPHEML |
| protein 2 | | RAKNGRVDSEDRRSRHCPYLDT | | QAVVLCSKKTFQITKQGDGV |
| | | INRSVLDEDFEKLCSISLSHIN | | DFLSWFLNALHSALGGTKKK |
| | | AYACLVCGKYFQGRGLKSHAYI | | KKTIVTDVFQGSMRIFTKKL |
| | | HSVQFSHHVELNLHTLKFYCLP | | PHPDLPAEEKEQLLHNDEYQ |
| | | DNYEIIDSSLEDITYVLKPTFT | | ETMVESTFMYLTLDLPTAPL |
| | | KQQIANLDKQAKLSRAYDGTTY | | YKDEKEQLIIPQVPLENILA |
| | | LPGIVGLNNIKANDYANAVLQA | | KENGITEKEYKTYKENFLKR |
| | | LSNVPPLRNYFLEEDNYKNIKR | | FQLTKLPPYLIFCIKRFTKN |
| | | PPGDIMFLLVQRFGELMRKLWN | | NFFVEKNPTIVNFPITNVDL |
| | | PRNFKAHVSPHEML | | REYLSEEVQAVHKNTTYDLI |
| | | QAVVLCSKKTFQITKQGDGVDE | | ANIVHDGKPSEGSYRIHVLH |
| | | LSWFLNALHSALGGTKKKKKTI | | HGTGKWYELQDLQVTDILPQ |
| | | VTDVFQGSMRIFTKKLPHPDLP | | MITLSEAYIQIWKRRD |
| | | AEEKEQLLHNDEYQETMVESTE | | |
| | | MYLTLDLPTAPLYKDEKEQLII | | |
| | | PQVPLENILAKENGITEKEYKT | | |
| | | YKENFLKRFQLTKLPPYLIFCI | | |
| | | KRFTKNNFFVEKNPTIVNEPIT | | |
| | | NVDLREYLSEEVQAVHKNTTYD | | |
| | | LIANIVHDGKPSEGSYRIHVLH | | |
| | | HGTGKWYELQDLQVTDILPQMI | | |
| | | TLSEAYIQIWKRRDNDETNQQG | | |
| | | A | | |
| UBP35_HUMAN | 52 | MDKILEAVVTSSYPVSVKQGLV | 164 | SDTGKIGLINLGNTCYVNSI |
| Ubiquitin | | RRVLEAARQPLEREQCLALLAL | | LQALFMASDERHCVLRLTEN |
| carboxyl- | | GARLYVGGAEELPRRVGCQLLH | | NSQPLMTKLQWLFGFLEHSQ |
| terminal | | VAGRHHPDVFAEFFSARRVLRL | | RPAISPENELSASWTPWESP |
| hydrolase 35 | | LQGGAGPPGPRALACVQLGLQL | | GTQQDCSEYLKYLLDRLHEE |
| | | LPEGPAADEVFALLRREVLRTV | | EKTGTRICQKLKQSSSPSPP |
| | | CERPGPAACAQVARLLARHPRC | | EEPPAPSSTSVEKMEGGKIV |
| | | VPDGPHRLLFCQQLVRCLGRER | | TRICCLCCLNVSSREEAFTD |
| | | CPAEGEEGAVEFLEQAQQVSGL | | LSLAFPPPERCRRRRLGSVM |
| | | LAQLWRAQPAAILPCLKELFAV | | RPTEDITARELPPPTSAQGP |
| | | ISCAEEEPPSSALASVVQHLPL | | GRVGPRRQRKHCITEDTPPT |
| | | ELMDGVVRNLSNDDSVTDSQML | | SLYIEGLDSKEAGGQSSQEE |
| | | TAISRMIDWVSWPLGKNIDKWI | | RIEREEEGKEERTEKEEVGE |
| | | IALLKGLAAVKKES | | EEESTRGEGEREKEEEVEEE |
| | | ILIEVSLTKIEKVESKLLYPIV | | EEKVE |
| | | RGAALSVLKYMLLTFQHSHEAF | | KETEKEAEQEKEEDSLGAGT |
| | | HLLLPHIPPMVASLVKEDSNSG | | HPDAAIPSGERTCGSEGSRS |
| | | TSCLEQLAELVHCMVFRFPGEP | | VLDLVNYFLSPEKLTAENRY |
| | | DLYEPVMEAIKDLHVPNEDRIK | | YCESCASLQDAEKVVELSQG |
| | | QLLGQDAWTSQKSELAGFYPRL | | PCYLILTLLRESFDLRTMRR |
| | | MAKSDTGKIGLINLGNTCYVNS | | RKILDDVSIPLLLRLPLAGG |
| | | ILQALFMASDERHCVLRLTENN | | RGQAYDLCSVVVHSGVSSES |
| | | SQPLMTKLQWLFGFLEHSQRPA | | GHYYCYAREGAARPAASLGT |
| | | ISPENFLSASWTPWFSPGTQQD | | ADRPEPENQWYLENDTRVSE |
| | | CSEYLKYLLDRLHEEEKTGTRI | | SSFESVSNVTSFFPKDTAYV |
| | | CQKLKQSSSPSPPEEPPAPSST | | LFYRQRP |
| | | SVEKMFGGKIVTRICCLCCLNV | | |
| | | SSREEAFTDLSLAF | | |
| | | PPPERCRRRRLGSVMRPTEDIT | | |
| | | ARELPPPTSAQGPGRVGPRRQR | | |
| | | KHCITEDTPPTSLYIEGLDSKE | | |
| | | AGGQSSQEERIEREEEGKEERT | | |
| | | EKEEVGEEEESTRGEGEREKEE | | |
| | | EVEEEEEKVEKETEKEAEQEKE | | |
| | | EDSLGAGTHPDAAIPSGERTCG | | |
| | | SEGSRSVLDLVNYFLSPEKLTA | | |
| | | ENRYYCESCASLQDAEKVVELS | | |
| | | QGPCYLILTLLRFSFDLRTMRR | | |
| | | RKILDDVSIPLLLRLPLAGGRG | | |
| | | QAYDLCSVVVHSGVSSESGHYY | | |
| | | CYAREGAARPAASLGTADRPEP | | |
| | | ENQWYLENDTRVSE | | |
| | | SSFESVSNVTSFFPKDTAYVLF | | |
| | | YRQRPREGPEAELGSSRVRTEP | | |
| | | TLHKDLMEAISKDNILYLQEQE | | |
| | | KEARSRAAYISALPTSPHWGRG | | |
| | | FDEDKDEDEGSPGGCNPAGGNG | | |
| | | GDFHRLVE | | |
| UBP15_HUMAN | 53 | MAEGGAADLDTQRSDIATLLKT | 165 | EQPGLCGLSNLGNTCEMNSA |
| Ubiquitin | | SLRKGDTWYLVDSRWFKQWKKY | | IQCLSNTPPLTEYELNDKYQ |
| carboxyl- | | VGFDSWDKYQMGDQNVYPGPID | | EELNFDNPLGMRGEIAKSYA |
| terminal | | NSGLLKDGDAQSLKEHLIDELD | | ELIKQMWSGKFSYVTPRAFK |
| hydrolase 15 | | YILLPTEGWNKLVSWYTLMEGQ | | TQVGRFAPQFSGYQQQDCQE |
| | | EPIARKVVEQGMFVKHCKVEVY | | LLAFLLDGLHEDLNRIRKKP |
| | | LTELKLCENGNMNNVVTRRESK | | YIQLKDADGRPDKVVAEEAW |
| | | ADTIDTIEKEIRKIFSIPDEKE | | ENHLKRNDSIIVDIFHGLFK |
| | | TRLWNKYMSNTFEPLNKPDSTI | | STLVCPECAKISVTFDPFCY |
| | | QDAGLYQGQVLVIEQKNEDGTW | | LTLPLPMKKERTLEVYLVRM |
| | | PRGPSTPKSPGASNESTLPKIS | | DPLTKPMQYKVVVPKIGNIL |
| | | PSSLSNNYNNMNNRNVKNSNYC | | DLCTALSALSGIPADKMIVT |
| | | LPSYTAYKNYDYSEPGRNNEQP | | DIYNHRFHRIFAMDENLSSI |
| | | GLCGLSNLGNTCEM | | MERDDIYVFEININRTEDTE |
| | | NSAIQCLSNTPPLTEYELNDKY | | HVIIPVCLREKFRHSSYTHH |
| | | QEELNFDNPLGMRGEIAKSYAE | | TGSSLFGQPFLMAVPRNNTE |
| | | LIKQMWSGKFSYVTPRAFKTQV | | DKLYNLLLLRMCRYVKISTE |
| | | GRFAPQFSGYQQQDCQELLAFL | | TEETEGSLHCCKDQNINGNG |
| | | LDGLHEDLNRIRKKPYIQLKDA | | PNGIHEEGSPSEMETDEPDD |
| | | DGRPDKVVAEEAWENHLKRNDS | | ESSQDQELPSENENSQSEDS |
| | | IIVDIFHGLFKSTLVCPECAKI | | VGGDNDSENGLCTEDTCKGQ |
| | | SVTFDPFCYLTLPLPMKKERTL | | LTGHKKRLFTFQFNNLGNTD |
| | | EVYLVRMDPLTKPMQYKVVVPK | | INYIKDDTRHIREDDRQLRL |
| | | IGNILDLCTALSALSGIPADKM | | DERSFLALDWDPDLKKRYED |
| | | IVTDIYNHRFHRIFAMDENLSS | | ENAAEDFEKHESVEYKPPKK |
| | | IMERDDIYVFEININRTEDTEH | | PFVKLKDCIELFTTKEKLGA |
| | | VIIPVCLREKFRHSSYTHHTGS | | EDPWYCPNCKEHQQATKKLD |
| | | SLFGQPFLMAVPRN | | LWSLPPVLVVHLKRESYSRY |
| | | NTEDKLYNLLLLRMCRYVKIST | | MRDKLDTLVDFPINDLDMSE |
| | | ETEETEGSLHCCKDQNINGNGP | | FLINPNAGPCRYNLIAVSNH |
| | | NGIHEEGSPSEMETDEPDDESS | | YGGMGGGHYTAFAKNKDDGK |
| | | QDQELPSENENSQSEDSVGGDN | | WYYFDDSSVSTASEDQIVSK |
| | | DSENGLCTEDTCKGQLTGHKKR | | AAYVLFYQRQD |
| | | LFTFQFNNLGNTDINYIKDDTR | | |
| | | HIREDDRQLRLDERSFLALDWD | | |
| | | PDLKKRYFDENAAEDFEKHESV | | |
| | | EYKPPKKPFVKLKDCIELFTTK | | |
| | | EKLGAEDPWYCPNCKEHQQATK | | |
| | | KLDLWSLPPVLVVHLKRESYSR | | |
| | | YMRDKLDTLVDFPINDLDMSEF | | |
| | | LINPNAGPCRYNLIAVSNHYGG | | |
| | | MGGGHYTAFAKNKD | | |
| | | DGKWYYFDDSSVSTASEDQIVS | | |
| | | KAAYVLFYQRQDTFSGTGFFPL | | |
| | | DRETKGASAATGIPLESDEDSN | | |
| | | DNDNDIENENCMHTN | | |
| UBP29_HUMAN | 54 | MISLKVCGFIQIWSQKTGMTKL | 166 | QLQQGFPNLGNTCYMNAVLQ |
| Ubiquitin | | KEALIETVQRQKEIKLVVTEKS | | SLFAIPSFADDLLTQGVPWE |
| carboxyl- | | GKFIRIFQLSNNIRSVVLRHCK | | YIPFEALIMTLTQLLALKDE |
| terminal | | KRQSHLRLTLKNNVELFIDKLS | | CSTKIKRELLGNVKKVISAV |
| hydrolase 29 | | YRDAKQLNMELDIIHQNKSQQP | | AEIFSGNMQNDAHEFLGQCL |
| | | MKSDDDWSVFESRNMLKEIDKT | | DQLKEDMEKLNATLNTGKEC |
| | | SFYSICNKPSYQKMPLFMSKSP | | GDENSSPQMHVGSAATKVEV |
| | | THVKKGILENQGGKGQNTLSSD | | CPVVANFEFELQLSLICKAC |
| | | VQTNEDILKEDNPVPNKKYKTD | | GHAVLKVEPNNYLSINLHQE |
| | | SLKYIQSNRKNPSSLEDLEKDR | | TKPLPLSIQNSLDLFFKEEE |
| | | DLKLGPSENTNCNGNPNLDETV | | LEYNCQMCKQKSCVARHTES |
| | | LATQTLNAKNGLTSPLEPEHSQ | | RLSRVLIIHLKRYSENNAWL |
| | | GDPRCNKAQVPLDSHSQQLQQG | | LVKNNEQVYIPKSLSLSSYC |
| | | FPNLGNTCYMNAVL | | NESTKPPLPLSSSAPVGKCE |
| | | QSLFAIPSFADDLLTQGVPWEY | | VLEVSQEMISEINSPLTPSM |
| | | IPFEALIMTLTQLLALKDECST | | KLTSESSDSLVLPVEPDKNA |
| | | KIKRELLGNVKKVISAVAEIFS | | DLQRFQRDCGDASQEQHQRD |
| | | GNMQNDAHEFLGQCLDQLKEDM | | LENGSALESELVHERDRAIG |
| | | EKLNATLNTGKECGDENSSPQM | | EKELPVADSLMDQGDISLPV |
| | | HVGSAATKVFVCPVVANFEFEL | | MYEDGGKLISSPDTRLVEVH |
| | | QLSLICKACGHAVLKVEPNNYL | | LQEVPQHPELQKYEKTNTFV |
| | | SINLHQETKPLPLSIQNSLDLF | | EFNFDSVTESTNGFYDCKEN |
| | | FKEEELEYNCQMCKQKSCVARH | | RIPEGSQGMAEQLQQCIEES |
| | | TFSRLSRVLIIHLKRYSENNAW | | IIDEFLQQAPPPGVRKLDAQ |
| | | LLVKNNEQVYIPKSLSLSSYCN | | EHTEETLNQSTELRLQKADL |
| | | ESTKPPLPLSSSAPVGKCEVLE | | NHLGALGSDNPGNKNILDAE |
| | | VSQEMISEINSPLTPSMKLTSE | | NTRGEAKELTRNVKMGDPLQ |
| | | SSDSLVLPVEPDKN | | AYRLISVVSHIGSSPNSGHY |
| | | ADLQRFQRDCGDASQEQHQRDL | | ISDVYDFQKQAWFTYNDLCV |
| | | ENGSALESELVHERDRAIGEKE | | SEISETKMQEARLHSGYIFF |
| | | LPVADSLMDQGDISLPVMYEDG | | YMHN |
| | | GKLISSPDTRLVEVHLQEVPQH | | |
| | | PELQKYEKTNTFVEFNEDSVTE | | |
| | | STNGFYDCKENRIPEGSQGMAE | | |
| | | QLQQCIEESIIDEFLQQAPPPG | | |
| | | VRKLDAQEHTEETLNQSTELRL | | |
| | | QKADLNHLGALGSDNPGNKNIL | | |
| | | DAENTRGEAKELTRNVKMGDPL | | |
| | | QAYRLISVVSHIGSSPNSGHYI | | |
| | | SDVYDFQKQAWFTYNDLCVSEI | | |
| | | SETKMQEARLHSGYIFFYMHNG | | |
| | | IFEELLRKAENSRLPSTQAGVI | | |
| | | PQGEYEGDSLYRPA | | |
| UBP6_HUMAN | 55 | MDMVENADSLQAQERKDILMKY | 167 | KGATGLSNLGNTCEMNSSIQ |
| Ubiquitin | | DKGHRAGLPEDKGPEPVGINSS | | CVSNTQPLTQYFISGRHLYE |
| carboxyl- | | IDRFGILHETELPPVTAREAKK | | LNRTNPIGMKGHMAKCYGDL |
| terminal | | IRREMTRTSKWMEMLGEWETYK | | VQELWSGTQKSVAPLKLRRT |
| hydrolase 6 | | HSSKLIDRVYKGIPMNIRGPVW | | IAKYAPKFDGFQQQDSQELL |
| | | SVLLNIQEIKLKNPGRYQIMKE | | AFLLDGLHEDLNRVHEKPYV |
| | | RGKRSSEHIHHIDLDVRTTLRN | | ELKDSDGRPDWE |
| | | HVFFRDRYGAKQRELFYILLAY | | VAAEAWDNHLRRNRSIIVDL |
| | | SEYNPEVGYCRDLSHITALFLL | | FHGQLRSQVKCKTCGHISVR |
| | | YLPEEDAFWALVQLLASERHSL | | FDPNFLSLPLPMDSYMDLEI |
| | | PGFHSPNGGTVQGLQDQQEHVV | | TVIKLDGTTPVRYGLRLNMD |
| | | PKSQPKTMWHQDKEGLCGQCAS | | EKYTGLKKQLRDLCGLNSEQ |
| | | LGCLLRNLIDGISLGLTLRLWD | | ILLAEVHDSNIKNFPQDNQK |
| | | VYLVEGEQVLMPIT | | VQLSVSGFLCAFEIPVPSSP |
| | | SIALKVQQKRLMKTSRCGLWAR | | ISASSPTQIDESSSPSTNGM |
| | | LRNQFFDTWAMNDDTVLKHLRA | | FTLTTNGDLPKPIFIPNGMP |
| | | STKKLTRKQGDLPPPAKREQGS | | NTVVPCGTEKNFTNGMVNGH |
| | | LAPRPVPASRGGKTLCKGYRQA | | MPSLPDSPFTGYIIAVHRKM |
| | | PPGPPAQFQRPICSASPPWASR | | MRTELYFLSPQENRPSLEGM |
| | | FSTPCPGGAVREDTYPVGTQGV | | PLIVPCTVHTRKKDLYDAVW |
| | | PSLALAQGGPQGSWRFLEWKSM | | IQVSWLARPLPPQEASIHAQ |
| | | PRLPTDLDIGGPWFPHYDFEWS | | DRDNCMGYQYPFTLRVVQKD |
| | | CWVRAISQEDQLATCWQAEHCG | | GNSCAWCPQYRFCRGCKIDC |
| | | EVHNKDMSWPEEMSFTANSSKI | | GEDRAFIGNAYIAVDWHPTA |
| | | DRQKVPTEKGATGLSNLGNTCF | | LHLRYQTSQERVVDKHESVE |
| | | MNSSIQCVSNTQPLTQYFISGR | | QSRRAQAEPINLDSCLRAFT |
| | | HLYELNRTNPIGMKGHMAKCYG | | SEEELGESEMYYCSKCKTHC |
| | | DLVQELWSGTQKSV | | LATKKLDLWRLPPFLIIHLK |
| | | APLKLRRTIAKYAPKEDGEQQQ | | RFQFVNDQWIKSQKIVRFLR |
| | | DSQELLAFLLDGLHEDLNRVHE | | ESFDPSAFLVPRDPALCQHK |
| | | KPYVELKDSDGRPDWEVAAEAW | | PLTPQGDELSKPRILAREVK |
| | | DNHLRRNRSIIVDLFHGQLRSQ | | KVDAQSSAGKEDMLLSKSPS |
| | | VKCKTCGHISVREDPENFLSLP | | SLSANISSSPKGSPSSSRKS |
| | | LPMDSYMDLEITVIKLDGTTPV | | GTSCPSSKNSSPNSSPRTLG |
| | | RYGLRLNMDEKYTGLKKQLRDL | | RSKGRLRLPQIGSKNKPSSS |
| | | CGLNSEQILLAEVHDSNIKNFP | | KKNLDASKENGAGQICELAD |
| | | QDNQKVQLSVSGFLCAFEIPVP | | ALSRGHMRGGSQPELVTPQD |
| | | SSPISASSPTQIDESSSPSTNG | | HEVALANGFLYEHEACGNGC |
| | | MFTLTINGDLPKPIFIPNGMPN | | GDGYSNGQLGNHSEEDSTDD |
| | | TVVPCGTEKNFTNGMVNGHMPS | | QREDTHIKPIYNLYAISCHS |
| | | LPDSPFTGYIIAVHRKMMRTEL | | GILSGGHYITYAKNPNCKWY |
| | | YFLSPQENRPSLFG | | CYNDSSCEELHPDEIDTDSA |
| | | MPLIVPCTVHTRKKDLYDAVWI | | YILFYEQQG |
| | | QVSWLARPLPPQEASIHAQDRD | | |
| | | NCMGYQYPFTLRVVQKDGNSCA | | |
| | | WCPQYRFCRGCKIDCGEDRAFI | | |
| | | GNAYIAVDWHPTALHLRYQTSQ | | |
| | | ERVVDKHESVEQSRRAQAEPIN | | |
| | | LDSCLRAFTSEEELGESEMYYC | | |
| | | SKCKTHCLATKKLDLWRLPPEL | | |
| | | IIHLKRFQFVNDQWIKSQKIVR | | |
| | | FLRESFDPSAFLVPRDPALCQH | | |
| | | KPLTPQGDELSKPRILAREVKK | | |
| | | VDAQSSAGKEDMLLSKSPSSLS | | |
| | | ANISSSPKGSPSSSRKSGTSCP | | |
| | | SSKNSSPNSSPRTL | | |
| | | GRSKGRLRLPQIGSKNKPSSSK | | |
| | | KNLDASKENGAGQICELADALS | | |
| | | RGHMRGGSQPELVTPQDHEVAL | | |
| | | ANGFLYEHEACGNGCGDGYSNG | | |
| | | QLGNHSEEDSTDDQREDTHIKP | | |
| | | IYNLYAISCHSGILSGGHYITY | | |
| | | AKNPNCKWYCYNDSSCEELHPD | | |
| | | EIDTDSAYILFYEQQGIDYAQF | | |
| | | LPKIDGKKMADTSSTDEDSESD | | |
| | | YEKYSMLQ | | |
| UBP53_HUMAN | 56 | MAWVKFLRKPGGNLGKVYQPGS | 168 | APTKGLLNEPGQNSCFLNSA |
| Inactive | | MLSLAPTKGLLNEPGQNSCFLN | | VQVLWQLDIFRRSLRVLTGH |
| ubiquitin | | SAVQVLWQLDIFRRSLRVLTGH | | VCQGDACIFCALKTIFAQFQ |
| carboxyl- | | VCQGDACIFCALKTIFAQFQHS | | HSREKALPSDNIRHALAESF |
| terminal | | REKALPSDNIRHALAESFKDEQ | | KDEQRFQLGLMDDAAECFEN |
| hydrolase 53 | | RFQLGLMDDAAECFENMLERIH | | MLERIHFHIVPSRDADMCTS |
| | | FHIVPSRDADMCTSKSCITHQK | | KSCITHQKFAMTLYEQCVCR |
| | | FAMTLYEQCVCRSCGASSDPLP | | SCGASSDPLPFTEFVRYIST |
| | | FTEFVRYISTTALCNEVERMLE | | TALCNEVERMLERHERFKPE |
| | | RHERFKPEMFAELLQAANTTDD | | MFAELLQAANTTDDYRKCPS |
| | | YRKCPSNCGQKIKIRRVLMNCP | | NCGQKIKIRRVLMNCPEIVT |
| | | EIVTIGLVWDSEHSDLTEAVVR | | IGLVWDSEHSDLTEAVVRNL |
| | | NLATHLYLPGLFYRVTDENAKN | | ATHLYLPGLFYRVTDENAKN |
| | | SELNLVGMICYTSQ | | SELNLVGMICYTSQHYCAFA |
| | | HYCAFAFHTKSSKWVFEDDANV | | FHTKSSKWVFEDDANVKEIG |
| | | KEIGTRWKDVVSKCIRCHFQPL | | TRWKDVVSKCIRCHFQPLLL |
| | | LLFYANPDGTAVSTEDALRQVI | | FYANPDGTAVSTEDALRQVI |
| | | SWSHYKSVAENMGCEKPVIHKS | | SWSHYKSVAENMGCEKPVIH |
| | | DNLKENGFGDQAKQRENQKEPT | | KSDNLKENGFGDQAKQRENQ |
| | | DNISSSNRSHSHTGVGKGPAKL | | KFPTDNISSSNRSHSHTGVG |
| | | SHIDQREKIKDISRECALKAIE | | KGPAKLSHIDQREKIKDISR |
| | | QKNLLSSQRKDLEKGQRKDLGR | | ECALKAIEQKNLLSSQRKDL |
| | | HRDLVDEDLSHFQSGSPPAPNG | | EKGQRK |
| | | FKQHGNPHLYHSQGKGSYKHDR | | |
| | | VVPQSRASAQIISSSKSQILAP | | |
| | | GEKITGKVKSDNGTGYDTDSSQ | | |
| | | DSRDRGNSCDSSSKSRNRGWKP | | |
| | | MRETLNVDSIFSES | | |
| | | EKRQHSPRHKPNISNKPKSSKD | | |
| | | PSFSNWPKENPKQKGLMTIYED | | |
| | | EMKQEIGSRSSLESNGKGAEKN | | |
| | | KGLVEGKVHGDNWQMQRTESGY | | |
| | | ESSDHISNGSTNLDSPVIDGNG | | |
| | | TVMDISGVKETVCFSDQITTSN | | |
| | | LNKERGDCTSLQSQHHLEGERK | | |
| | | ELRNLEAGYKSHEFHPESHLQI | | |
| | | KNHLIKRSHVHEDNGKLFPSSS | | |
| | | LQIPKDHNAREHIHQSDEQKLE | | |
| | | KPNECKESEWLNIENSERTGLP | | |
| | | FHVDNSASGKRVNSNEPSSLWS | | |
| | | SHLRTVGLKPETAPLIQQQNIM | | |
| | | DQCYFENSLSTECI | | |
| | | IRSASRSDGCQMPKLFCQNLPP | | |
| | | PLPPKKYAITSVPQSEKSESTP | | |
| | | DVKLTEVFKATSHLPKHSLSTA | | |
| | | SEPSLEVSTHMNDERHKETFQV | | |
| | | RECFGNTPNCPSSSSTNDEQAN | | |
| | | SGAIDAFCQPELDSISTCPNET | | |
| | | VSLTTYFSVDSCMTDTYRLKYH | | |
| | | QRPKLSFPESSGFCNNSLS | | |
| U17LO_HUMAN | 57 | MEDDSLYLRGEWQFNHESKLTS | 169 | AVGAGLQNMGNTCYVNASLQ |
| Ubiquitin | | SRPDAAFAEIQRTSLPEKSPLS | | CLTYTPPLANYMLSREHSQT |
| carboxyl- | | CETRVDLCDDLAPVARQLAPRE | | CHRHKGCMLCTMQAHITRAL |
| terminal | | KLPLSSRRPAAVGAGLQNMGNT | | HNPGHVIQPSQALAAGFHRG |
| hydrolase 17- | | CYVNASLQCLTYTPPLANYMLS | | KQEDAHEFLMFTVDAMKKAC |
| like protein 24 | | REHSQTCHRHKGCMLCTMQAHI | | LPGHKQVDHHSKDTTLIHQI |
| | | TRALHNPGHVIQPSQALAAGFH | | FGGYWRSQIKCLHCHGISDT |
| | | RGKQEDAHEFLMFTVDAMKKAC | | FDPYLDIALDIQAAQSVQQA |
| | | LPGHKQVDHHSKDTTLIHQIFG | | LEQLVKPEELNGENAYHCGV |
| | | GYWRSQIKCLHCHGISDTEDPY | | CLQRAPASKTLTLHTSAKVL |
| | | LDIALDIQAAQSVQQALEQLVK | | ILVLKRFSDVTGNKIAKNVQ |
| | | PEELNGENAYHCGVCLQRAPAS | | YPECLDMQPYMSQPNTGPLV |
| | | KTLTLHTSAKVLILVLKRESDV | | YVLYAVLVHAGWSCHNGHYF |
| | | TGNKIAKNVQYPEC | | SYVKAQEGQWYKMDDAEVTA |
| | | LDMQPYMSQPNTGPLVYVLYAV | | SSITSVLSQQAYVLFYIQKS |
| | | LVHAGWSCHNGHYFSYVKAQEG | | |
| | | QWYKMDDAEVTASSITSVLSQQ | | |
| | | AYVLFYIQKSEWERHSESVSRG | | |
| | | REPRALGAEDTDRRATQGELKR | | |
| | | DHPCLQAPELDEHLVERATQES | | |
| | | TLDHWKFLQEQNKTKPEFNVRK | | |
| | | VEGTLPPDVLVIHQSKYKCGMK | | |
| | | NHHPEQQSSLLNLSSSTPTHQE | | |
| | | SMNTGTLASLRGRARRSKGKNK | | |
| | | HSKRALLVCQ | | |
| U17LM_ | | MEDDSLYLGGEWQFNHFSKLTS | | AVGAGLQNMGNTCYVNASLQ |
| HUMAN | | SRPDAAFAEIQRTSLPEKSPLS | | CLTYTPPLANYMLSREHSQT |
| Ubiquitin | | CETRVDLCDDLAPVARQLAPRE | | CHRHKGCMLCTMQAHITRAL |
| carboxyl- | | KLPLSSRRPAAVGAGLQNMGNT | | HNPGHVIQPSQALAAGFHRG |
| terminal | | CYVNASLQCLTYTPPLANYMLS | | KQEDAHEFLMFTVDAMKKAC |
| hydrolase 17- | | REHSQTCHRHKGCMLCTMQAHI | | LPGHKQVDHHSKDTTLIHQI |
| like protein 22 | | TRALHNPGHVIQPSQALAAGFH | | FGGYWRSQIKCLHCHGISDT |
| | | RGKQEDAHEFLMFTVDAMKKAC | | FDPYLDIALDIQAAQSVQQA |
| | | LPGHKQVDHHSKDTTLIHQIFG | | LEQLVKPEELNGENAYHCGV |
| | | GYWRSQIKCLHCHGISDTFDPY | | CLQRAPASKTLTLHTSAKVL |
| | | LDIALDIQAAQSVQQALEQLVK | | ILVLKRFSDVTGNKIAKNVQ |
| | | PEELNGENAYHCGVCLQRAPAS | | YPECLDMQPYMSQQNTGPLV |
| | | KTLTLHTSAKVLILVLKRESDV | | YVLYAVLVHAGWSCHNGHYF |
| | | TGNKIAKNVQYPEC | | SYVKAQEGQWYKMDDAEVTA |
| | | LDMQPYMSQQNTGPLVYVLYAV | | SSITSVLSQQAYVLFYIQKS |
| | | LVHAGWSCHNGHYFSYVKAQEG | | |
| | | QWYKMDDAEVTASSITSVLSQQ | | |
| | | AYVLFYIQKSEWERHSESVSRG | | |
| | | REPRALGAEDTDRRATQGELKR | | |
| | | DHPCLQAPELDEHLVERATQES | | |
| | | TLDHWKFLQEQNKTKPEFNVRK | | |
| | | VEGTLPPDVLVIHQSKYKCGMK | | |
| | | NHHPEQQSSLLKLSSTTPTHQE | | |
| | | SMNTGTLASLRGRARRSKGKNK | | |
| | | HSKRALLVCQ | | |
| UBP5_HUMAN | 58 | MAELSEEALLSVLPTIRVPKAG | 170 | FGPGYTGIRNLGNSCYLNSV |
| Ubiquitin | | DRVHKDECAFSEDTPESEGGLY | | VQVLFSIPDFQRKYVDKLEK |
| carboxyl- | | ICMNTFLGFGKQYVERHENKTG | | IFQNAPTDPTQDESTQVAKL |
| terminal | | QRVYLHLRRTRRPKEEDPATGT | | GHGLLSGEYSKPVPESGDGE |
| hydrolase 5 | | GDPPRKKPTRLAIGVEGGEDLS | | RVPEQKEVQDGIAPRMEKAL |
| | | EEKFELDEDVKIVILPDYLEIA | | IGKGHPEFSTNRQQDAQEFF |
| | | RDGLGGLPDIVRDRVTSAVEAL | | LHLINMVERNCRSSENPNEV |
| | | LSADSASRKQEVQAWDGEVRQV | | FRELVEEKIKCLATEKVKYT |
| | | SKHAFSLKQLDNPARIPPCGWK | | QRVDYIMQLPVPMDAALNKE |
| | | CSKCDMRENLWLNLTDGSILCG | | ELLEYEEKKRQAEEEKMALP |
| | | RRYFDGSGGNNHAVEHYRETGY | | ELVRAQVPESSCLEAYGAPE |
| | | PLAVKLGTITPDGADVYSYDED | | QVDDFWSTALQAKSVAVKTT |
| | | DMVLDPSLAEHLSHFGIDMLKM | | RFASFPDYLVIQIKKFTFGL |
| | | QKTDKTMTELEIDM | | DWVPKKLDVSIEMPEELDIS |
| | | NQRIGEWELIQESGVPLKPLFG | | QLRGTGLQPGEEELPDIAPP |
| | | PGYTGIRNLGNSCYLNSVVQVL | | LVTPDEPKGSLGFYGNEDED |
| | | FSIPDFQRKYVDKLEKIFQNAP | | SFCSPHESSPTSPMLDESVI |
| | | TDPTQDFSTQVAKLGHGLLSGE | | IQLVEMGFPMDACRKAVYYT |
| | | YSKPVPESGDGERVPEQKEVQD | | GNSGAEAAMNWVMSHMDDPD |
| | | GIAPRMFKALIGKGHPEFSTNR | | FANPLILPGSSGPGSTSAAA |
| | | QQDAQEFFLHLINMVERNCRSS | | DPPPEDCVTTIVSMGESRDQ |
| | | ENPNEVERELVEEKIKCLATEK | | ALKALRATNNSLERAVDWIE |
| | | VKYTQRVDYIMQLPVPMDAALN | | SHIDDLDAEAAMDISEGRSA |
| | | KEELLEYEEKKRQAEEEKMALP | | ADSISESVPVGPKVRDGPGK |
| | | ELVRAQVPFSSCLEAYGAPEQV | | YQLFAFISHMGTSTMCGHYV |
| | | DDFWSTALQAKSVAVKTTRFAS | | CHIKKEGRWVIYNDQKVCAS |
| | | FPDYLVIQIKKFTFGLDWVPKK | | EKPPKDLGYIYFYQRVA |
| | | LDVSIEMPEELDIS | | |
| | | QLRGTGLQPGEEELPDIAPPLV | | |
| | | TPDEPKGSLGFYGNEDEDSFCS | | |
| | | PHESSPTSPMLDESVIIQLVEM | | |
| | | GFPMDACRKAVYYTGNSGAEAA | | |
| | | MNWVMSHMDDPDFANPLILPGS | | |
| | | SGPGSTSAAADPPPEDCVTTIV | | |
| | | SMGFSRDQALKALRATNNSLER | | |
| | | AVDWIFSHIDDLDAEAAMDISE | | |
| | | GRSAADSISESVPVGPKVRDGP | | |
| | | GKYQLFAFISHMGTSTMCGHYV | | |
| | | CHIKKEGRWVIYNDQKVCASEK | | |
| | | PPKDLGYIYFYQRVAS | | |
| UBP25_HUMAN | 59 | MTVEQNVLQQSAAQKHQQTELN | | KAPVGLKNVGNTCWFSAVIQ |
| Ubiquitin | | QLREITGINDTQILQQALKDSN | | SLENLLEFRRLVLNYKPPSN |
| carboxyl- | | GNLELAVAFLTAKNAKTPQQEE | | AQDLPRNQKEHRNLPEMREL |
| terminal | | TTYYQTALPGNDRYISVGSQAD | | RYLFALLVGTKRKYVDPSRA |
| hydrolase 25 | | TNVIDLTGDDKDDLQRAIALSL | | VEILKDAFKSNDSQQQDVSE |
| | | AESNRAFRETGITDEEQAISRV | | FTHKLLDWLEDAFQMKAEEE |
| | | LEASIAENKACLKRTPTEVWRD | | TDEEKPKNPMVELFYGRELA |
| | | SRNPYDRKRQDKAPVGLKNVGN | | VGVLEGKKFENTEMFGQYPL |
| | | TCWFSAVIQSLENLLEFRRLVL | | QVNGFKDLHECLEAAMIEGE |
| | | NYKPPSNAQDLPRNQKEHRNLP | | IESLHSENSGKSGQEHWFTE |
| | | FMRELRYLFALLVGTKRKYVDP | | LPPVLTFELSRFEFNQALGR |
| | | SRAVEILKDAFKSNDSQQQDVS | | PEKIHNKLEFPQVLYLDRYM |
| | | EFTHKLLDWLEDAFQMKAEEET | | HRNREITRIKREEIKRLKDY |
| | | DEEKPKNPMVELFY | | LTVLQQRLERYLSYGSGPKR |
| | | GRFLAVGVLEGKKFENTEMFGQ | | FPLVDVLQYALEFASSKPVC |
| | | YPLQVNGFKDLHECLEAAMIEG | | TSPVDDIDASSPPSGSIPSQ |
| | | EIESLHSENSGKSGQEHWFTEL | | TLPSTTEQQGALSSELPSTS |
| | | PPVLTFELSRFEFNQALGRPEK | | PSSVAAISSRSVIHKPFTQS |
| | | IHNKLEFPQVLYLDRYMHRNRE | | RIPPDLPMHPAPRHITEEEL |
| | | ITRIKREEIKRLKDYLTVLQQR | | SVLESCLHRWRTEIENDTRD |
| | | LERYLSYGSGPKRFPLVDVLQY | | LQESISRIHRTIELMYSDKS |
| | | ALEFASSKPVCTSPVDDIDASS | | MIQVPYRLHAVLVHEGQANA |
| | | PPSGSIPSQTLPSTTEQQGALS | | GHYWAYIFDHRESRWMKYND |
| | | SELPSTSPSSVAAISSRSVIHK | | IAVTKSSWEELVRDSEGGYR |
| | | PFTQSRIPPDLPMHPAPRHITE | | NAS |
| | | EELSVLESCLHRWRTEIENDTR | | |
| | | DLQESISRIHRTIELMYSDKSM | | |
| | | IQVPYRLHAVLVHE | | |
| | | GQANAGHYWAYIFDHRESRWMK | | |
| | | YNDIAVTKSSWEELVRDSFGGY | | |
| | | RNASAYCLMYINDKAQFLIQEE | | |
| | | FNKETGQPLVGIETLPPDLRDE | | |
| | | VEEDNQRFEKELEEWDAQLAQK | | |
| | | ALQEKLLASQKLRESETSVTTA | | |
| | | QAAGDPEYLEQPSRSDESKHLK | | |
| | | EETIQIITKASHEHEDKSPETV | | |
| | | LQSAIKLEYARLVKLAQEDTPP | | |
| | | ETDYRLHHVVVYFIQNQAPKKI | | |
| | | IEKTLLEQFGDRNLSFDERCHN | | |
| | | IMKVAQAKLEMIKPEEVNLEEY | | |
| | | EEWHQDYRKERETTMYLIIGLE | | |
| | | NFQRESYIDSLLEL | | |
| | | ICAYQNNKELLSKGLYRGHDEE | | |
| | | LISHYRRECLLKLNEQAAELFE | | |
| | | SGEDREVNNGLIIMNEFIVPEL | | |
| | | PLLLVDEMEEKDILAVEDMRNR | | |
| | | WCSYLGQEMEPHLQEKLTDELP | | |
| | | KLLDCSMEIKSFHEPPKLPSYS | | |
| | | THELCERFARIMLSLSRTPADG | | |
| | | R | | |
| UBP33_HUMAN | 60 | MTGSNSHITILTLKVLPHFESL | 171 | ARGLTGLKNIGNTCYMNAAL |
| Ubiquitin | | GKQEKIPNKMSAFRNHCPHLDS | | QALSNCPPLTQFELDCGGLA |
| carboxyl- | | VGEITKEDLIQKSLGTCQDCKV | | RTDKKPAICKSYLKLMTELW |
| terminal | | QGPNLWACLENRCSYVGCGESQ | | HKSRPGSVVPTTLFQGIKTV |
| hydrolase 33 | | VDHSTIHSQETKHYLTVNLTTL | | NPTFRGYSQQDAQEFLRCLM |
| | | RVWCYACSKEVELDRKLGTQPS | | DLLHEELKEQVMEVEEDPQT |
| | | LPHVRQPHQIQENSVQDEKIPS | | ITTEETMEEDKSQSDVDFQS |
| | | NTTLKTPLVAVEDDLDIEADEE | | CESCSNSDRAENENGSRCES |
| | | DELRARGLTGLKNIGNTCYMNA | | EDNNETTMLIQDDENNSEMS |
| | | ALQALSNCPPLTQFFLDCGGLA | | KDWQKEKMCNKINKVNSEGE |
| | | RTDKKPAICKSYLKLMTELWHK | | FDKDRDSISETVDLNNQETV |
| | | SRPGSVVPTTLFQGIKTVNPTF | | KVQIHSRASEYITDVHSNDL |
| | | RGYSQQDAQEFLRCLMDLLHEE | | STPQILPSNEGVNPRLSASP |
| | | LKEQVMEVEEDPQT | | PKSGNLWPGLAPPHKKAQSA |
| | | ITTEETMEEDKSQSDVDFQSCE | | SPKRKKQHKKYRSVISDIED |
| | | SCSNSDRAENENGSRCESEDNN | | GTIISSVQCLTCDRVSVTLE |
| | | ETTMLIQDDENNSEMSKDWQKE | | TFQDLSLPIPGKEDLAKLHS |
| | | KMCNKINKVNSEGEFDKDRDSI | | SSHPTSIVKAGSCGEAYAPQ |
| | | SETVDLNNQETVKVQIHSRASE | | GWIAFFMEYVKRFVVSCVPS |
| | | YITDVHSNDLSTPQILPSNEGV | | WFWGPVVTLQDCLAAFFARD |
| | | NPRLSASPPKSGNLWPGLAPPH | | ELKGDNMYSCEKCKKLRNGV |
| | | KKAQSASPKRKKQHKKYRSVIS | | KFCKVQNFPEILCIHLKRER |
| | | DIFDGTIISSVQCLTCDRVSVT | | HELMESTKISTHVSFPLEGL |
| | | LETFQDLSLPIPGKEDLAKLHS | | DLQPFLAKDSPAQIVTYDLL |
| | | SSHPTSIVKAGSCGEAYAPQGW | | SVICHHGTASSGHYIAYCRN |
| | | IAFFMEYVKRFVVSCVPSWEWG | | NLNNLWYEFDDQSVTEVSES |
| | | PVVTLQDCLAAFFARDELKGDN | | TVQNAEAYVLFYRKSS |
| | | MYSCEKCKKLRNGV | | |
| | | KFCKVQNFPEILCIHLKRFRHE | | |
| | | LMFSTKISTHVSFPLEGLDLQP | | |
| | | FLAKDSPAQIVTYDLLSVICHH | | |
| | | GTASSGHYIAYCRNNLNNLWYE | | |
| | | FDDQSVTEVSESTVQNAEAYVL | | |
| | | FYRKSSEEAQKERRRISNLLNI | | |
| | | MEPSLLQFYISRQWLNKFKTFA | | |
| | | EPGPISNNDFLCIHGGVPPRKA | | |
| | | GYIEDLVLMLPQNIWDNLYSRY | | |
| | | GGGPAVNHLYICHTCQIEAEKI | | |
| | | EKRRKTELEIFIRLNRAFQKED | | |
| | | SPATFYCISMQWFREWESFVKG | | |
| | | KDGDPPGPIDNTKIAVTKCGNV | | |
| | | MLRQGADSGQISEETWNFLQSI | | |
| | | YGGGPEVILRPPVVHVDPDILQ | | |
| | | AEEKIEVETRSL | | |
| UBP21_HUMAN | 61 | MPQASEHRLGRTREPPVNIQPR | 172 | LGSGHVGLRNLGNTCFLNAV |
| Ubiquitin | | VGSKLPFAPRARSKERRNPASG | | LQCLSSTRPLRDFCLRRDER |
| carboxyl- | | PNPMLRPLPPRPGLPDERLKKL | | QEVPGGGRAQELTEAFADVI |
| terminal | | ELGRGRTSGPRPRGPLRADHGV | | GALWHPDSCEAVNPTRFRAV |
| hydrolase 21 | | PLPGSPPPTVALPLPSRTNLAR | | FQKYVPSFSGYSQQDAQEFL |
| | | SKSVSSGDLRPMGIALGGHRGT | | KLLMERLHLEINRRGRRAPP |
| | | GELGAALSRLALRPEPPTLRRS | | ILANGPVPSPPRRGGALLEE |
| | | TSLRRLGGFPGPPTLESIRTEP | | PELSDDDRANLMWK |
| | | PASHGSFHMISARSSEPFYSDD | | RYLEREDSKIVDLFVGQLKS |
| | | KMAHHTLLLGSGHVGLRNLGNT | | CLKCQACGYRSTTFEVECDL |
| | | CFLNAVLQCLSSTRPLRDFCLR | | SLPIPKKGFAGGKVSLRDCF |
| | | RDFRQEVPGGGRAQELTEAFAD | | NLFTKEEELESENAPVCDRC |
| | | VIGALWHPDSCEAVNPTRFRAV | | RQKTRSTKKLTVQRFPRILV |
| | | FQKYVPSFSGYSQQ | | LHLNRFSASRGSIKKSSVGV |
| | | DAQEFLKLLMERLHLEINRRGR | | DFPLQRLSLGDFASDKAGSP |
| | | RAPPILANGPVPSPPRRGGALL | | VYQLYALCNHSGSVHYGHYT |
| | | EEPELSDDDRANLMWKRYLERE | | ALCRCQTGWHVYNDSRVSPV |
| | | DSKIVDLFVGQLKSCLKCQACG | | SENQVASSEGYVLFYQLMQ |
| | | YRSTTFEVFCDLSLPIPKKGFA | | |
| | | GGKVSLRDCENLFTKEEELESE | | |
| | | NAPVCDRCRQKTRSTKKLTVQR | | |
| | | FPRILVLHLNRESASRGSIKKS | | |
| | | SVGVDFPLQRLSLGDFASDKAG | | |
| | | SPVYQLYALCNHSGSVHYGHYT | | |
| | | ALCRCQTGWHVYNDSRVSPVSE | | |
| | | NQVASSEGYVLFYQLMQEPPRC | | |
| | | L | | |
| U17L4_HUMAN | 62 | MGDDSLYLGGEWQFNHESKLTS | 173 | AVGAGLQNMGNTCYENASLQ |
| Inactive | | SRPDAAFAEIQRTSLPEKSPLS | | CLTYTLPLANYMLSREHSQT |
| ubiquitin | | SETRVDLCDDLAPVARQLAPRE | | CQRPKCCMLCTMQAHITWAL |
| carboxyl- | | KLPLSSRRPAAVGAGLQNMGNT | | HSPGHVIQPSQALAAGFHRG |
| terminal | | CYENASLQCLTYTLPLANYMLS | | KQEDVHEFLMFTVDAMKKAC |
| hydrolase 17- | | REHSQTCQRPKCCMLCTMQAHI | | LPGHKQVDHHSKDTTLIHQI |
| like protein 4 | | TWALHSPGHVIQPSQALAAGFH | | FGGCWRSQIKCLHCHGISDT |
| | | RGKQEDVHEFLMFTVDAMKKAC | | FDPYLDIALDIQAAQSVKQA |
| | | LPGHKQVDHHSKDTTLIHQIFG | | LEQLVKPEELNGENAYHCGL |
| | | GCWRSQIKCLHCHGISDTFDPY | | CLQRAPASNTLTLHTSAKVL |
| | | LDIALDIQAAQSVKQALEQLVK | | ILVLKRESDVAGNKLAKNVQ |
| | | PEELNGENAYHCGLCLQRAPAS | | YPECLDMQPYMSQQNTGPLV |
| | | | | YVLYAVLVHAGWSCHDGYYE |
| | | NTLTLHTSAKVLILVLKRFSDV | 174 | SYVKAQEGQWYKMDDAEVTV |
| | | AGNKLAKNVQYPEC | | CSITSVLSQQAYVLFYIQKS |
| | | LDMQPYMSQQNTGPLVYVLYAV | | AVGAGLQNMGNTCYVNASLQ |
| | | LVHAGWSCHDGYYFSYVKAQEG | | CLTYTPPLANYMLSREHSQT |
| | | QWYKMDDAEVTVCSITSVLSQQ | | CHRHKGCMLCTMQAHITRAL |
| | | AYVLFYIQKSEWERHSESVSRG | | HNPGHVIQPSQALAAGFHRG |
| | | REPRALGAEDTDRPATQGELKR | | KQEDAHEFLMFTVDAMKKAC |
| | | DHPCLQVPELDEHLVERATEES | | LPGHKQVDHHSKDTTLIHQI |
| | | TLDHWKFPQEQNKMKPEFNVRK | | FGGYWRSQIKCLHCHGISDT |
| | | VEGTLPPNVLVIHQSKYKCGMK | | |
| | | NHHPEQQSSLLNLSSMNSTDQE | | |
| | | SMNTGTLASLQGRTRRSKGKNK | | |
| | | HSKRSLLVCQ | | |
| | | MEDDSLYLGGEWQFNHFSKLTS | | |
| | | SRPDAAFAEIQRTSLPEKSPLS | | |
| | | CETRVDLCDDLAPVARQLAPRE | | |
| | | KLPLSSRRPAAVGAGLQNMGNT | | |
| | | CYVNASLQCLTYTPPLANYMLS | | |
| | | REHSQTCHRHKGCMLCTMQAHI | | |
| U17LK_HUMAN | 63 | TRALHNPGHVIQPSQALAAGFH | | FDPYLDIALDIQAAQSVQQA |
| Ubiquitin | | RGKQEDAHEFLMFTVDAMKKAC | | LEQLVKPEELNGENAYHCGV |
| carboxyl- | | LPGHKQVDHHSKDTTLIHQIFG | | CLQRAPASKTLTLHTSAKVL |
| terminal | | GYWRSQIKCLHCHGISDTEDPY | | ILVLKRFSDVTGNKIAKNVQ |
| hydrolase 17- | | LDIALDIQAAQSVQQALEQLVK | | YPECLDMQPYMSQPNTGPLV |
| like protein 20 | | PEELNGENAYHCGVCLQRAPAS | | YVLYAVLVHAGWSCHNGHYF |
| | | KTLTLHTSAKVLILVLKRFSDV | | SYVKAQEGQWYKMDDAEVTA |
| | | TGNKIAKNVQYPECLDMQPYMS | | SSITSVLSQQAYVLFYIQKS |
| | | QPNTGPLVYVLYAVLVHAGWSC | | |
| | | HNGHYFSYVKAQEGQWYKMDDA | | |
| | | EVTASSITSVLSQQAYVLFYIQ | | |
| | | KSEWERHSESVSRGREPRALGA | | |
| | | EDTDRRATQGELKRDHPCLQAP | | |
| | | ELDEHLVERATQESTLDHWKEL | | |
| | | QEQNKTKPEFNVRKVEGTLPPD | | |
| | | VLVIHQSKYKCGMKNHHPEQQS | | |
| | | SLLNLSSTTPTHQESMNTGTLA | | |
| | | SLRGRARRSKGKNKHSKRALLV | | |
| | | CQ | | |
| UBP12_HUMAN | 64 | MEILMTVSKFASICTMGANASA | 175 | EHYFGLVNFGNTCYCNSVLQ |
| Ubiquitin | | LEKEIGPEQFPVNEHYFGLVNF | | ALYFCRPFREKVLAYKSQPR |
| carboxyl- | | GNTCYCNSVLQALYFCRPFREK | | KKESLLTCLADLFHSIATQK |
| terminal | | VLAYKSQPRKKESLLTCLADLF | | KKVGVIPPKKFITRLRKENE |
| hydrolase 12 | | HSIATQKKKVGVIPPKKFITRL | | LFDNYMQQDAHEFLNYLLNT |
| | | RKENELFDNYMQQDAHEFLNYL | | IADILQEERKQEKQNGRLPN |
| | | LNTIADILQEERKQEKQNGRLP | | GNIDNENNNSTPDPTWVHEI |
| | | NGNIDNENNNSTPDPTWVHEIF | | FQGTLTNETRCLTCETISSK |
| | | QGTLTNETRCLTCETISSKDED | | DEDFLDLSVDVEQNTSITHC |
| | | FLDLSVDVEQNTSITHCLRGES | | LRGESNTETLCSEYKYYCEE |
| | | NTETLCSEYKYYCEECRSKQEA | | CRSKQEAHKRMKVKKLPMIL |
| | | HKRMKVKKLPMILALHLKRFKY | | ALHLKRFKYMDQLHRYTKLS |
| | | | | YRVVFPLELRLENTSGDATN |
| | | MDQLHRYTKLSYRVVFPLELRL | | PDRMYDLVAVVVHCGSGPNR |
| | | FNTSGDATNPDRMY | | GHYIAIVKSHDFWLLEDDDI |
| | | DLVAVVVHCGSGPNRGHYIAIV | | VEKIDAQAIEEFYGLTSDIS |
| | | KSHDEWLLEDDDIVEKIDAQAI | | KNSESGYILFYQSR |
| | | EEFYGLTSDISKNSESGYILFY | | |
| | | QSRD | | |
| UL17C_HUMAN | 65 | MEEDSLYLGGEWQENHESKLTS | 176 | AVGAGLQNMGNTCYVNASLQ |
| Ubiquitin | | SRPDAAFAEIQRTSLPEKSPLS | | CLTYTPPLANYMLSREHSQT |
| carboxyl- | | CETRVDLCDDLAPVARQLAPRE | | CHRHKGCMLCTMQAHITRAL |
| terminal | | KLPLSNRRPAAVGAGLQNMGNT | | HNPGHVIQPSQALAAGFHRG |
| hydrolase 17- | | CYVNASLQCLTYTPPLANYMLS | | KQEDAHEFLMFTVDAMKKAC |
| like protein 12 | | REHSQTCHRHKGCMLCTMQAHI | | LPGHKQVDHHSKDTTLIHQI |
| | | TRALHNPGHVIQPSQALAAGFH | | FGGYWRSQIKCLHCHGISDT |
| | | RGKQEDAHEFLMFTVDAMKKAC | | FDPYLDIALDIQAAQSVQQA |
| | | LPGHKQVDHHSKDTTLIHQIFG | | LEQLVKPEELNGENAYHCGV |
| | | GYWRSQIKCLHCHGISDTFDPY | | CLQRAPASKMLTLLTSAKVL |
| | | LDIALDIQAAQSVQQALEQLVK | | ILVLKRFSDVTGNKIAKNVQ |
| | | PEELNGENAYHCGVCLQRAPAS | | YPECLDMQPYMSQPNTGPLV |
| | | KMLTLLTSAKVLILVLKRESDV | | YVLYAVLVHAGWSCHNGHYF |
| | | TGNKIAKNVQYPEC | | SYVKAQEGQWYKMDDAEVTA |
| | | LDMQPYMSQPNTGPLVYVLYAV | | SSITSVLSQQAYVLFYIQKS |
| | | LVHAGWSCHNGHYFSYVKAQEG | | |
| | | QWYKMDDAEVTASSITSVLSQQ | | |
| | | AYVLFYIQKSEWERHSESVSRG | | |
| | | REPRALGAEDTDRRATQGELKR | | |
| | | DHPCLQAPELDEHLVERATQES | | |
| | | TLDHWKFLQEQNKTKPEFNVRK | | |
| | | VEGTLPPDVLVIHQSKYKCGMK | | |
| | | NHHPEQQSSLLKLSSTTPTHQE | | |
| | | SMNTGTLASLRGRARRSKGKNK | | |
| | | HSKRALLVCQ | | |
| UBP20_HUMAN | 66 | MGDSRDLCPHLDSIGEVTKEDL | 177 | PRGLTGMKNLGNSCYMNAAL |
| Ubiquitin | | LLKSKGTCQSCGVTGPNLWACL | | QALSNCPPLTQFFLECGGLV |
| carboxyl- | | QVACPYVGCGESFADHSTIHAQ | | RTDKKPALCKSYQKLVSEVW |
| terminal | | AKKHNLTVNLTTFRLWCYACEK | | HKKRPSYVVPTSLSHGIKLV |
| hydrolase | | EVFLEQRLAAPLLGSSSKFSEQ | | NPMFRGYAQQDTQEFLRCLM |
| | | DSPPPSHPLKAVPIAVADEGES | | DQLHEELKEPVVATVALTEA |
| | | ESEDDDLKPRGLTGMKNLGNSC | | RDSDSSDTDEKREGDRSPSE |
| | | YMNAALQALSNCPPLTQFFLEC | | DEFLSCDSSSDRGEGDGQGR |
| | | GGLVRTDKKPALCKSYQKLVSE | | GGGSSQAETELLIPDEAGRA |
| | | VWHKKRPSYVVPTSLSHGIKLV | | ISEKERMKDRKESWGQQRTN |
| | | NPMFRGYAQQDTQEFLRCLMDQ | | SEQVDEDADVDTAMAALDDQ |
| | | LHEELKEPVVATVALTEARDSD | | PAEAQPPSPRSSSPCRTPEP |
| | | SSDTDEKREGDRSPSEDEFLSC | | DNDAHLRSSSRPCSPVHHHE |
| | | DSSSDRGEGDGQGR | | GHAKLSSSPPRASPVRMAPS |
| | | GGGSSQAETELLIPDEAGRAIS | | YVLKKAQVLSAGSRRRKEQR |
| | | EKERMKDRKFSWGQQRTNSEQV | | YRSVISDIFDGSILSLVQCL |
| | | DEDADVDTAMAALDDQPAEAQP | | TCDRVSTTVETFQDLSLPIP |
| | | PSPRSSSPCRTPEPDNDAHLRS | | GKEDLAKLHSAIYQNVPAKP |
| | | SSRPCSPVHHHEGHAKLSSSPP | | GACGDSYAAQGWLAFIVEYI |
| | | RASPVRMAPSYVLKKAQVLSAG | | RRFVVSCTPSWFWGPVVTLE |
| | | SRRRKEQRYRSVISDIFDGSIL | | DCLAAFFAADELKGDNMYSC |
| | | SLVQCLTCDRVSTTVETFQDLS | | ERCKKLRNGVKYCKVLRLPE |
| | | LPIPGKEDLAKLHSAIYQNVPA | | ILCIHLKRFRHEVMYSFKIN |
| | | KPGACGDSYAAQGWLAFIVEYI | | SHVSFPLEGLDLRPFLAKEC |
| | | RRFVVSCTPSWFWGPVVTLEDC | | TSQITTYDLLSVICHHGTAG |
| | | LAAFFAADELKGDNMYSCERCK | | SGHYIAYCQNVINGQWYEFD |
| | | KLRNGVKYCKVLRLPEILCIHL | | DQYVTEVHETVVQNAEGYVL |
| | | KRFRHEVMYSEKIN | | FYRKSS |
| | | SHVSFPLEGLDLRPFLAKECTS | | |
| | | QITTYDLLSVICHHGTAGSGHY | | |
| | | IAYCQNVINGQWYEFDDQYVTE | | |
| | | VHETVVQNAEGYVLFYRKSSEE | | |
| | | AMRERQQVVSLAAMREPSLLRE | | |
| | | YVSREWLNKENTFAEPGPITNQ | | |
| | | TELCSHGGIPPHKYHYIDDLVV | | |
| | | ILPQNVWEHLYNRFGGGPAVNH | | |
| | | LYVCSICQVEIEALAKRRRIEI | | |
| | | DTFIKLNKAFQAEESPGVIYCI | | |
| | | SMQWFREWEAFVKGKDNEPPGP | | |
| | | IDNSRIAQVKGSGHVQLKQGAD | | |
| | | YGQISEETWTYLNSLYGGGPEI | | |
| | | AIRQSVAQPLGPENLHGEQKIE | | |
| | | AETRAV | | |
| UBP46_HUMAN | 67 | MTVRNIASICNMGTNASALEKD | 178 | EHYFGLVNFGNTCYCNSVLQ |
| Ubiquitin | | IGPEQFPINEHYFGLVNFGNTC | | ALYFCRPFRENVLAYKAQQK |
| carboxyl- | | YCNSVLQALYFCRPERENVLAY | | KKENLLTCLADLFHSIATQK |
| terminal | | KAQQKKKENLLTCLADLFHSIA | | KKVGVIPPKKFISRLRKEND |
| hydrolase 46 | | TQKKKVGVIPPKKFISRLRKEN | | LFDNYMQQDAHEFLNYLLNT |
| | | DLFDNYMQQDAHEFLNYLLNTI | | IADILQEEKKQEKQNGKLKN |
| | | ADILQEEKKQEKQNGKLKNGNM | | GNMNEPAENNKPELTWVHEI |
| | | NEPAENNKPELTWVHEIFQGTL | | FQGTLTNETRCLNCETVSSK |
| | | TNETRCLNCETVSSKDEDELDL | | DEDFLDLSVDVEQNTSITHC |
| | | SVDVEQNTSITHCLRDESNTET | | LRDESNTETLCSEQKYYCET |
| | | LCSEQKYYCETCCSKQEAQKRM | | CCSKQEAQKRMRVKKLPMIL |
| | | RVKKLPMILALHLKRFKYMEQL | | ALHLKRFKYMEQLHRYTKLS |
| | | HRYTKLSYRVVFPLELRLENTS | | YRVVFPLELRLENTSSDAVN |
| | | SDAVNLDRMYDLVA | | LDRMYDLVAVVVHCGSGPNR |
| | | VVVHCGSGPNRGHYITIVKSHG | | GHYITIVKSHGFWLLFDDDI |
| | | FWLLEDDDIVEKIDAQAIEEFY | | VEKIDAQAIEEFYGLTSDIS |
| | | GLTSDISKNSESGYILFYQSRE | | KNSESGYILFYQSR |
| CYLD_HUMAN | 68 | MSSGLWSQEKVTSPYWEERIFY | 179 | GKKKGIQGHYNSCYLDSTLF |
| Ubiquitin | | LLLQECSVTDKQTQKLLKVPKG | | CLFAFSSVLDTVLLRPKEKN |
| carboxyl- | | SIGQYIQDRSVGHSRIPSAKGK | | DVEYYSETQELLRTEIVNPL |
| terminal | | KNQIGLKILEQPHAVLEVDEKD | | RIYGYVCATKIMKLRKILEK |
| hydrolase | | VVEINEKFTELLLAITNCEERF | | VEAASGFTSEEKDPEEFLNI |
| CYLD | | SLFKNRNRLSKGLQIDVGCPVK | | LFHHILRVEPLLKIRSAGQK |
| | | VQLRSGEEKFPGVVRERGPLLA | | VQDCYFYQIFME |
| | | ERTVSGIFFGVELLEEGRGQGF | | KNEKVGVPTIQQLLEWSFIN |
| | | TDGVYQGKQLFQCDEDCGVEVA | | SNLKFAEAPSCLIIQMPREG |
| | | LDKLELIEDDDTALESDYAGPG | | KDFKLFKKIFPSLELNITDL |
| | | DTMQVELPPLEINSRVSLKVGE | | LEDTPRQCRICGGLAMYECR |
| | | TIESGTVIFCDVLPGKESLGYE | | ECYDDPDISAGKIKQFCKTC |
| | | VGVDMDNPIGNWDGREDGVQLC | | NTQVHLHPKRLNHKYNPVSL |
| | | SFACVESTILLHIN | | PKDLPDWDWRHGCIPCQNME |
| | | DIIPALSESVTQERRPPKLAFM | | LFAVLCIETSHYVAFVKYGK |
| | | SRGVGDKGSSSHNKPKATGSTS | | DDSAWLFFDSMADRDGGQNG |
| | | DPGNRNRSELFYTLNGSSVDSQ | | FNIPQVTPCPEVGEYLKMSL |
| | | PQSKSKNTWYIDEVAEDPAKSL | | EDLHSLDSRRIQGCARRLLC |
| | | TEISTDEDRSSPPLQPPPVNSL | | DAYMCMYQSPT |
| | | TTENRFHSLPFSLTKMPNINGS | | |
| | | IGHSPLSLSAQSVMEELNTAPV | | |
| | | QESPPLAMPPGNSHGLEVGSLA | | |
| | | EVKENPPFYGVIRWIGQPPGLN | | |
| | | EVLAGLELEDECAGCTDGTERG | | |
| | | TRYFTCALKKALFVKLKSCRPD | | |
| | | SRFASLQPVSNQIERCNSLAFG | | |
| | | GYLSEVVEENTPPKMEKEGLEI | | |
| | | MIGKKKGIQGHYNS | | |
| | | CYLDSTLFCLFAFSSVLDTVLL | | |
| | | RPKEKNDVEYYSETQELLRTEI | | |
| | | VNPLRIYGYVCATKIMKLRKIL | | |
| | | EKVEAASGFTSEEKDPEEFLNI | | |
| | | LFHHILRVEPLLKIRSAGQKVQ | | |
| | | DCYFYQIFMEKNEKVGVPTIQQ | | |
| | | LLEWSFINSNLKFAEAPSCLII | | |
| | | QMPREGKDEKLFKKIFPSLELN | | |
| | | ITDLLEDTPRQCRICGGLAMYE | | |
| | | CRECYDDPDISAGKIKQFCKTC | | |
| | | NTQVHLHPKRLNHKYNPVSLPK | | |
| | | DLPDWDWRHGCIPCQNMELFAV | | |
| | | LCIETSHYVAFVKYGKDDSAWL | | |
| | | FFDSMADRDGGQNGFNIPQVTP | | |
| | | CPEVGEYLKMSLEDLHSLDSRR | | |
| | | IQGCARRLLCDAYMCMYQSPTM | | |
| | | SLYK | | |
| UBP16_HUMAN | 69 | MGKKRTKGKTVPIDDSSETLEP | 180 | ITVKGLSNLGNTCFFNAVMQ |
| Ubiquitin | | VCRHIRKGLEQGNLKKALVNVE | | NLSQTPVLRELLKEVKMSGT |
| carboxyl- | | WNICQDCKTDNKVKDKAEEETE | | IVKIEPPDLALTEPLEINLE |
| terminal | | EKPSVWLCLKCGHQGCGRNSQE | | PPGPLTLAMSQFLNEMQETK |
| hydrolase 16 | | QHALKHYLTPRSEPHCLVLSLD | | KGVVTPKELFSQVCKKAVRE |
| | | NWSVWCYVCDNEVQYCSSNQLG | | KGYQQQDSQELLRYLLDGMR |
| | | QVVDYVRKQASITTPKPAEKDN | | AEEHQRVSKGILKAFGNSTE |
| | | GNIELENKKLEKESKNEQEREK | | KLDEELKNKVKDYEKKKSMP |
| | | KENMAKENPPMNSPCQITVKGL | | SFVDRIFGGELTSMIMCDQC |
| | | SNLGNTCFFNAVMQNLSQTPVL | | RTVSLVHESELDLSLPVLDD |
| | | RELLKEVKMSGTIVKIEPPDLA | | QSGKKSVNDKNLKKTVEDED |
| | | LTEPLEINLEPPGPLTLAMSQF | | QDSEEEKDNDSYIKERSDIP |
| | | LNEMQETKKGVVTPKELFSQVC | | SGTSKHLQKKAKKQAKKQAK |
| | | KKAVRFKGYQQQDS | | NQRRQQKIQGKVLHLNDICT |
| | | QELLRYLLDGMRAEEHQRVSKG | | IDHPEDSEYEAEMSLQGEVN |
| | | ILKAFGNSTEKLDEELKNKVKD | | IKSNHISQEGVMHKEYCVNQ |
| | | YEKKKSMPSFVDRIFGGELTSM | | KDLNGQAKMIESVTDNQKST |
| | | IMCDQCRTVSLVHESFLDLSLP | | EEVDMKNINMDNDLEVLTSS |
| | | VLDDQSGKKSVNDKNLKKTVED | | PTRNLNGAYLTEGSNGEVDI |
| | | EDQDSEEEKDNDSYIKERSDIP | | SNGFKNLNLNAALHPDEINI |
| | | SGTSKHLQKKAKKQAKKQAKNQ | | EILNDSHTPGTKVYEVVNED |
| | | RRQQKIQGKVLHLNDICTIDHP | | PETAFCTLANREVENTDECS |
| | | EDSEYEAEMSLQGEVNIKSNHI | | IQHCLYQFTRNEKLRDANKL |
| | | SQEGVMHKEYCVNQKDLNGQAK | | LCEVCTRRQCNGPKANIKGE |
| | | MIESVTDNQKSTEEVDMKNINM | | RKHVYTNAKKQMLISLAPPV |
| | | DNDLEVLTSSPTRNLNGAYLTE | | LTLHLKRFQQAGENLRKVNK |
| | | GSNGEVDISNGFKNLNLNAALH | | HIKFPEIL |
| | | PDEINIEILNDSHT | | DLAPFCTLKCKNVAEENTRV |
| | | PGTKVYEVVNEDPETAFCTLAN | | LYSLYGVVEHSGTMRSGHYT |
| | | REVENTDECSIQHCLYQFTRNE | | AYAKARTANSHLSNLVLHGD |
| | | KLRDANKLLCEVCTRRQCNGPK | | IPQDFEMESKGQWFHISDTH |
| | | ANIKGERKHVYTNAKKQMLISL | | VQAVPTTKVLNSQAYLLFYE |
| | | APPVLTLHLKRFQQAGENLRKV | | RIL |
| | | NKHIKFPEILDLAPFCTLKCKN | | |
| | | VAEENTRVLYSLYGVVEHSGTM | | |
| | | RSGHYTAYAKARTANSHLSNLV | | |
| | | LHGDIPQDFEMESKGQWFHISD | | |
| | | THVQAVPTTKVLNSQAYLLFYE | | |
| | | RIL | | |
| ALG13_HUMAN | 70 | MKCVFVTVGTTSEDDLIACVSA | 181 | YRYKDSLKEDIQKADLVISH |
| Putative | | PDSLQKIESLGYNRLILQIGRG | | AGAGSCLETLEKGKPLVVVI |
| bifunctional | | TVVPEPESTESFTLDVYRYKDS | | NEKLMNNHQLELAKQLHKEG |
| UDP-N- | | LKEDIQKADLVISHAGAGSCLE | | HLFYCTCRVLTCPGQAKSIA |
| acetylglucosa | | TLEKGKPLVVVINEKLMNNHQL | | SAPGKCQDSAALTSTAFSGL |
| mine | | ELAKQLHKEGHLFYCTCRVLTC | | DFGLLSGYLHKQALVTATHP |
| transferase | | PGQAKSIASAPGKCQDSAALTS | | TCTLLFPSCHAFFPLPLTPT |
| and | | TAFSGLDFGLLSGYLHKQALVT | | LYKMHKGWKNYCSQKSLNEA |
| deubiquitinase | | ATHPTCTLLFPSCHAFFPLPLT | | SMDEYLGSLGLFRKLTAKDA |
| ALG13 | | PTLYKMHKGWKNYCSQKSLNEA | | SCLFRAISEQLFCSQVHHLE |
| | | SMDEYLGSLGLFRKLTAKDASC | | IRKACVSYMRENQQTFESYV |
| | | LFRAISEQLFCSQVHHLEIRKA | | EGSFEKYLERLGDPKESAGQ |
| | | CVSYMRENQQTFESYVEGSFEK | | LEIRALSLIYNRDFILYRFP |
| | | YLERLGDPKESAGQ | | GKPPTYVTDNGYEDKILLCY |
| | | LEIRALSLIYNRDFILYRFPGK | | SSSGHYDSVYS |
| | | PPTYVTDNGYEDKILLCYSSSG | | |
| | | HYDSVYSKQFQSSAAVCQAVLY | | |
| | | EILYKDVFVVDEEELKTAIKLE | | |
| | | RSGSKKNRNNAVTGSEDAHTDY | | |
| | | KSSNQNRMEEWGACYNAENIPE | | |
| | | GYNKGTEETKSPENPSKMPFPY | | |
| | | KVLKALDPEIYRNVEFDVWLDS | | |
| | | RKELQKSDYMEYAGRQYYLGDK | | |
| | | CQVCLESEGRYYNAHIQEVGNE | | |
| | | NNSVTVFIEELAEKHVVPLANL | | |
| | | KPVTQVMSVPAWNAMPSRKGRG | | |
| | | YQKMPGGYVPEIVISEMDIKQQ | | |
| | | KKMFKKIRGKEVYM | | |
| | | TMAYGKGDPLLPPRLQHSMHYG | | |
| | | HDPPMHYSQTAGNVMSNEHFHP | | |
| | | QHPSPRQGRGYGMPRNSSRFIN | | |
| | | RHNMPGPKVDFYPGPGKRCCQS | | |
| | | YDNESYRSRSFRRSHRQMSCVN | | |
| | | KESQYGFTPGNGQMPRGLEETI | | |
| | | TFYEVEEGDETAYPTLPNHGGP | | |
| | | STMVPATSGYCVGRRGHSSGKQ | | |
| | | TLNLEEGNGQSENGRYHEEYLY | | |
| | | RAEPDYETSGVYSTTASTANLS | | |
| | | LQDRKSCSMSPQDTVTSYNYPQ | | |
| | | KMMGNIAAVAASCANNVPAPVL | | |
| | | SNGAAANQAISTTSVSSQNAIQ | | |
| | | PLFVSPPTHGRPVI | | |
| | | ASPSYPCHSAIPHAGASLPPPP | | |
| | | PPPPPPPPPPPPPPPPPPPPPP | | |
| | | PALDVGETSNLQPPPPLPPPPY | | |
| | | SCDPSGSDLPQDTKVLQYYENL | | |
| | | GLQCYYHSYWHSMVYVPQMQQQ | | |
| | | LHVENYPVYTEPPLVDQTVPQC | | |
| | | YSEVRREDGIQAEASANDTEPN | | |
| | | ADSSSVPHGAVYYPVMSDPYGQ | | |
| | | PPLPGFDSCLPVVPDYSCVPPW | | |
| | | HPVGTAYGGSSQIHGAINPGPI | | |
| | | GCIAPSPPASHYVPQGM | | |
| OTU1_HUMAN | 71 | MFGPAKGRHFGVHPAPGFPGGV | 182 | QGLSSRTRVRELQGQIAAIT |
| Ubiquitin | | SQQAAGTKAGPAGAWPVGSRTD | | GIAPGGQRILVGYPPECLDL |
| thioesterase | | TMWRLRCKAKDGTHVLQGLSSR | | SNGDTILEDLPIQSGDMLII |
| OTU1 | | TRVRELQGQIAAITGIAPGGQR | | EEDQTRPRSSPAFTKRGASS |
| | | ILVGYPPECLDLSNGDTILEDL | | YVRETLPVLTRTVVPADNSC |
| | | PIQSGDMLIIEEDQTRPRSSPA | | LETSVYYVVEGGVLNPACAP |
| | | FTKRGASSYVRETLPVLTRTVV | | EMRRLIAQIVASDPDFYSEA |
| | | PADNSCLFTSVYYVVEGGVLNP | | ILGKTNQEYCDWIKRDDTWG |
| | | ACAPEMRRLIAQIVASDPDFYS | | GAIEISILSKFYQCEICVVD |
| | | EAILGKTNQEYCDWIKRDDTWG | | TQTVRIDRFGEDAGYTKRVL |
| | | GAIEISILSKFYQCEICVVDTQ | | LIYDGIHYDPLQ |
| | | TVRIDRFGEDAGYTKRVLLIYD | | |
| | | GIHYDPLQRNFPDPDTPPLTIF | | |
| | | SSNDDIVLVQALELADEARRRR | | |
| | | QFTDVNRFTLRCMVCQKGLTGQ | | |
| | | AEAREHAKETGHTNEGEV | | |
| OTUD1_HUMAN | 72 | MQLYSSVCTHYPAGAPGPTAAA | 183 | HREAAAVPAAKMPAFSSCFE |
| MOTU | | PAPPAAATPFKVSLQPPGAAGA | | VVSGAAAPASAAAGPPGASC |
| domain- | | APEPETGECQPAAAAEHREAAA | | KPPLPPHYTSTAQITVRALG |
| containing | | VPAAKMPAFSSCFEVVSGAAAP | | ADRLLLHGPDPVPGAAGSAA |
| protein 1 | | ASAAAGPPGASCKPPLPPHYTS | | APRGRCLLLAPAPAAPVPPR |
| | | TAQITVRALGADRLLLHGPDPV | | RGSSAWLLEELLRPDCPEPA |
| | | PGAAGSAAAPRGRCLLLAPAPA | | GLDATREGPDRNERLSEHRQ |
| | | APVPPRRGSSAWLLEELLRPDC | | ALAAAKHRGPAATPGSPDPG |
| | | PEPAGLDATREGPDRNERLSEH | | PGPWGEEHLAERGPRGWERG |
| | | RQALAAAKHRGPAATPGSPDPG | | GDRCDAPGGDAARRPDPEAE |
| | | PGPWGEEHLAERGPRGWERGGD | | APPAGSIEAAPSSAAEPVIV |
| | | RCDAPGGDAARRPDPEAEAPPA | | SRSDPRDEKLALYLAEVEKQ |
| | | GSIEAAPSSAAEPVIVSRSDPR | | DKYLRQRNKYRFHIIPDGNC |
| | | DEKLALYLAEVEKQ | | LYRAVSKTVYGDQSLHRELR |
| | | DKYLRQRNKYRFHIIPDGNCLY | | EQTVHYIADHLDHFSPLIEG |
| | | RAVSKTVYGDQSLHRELREQTV | | DVGEFIIAAAQDGAWAGYPE |
| | | HYIADHLDHESPLIEGDVGEFI | | LLAMGQMLNVNIHLTTGGRL |
| | | IAAAQDGAWAGYPELLAMGQML | | ESPTVSTMIHYLGPEDSLRP |
| | | NVNIHLTTGGRLESPTVSTMIH | | SIWLSWLSNGHYDAV |
| | | YLGPEDSLRPSIWLSWLSNGHY | | |
| | | DAVFDHSYPNPEYDNWCKQTQV | | |
| | | QRKRDEELAKSMAISLSKMYIE | | |
| | | QNACS | | |
| OTU6B_HUMAN | 73 | MEAVLTEELDEEEQLLRRHRKE | 184 | QKHREELEQLKLTTKENKID |
| MAN | | KKELQAKIQGMKNAVPKNDKKR | | SVAVNISNLVLENQPPRISK |
| Deubiquitinas | | RKQLTEDVAKLEKEMEQKHREE | | AQKRREKKAALEKEREERIA |
| e OTUD6B | | LEQLKLTTKENKIDSVAVNISN | | EAEIENLTGARHMESEKLAQ |
| | | LVLENQPPRISKAQKRREKKAA | | ILAARQLEIKQIPSDGHCMY |
| | | LEKEREERIAEAEIENLTGARH | | KAIEDQLKEKDCALTVVALR |
| | | MESEKLAQILAARQLEIKQIPS | | SQTAEYMQSHVEDFLPFLTN |
| | | DGHCMYKAIEDQLKEKDCALTV | | PNTGDMYTPEEFQKYCEDIV |
| | | VALRSQTAEYMQSHVEDELPEL | | NTAAWGGQLELRALSHILQT |
| | | TNPNTGDMYTPEEFQKYCEDIV | | PIEIIQADSPPIIVGEEYSK |
| | | NTAAWGGQLELRALSHILQTPI | | KPLILVYMRHAYG |
| | | EIIQADSPPIIVGEEYSKKPLI | | |
| | | LVYMRHAYGLGEHYNSVTRLVN | | |
| | | IVTENCS | | |
| OTU6A_HUMAN | 74 | MDDPKSEQQRILRRHQRERQEL | 185 | QELEKFQDDSSIESVVEDLA |
| MOTU | | QAQIRSLKNSVPKTDKTKRKQL | | KMNLENRPPRSSKAHRKRER |
| domain- | | LQDVARMEAEMAQKHRQELEKF | | MESEERERQESIFQAEMSEH |
| containing | | QDDSSIESVVEDLAKMNLENRP | | LAGFKREEEEKLAAILGARG |
| protein 6A | | PRSSKAHRKRERMESEERERQE | | LEMKAIPADGHCMYRAIQDQ |
| | | SIFQAEMSEHLAGFKREEEEKL | | LVFSVSVEMLRCRTASYMKK |
| | | AAILGARGLEMKAIPADGHCMY | | HVDEFLPFFSNPETSDSEGY |
| | | RAIQDQLVFSVSVEMLRCRTAS | | DDFMIYCDNIVRTTAWGGQL |
| | | YMKKHVDEFLPFFSNPETSDSF | | ELRALSHVLKTPIEVIQADS |
| | | GYDDFMIYCDNIVRTTAWGGQL | | PTLIIGEEYVKKPIILVYLR |
| | | ELRALSHVLKTPIEVIQADSPT | | YAYS |
| | | LIIGEEYVKKPIILVYLRYAYS | | |
| | | LGEHYNSVTPLEAGAAGGVLPR | | |
| | | LL | | |
| OTUB1_ | 75 | MAAEEPQQQKQEPLGSDSEGVN | 75 | MAAEEPQQQKQEPLGSDSEG |
| HUMAN | | CLAYDEAIMAQQDRIQQEIAVQ | | VNCLAYDEAIMAQQDRIQQE |
| Ubiquitin | | NPLVSERLELSVLYKEYAEDDN | | IAVQNPLVSERLELSVLYKE |
| thioesterase | | IYQQKIKDLHKKYSYIRKTRPD | | YAEDDNIYQQKIKDLHKKYS |
| OTUB1 | | GNCFYRAFGFSHLEALLDDSKE | | YIRKTRPDGNCFYRAFGESH |
| | | LQRFKAVSAKSKEDLVSQGFTE | | LEALLDDSKELQRFKAVSAK |
| | | FTIEDEHNTFMDLIEQVEKQTS | | SKEDLVSQGFTEFTIEDEHN |
| | | VADLLASENDQSTSDYLVVYLR | | TEMDLIEQVEKQTSVADLLA |
| | | LLTSGYLQRESKFFEHFIEGGR | | SENDQSTSDYLVVYLRLLTS |
| | | TVKEFCQQEVEPMCKESDHIHI | | GYLQRESKFFEHFIEGGRTV |
| | | IALAQALSVSIQVEYMDRGEGG | | KEFCQQEVEPMCKESDHIHI |
| | | TTNPHIFPEGSEPKVYLLYRPG | | IALAQALSVSIQVEYMDRGE |
| | | HYDILYK | | GGTTNPHIFPEGSEPKVYLL |
| | | | | YRPGHYDILYK |
| OTU7A_HUMAN | 76 | MVSSVLPNPTSAECWAALLHDP | 186 | SDYEQLRQVHTANLPHVENE |
| MOTU | | MTLDMDAVLSDFVRSTGAEPGL | | GRGPKQPEREPQPGHKVERP |
| domain- | | ARDLLEGKNWDLTAALSDYEQL | | CLQRQDDIAQEKRLSRGISH |
| containing | | RQVHTANLPHVENEGRGPKQPE | | ASSAIVSLARSHVASECNNE |
| protein 7A | | REPQPGHKVERPCLQRQDDIAQ | | QFPLEMPIYTFQLPDLSVYS |
| | | EKRLSRGISHASSAIVSLARSH | | EDERSFIERDLIEQATMVAL |
| | | VASECNNEQFPLEMPIYTFQLP | | EQAGRLNWWSTVCTSCKRLL |
| | | DLSVYSEDERSFIERDLIEQAT | | PLATTGDGNCLLHAASLGMW |
| | | MVALEQAGRLNWWSTVCTSCKR | | GFHDRDLVLRKALYTMMRTG |
| | | LLPLATTGDGNCLLHAASLGMW | | AEREALKRRWRWQQTQQNKE |
| | | GFHDRDLVLRKALYTMMRTGAE | | EEWEREWTELLKLASSEPRT |
| | | REALKRRWRWQQTQQNKEEEWE | | HFSKNGGTGGGVDNSEDPVY |
| | | REWTELLKLASSEPRTHESKNG | | ESLEEFHVEVLAHILRRPIV |
| | | GTGGGVDNSEDPVY | | VVADTMLRDSGGEAFAPIPE |
| | | ESLEEFHVEVLAHILRRPIVVV | | GGIYLPLEVPPNRCHCSPLV |
| | | ADTMLRDSGGEAFAPIPEGGIY | | LAYDQAHFSAL |
| | | LPLEVPPNRCHCSPLVLAYDQA | | |
| | | HFSALVSMEQRDQQREQAVIPL | | |
| | | TDSEHKLLPLHFAVDPGKDWEW | | |
| | | GKDDNDNARLAHLILSLEAKLN | | |
| | | LLHSYMNVTWIRIPSETRAPLA | | |
| | | QPESPTASAGEDVQSLADSLDS | | |
| | | DRDSVCSNSNSNNGKNGKDKEK | | |
| | | EKQRKEKDKTRADSVANKLGSF | | |
| | | SKTLGIKLKKNMGGLGGLVHGK | | |
| | | MGRANSANGKNGDSAERGKEKK | | |
| | | AKSRKGSKEESGASASTSPSEK | | |
| | | TTPSPTDKAAGASP | | |
| | | AEKGGGPRGDAWKYSTDVKLSL | | |
| | | NILRAAMQGERKFIFAGLLLTS | | |
| | | HRHQFHEEMIGYYLTSAQERES | | |
| | | AEQEQRRRDAATAAAAAAAAAA | | |
| | | ATAKRPPRRPETEGVPVPERAS | | |
| | | PGPPTQLVLKLKERPSPGPAAG | | |
| | | RAARAAAGGTASPGGGARRASA | | |
| | | SGPVPGRSPPAPARQSVIHVQA | | |
| | | SGARDEACAPAVGALRPCATYP | | |
| | | QQNRSLSSQSYSPARAAALRTV | | |
| | | NTVESLARAVPGALPGAAGTAG | | |
| | | AAEHKSQTYTNGFGALRDGLEF | | |
| | | ADADAPTARSNGECGRGGPGPV | | |
| | | QRRCQRENCAFYGRAETEHYCS | | |
| | | YCYREELRRRREARGARP | | |
| OTUD4_HUMAN | 77 | MEAAVGVPDGGDQGGAGPREDA | 187 | MEAAVGVPDGGDQGGAGPRE |
| MOTU | | TPMDAYLRKLGLYRKLVAKDGS | | DATPMDAYLRKLGLYRKLVA |
| domain- | | CLFRAVAEQVLHSQSRHVEVRM | | KDGSCLFRAVAEQVLHSQSR |
| containing | | ACIHYLRENREKFEAFIEGSFE | | HVEVRMACIHYLRENREKFE |
| protein | | EYLKRLENPQEWVGQVEISALS | | AFIEGSFEEYLKRLENPQEW |
| 4 | | LMYRKDFIIYREPNVSPSQVTE | | VGQVEISALSLMYRKDFIIY |
| | | NNFPEKVLLCESNGNHYDIVYP | | REPNVSPSQVTENNFPEKVL |
| | | IKYKESSAMCQSLLYELLYEKV | | LCFSNGNHYDIVYP |
| | | FKTDVSKIVMELDTLEVADEDN | | |
| | | SEISDSEDDSCKSKTAAAAADV | | |
| | | NGFKPLSGNEQLKNNGNSTSLP | | |
| | | LSRKVLKSLNPAVYRNVEYEIW | | |
| | | LKSKQAQQKRDYSIAAGLQYEV | | |
| | | GDKCQVRLDHNGKF | | |
| | | LNADVQGIHSENGPVLVEELGK | | |
| | | KHTSKNLKAPPPESWNTVSGKK | | |
| | | MKKPSTSGQNFHSDVDYRGPKN | | |
| | | PSKPIKAPSALPPRLQHPSGVR | | |
| | | QHAFSSHSSGSQSQKESSEHKN | | |
| | | LSRTPSQIIRKPDRERVEDEDH | | |
| | | TSRESNYFGLSPEERREKQAIE | | |
| | | ESRLLYEIQNRDEQAFPALSSS | | |
| | | SVNQSASQSSNPCVQRKSSHVG | | |
| | | DRKGSRRRMDTEERKDKDSIHG | | |
| | | HSQLDKRPEPSTLENITDDKYA | | |
| | | TVSSPSKSKKLECPSPAEQKPA | | |
| | | EHVSLSNPAPLLVSPEVHLTPA | | |
| | | VPSLPATVPAWPSE | | |
| | | PTTFGPTGVPAPIPVLSVTQTL | | |
| | | TTGPDSAVSQAHLTPSPVPVSI | | |
| | | QAVNQPLMPLPQTLSLYQDPLY | | |
| | | PGFPCNEKGDRAIVPPYSLCQT | | |
| | | GEDLPKDKNILRFFFNLGVKAY | | |
| | | SCPMWAPHSYLYPLHQAYLAAC | | |
| | | RMYPKVPVPVYPHNPWFQEAPA | | |
| | | AQNESDCTCTDAHFPMQTEASV | | |
| | | NGQMPQPEIGPPTFSSPLVIPP | | |
| | | SQVSESHGQLSYQADLESETPG | | |
| | | QLLHADYEESLSGKNMFPQSFG | | |
| | | PNPFLGPVPIAPPFFPHVWYGY | | |
| | | PFQGFIENPVMRQNIVLPSDEK | | |
| | | GELDLSLENLDLS | | |
| | | KDCGSVSTVDEFPEARGEHVHS | | |
| | | LPEASVSSKPDEGRTEQSSQTR | | |
| | | KADTALASIPPVAEGKAHPPTQ | | |
| | | ILNRERETVPVELEPKRTIQSL | | |
| | | KEKTEKVKDPKTAADVVSPGAN | | |
| | | SVDSRVQRPKEESSEDENEVSN | | |
| | | ILRSGRSKQFYNQTYGSRKYKS | | |
| | | DWGYSGRGGYQHVRSEESWKGQ | | |
| | | PSRSRDEGYQYHRNVRGRPERG | | |
| | | DRRRSGMGDGHRGQHT | | |
| OTUB2_ | 78 | MSETSENLISEKCDILSILRDH | 78 | MSETSENLISEKCDILSILR |
| HUMAN | | PENRIYRRKIEELSKRFTAIRK | | DHPENRIYRRKIEELSKRFT |
| Ubiquitin | | TKGDGNCFYRALGYSYLESLLG | | AIRKTKGDGNCFYRALGYSY |
| thioesterase | | KSREIFKFKERVLQTPNDLLAA | | LESLLGKSREIFKFKERVLQ |
| OTUB2 | | GFEEHKERNFFNAFYSVVELVE | | TPNDLLAAGFEEHKERNFEN |
| | | KDGSVSSLLKVENDQSASDHIV | | AFYSVVELVEKDGSVSSLLK |
| | | QFLRLLTSAFIRNRADFFRHFI | | VENDQSASDHIVQFLRLLTS |
| | | DEEMDIKDFCTHEVEPMATECD | | AFIRNRADFFRHFIDEEMDI |
| | | HIQITALSQALSIALQVEYVDE | | KDFCTHEVEPMATECDHIQI |
| | | MDTALNHHVFPEAATPSVYLLY | | TALSQALSIALQVEYVDEMD |
| | | KTSHYNILYAADKH | | TALNHHVFPEAATPSVYLLY |
| | | | | KTSHYNILYAADKH |
| OTUD3_HUMAN | 79 | MSRKQAAKSRPGSGSRKAEAER | 188 | MSRKQAAKSRPGSGSRKAEA |
| MOTU | | KRDERAARRALAKERRNRPESG | | ERKRDERAARRALAKERRNR |
| domain- | | GGGGCEEEFVSFANQLQALGLK | | PESGGGGGCEEEFVSFANQL |
| containing | | LREVPGDGNCLFRALGDQLEGH | | QALGLKLREVPGDGNCLFRA |
| protein 3 | | SRNHLKHRQETVDYMIKQREDE | | LGDQLEGHSRNHLKHRQETV |
| | | EPFVEDDIPFEKHVASLAKPGT | | DYMIKQREDFEPFVEDDIPF |
| | | FAGNDAIVAFARNHQLNVVIHQ | | EKHVASLAKPGTFAGNDAIV |
| | | LNAPLWQIRGTEKSSVRELHIA | | AFARNHQLNVVIHQLNAPLW |
| | | YRYGEHYDSVRRINDNSEAPAH | | QIRGTEKSSVRELHIAYRYG |
| | | LQTDFQMLHQDESNKREKIKTK | | EHYDSVRR |
| | | GMDSEDDLRDEVEDAVQKVCNA | | |
| | | TGCSDENLIVQNLEAENYNIES | | |
| | | AIIAVLRMNQGKRNNAEENLEP | | |
| | | SGRVLKQCGPLWEE | | |
| | | GGSGARIFGNQGLNEGRTENNK | | |
| | | AQASPSEENKANKNQLAKVINK | | |
| | | QRREQQWMEKKKRQEERHRHKA | | |
| | | LESRGSHRDNNRSEAEANTQVT | | |
| | | LVKTFAALNI | | |
| OTU7B_HUMAN | 80 | MTLDMDAVLSDFVRSTGAEPGL | 189 | MTLDMDAVLSDFVRSTGAEP |
| MOTU | | ARDLLEGKNWDVNAALSDFEQL | | GLARDLLEGKNWDVNAALSD |
| domain- | | RQVHAGNLPPSFSEGSGGSRTP | | FEQLRQVHAGNLPPSFSEGS |
| containing | | EKGFSDREPTRPPRPILQRQDD | | GGSRTPEKGFSDREPTRPPR |
| | | IVQEKRLSRGISHASSSIVSLA | | PILQRQDDIVQEKRLSRGIS |
| | | RSHVSSNGGGGGSNEHPLEMPI | | HASSSIVSLARSHVSSNGGG |
| | | CAFQLPDLTVYNEDERSFIERD | | GGSNEHPLEMPICAFQLPDL |
| | | LIEQSMLVALEQAGRLNWWVSV | | TVYNEDERSFIERDLIEQSM |
| | | DPTSQRLLPLATTGDGNCLLHA | | LVALEQAGRLNWWVSVDPTS |
| | | ASLGMWGFHDRDLMLRKALYAL | | QRLLPLATTGDGNCLLHAAS |
| | | MEKGVEKEALKRRWRWQQTQQN | | LGMWGFHDRDLMLRKALYAL |
| | | KESGLVYTEDEWQKEWNELIKL | | MEKGVEKEALKRRWRWQQTQ |
| | | ASSEPRMHLGTNGANCGGVESS | | QNKESGLVYTEDEWQKEWNE |
| | | EEPVYESLEEFHVEVLAHVLRR | | LIKLASSEPRMHLGTNGANC |
| | | PIVVVADTMLRDSGGEAFAPIP | | GGVESSEEPVYESLEEFHVE |
| | | FGGIYLPLEVPASQCHRSPLVL | | VLAHVLRRPIVVVADTMLRD |
| | | AYDQAHFSALVSMEQKENTKEQ | | SGGEAFAPIPFGGIYLPLEV |
| | | AVIPLTDSEYKLLPLHFAVDPG | | PASQCHRSPLVLAYDQAHES |
| | | KGWEWGKDDSDNVRLASVILSL | | AL |
| protein 7B | | EVKLHLLHSYMNVKWIPLSSDA | 293 | PPSFSEGSGGSRTPEKGFSD |
| (Also referred | | QAPLAQPESPTASAGDEPRSTP | | REPTRPPRPILQRQDDIVQE |
| to herein as | | ESGDSDKESVGSSSTSNEGGRR | | KRLSRGISHASSSIVSLARS |
| Cezanne) | | KEKSKRDREKDKKRADSVANKL | | HVSSNGGGGGSNEHPLEMPI |
| | | GSFGKTLGSKLKKNMGGLMHSK | 190 | CAFQLPDLTVYNEDERSFIE |
| | | GSKPGGVGTGLGGSSGTETLEK | | RDLIEQSMLVALEQAGRLNW |
| | | KKKNSLKSWKGGKEEAAGDGPV | | WVSVDPTSQRLLPLATTGDG |
| | | SEKPPAESVGNGGSKYSQEVMQ | | NCLLHAASLGMWGFHDRDLM |
| | | SLSILRTAMQGEGKFIFVGTLK | | LRKALYALMEKGVEKEALKR |
| | | MGHRHQYQEEMIQRYLSDAEER | | RWRWQQTQQNKESGLVYTED |
| | | FLAEQKQKEAERKIMNGGIGGG | | EWQKEWNELIKLASSEPRMH |
| | | PPPAKKPEPDAREEQPTGPPAE | | LGTNGANCGGVESSEEPVYE |
| | | SRAMAFSTGYPGDFTIPRPSGG | | SLEEFHVFVLAHVLRRPIVV |
| | | GVHCQEPRRQLAGGPCVGGLPP | | VADTMLRDSGGEAFAPIPFG |
| | | YATFPRQCPPGRPYPHQDSIPS | | GIYLPLEVPASQCHRSPLVL |
| | | LEPGSHSKDGLHRGALLPPPYR | | AYDQAHFSALVSMEQKENTK |
| | | VADSYSNGYREPPEPDGWAGGL | | EQAVIPLTDSEYKLLPLHFA |
| | | RGLPPTQTKCKQPNCSFYGHPE | | VDPGKGWEWGKDDSDNVRLA |
| | | TNNFCSCCYREELRRREREPDG | | SVILSLEVKLHLLHSYMNVK |
| | | ELLVHRE | | WIPLSSDAQAPLAQ |
| | | MTILPKKKPPPPDADPANEPPP | | |
| | | PGPMPPAPRRGGGVGVGGGGTG | | |
| | | VGGGDRDRDSGVVGARPRASPP | | |
| | | PQGPLPGPPGALHRWALAVPPG | | |
| | | AVAGPRPQQASPPPCGGPGGPG | | |
| | | GGPGDALGAAAAGVGAAGVVVG | | |
| OTUD5_HUMAN | 81 | VGGAVGVGGCCSGPGHSKRRRQ | | MTILPKKKPPPPDADPANEP |
| MOTU | | APGVGAVGGGSPEREEVGAGYN | | PPPGPMPPAPRRGGGVGVGG |
| domain- | | SEDEYEAAAARIEAMDPATVEQ | | GGTGVGGGDRDRDSGVVGAR |
| containing | | QEHWFEKALRDKKGFIIKQMKE | | PRASPPPQGPLPGPPGALHR |
| protein 5 | | DGACLFRAVADQVYGDQDMHEV | | WALAVPPGAVAGPRPQQASP |
| | | VRKHCMDYLMKNADYFSNYVTE | | PPCGGPGGPGGGPGDALGAA |
| | | DETTYINRKRKNNCHGNHIEMQ | | AAGVGAAGVVVGVGGAVGVG |
| | | AMAEMYNRPVEVYQ | | GCCSGPGHSKRRRQAPGVGA |
| | | YSTGTSAVEPINTFHGIHQNED | | VGGGSPEREEVGAGYNSEDE |
| | | EPIRVSYHRNIHYNSVVNPNKA | | YEAAAARIEAMDPATVEQQE |
| | | TIGVGLGLPSFKPGFAEQSLMK | | HWFEKALRDKKGFIIKQMKE |
| | | NAIKTSEESWIEQQMLEDKKRA | | DGACLFRAVADQVYGDQDMH |
| | | TDWEATNEAIEEQVARESYLQW | | EVVRKHCMDYLMKNADYFSN |
| | | LRDQEKQARQVRGPSQPRKASA | | YVTEDETTYINRKRKNNCHG |
| | | TCSSATAAASSGLEEWTSRSPR | | NHIEMQAMAEMYNRPVEVYQ |
| | | QRSSASSPEHPELHAELGMKPP | | YSTGTSAVEPINTFHGIHQN |
| | | SPGTVLALAKPPSPCAPGTSSQ | | EDEPIRVSYHRNIHYNSV |
| | | FSAGADRATSPLVSLYPALECR | | |
| | | ALIQQMSPSAFGLNDWDDDEIL | | |
| | | ASVLAVSQQEYLDSMKKNKVHR | | |
| | | DPPPDKS | | |
| TNAP3_HUMAN | 82 | MAEQVLPQALYLSNMRKAVKIR | 191 | MAEQVLPQALYLSNMRKAVK |
| Tumor | | ERTPEDIFKPTNGIIHHFKTMH | | IRERTPEDIFKPTNGIIHHF |
| necrosis factor | | RYTLEMERTCQFCPQFREIIHK | | KTMHRYTLEMERTCQFCPQF |
| alpha-induced | | ALIDRNIQATLESQKKLNWCRE | | REIIHKALIDRNIQATLESQ |
| protein 3 | | VRKLVALKTNGDGNCLMHATSQ | | KKLNWCREVRKLVALKTNGD |
| | | YMWGVQDTDLVLRKALFSTLKE | | GNCLMHATSQYMWGVQDTDL |
| | | TDTRNFKFRWQLESLKSQEFVE | | VLRKALFSTLKETDTRNFKF |
| | | TGLCYDTRNWNDEWDNLIKMAS | | RWQLESLKSQEFVETGLCYD |
| | | TDTPMARSGLQYNSLEEIHIFV | | TRNWNDEWDNLIKMASTDTP |
| | | LCNILRRPIIVISDKMLRSLES | | MARSGLQYNSLEEIHIFVLC |
| | | GSNFAPLKVGGIYLPLHWPAQE | | NILRRPIIVISDKMLRSLES |
| | | CYRYPIVLGYDSHHFVPLVTLK | | GSNFAPLKVGGIYLPLHWPA |
| | | DSGPEIRAVPLVNRDRGRFEDL | | QECYRYPIVLGYDSHHFVPL |
| | | KVHELTDPENEMKE | | |
| | | KLLKEYLMVIEIPVQGWDHGTT | | |
| | | HLINAAKLDEANLPKEINLVDD | | |
| | | YFELVQHEYKKWQENSEQGRRE | | |
| | | GHAQNPMEPSVPQLSLMDVKCE | | |
| | | TPNCPFFMSVNTQPLCHECSER | | |
| | | RQKNQNKLPKLNSKPGPEGLPG | | |
| | | MALGASRGEAYEPLAWNPEEST | | |
| | | GGPHSAPPTAPSPELFSETTAM | | |
| | | KCRSPGCPFTLNVQHNGFCERC | | |
| | | HNARQLHASHAPDHTRHLDPGK | | |
| | | CQACLQDVTRTENGICSTCFKR | | |
| | | TTAEASSSLSTSLPPSCHQRSK | | |
| | | SDPSRLVRSPSPHSCHRAGNDA | | |
| | | PAGCLSQAARTPGD | | |
| | | RTGTSKCRKAGCVYFGTPENKG | | |
| | | FCTLCFIEYRENKHFAAASGKV | | |
| | | SPTASRFQNTIPCLGRECGTLG | | |
| | | STMFEGYCQKCFIEAQNQRFHE | | |
| | | AKRTEEQLRSSQRRDVPRTTQS | | |
| | | TSRPKCARASCKNILACRSEEL | | |
| | | CMECQHPNQRMGPGAHRGEPAP | | |
| | | EDPPKQRCRAPACDHEGNAKCN | | |
| | | GYCNECFQFKQMYG | | |
| ZRAN1_ | 83 | MSERGIKWACEYCTYENWPSAI | 192 | MSERGIKWACEYCTYENWPS |
| HUMAN | | KCTMCRAQRPSGTIITEDPFKS | | AIKCTMCRAQRPSGTIITED |
| Ubiquitin | | GSSDVGRDWDPSSTEGGSSPLI | | PFKSGSSDVGRDWDPSSTEG |
| thioesterase | | CPDSSARPRVKSSYSMENANKW | | GSSPLICPDSSARPRVKSSY |
| ZRANB1 | | SCHMCTYLNWPRAIRCTQCLSQ | | SMENANKWSCHMCTYLNWPR |
| | | RRTRSPTESPQSSGSGSRPVAF | | AIRCTQCLSQRRTRSPTESP |
| | | SVDPCEEYNDRNKLNTRTQHWT | | QSSGSGSRPVAFSVDPCEEY |
| | | CSVCTYENWAKAKRCVVCDHPR | | NDRNKLNTRTQHWTCSVCTY |
| | | PNNIEAIELAETEEASSIINEQ | | ENWAKAKRCVVCDHPRPNNI |
| | | DRARWRGSCSSGNSQRRSPPAT | | EAIELAETEEASSIINEQDR |
| | | KRDSEVKMDFQRIELAGAVGSK | | ARWRGSCSSGNSQRRSPPAT |
| | | EELEVDEKKLKQIKNRMKKTDW | | KRDSEVKMDFQRIELAGAVG |
| | | LFLNACVGVVEGDLAAIEAYKS | | SKEELEVDEKKLKQIKNRMK |
| | | SGGDIARQLTADEV | | KTDWLFLNACVGVVEGDLAA |
| | | RLLNRPSAFDVGYTLVHLAIRE | | IEAYKSSGGDIARQLTADEV |
| | | QRQDMLAILLTEVSQQAAKCIP | | RLLNRPSAFDVGYTLVHLAI |
| | | AMVCPELTEQIRREIAASLHQR | | RFQRQDMLAILLTEVSQQAA |
| | | KGDFACYFLTDLVTFTLPADIE | | KCIPAMVCPELTEQIRREIA |
| | | DLPPTVQEKLEDEVLDRDVQKE | | ASLHQRKGDFACYFLTDLVT |
| | | LEEESPIINWSLELATRLDSRL | | FTLPADIEDLPPTVQEKLED |
| | | YALWNRTAGDCLLDSVLQATWG | | EVLDRDVQKELEEESPIINW |
| | | IYDKDSVLRKALHDSLHDCSHW | | SLELATRLDSRLYALWNRTA |
| | | FYTRWKDWESWYSQSFGLHESL | | GDCLLDSVLQATWGIYDKDS |
| | | REEQWQEDWAFILSLASQPGAS | | VLRKALHDSLHDCSHWFYTR |
| | | LEQTHIFVLAHILRRPIIVYGV | | WKDWESWYSQSFGLHESLRE |
| | | KYYKSFRGETLGYTRFQGVYLP | | EQWQEDWAFILSLASQPGAS |
| | | LLWEQSFCWKSPIALGYTRGHF | | LEQTHIFVLAHILRRPIIVY |
| | | SALVAMENDGYGNR | | GVKYYKSFRGETLGYTREQG |
| | | GAGANLNTDDDVTITELPLVDS | | VYLPLLWEQSFCWKSPIALG |
| | | ERKLLHVHELSAQELGNEEQQE | | YTRGHFSAL |
| | | KLLREWLDCCVTEGGVLVAMQK | | |
| | | SSRRRNHPLVTQMVEKWLDRYR | | |
| | | QIRPCTSLSDGEEDEDDEDE | | |
| VCIP1_ | 84 | MSQPPPPPPPLPPPPPPPEAPQ | 193 | PASGSVSIECTECGQRHEQQ |
| HUMAN | | TPSSLASAAASGGLLKRRDRRI | | QLLGVEEVTDPDVVLHNLLR |
| Deubiquitinating | | LSGSCPDPKCQARLFFPASGSV | | NALLGVTGAPKKNTELVKVM |
| protein | | SIECTECGQRHEQQQLLGVEEV | | GLSNYHCKLLSPILARYGMD |
| VCIP135 | | TDPDVVLHNLLRNALLGVTGAP | | KQTGRAKLLRDMNQGELEDC |
| | | KKNTELVKVMGLSNYHCKLLSP | | ALLGDRAFLIEPEHVNTVGY |
| | | ILARYGMDKQTGRAKLLRDMNQ | | GKDRSGSLLYLHDTLEDIKR |
| | | GELFDCALLGDRAFLIEPEHVN | | ANKSQECLIPVHVDGDGHCL |
| | | TVGYGKDRSGSLLYLHDTLEDI | | VHAVSRALVGRELFWHALRE |
| | | KRANKSQECLIPVHVDGDGHCL | | NLKQHFQQHLARYQALFHDF |
| | | VHAVSRALVGRELFWHALRENL | | IDAAEWEDIINECDPLFVPP |
| | | KQHFQQHLARYQALFHDFIDAA | | EGVPLGLRNIHIFGLANVLH |
| | | EWEDIINECDPLFVPPEGVPLG | | RPIILLDSLSGMRSSGDYSA |
| | | LRNIHIFGLANVLH | | TFLPGLIPAEKCTGKDGHLN |
| | | RPIILLDSLSGMRSSGDYSATF | | KPICIAWSSSGRNHYIPL |
| | | LPGLIPAEKCTGKDGHLNKPIC | | |
| | | IAWSSSGRNHYIPLVGIKGAAL | | |
| | | PKLPMNLLPKAWGVPQDLIKKY | | |
| | | IKLEEDGGCVIGGDRSLQDKYL | | |
| | | LRLVAAMEEVEMDKHGIHPSLV | | |
| | | ADVHQYFYRRTGVIGVQPEEVT | | |
| | | AAAKKAVMDNRLHKCLLCGALS | | |
| | | ELHVPPEWLAPGGKLYNLAKST | | |
| | | HGQLRTDKNYSFPLNNLVCSYD | | |
| | | SVKDVLVPDYGMSNLTACNWCH | | |
| | | GTSVRKVRGDGSIVYLDGDRTN | | |
| | | SRSTGGKCGCGFKHFWDGKEYD | | |
| | | NLPEAFPITLEWGG | | |
| | | RVVRETVYWFQYESDSSLNSNV | | |
| | | YDVAMKLVTKHFPGEFGSEILV | | |
| | | QKVVHTILHQTAKKNPDDYTPV | | |
| | | NIDGAHAQRVGDVQGQESESQL | | |
| | | PTKIILTGQKTKTLHKEELNMS | | |
| | | KTERTIQQNITEQASVMQKRKT | | |
| | | EKLKQEQKGQPRTVSPSTIRDG | | |
| | | PSSAPATPTKAPYSPTTSKEKK | | |
| | | IRITTNDGRQSMVTLKSSTTFF | | |
| | | ELQESIAREFNIPPYLQCIRYG | | |
| | | FPPKELMPPQAGMEKEPVPLQH | | |
| | | GDRITIEILKSKAEGGQSAAAH | | |
| | | SAHTVKQEDIAVTGKLSSKELQ | | |
| | | EQAEKEMYSLCLLA | | |
| | | TLMGEDVWSYAKGLPHMFQQGG | | |
| | | VFYSIMKKTMGMADGKHCTFPH | | |
| | | LPGKTFVYNASEDRLELCVDAA | | |
| | | GHFPIGPDVEDLVKEAVSQVRA | | |
| | | EATTRSRESSPSHGLLKLGSGG | | |
| | | VVKKKSEQLHNVTAFQGKGHSL | | |
| | | GTASGNPHLDPRARETSVVRKH | | |
| | | NTGTDFSNSSTKTEPSVFTASS | | |
| | | SNSELIRIAPGVVTMRDGRQLD | | |
| | | PDLVEAQRKKLQEMVSSIQASM | | |
| | | DRHLRDQSTEQSPSDLPQRKTE | | |
| | | VVSSSAKSGSLQTGLPESEPLT | | |
| | | GGTENLNTETTDGCVADALGAA | | |
| | | FATRSKAQRGNSVEELEEMDSQ | | |
| | | DAEMTNTTEPMDHS | | |
| UCHL3_ | 85 | MEGQRWLPLEANPEVTNQFLKQ | 194 | QRWLPLEANPEVTNQFLKQL |
| HUMAN | | LGLHPNWQFVDVYGMDPELLSM | | GLHPNWQFVDVYGMDPELLS |
| Ubiquitin | | VPRPVCAVLLLFPITEKYEVER | | MVPRPVCAVLLLFPITEKYE |
| carboxyl- | | TEEEEKIKSQGQDVTSSVYFMK | | VERTEEEEKIKSQGQDVTSS |
| terminal | | QTISNACGTIGLIHAIANNKDK | | VYFMKQTISNACGTIGLIHA |
| hydrolase | | MHFESGSTLKKFLEESVSMSPE | | IANNKDKMHFESGSTLKKEL |
| isozyme L3 | | ERARYLENYDAIRVTHETSAHE | | EESVSMSPEERARYLENYDA |
| | | GQTEAPSIDEKVDLHFIALVHV | | IRVTHETSAHEGQTEAPSID |
| | | DGHLYELDGRKPFPINHGETSD | | EKVDLHFIALVHVDGHLYEL |
| | | ETLLEDAIEVCKKEMERDPDEL | | DGRKPFPINHGETSDETLLE |
| | | RENAIALSAA | | DAIEVCKKEMERDPDELREN |
| | | | | AIALSAA |
| UCHL1_ | 86 | MQLKPMEINPEMLNKVLSRLGV | 86 | MQLKPMEINPEMLNKVLSRL |
| HUMAN | | AGQWRFVDVLGLEEESLGSVPA | | GVAGQWRFVDVLGLEEESLG |
| Ubiquitin | | PACALLLLFPLTAQHENFRKKQ | | SVPAPACALLLLFPLTAQHE |
| carboxyl- | | IEELKGQEVSPKVYFMKQTIGN | | NFRKKQIEELKGQEVSPKVY |
| terminal | | SCGTIGLIHAVANNQDKLGFED | | FMKQTIGNSCGTIGLIHAVA |
| hydrolase | | GSVLKQFLSETEKMSPEDRAKC | | NNQDKLGFEDGSVLKQFLSE |
| isozyme L1 | | FEKNEAIQAAHDAVAQEGQCRV | | TEKMSPEDRAKCFEKNEAIQ |
| | | DDKVNFHFILENNVDGHLYELD | | AAHDAVAQEGQCRVDDKVNF |
| | | GRMPFPVNHGASSEDTLLKDAA | | HFILENNVDGHLYELDGRMP |
| | | KVCREFTEREQGEVRESAVALC | | FPVNHGASSEDTLLKDAAKV |
| | | KAA | | CREFTEREQGEVRESAVALC |
| | | | | KAA |
| UCHL5_ | 87 | MTGNAGEWCLMESDPGVFTELI | 195 | GEWCLMESDPGVFTELIKGF |
| HUMAN | | KGFGCRGAQVEEIWSLEPENFE | | GCRGAQVEEIWSLEPENFEK |
| Ubiquitin | | KLKPVHGLIFLFKWQPGEEPAG | | LKPVHGLIFLFKWQPGEEPA |
| carboxyl- | | SVVQDSRLDTIFFAKQVINNAC | | GSVVQDSRLDTIFFAKQVIN |
| terminal | | ATQAIVSVLLNCTHQDVHLGET | | NACATQAIVSVLLNCTHQDV |
| hydrolase | | LSEFKEFSQSFDAAMKGLALSN | | HLGETLSEFKEFSQSEDAAM |
| isozyme L5 | | SDVIRQVHNSFARQQMFEEDTK | | KGLALSNSDVIRQVHNSFAR |
| | | TSAKEEDAFHFVSYVPVNGRLY | | QQMFEFDTKTSAKEEDAFHF |
| | | ELDGLREGPIDLGACNQDDWIS | | VSYVPVNGRLYELDGLREGP |
| | | AVRPVIEKRIQKYSEGEIRENL | | IDLGACNQDDWISAVRPVIE |
| | | MAIVSDRKMIYEQKIAELQRQL | | KRIQKYSEGEIRFNLMAIVS |
| | | AEEEPMDTDQGNSMLSAIQSEV | | DRK |
| | | AKNQMLIEEEVQKLKRYKIENI | | |
| | | RRKHNYLPFIMELLKTLAEHQQ | | |
| | | LIPLVEKAKEKQNAKKAQETK | | |
| ATX3_HUMAN | 88 | MESIFHEKQEGSLCAQHCLNNL | 196 | ESIFHEKQEGSLCAQHCLNN |
| Ataxin-3 | | LQGEYFSPVELSSIAHQLDEEE | | LLQGEYFSPVELSSIAHQLD |
| | | RMRMAEGGVTSEDYRTFLQQPS | | EEERMRMAEGGVTSEDYRTE |
| | | GNMDDSGFFSIQVISNALKVWG | | LQQPSGNMDDSGFFSIQVIS |
| | | LELILENSPEYQRLRIDPINER | | NALKVWGLELILENSPEYQR |
| | | SFICNYKEHWFTVRKLGKQWEN | | LRIDPINERSFICNYKEHWE |
| | | LNSLLTGPELISDTYLALFLAQ | | TVRKLGKQWENLNSLLTGPE |
| | | LQQEGYSIFVVKGDLPDCEADQ | | LISDTYLALFLAQLQQEGYS |
| | | LLQMIRVQQMHRPKLIGEELAQ | | IFVVK |
| | | LKEQRVHKTDLERVLEANDGSG | | |
| | | MLDEDEEDLQRALALSRQEIDM | | |
| | | EDEEADLRRAIQLSMQGSSRNI | | |
| | | SQDMTQTSGTNLTSEELRKRRE | | |
| | | AYFEKQQQKQQQQGDL | | |
| | | SGQSSHPCERPATSSGALGSDL | | |
| | | GDAMSEEDMLQAAVTMSLETVR | | |
| | | NDLKTEGKK | | |
| JOS2_HUMAN | 89 | MSQAPGAQPSPPTVYHERQRLE | 197 | PTVYHERQRLELCAVHALNN |
| Josephin-2 | | LCAVHALNNVLQQQLFSQEAAD | | VLQQQLFSQEAADEICKRLA |
| | | EICKRLAPDSRLNPHRSLLGTG | | PDSRLNPHRSLLGTGNYDVN |
| | | NYDVNVIMAALQGLGLAAVWWD | | VIMAALQGLGLAAVWWDRRR |
| | | RRRPLSQLALPQVLGLILNLPS | | PLSQLALPQVLGLILNLPSP |
| | | PVSLGLLSLPLRRRHWVALRQV | | VSLGLLSLPLRRRHWVALRQ |
| | | DGVYYNLDSKLRAPEALGDEDG | | VDGVYYNLDSKLRAPEALGD |
| | | VRAFLAAALAQGLCEVLLVVTK | | EDGVRAFLAAALAQGLCEVL |
| | | EVEEKGSWLRTD | | LVV |
| JOS1_HUMAN | 90 | MSCVPWKGDKAKSESLELPQAA | 198 | PQAAPPQIYHEKQRRELCAL |
| Josephin-1 | | PPQIYHEKQRRELCALHALNNV | | HALNNVFQDSNAFTRDTLQE |
| | | FQDSNAFTRDTLQEIFQRLSPN | | IFQRLSPNTMVTPHKKSMLG |
| | | TMVTPHKKSMLGNGNYDVNVIM | | NGNYDVNVIMAALQTKGYEA |
| | | AALQTKGYEAVWWDKRRDVGVI | | VWWDKRRDVGVIALTNVMGF |
| | | ALTNVMGFIMNLPSSLCWGPLK | | IMNLPSSLCWGPLKLPLKRQ |
| | | LPLKRQHWICVREVGGAYYNLD | | HWICVREVGGAYYNLDSKLK |
| | | SKLKMPEWIGGESELRKELKHH | | MPEWIGGESELRKELKHHLR |
| | | LRGKNCELLLVVPEEVEAHQSW | | GKNCELLLVV |
| | | RTDV | | |
| ATX3L_HUMAN | 91 | MDFIFHEKQEGFLCAQHCLNNL | 199 | DFIFHEKQEGFLCAQHCLNN |
| taxin- | | LQGEYFSPVELASIAHQLDEEE | | LLQGEYFSPVELASIAHQLD |
| 3-like protein | | RMRMAEGGVTSEEYLAFLQQPS | | EEERMRMAEGGVTSEEYLAF |
| | | ENMDDTGFFSIQVISNALKEWG | | LQQPSENMDDTGFFSIQVIS |
| | | LEIIHENNPEYQKLGIDPINER | | NALKFWGLEIIHENNPEYQK |
| | | SFICNYKQHWFTIRKFGKHWFN | | LGIDPINERSFICNYKQHWF |
| | | LNSLLAGPELISDTCLANFLAR | | TIRKFGKHWENLNSLLAGPE |
| | | LQQQAYSVFVVKGDLPDCEADQ | | LISDTCLANFLARLQQQAYS |
| | | LLQIISVEEMDTPKLNGKKLVK | | VFVVK |
| | | QKEHRVYKTVLEKVSEESDESG | | |
| | | TSDQDEEDFQRALELSRQETNR | | |
| | | EDEHLRSTIELSMQGSSGNTSQ | | |
| | | DLPKTSCVTPASEQPKKIKEDY | | |
| | | FEKHQQEQKQQQQQSDLPGHSS | | |
| | | YLHERPTTSSRAIESDLSDDIS | | |
| | | EGTVQAAVDTILEIMRKNLKIK | | |
| | | GEK | | |
| MINY3_ | 92 | MSELTKELMELVWGTKSSPGLS | 200 | CRWTQGFVFSESEGSALEQF |
| HUMAN | | DTIFCRWTQGFVFSESEGSALE | | EGGPCAVIAPVQAFLLKKLL |
| Ubiquitin | | QFEGGPCAVIAPVQAFLLKKLL | | FSSEKSSWRDCSEEEQKELL |
| carboxyl- | | FSSEKSSWRDCSEEEQKELLCH | | CHTLCDILESACCDHSGSYC |
| terminal | | TLCDILESACCDHSGSYCLVSW | | LVSWLRGKTTEETASISGSP |
| hydrolase | | LRGKTTEETASISGSPAESSCQ | | AESSCQVEHSSALAVEELGE |
| MINDY-3 | | VEHSSALAVEELGFERFHALIQ | | ERFHALIQKRSFRSLPELKD |
| | | KRSFRSLPELKDAVLDQYSMWG | | AVLDQYSMWGNKFG |
| | | NKFGVLLFLYSVLLTKGIENIK | | VLLFLYSVLLTKGIENIKNE |
| | | NEIEDASEPLIDPVYGHGSQSL | | IEDASEPLIDPVYGHGSQSL |
| | | INLLLTGHAVSNVWDGDRECSG | | INLLLTGHAVSNVWDGDREC |
| | | MKLLGIHEQAAVGELTLMEALR | | SGMKLLGIHEQAAVGFLTLM |
| | | YCKVGSYLKSPKFPIWIVGSET | | EALRYCKVGSYLKSPKFPIW |
| | | HLTVFFAKDMALVA | | IVGSETHLTVFFAKDMALVA |
| | | PEAPSEQARRVFQTYDPEDNGE | | PEAPSEQARRVFQTYDPEDN |
| | | IPDSLLEDVMKALDLVSDPEYI | | GFIPDSLLEDVMKALDLVSD |
| | | NLMKNKLDPEGLGIILLGPFLQ | | PEYINLMKNKLDPEGLGIIL |
| | | EFFPDQGSSGPESFTVYHYNGL | | LGPFLQEFFPDQGSSGPESF |
| | | KQSNYNEKVMYVEGTAVVMGFE | | TVYHYNGLKQSNYNEKVMYV |
| | | DPMLQTDDTPIKRCLQTKWPYI | | EGTAVVMGFEDPMLQTDDTP |
| | | ELLWTTDRSPSLN | | IKRCLQTKWPYIELLWTTDR |
| | | | | SPSLN |
| MINY1_ | 93 | MEYHQPEDPAPGKAGTAEAVIP | 201 | YCVKWIPWKGEQTPIITQST |
| HUMAN | | ENHEVLAGPDEHPQDTDARDAD | | NGPCPLLAIMNILFLQWKVK |
| Ubiquitin | | GEAREREPADQALLPSQCGDNL | | LPPQKEVITSDELMAHLGNC |
| carboxyl- | | ESPLPEASSAPPGPTLGTLPEV | | LLSIKPQEKSEGLQLNFQQN |
| terminal | | ETIRACSMPQELPQSPRTRQPE | | VDDAMTVLPKLATGLDVNVR |
| hydrolase | | PDFYCVKWIPWKGEQTPIITQS | | FTGVSDFEYTPECSVEDLLG |
| MINDY-1 | | TNGPCPLLAIMNILFLQWKVKL | | IPLYHGWLVDPQSPEAVRAV |
| | | PPQKEVITSDELMAHLGNCLLS | | GKLSYNQLVERIITCKHSSD |
| | | IKPQEKSEGLQLNFQQNVDDAM | | TNLVTEGLIAEQFLETTAAQ |
| | | TVLPKLATGLDVNVRFTGVSDE | | LTYHGLCELTAAAKEGELSV |
| | | EYTPECSVEDLLGIPLYHGWLV | | FFRNNHESTMTKHKSHLYLL |
| | | DPQSPEAVRAVGKLSYNQLVER | | VTDQGFLQEEQVVWESLHNV |
| | | IITCKHSSDTNLVTEGLIAEQF | | DGDSCFCDSDFHLSHSLGKG |
| | | LETTAAQLTYHGLC | | PGAEGGSGSPETQLQVDQDY |
| | | ELTAAAKEGELSVFFRNNHEST | | LIALSLQQQQPRGPLGLTDL |
| | | MTKHKSHLYLLVTDQGELQEEQ | | ELAQQLQQEEYQQQQAAQPV |
| | | VVWESLHNVDGDSCFCDSDFHL | | RMRTRVLSLQGRGATSGRPA |
| | | SHSLGKGPGAEGGSGSPETQLQ | | GERRQRPKHESDCILL |
| | | VDQDYLIALSLQQQQPRGPLGL | | |
| | | TDLELAQQLQQEEYQQQQAAQP | | |
| | | VRMRTRVLSLQGRGATSGRPAG | | |
| | | ERRQRPKHESDCILL | | |
| MINY2_ | 94 | MESSPESLQPLEHGVAAGPASG | 202 | YHIKWIQWKEENTPIITQNE |
| HUMAN | | TGSSQEGLQETRLAAGDGPGVW | | NGPCPLLAILNVLLLAWKVK |
| Ubiquitin | | AAETSGGNGLGAAAARRSLPDS | | LPPMMEIITAEQLMEYLGDY |
| carboxyl- | | ASPAGSPEVPGPCSSSAGLDLK | | MLDAKPKEISEIQRLNYEQN |
| terminal | | DSGLESPAAAEAPLRGQYKVTA | | MSDAMAILHKLQTGLDVNVR |
| hydrolase | | SPETAVAGVGHELGTAGDAGAR | | FTGVRVFEYTPECIVEDLLD |
| MINDY-2 | | PDLAGTCQAELTAAGSEEPSSA | | IPLYHGWLVDPQIDDIVKAV |
| | | GGLSSSCSDPSPPGESPSLDSL | | GNCSYNQLVEKIISCKQSDN |
| | | ESFSNLHSFPSSCEENSEEGAE | | SELVSEGEVAEQFLNNTATQ |
| | | NRVPEEEEGAAVLPGAVPLCKE | | LTYHGLCELTSTVQEGELCV |
| | | EEGEETAQVLAASKERFPGQSV | | FFRNNHFSTMTKYKGQLYLL |
| | | YHIKWIQWKEENTPIITQNENG | | VTDQGFLTEEKVVWESLHNV |
| | | PCPLLAILNVLLLAWKVKLPPM | | DGDGNFCDSEFHLRPPSDPE |
| | | MEIITAEQLMEYLG | | TVYKGQQDQIDQDYLMALSL |
| | | DYMLDAKPKEISEIQRLNYEQN | | QQEQQSQEINWEQIPEGISD |
| | | MSDAMAILHKLQTGLDVNVRFT | | LELAKKLQEEEDRRASQYYQ |
| | | GVRVFEYTPECIVEDLLDIPLY | | EQEQAAAAAAAASTQAQQGQ |
| | | HGWLVDPQIDDIVKAVGNCSYN | | PAQASPSSGRQSGNSERKRK |
| | | QLVEKIISCKQSDNSELVSEGE | | EPREKDKEKEKEKNSCVIL |
| | | VAEQFLNNTATQLTYHGLCELT | | |
| | | STVQEGELCVFERNNHFSTMTK | | |
| | | YKGQLYLLVTDQGFLTEEKVVW | | |
| | | ESLHNVDGDGNFCDSEFHLRPP | | |
| | | SDPETVYKGQQDQIDQDYLMAL | | |
| | | SLQQEQQSQEINWEQIPEGISD | | |
| | | LELAKKLQEEEDRRASQYYQEQ | | |
| | | EQAAAAAAAASTQAQQGQPAQA | | |
| | | SPSSGRQSGNSERKRKEPREKD | | |
| | | KEKEKEKNSCVIL | | |
| MINY4_ | 95 | MDSLFVEEVAASLVREFLSRKG | 203 | FCCFNEEWKLQSFSFSNTAS |
| HUMAN | | LKKTCVTMDQERPRSDLSINNR | | LKYGIVQNKGGPCGVLAAVQ |
| Probable | | NDLRKVLHLEFLYKENKAKENP | | GCVLQKLLFEGDSKADCAQG |
| ubiquitin | | LKTSLELITRYFLDHEGNTANN | | LQPSDAHRTRCLVLALADIV |
| carboxyl- | | FTQDTPIPALSVPKKNNKVPSR | | WRAGGRERAVVALASRTQQF |
| terminal | | CSETTLVNIYDLSDEDAGWRTS | | SPTGKYKADGVLETLTLHSL |
| hydrolase | | LSETSKARHDNLDGDVLGNFVS | | TCYEDLVTFLQQSIHQFEVG |
| MINDY-4 | | SKRPPHKSKPMQTVPGETPVLT | | PYGCILLTLSAILSRSTELI |
| | | SAWEKIDKLHSEPSLDVKRMGE | | RQDFDVPTSHLIGAHGYCTQ |
| | | NSRPKSGLIVRGMMSGPIASSP | | ELVNLLLTGKAVSNVENDVV |
| | | QDSFHRHYLRRSSPSSSSTQPQ | | ELDSGDGNITLLRGIAARSD |
| | | EESRKVPELFVCTQQDILASSN | | IGFLSLFEHYNMCQVGCFLK |
| | | SSPSRTSLGQLSELTVERQKTT | | TPRFPIWVVCSESHESILES |
| | | ASSPPHLPSKRLPP | | LQPGLLRDWRTERLEDLYYY |
| | | WDRARPRDPSEDTPAVDGSTDT | | DGLANQQEQIRLTIDTTQTI |
| | | DRMPLKLYLPGGNSRMTQERLE | | SEDTDNDLVPPLELCIRTKW |
| | | RAFKRQGSQPAPVRKNQLLPSD | | KGASVNWNGSDPIL |
| | | KVDGELGALRLEDVEDELIREE | | |
| | | VILSPVPSVLKLQTASKPIDLS | | |
| | | VAKEIKTLLFGSSFCCENEEWK | | |
| | | LQSFSFSNTASLKYGIVQNKGG | | |
| | | PCGVLAAVQGCVLQKLLFEGDS | | |
| | | KADCAQGLQPSDAHRTRCLVLA | | |
| | | LADIVWRAGGRERAVVALASRT | | |
| | | QQFSPTGKYKADGVLETLTLHS | | |
| | | LTCYEDLVTFLQQSIHQFEVGP | | |
| | | YGCILLTLSAILSRSTELIRQD | | |
| | | FDVPTSHLIGAHGY | | |
| | | CTQELVNLLLTGKAVSNVENDV | | |
| | | VELDSGDGNITLLRGIAARSDI | | |
| | | GFLSLFEHYNMCQVGCFLKTPR | | |
| | | FPIWVVCSESHFSILFSLQPGL | | |
| | | LRDWRTERLEDLYYYDGLANQQ | | |
| | | EQIRLTIDTTQTISEDTDNDLV | | |
| | | PPLELCIRTKWKGASVNWNGSD | | |
| | | PIL | | |
| STABP_HUMAN | 96 | MSDHGDVSLPPEDRVRALSQLG | 204 | VVPGRLCPQFLQLASANTAR |
| STAM- | | SAVEVNEDIPPRRYFRSGVEII | | GVETCGILCGKLMRNEFTIT |
| binding | | RMASIYSEEGNIEHAFILYNKY | | HVLIPKQSAGSDYCNTENEE |
| protein | | ITLFIEKLPKHRDYKSAVIPEK | | ELFLIQDQQGLITLGWIHTH |
| | | KDTVKKLKEIAFPKAEELKAEL | | PTQTAFLSSVDLHTHCSYQM |
| | | LKRYTKEYTEYNEEKKKEAEEL | | MLPESVAIVCSPKFQETGFF |
| | | ARNMAIQQELEKEKQRVAQQKQ | | KLTDHGLEEISSCRQKGFHP |
| | | QQLEQEQFHAFEEMIRNQELEK | | HSKDPPLFCSCSHVTVVDRA |
| | | ERLKIVQEFGKVDPGLGGPLVP | | VTITDLR |
| | | DLEKPSLDVFPTLTVSSIQPSD | | |
| | | CHTTVRPAKPPVVDRSLKPGAL | | |
| | | SNSESIPTIDGLRHVVVPGRLC | | |
| | | PQFLQLASANTARGVETCGILC | | |
| | | GKLMRNEFTITHVL | | |
| | | IPKQSAGSDYCNTENEEELFLI | | |
| | | QDQQGLITLGWIHTHPTQTAFL | | |
| | | SSVDLHTHCSYQMMLPESVAIV | | |
| | | CSPKFQETGFFKLTDHGLEEIS | | |
| | | SCRQKGFHPHSKDPPLFCSCSH | | |
| | | VTVVDRAVTITDLR | | |
| MPND_HUMAN | 97 | MAAPEPLSPAGGAGEEAPEEDE | 205 | VAVSSNVLFLLDFHSHLTRS |
| MPN | | DEAEAEDPERPNAGAGGGRSGG | | EVVGYLGGRWDVNSQMLTVL |
| domain- | | GGSSVSGGGGGGGAGAGGCGGP | | RAFPCRSRLGDAETAAAIEE |
| containing | | GGALTRRAVTLRVLLKDALLEP | | EIYQSLFLRGLSLVGWYHSH |
| protein | | GAGVLSIYYLGKKFLGDLQPDG | | PHSPALPSLQDIDAQMDYQL |
| | | RIMWQETGQTENSPSAWATHCK | | RLQGSSNGFQPCLALLCSPY |
| | | KLVNPAKKSGCGWASVKYKGQK | | YSGNPGPESKISPFWVMPPP |
| | | LDKYKATWLRLHQLHTPATAAD | | EMLLVEFYKGSPDLVRLQEP |
| | | ESPASEGEEEELLMEEEEEDVL | | WSQEHTYLDKLKISLASRTP |
| | | AGVSAEDKSRRPLGKSPSEPAH | | KDQSLCHVLEQVCGVLKQGS |
| | | PEATTPGKRVDSKIRVPVRYCM | | |
| | | LGSRDLARNPHTLVEVTSFAAI | | |
| | | NKFQPFNVAVSSNVLFLLDFHS | | |
| | | HLTRSEVVGYLGGR | | |
| | | WDVNSQMLTVLRAFPCRSRLGD | | |
| | | AETAAAIEEEIYQSLFLRGLSL | | |
| | | VGWYHSHPHSPALPSLQDIDAQ | | |
| | | MDYQLRLQGSSNGFQPCLALLC | | |
| | | SPYYSGNPGPESKISPFWVMPP | | |
| | | PEMLLVEFYKGSPDLVRLQEPW | | |
| | | SQEHTYLDKLKISLASRTPKDQ | | |
| | | SLCHVLEQVCGVLKQGS | | |
| EMC9_HUMAN | 98 | MGEVEISALAYVKMCLHAARYP | 206 | ALAYVKMCLHAARYPHAAVN |
| ER | | HAAVNGLFLAPAPRSGECLCLT | | GLFLAPAPRSGECLCLTDCV |
| membrane | | DCVPLFHSHLALSVMLEVALNQ | | PLFHSHLALSVMLEVALNQV |
| protein | | VDVWGAQAGLVVAGYYHANAAV | | DVWGAQAGLVVAGYYHANAA |
| complex | | NDQSPGPLALKIAGRIAEFFPD | | VNDQSPGPLALKIAGRIAEF |
| subunit 9 | | AVLIMLDNQKLVPQPRVPPVIV | | FPDAVLIMLDNQKLVPQPRV |
| | | LENQGLRWVPKDKNLVMWRDWE | | PPVIVLENQGLRWVPKDKNL |
| | | ESRQMVGALLEDRAHQHLVDED | | VMWRDWEESRQMVGALLEDR |
| | | CHLDDIRQDWTNQRLNTQITQW | | AHQHLVDEDCHLDDIRQDWT |
| | | VGPTNGNGNA | | NQRLNTQITQWVGPTNGNGN |
| | | | | A |
| PSDE_HUMAN | 99 | MDRLLRLGGGMPGLGQGPPTDA | 207 | QVYISSLALLKMLKHGRAGV |
| 26S | | PAVDTAEQVYISSLALLKMLKH | | PMEVMGLMLGEFVDDYTVRV |
| proteasome | | GRAGVPMEVMGLMLGEFVDDYT | | IDVFAMPQSGTGVSVEAVDP |
| non-ATPase | | VRVIDVFAMPQSGTGVSVEAVD | | VFQAKMLDMLKQTGRPEMVV |
| regulatory | | PVFQAKMLDMLKQTGRPEMVVG | | GWYHSHPGFGCWLSGVDINT |
| subunit 14 | | WYHSHPGFGCWLSGVDINTQQS | | QQSFEALSERAVAVVVDPIQ |
| | | FEALSERAVAVVVDPIQSVKGK | | SVKGKVVIDAFRLINANMMV |
| | | VVIDAFRLINANMMVLGHEPRQ | | LGHEPRQTTSNLGHLNKPSI |
| | | TTSNLGHLNKPSIQALIHGLNR | | QALIHGLNRHYYSITINYRK |
| | | HYYSITINYRKNELEQKMLLNL | | NELEQKMLLNLHKKSWMEGL |
| | | HKKSWMEGLTLQDYSEHCKHNE | | TLQDYSEHCKHNESVVKEML |
| | | SVVKEMLELAKNYNKAVEEEDK | | ELAKNYNKAVEEEDKMTPEQ |
| | | MTPEQLAIKNVGKQDPKRHLEE | | LAIKNVGKQDPKRHLEEHVD |
| | | HVDVLMTSNIVQCLAAMLDTVV | | VLMTSNIVQCLAAMLDTVVE |
| | | FK | | K |
| MYSM1_HUMAN | 100 | MAAEEADVDIEGDVVAAAGAQP | 208 | QVKVASEALLIMDLHAHVSM |
| MHistone | | GSGENTASVLQKDHYLDSSWRT | | AEVIGLLGGRYSEVDKVVEV |
| H2A | | ENGLIPWTLDNTISEENRAVIE | | CAAEPCNSLSTGLQCEMDPV |
| deubiquitinase | | KMLLEEEYYLSKKSQPEKVWLD | | SQTQASETLAVRGESVIGWY |
| MYSM1 | | QKEDDKKYMKSLQKTAKIMVHS | | HSHPAFDPNPSLRDIDTQAK |
| | | PTKPASYSVKWTIEEKELFEQG | | YQSYFSRGGAKFIGMIVSPY |
| | | LAKFGRRWTKISKLIGSRTVLQ | | NRNNPLPYSQITCLVISEEI |
| | | VKSYARQYFKNKVKCGLDKETP | | SPDGSYRLPYKFEVQQMLEE |
| | | NQKTGHNLQVKNEDKGTKAWTP | | PQWGLVFEKTRWIIEKYRLS |
| | | SCLRGRADPNLNAVKIEKLSDD | | HSSVPMDKIFRRDSDLTCLQ |
| | | EEVDITDEVDELSSQTPQKNSS | | KLLECMRKTLSKVTNCFMAE |
| | | SDLLLDEPNSKMHETNQGEFIT | | EFLTEIENLFLSNYKSNQEN |
| | | SDSQEALESKSSRGCLQNEKQD | | GVTEENCTKELLM |
| | | ETLSSSEITLWTEK | | |
| | | QSNGDKKSIELNDQKENELIKN | | |
| | | CNKHDGRGIIVDARQLPSPEPC | | |
| | | EIQKNLNDNEMLFHSCQMVEES | | |
| | | HEEEELKPPEQEIEIDRNIIQE | | |
| | | EEKQAIPEFFEGRQAKTPERYL | | |
| | | KIRNYILDQWEICKPKYLNKTS | | |
| | | VRPGLKNCGDVNCIGRIHTYLE | | |
| | | LIGAINFGCEQAVYNRPQTVDK | | |
| | | VRIRDRKDAVEAYQLAQRLQSM | | |
| | | RTRRRRVRDPWGNWCDAKDLEG | | |
| | | QTFEHLSAEELAKRREEEKGRP | | |
| | | VKSLKVPRPTKSSFDPFQLIPC | | |
| | | NFFSEEKQEPFQVKVASEALLI | | |
| | | MDLHAHVSMAEVIG | | |
| | | LLGGRYSEVDKVVEVCAAEPCN | | |
| | | SLSTGLQCEMDPVSQTQASETL | | |
| | | AVRGFSVIGWYHSHPAFDPNPS | | |
| | | LRDIDTQAKYQSYFSRGGAKFI | | |
| | | GMIVSPYNRNNPLPYSQITCLV | | |
| | | ISEEISPDGSYRLPYKFEVQQM | | |
| | | LEEPQWGLVFEKTRWIIEKYRL | | |
| | | SHSSVPMDKIFRRDSDLTCLQK | | |
| | | LLECMRKTLSKVINCFMAEEFL | | |
| | | TEIENLELSNYKSNQENGVTEE | | |
| | | NCTKELLM | | |
| ABRX2_HUMAN | 101 | MAASISGYTFSAVCFHSANSNA | 209 | AVCFHSANSNADHEGELLGE |
| MBRISC | | DHEGELLGEVRQEETFSISDSQ | | VRQEETFSISDSQISNTEFL |
| complex | | ISNTEFLQVIEIHNHQPCSKLF | | QVIEIHNHQPCSKLESFYDY |
| subunit | | SFYDYASKVNEESLDRILKDRR | | ASKVNEESLDRILKDRRKKV |
| Abraxas 2 | | KKVIGWYRFRRNTQQQMSYREQ | | IGWYRFRRNTQQQMSYREQV |
| | | VLHKQLTRILGVPDLVELLESF | | LHKQLTRIL |
| | | ISTANNSTHALEYVLERPNRRY | | GVPDLVELLESFISTANNST |
| | | NQRISLAIPNLGNTSQQEYKVS | | HALEYVLERPNRRYNQRISL |
| | | SVPNTSQSYAKVIKEHGTDFED | | AIPNLGNTSQQEYKVSSVPN |
| | | KDGVMKDIRAIYQVYNALQEKV | | TSQSYAKVIKEHGTDFFDKD |
| | | QAVCADVEKSERVVESCQAEVN | | GVMKDIRAIYQVYNALQEKV |
| | | KLRRQITQRKNEKEQERRLQQA | | QAVCADVEKSERVVESCQAE |
| | | VLSRQMPSESLDPAFSPRMPSS | | VNKLRRQITQRKNEKEQERR |
| | | GFAAEGRSTLGDAE | | LQQAVLSRQMPSESLDPAFS |
| | | ASDPPPPYSDFHPNNQESTLSH | | PRMPSSGFAAEGRSTLGDAE |
| | | SRMERSVEMPRPQAVGSSNYAS | | ASDPPPPYSDFHPNNQESTL |
| | | TSAGLKYPGSGADLPPPQRAAG | | SHSRMERSVEMPRPQAVGSS |
| | | DSGEDSDDSDYENLIDPTEPSN | | NYASTSAGLKYPGSGADLPP |
| | | SEYSHSKDSRPMAHPDEDPRNT | | PQRAAGDSGEDSDDSDYENL |
| | | QTSQI | | IDPTEPSNSEYSHSKDSRPM |
| | | | | AHPDEDPRNTQTSQI |
| PRP8_HUMAN | 102 | MAGVFPYRGPGNPVPGPLAPLP | 210 | FNPRTGQLELKIIHTSVWAG |
| Pre-mRNA- | | DYMSEEKLQEKARKWQQLQAKR | | QKRLGQLAKWKTAEEVAALI |
| processing- | | YAEKRKFGFVDAQKEDMPPEHV | | RSLPVEEQPKQIIVTRKGML |
| splicing factor | | RKIIRDHGDMTNRKFRHDKRVY | | DPLEVHLLDEPNIVIKGSEL |
| 8 | | LGALKYMPHAVLKLLENMPMPW | | QLPFQACLKVEKFGDLILKA |
| | | EQIRDVPVLYHITGAISFVNEI | | TEPQMVLENLYDDWLKTISS |
| | | PWVIEPVYISQWGSMWIMMRRE | | YTAFSRLILILRALHVNNDR |
| | | KRDRRHFKRMRFPPEDDEEPPL | | AKVILKPDKTTITEPHHIWP |
| | | DYADNILDVEPLEAIQLELDPE | | TLTDEEWIKVEVQLKDLILA |
| | | EDAPVLDWFYDHQPLRDSRKYV | | DYGKKNNVNVASLTQSEIRD |
| | | NGSTYQRWQFTLPMMSTLYRLA | | IILGMEISAPSQQRQQIAEI |
| | | NQLLTDLVDDNYFYLFDLKAFF | | EKQTKEQSQLTATQTRTVNK |
| | | TSKALNMAIPGGPKFEPLVRDI | | HGDEIITSTTSNYETQTFSS |
| | | NLQDEDWNEFNDIN | | KTEWRVRAISAANLHLRTNH |
| | | KIIIRQPIRTEYKIAFPYLYNN | | IYVSSDDIKETGYTYILPKN |
| | | LPHHVHLTWYHTPNVVFIKTED | | VLKKFICISDLRAQIAGYLY |
| | | PDLPAFYFDPLINPISHRHSVK | | GVSPPDNPQVKEIRCIVMVP |
| | | SQEPLPDDDEEFELPEFVEPFL | | QWGTHQTVHLPGQLPQHEYL |
| | | KDTPLYTDNTANGIALLWAPRP | | KEMEPLGWIHTQPNESPQLS |
| | | FNLRSGRTRRALDIPLVKNWYR | | PQDVTTHAKIMADNPSWDGE |
| | | EHCPAGQPVKVRVSYQKLLKYY | | KTIIITCSFTPGSCTLTAYK |
| | | VLNALKHRPPKAQKKRYLFRSF | | LTPSGYEWGRQNTDKGNNPK |
| | | KATKFFQSTKLDWVEVGLQVCR | | GYLPSHYERVQMLLSDRFLG |
| | | QGYNMLNLLIHRKNLNYLHLDY | | FFMVPAQSSWNYNFMGVRHD |
| | | NFNLKPVKTLTTKERKKSREGN | | PNMKYELQLANPKEFYHEVH |
| | | AFHLCREVLRLTKLVVDSHVQY | | RPSHFLNFALLQEGEVYSAD |
| | | RLGNVDAFQLADGLQYIFAHVG | | REDLYA |
| | | QLTGMYRYKYKLMR | | |
| | | QIRMCKDLKHLIYYRFNTGPVG | | |
| | | KGPGCGFWAAGWRVWLFFMRGI | | |
| | | TPLLERWLGNLLARQFEGRHSK | | |
| | | GVAKTVTKQRVESHFDLELRAA | | |
| | | VMHDILDMMPEGIKQNKARTIL | | |
| | | QHLSEAWRCWKANIPWKVPGLP | | |
| | | TPIENMILRYVKAKADWWTNTA | | |
| | | HYNRERIRRGATVDKTVCKKNL | | |
| | | GRLTRLYLKAEQERQHNYLKDG | | |
| | | PYITAEEAVAVYTTTVHWLESR | | |
| | | RFSPIPFPPLSYKHDTKLLILA | | |
| | | LERLKEAYSVKSRLNQSQREEL | | |
| | | GLIEQAYDNPHEALSRIKRHLL | | |
| | | TQRAFKEVGIEFMD | | |
| | | LYSHLVPVYDVEPLEKITDAYL | | |
| | | DQYLWYEADKRRLFPPWIKPAD | | |
| | | TEPPPLLVYKWCQGINNLQDVW | | |
| | | ETSEGECNVMLESRFEKMYEKI | | |
| | | DLTLLNRLLRLIVDHNIADYMT | | |
| | | AKNNVVINYKDMNHTNSYGIIR | | |
| | | GLQFASFIVQYYGLVMDLLVLG | | |
| | | LHRASEMAGPPQMPNDFLSFQD | | |
| | | IATEAAHPIRLFCRYIDRIHIF | | |
| | | FRFTADEARDLIQRYLTEHPDP | | |
| | | NNENIVGYNNKKCWPRDARMRL | | |
| | | MKHDVNLGRAVFWDIKNRLPRS | | |
| | | VTTVQWENSFVSVYSKDNPNLL | | |
| | | FNMCGFECRILPKC | | |
| | | RTSYEEFTHKDGVWNLQNEVTK | | |
| | | ERTAQCFLRVDDESMQRFHNRV | | |
| | | RQILMASGSTTFTKIVNKWNTA | | |
| | | LIGLMTYFREAVVNTQELLDLL | | |
| | | VKCENKIQTRIKIGLNSKMPSR | | |
| | | FPPVVFYTPKELGGLGMLSMGH | | |
| | | VLIPQSDLRWSKQTDVGITHER | | |
| | | SGMSHEEDQLIPNLYRYIQPWE | | |
| | | SEFIDSQRVWAEYALKRQEAIA | | |
| | | QNRRLTLEDLEDSWDRGIPRIN | | |
| | | TLFQKDRHTLAYDKGWRVRTDE | | |
| | | KQYQVLKQNPFWWTHQRHDGKL | | |
| | | WNLNNYRTDMIQALGGVEGILE | | |
| | | HTLFKGTYFPTWEG | | |
| | | LEWEKASGFEESMKWKKLTNAQ | | |
| | | RSGLNQIPNRRFTLWWSPTINR | | |
| | | ANVYVGFQVQLDLTGIFMHGKI | | |
| | | PTLKISLIQIFRAHLWQKIHES | | |
| | | IVMDLCQVEDQELDALEIETVQ | | |
| | | KETIHPRKSYKMNSSCADILLE | | |
| | | ASYKWNVSRPSLLADSKDVMDS | | |
| | | TTTQKYWIDIQLRWGDYDSHDI | | |
| | | ERYARAKFLDYTTDNMSIYPSP | | |
| | | TGVLIAIDLAYNLHSAYGNWFP | | |
| | | GSKPLIQQAMAKIMKANPALYV | | |
| | | LRERIRKGLQLYSSEPTEPYLS | | |
| | | SQNYGELFSNQIIWFVDDTNVY | | |
| | | RVTIHKTFEGNLTT | | |
| | | KPINGAIFIFNPRTGQLELKII | | |
| | | HTSVWAGQKRLGQLAKWKTAEE | | |
| | | VAALIRSLPVEEQPKQIIVTRK | | |
| | | GMLDPLEVHLLDFPNIVIKGSE | | |
| | | LQLPFQACLKVEKFGDLILKAT | | |
| | | EPQMVLENLYDDWLKTISSYTA | | |
| | | FSRLILILRALHVNNDRAKVIL | | |
| | | KPDKTTITEPHHIWPTLTDEEW | | |
| | | IKVEVQLKDLILADYGKKNNVN | | |
| | | VASLTQSEIRDIILGMEISAPS | | |
| | | QQRQQIAEIEKQTKEQSQLTAT | | |
| | | QTRTVNKHGDEIITSTTSNYET | | |
| | | QTFSSKTEWRVRAISAANLHLR | | |
| | | TNHIYVSSDDIKET | | |
| | | GYTYILPKNVLKKFICISDLRA | | |
| | | QIAGYLYGVSPPDNPQVKEIRC | | |
| | | IVMVPQWGTHQTVHLPGQLPQH | | |
| | | EYLKEMEPLGWIHTQPNESPQL | | |
| | | SPQDVTTHAKIMADNPSWDGEK | | |
| | | TIIITCSFTPGSCTLTAYKLTP | | |
| | | SGYEWGRQNTDKGNNPKGYLPS | | |
| | | HYERVQMLLSDRFLGFFMVPAQ | | |
| | | SSWNYNFMGVRHDPNMKYELQL | | |
| | | ANPKEFYHEVHRPSHELNFALL | | |
| | | QEGEVYSADREDLYA | | |
| NPL4_HUMAN | 103 | MAESIIIRVQSPDGVKRITATK | 211 | QPSAITLNRQKYRHVDNIMF |
| Membrane | | RETAATFLKKVAKEFGFQNNGE | | ENHTVADRFLDFWRKTGNQH |
| protein | | SVYINRNKTGEITASSNKSLNL | | FGYLYGRYTEHKDIPLGIRA |
| localization | | LKIKHGDLLFLFPSSLAGPSSE | | EVAAIYEPPQIGTQNSLELL |
| protein 4 | | METSVPPGFKVEGAPNVVEDEI | | EDPKAEVVDEIAAKLGLRKV |
| homolog | | DQYLSKQDGKIYRSRDPQLCRH | | GWIFTDLVSEDTRKGTVRYS |
| | | GPLGKCVHCVPLEPFDEDYLNH | | RNKDTYFLSSEECITAGDFQ |
| | | LEPPVKHMSFHAYIRKLTGGAD | | NKHPNMCRLSPDGHFGSKFV |
| | | KGKFVALENISCKIKSGCEGHL | | TAVATGGPDNQVHFEGYQVS |
| | | PWPNGICTKCQPSAITLNRQKY | | NQCMALVRDECLLPCKDAPE |
| | | RHVDNIMFENHTVADRELDEWR | | LGYAKESSSEQYVPDVFYKD |
| | | KTGNQHFGYLYGRYTEHKDIPL | | VDKFGNEITQLARPLPVEYL |
| | | GIRAEVAAIYEPPQIGTQNSLE | | IIDITTTFPKDPVYTFSISQ |
| | | LLEDPKAEVVDEIA | | NPFPIENRDVLGETQDFHSL |
| | | AKLGLRKVGWIFTDLVSEDTRK | | ATYLSQNTSSVELDTISDFH |
| | | GTVRYSRNKDTYFLSSEECITA | | LLLFLVTNEVMPLQDSISLL |
| | | GDFQNKHPNMCRLSPDGHFGSK | | LEAVRTRNEELAQTWKRSEQ |
| | | FVTAVATGGPDNQVHFEGYQVS | | WATIEQLCSTVGGQLPGLHE |
| | | NQCMALVRDECLLPCKDAPELG | | YGAVGGSTHTATAAMWACQH |
| | | YAKESSSEQYVPDVFYKDVDKF | | CTFMNQPGTGHCEMCSLPRT |
| | | GNEITQLARPLPVEYLIIDITT | | |
| | | TFPKDPVYTFSISQNPFPIENR | | |
| | | DVLGETQDFHSLATYLSQNTSS | | |
| | | VELDTISDFHLLLFLVTNEVMP | | |
| | | LQDSISLLLEAVRTRNEELAQT | | |
| | | WKRSEQWATIEQLCSTVGGQLP | | |
| | | GLHEYGAVGGSTHTATAAMWAC | | |
| | | QHCTFMNQPGTGHCEMCSLPRT | | |
| EMC8_HUMAN | 104 | MPGVKLTTQAYCKMVLHGAKYP | 212 | TQAYCKMVLHGAKYPHCAVN |
| ER | | HCAVNGLLVAEKQKPRKEHLPL | | GLLVAEKQKPRKEHLPLGGP |
| membrane | | GGPGAHHTLFVDCIPLFHGTLA | | GAHHTLFVDCIPLFHGTLAL |
| protein | | LAPMLEVALTLIDSWCKDHSYV | | APMLEVALTLIDSWCKDHSY |
| complex | | IAGYYQANERVKDASPNQVAEK | | VIAGYYQANERVKDASPNQV |
| subunit 8 | | VASRIAEGFSDTALIMVDNTKF | | AEKVASRIAEGFSDTALIMV |
| | | TMDCVAPTIHVYEHHENRWRCR | | DNTKFTMDCVAPTIHVYEHH |
| | | DPHHDYCEDWPEAQRISASLLD | | ENRWRCRDPHHDYCEDWPEA |
| | | SRSYETLVDFDNHLDDIRNDWT | | QRISASLLDSRSYETLVDED |
| | | NPEINKAVLHLC | | NHLDDIRNDWTNPEINKAVL |
| | | | | HLC |
| ABRX1_ | 105 | MEGESTSAVLSGFVLGALAFQH | 213 | GFVLGALAFQHLNTDSDTEG |
| HUMAN | | LNTDSDTEGELLGEVKGEAKNS | | FLLGEVKGEAKNSITDSQMD |
| BRCA1-A | | ITDSQMDDVEVVYTIDIQKYIP | | DVEVVYTIDIQKYIPCYQLF |
| complex | | CYQLFSFYNSSGEVNEQALKKI | | SFYNSSGEVNEQALKKILSN |
| subunit | | LSNVKKNVVGWYKERRHSDQIM | | VKKNVVGWYKFRRHSDQIMT |
| Abraxas 1 | | TFRERLLHKNLQEHFSNQDLVE | | FRERLLHKNLQEHFSNQDLV |
| | | LLLTPSIITESCSTHRLEHSLY | | FLLLTPSIITESCSTHRLEH |
| | | KPQKGLFHRVPLVVANLGMSEQ | | SLYKPQKGLFHRVPLVVANL |
| | | LGYKTVSGSCMSTGFSRAVQTH | | GMSEQLGYKTVSGSCMSTGF |
| | | SSKFFEEDGSLKEVHKINEMYA | | SRAVQTHSSKFFEEDGSLKE |
| | | SLQEELKSICKKVEDSEQAVDK | | VHKINEMYASLQEELKSICK |
| | | LVKDVNRLKREIEKRRGAQIQA | | KVEDSEQAVDKLVKDVNRLK |
| | | AREKNIQKDPQENIFLCQALRT | | REIEKRRGAQIQAAREKNIQ |
| | | FFPNSEFLHSCVMS | | KDPQENIFLCQALRTFFPNS |
| | | LKNRHVSKSSCNYNHHLDVVDN | | EFLHSCVMSLKNRHVSKSSC |
| | | LTLMVEHTDIPEASPASTPQII | | NYNHHLDVVDNLTLMVEHTD |
| | | KHKALDLDDRWQFKRSRLLDTQ | | IPEASPASTPQIIKHKALDL |
| | | DKRSKADTGSSNQDKASKMSSP | | DDRWQFKRSRLLDTQDKRSK |
| | | ETDEEIEKMKGFGEYSRSPTF | | ADTGSSNQDKASKMSSPETD |
| | | | | EEIEKMKGFGEYSRSPTF |
| STALP_HUMAN | 106 | MDQPFTVNSLKKLAAMPDHTDV | 214 | VVLPEDLCHKELQLAESNTV |
| AMSH- | | SLSPEERVRALSKLGCNITISE | | RGIETCGILCGKLTHNEFTI |
| like protease | | DITPRRYFRSGVEMERMASVYL | | THVIVPKQSAGPDYCDMENV |
| | | EEGNLENAFVLYNKFITLFVEK | | EELFNVQDQHDLLTLGWIHT |
| | | LPNHRDYQQCAVPEKQDIMKKL | | HPTQTAFLSSVDLHTHCSYQ |
| | | KEIAFPRTDELKNDLLKKYNVE | | LMLPEAIAIVCSPKHKDTGI |
| | | YQEYLQSKNKYKAEILKKLEHQ | | FRLTNAGMLEVSACKKKGFH |
| | | RLIEAERKRIAQMRQQQLESEQ | | PHTKEPRLFSICKHVLVKDI |
| | | FLFFEDQLKKQELARGQMRSQQ | | KIIVLDLR |
| | | TSGLSEQIDGSALSCFSTHQNN | | |
| | | SLLNVFADQPNKSDATNYASHS | | |
| | | PPVNRALTPAATLSAVQNLVVE | | |
| | | GLRCVVLPEDLCHKELQLAESN | | |
| | | TVRGIETCGILCGK | | |
| | | LTHNEFTITHVIVPKQSAGPDY | | |
| | | CDMENVEELFNVQDQHDLLTLG | | |
| | | WIHTHPTQTAFLSSVDLHTHCS | | |
| | | YQLMLPEAIAIVCSPKHKDTGI | | |
| | | FRLTNAGMLEVSACKKKGFHPH | | |
| | | TKEPRLESICKHVLVKDIKIIV | | |
| | | LDLR | | |
| CSN6_HUMAN | 107 | MAAAAAAAAATNGTGGSSGMEV | 215 | VALHPLVILNISDHWIRMRS |
| COP9 | | DAAVVPSVMACGVTGSVSVALH | | QEGRPVQVIGALIGKQEGRN |
| signalosome | | PLVILNISDHWIRMRSQEGRPV | | IEVMNSFELLSHTVEEKIII |
| complex | | QVIGALIGKQEGRNIEVMNSFE | | DKEYYYTKEEQFKQVFKELE |
| subunit 6 | | LLSHTVEEKIIIDKEYYYTKEE | | FLGWYTTGGPPDPSDIHVHK |
| | | QFKQVFKELEFLGWYTTGGPPD | | QVCEIIESPLFLKLNPMTKH |
| | | PSDIHVHKQVCEIIESPLFLKL | | TDLPVSVFESVIDIINGEAT |
| | | NPMTKHTDLPVSVFESVIDIIN | | MLFAELTYTLATEEAERIGV |
| | | GEATMLFAELTYTLATEEAERI | | DHVARMTATGSGENSTVAEH |
| | | GVDHVARMTATGSGENSTVAEH | | LIAQHSAIKMLHSRVKLILE |
| | | LIAQHSAIKMLHSRVKLILEYV | | YVKASEAGEVPENHEILREA |
| | | KASEAGEVPENHEILREAYALC | | YALCHCLPVLSTDKFKTDFY |
| | | HCLPVLSTDKFKTDFYDQCNDV | | DQCNDVGLMAYLGTITKTCN |
| | | GLMAYLGTITKTCNTMNQFVNK | | TMNQFVNKFNVLYDRQGIGR |
| | | FNVLYDRQGIGRRMRGLFF | | RMRGLFF |
| EIF3F_ | 108 | MATPAVPVSAPPATPTPVPAAA | 216 | VRLHPVILASIVDSYERRNE |
| HUMAN | | PASVPAPTPAPAAAPVPAAAPA | | GAARVIGTLLGTVDKHSVEV |
| Eukaryotic | | SSSDPAAAAAATAAPGQTPASA | | TNCFSVPHNESEDEVAVDME |
| translation | | QAPAQTPAPALPGPALPGPFPG | | FAKNMYELHKKVSPNELILG |
| initiation | | GRVVRLHPVILASIVDSYERRN | | WYATGHDITEHSVLIHEYYS |
| factor 3 | | EGAARVIGTLLGTVDKHSVEVT | | REAPNPIHLTVDTSLQNGRM |
| subunit F | | NCFSVPHNESEDEVAVDMEFAK | | SIKAYVSTLMGVPGRTMGVM |
| | | NMYELHKKVSPNELILGWYATG | | FTPLTVKYAYYDTERIGVDL |
| | | HDITEHSVLIHEYYSREAPNPI | | IMKTCFSPNRVIGLSSDLQQ |
| | | HLTVDTSLQNGRMSIKAYVSTL | | VGGASARIQDALSTVLQYAE |
| | | MGVPGRTMGVMFTPLTVKYAYY | | DVLSGKVSADNTVGRFLMSL |
| | | DTERIGVDLIMKTCFSPNRVIG | | VNQVPKIVPDDFETMLNSNI |
| | | LSSDLQQVGGASARIQDALSTV | | NDLLMVTYLANLTQSQIALN |
| | | LQYAEDVLSGKVSADNTVGREL | | EKLVNL |
| | | MSLVNQVPKIVPDDFETMLNSN | | |
| | | INDLLMVTYLANLTQSQIALNE | | |
| | | KLVNL | | |
| PSMD7_HUMAN | 109 | MPELAVQKVVVHPLVLLSVVDH | 217 | VVVHPLVLLSVVDHENRIGK |
| M26S | | FNRIGKVGNQKRVVGVLLGSWQ | | VGNQKRVVGVLLGSWQKKVL |
| proteasome | | KKVLDVSNSFAVPFDEDDKDDS | | DVSNSFAVPFDEDDKDDSVW |
| non-ATPase | | VWFLDHDYLENMYGMFKKVNAR | | FLDHDYLENMYGMFKKVNAR |
| regulatory | | ERIVGWYHTGPKLHKNDIAINE | | ERIVGWYHTGPKLHKNDIAI |
| subunit 7 | | LMKRYCPNSVLVIIDVKPKDLG | | NELMKRYCPNSVLVIIDVKP |
| | | LPTEAYISVEEVHDDGTPTSKT | | KDLGLPTEAYISVEEVHDDG |
| | | FEHVTSEIGAEEAEEVGVEHLL | | TPTSKTFEHVTSEIGAEEAE |
| | | RDIKDTTVGTLSQRITNQVHGL | | EVGVEHLLRDIKDTTVGTLS |
| | | KGLNSKLLDIRSYLEKVATGKL | | QRITNQVHGLKGLNSKLLDI |
| | | PINHQIIYQLQDVENLLPDVSL | | RSYLEKVATGKLPINHQIIY |
| | | QEFVKAFYLKTNDQMVVVYLAS | | QLQDVFNLLPDVSLQEFVKA |
| | | LIRSVVALHNLINNKIANRDAE | | FYLKTNDQMVVVYLASLIRS |
| | | KKEGQEKEESKKDRKEDKEKDK | | VVALHNLINNKIANRDAEKK |
| | | DKEKSDVKKEEKKEKK | | EGQEKEESKKDRKEDKEKDK |
| | | | | DKEKSDVKKEEKKEKK |
| EIF3H_HUMAN | 110 | MASRKEGTGSTATSSSSTAGAA | 218 | VQIDGLVVLKIIKHYQEEGQ |
| AN | | GKGKGKGGSGDSAVKQVQIDGL | | GTEVVQGVLLGLVVEDRLEI |
| Eukaryotic | | VVLKIIKHYQEEGQGTEVVQGV | | TNCFPFPQHTEDDADEDEVQ |
| translation | | LLGLVVEDRLEITNCFPFPQHT | | YQMEMMRSLRHVNIDHLHVG |
| initiation | | EDDADFDEVQYQMEMMRSLRHV | | WYQSTYYGSFVTRALLDSQF |
| factor 3 | | NIDHLHVGWYQSTYYGSFVTRA | | SYQHAIEESVVLIYDPIKTA |
| subunit H | | LLDSQFSYQHAIEESVVLIYDP | | QGSLSLKAYRLTPKLMEVCK |
| | | IKTAQGSLSLKAYRLTPKLMEV | | EKDESPEALKKANITFEYME |
| | | CKEKDESPEALKKANITFEYME | | EEVPIVIKNSHLINVLMWEL |
| | | EEVPIVIKNSHLINVLMWELEK | | EKKSAVADKHELLSLASSNH |
| | | KSAVADKHELLSLASSNHLG | | LGKNLQLLMDRVDEMSQDIV |
| | | KNLQLLMDRVDEMSQDIVKYNT | | KYNTYMRNTSKQQQQKHQYQ |
| | | YMRNTSKQQQQKHQYQQRRQQE | | QRRQQENMQRQSRGEPPLPE |
| | | NMQRQSRGEPPLPEEDLSKLFK | | EDLSKLFKPPQPPARMDSLL |
| | | PPQPPARMDSLLIAGQINTYCQ | | IAGQINTYCQNIKEFTAQNL |
| | | NIKEFTAQNLGKLFMAQALQEY | | GKLEMAQALQEYNN |
| | | NN | | |
| CSN5_HUMAN | 111 | MAASGSGMAQKTWELANNMQEA | 219 | YCKISALALLKMVMHARSGG |
| COP9 | | QSIDEIYKYDKKQQQEILAAKP | | NLEVMGLMLGKVDGETMIIM |
| signalosome | | WTKDHHYFKYCKISALALLKMV | | DSFALPVEGTETRVNAQAAA |
| complex | | MHARSGGNLEVMGLMLGKVDGE | | YEYMAAYIENAKQVGRLENA |
| subunit 5 | | TMIIMDSFALPVEGTETRVNAQ | | IGWYHSHPGYGCWLSGIDVS |
| | | AAAYEYMAAYIENAKQVGRLEN | | TQMLNQQFQEPFVAVVIDPT |
| | | AIGWYHSHPGYGCWLSGIDVST | | RTISAGKVNLGAFRTYPKGY |
| | | QMLNQQFQEPFVAVVIDPTRTI | | KPPDEGPSEYQTIPLNKIED |
| | | SAGKVNLGAFRTYPKGYKPPDE | | FGVHCKQYYALEVSYFKSSL |
| | | GPSEYQTIPLNKIEDFGVHCKQ | | DRKLLELLWNKYWVNTLSSS |
| | | YYALEVSYFKSSLDRKLLELLW | | SLLTNADYTTGQVEDLSEKL |
| | | NKYWVNTLSSSSLLTNADYTTG | | EQSEAQLGRGSFMLGLETHD |
| | | QVFDLSEKLEQSEAQLGRGSFM | | RKSEDKLAKATRDSCKTTIE |
| | | LGLETHDRKSEDKLAKATRDSC | | AIHGLMSQVIKDKLENQINI |
| | | KTTIEAIHGLMSQVIKDKLENQ | | S |
| | | INIS | | |
| BRCC3_HUMAN | 112 | MAVQVVQAVQAVHLESDAFLVC | 220 | VHLESDAFLVCLNHALSTEK |
| MLys-63- | | LNHALSTEKEEVMGLCIGELND | | EEVMGLCIGELNDDTRSDSK |
| specific | | DTRSDSKFAYTGTEMRTVAEKV | | FAYTGTEMRTVAEKVDAVRI |
| deubiquitinase | | DAVRIVHIHSVIILRRSDKRKD | | VHIHSVIILRRSDKRKDRVE |
| BRCC36 | | RVEISPEQLSAASTEAERLAEL | | ISPEQLSAASTEAERLAELT |
| | | TGRPMRVVGWYHSHPHITVWPS | | GRPMRVVGWYHSHPHITVWP |
| | | HVDVRTQAMYQMMDQGFVGLIF | | SHVDVRTQAMYQMMDQGFVG |
| | | SCFIEDKNTKTGRVLYTCFQSI | | LIFSCFIEDKNTKTGRVLYT |
| | | QAQKSSESLHGPRDFWSSSQHI | | CFQSIQAQKSSESLHGPRDE |
| | | SIEGQKEEERYERIEIPIHIVP | | WSSSQHISIEGQKEEERYER |
| | | HVTIGKVCLESAVELPKILCQE | | IEIPIHIVPHVTIGKVCLES |
| | | EQDAYRRIHSLTHLDSVTKIHN | | AVELPKILCQEEQDAYRRIH |
| | | GSVFTKNLCSQMSAVSGPLLQW | | SLTHLDSVTKIHNGSVETKN |
| | | LEDRLEQNQQHLQELQQEKEEL | | LCSQMSAVSGPLLQWLEDRL |
| | | MQELSSLE | | EQNQQHLQELQQEKEELMQE |
| | | | | LSSLE |

### 5.3.2 Targeting Domain

In some embodiments, the targeting domain comprises a targeting moiety that specifically binds to a target membrane protein. In some embodiments, the targeting moiety comprises an antibody (or antigen binding fragment thereof). In some embodiments, the antibody is a full-length antibody, a single chain variable fragment (scFv), a (scFv)₂, a scFv-Fc, a Fab, a Fab′, a (Fab′)₂, a F(v), a single domain antibody, a single chain antibody, a VHH, or a (VHH)₂. In some embodiments the targeting moiety comprises a VHH. In some embodiments the targeting moiety comprises a (VHH)₂.

In some embodiments, the targeting moiety specifically binds to a wild type target membrane protein. In some embodiments, the targeting moiety specifically binds to a wild type target membrane protein, but does not specifically binds to a variant of the target membrane protein associated with a genetic disease. In some embodiments, the targeting moiety specifically binds to a naturally occurring variant of a target membrane protein. In some embodiments, the targeting moiety specifically binds to a naturally occurring variant of a target membrane protein that is associated with a genetic disease (e.g., a genetic disease described herein). In some embodiments, the targeting moiety specifically binds to a naturally occurring variant of a target membrane protein that is a cause of a genetic disease (e.g., a genetic disease described herein). In some embodiments, the targeting moiety specifically binds a naturally occurring variant of a target membrane protein that is a loss of a function variant. In some embodiments, the targeting moiety specifically binds a naturally occurring variant of a target membrane protein that is a loss of a function variant associated with a genetic disease (e.g., a genetic disease described herein). In some embodiments, the targeting moiety specifically binds a naturally occurring variant of a target membrane protein that is a loss of a function variant that causes a genetic disease (e.g., a genetic disease described herein).

### 5.3.2.1 Exemplary Target Membrane Proteins

In some embodiments, targeting moiety specifically binds a target membrane protein (e.g., a membrane protein described herein). Exemplary target membrane proteins include, but are not limited to, glutamate receptor ionotropic NMDA 2B (GRIN2B), cystic fibrosis transmembrane conductance regulator (CFTR), sodium channel protein type 1 subunit alpha (SCN1A), copper-transporting ATPase 2 (ATP7B), potassium voltage-gated channel subfamily KQT member 2 (KCNQ2), sodium channel protein type 2 subunit alpha (SCN2A), voltage-dependent P/Q-type calcium channel subunit alpha-1A (CACNA1A), solute carrier family 2, facilitated glucose transporter member 1 (SLC2A1), sodium channel protein type 8 subunit alpha (SCN8A), proline-rich transmembrane protein 2 (PRRT2), glutamate receptor ionotropic, NMDA 2A (GRIN2A), sodium- and chloride-dependent GABA transporter 1 (SLC6A1), usherin (USH2A), sodium/potassium-transporting ATPase subunit alpha-2 (ATP1A2), sodium/potassium-transporting ATPase subunit alpha-3 (ATP1A3), sodium channel protein type 9 subunit alpha (SCN9A), protocadherin-19 (PCDH19), gamma-aminobutyric acid receptor subunit beta-3 (GABRB3), tuberin (TSC2), hamartin (TSC1), potassium voltage-gated channel subfamily KQT member 3 (KCNQ3), dystrophin (DMD), rhodopsin (RHO), protein jagged-1 (JAG1), inositol 1,4,5-trisphosphate receptor type 1 (ITPR1), sugar transporter SWEET1 (SLC50A1), transmembrane protein 258 (TMEM258), and follicle-stimulating hormone receptor (FSHR).

In some embodiments, the target membrane protein is GRIN2B. In some embodiments, the target membrane protein is CFTR. In some embodiments, the target membrane protein is SCN1A. In some embodiments, the target membrane protein is ATP7B. In some embodiments, the target membrane protein is KCNQ2. In some embodiments, the target membrane protein is SCN2A. In some embodiments, the target membrane protein is CACNA1A. In some embodiments, the target membrane protein is SLC2A1. In some embodiments, the target membrane protein is SCN8A. In some embodiments, the target membrane protein is PRRT2. In some embodiments, the target membrane protein is GRIN2A. In some embodiments, the target membrane protein is SLC6A1. In some embodiments, the target membrane protein is USH2A. In some embodiments, the target membrane protein is ATP1A2. In some embodiments, the target membrane protein is ATP1A3. In some embodiments, the target membrane protein is SCN9A. In some embodiments, the target membrane protein is PCDH19. In some embodiments, the target membrane protein is GABRB3. In some embodiments, the target membrane protein is TSC2. In some embodiments, the target membrane protein is TSC1. In some embodiments, the target membrane protein is KCNQ3. In some embodiments, the target membrane protein is DMD. In some embodiments, the target membrane protein is RHO. In some embodiments, the target membrane protein is JAG1. In some embodiments, the target membrane protein is ITPR1. In some embodiments, the target membrane protein is sugar transporter SWEET1 (SLC50A1). In some embodiments, the target membrane protein is transmembrane protein 258 (TMEM258). In some embodiments, the target membrane protein is follicle-stimulating hormone receptor (FSHR).

In some embodiments, the target membrane protein comprises an amino acid sequence at least 90%, 91%, 92%, 93%, 94%, 95%, 96%, 97%, 98%, 99%, or 100% identical to the amino acid sequence of any one of SEQ ID NOS: 221-245. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 221. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 222. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 223. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 224. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 225. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 226. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 227. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 228. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 229. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 230. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 240. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 241. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 242. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 243. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 244. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 245. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 294. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 295. In some embodiments, the target membrane protein comprises an amino acid sequence at least 90%, 91%, 92%, 93%, 94%, 95%, 96%, 97%, 98%, 99%, or 100% identical to the amino acid sequence of SEQ ID NO: 296.

Table 2 below, provides the wild type amino acid sequence of exemplary proteins to target for deubiquitination utilizing the fusion proteins described herein.

TABLE 2
The amino acid sequence of exemplary membrane proteins to
target for deubiquitination utilizing the fusion proteins
described herein and exemplary disease associations

| | Disease | SEQ ID | |
|---|---|---|---|
| Description | Associations | NO | WT Amino Acid Sequence |
| Solute carrier | GLUT1 | 221 | MEPSSKKLTGRLMLAVGGAVLGSLQFGYNTGVINA |
| family 2, | Deficiency | | PQKVIEEFYNQTWVHRYGESILPTTLTTLWSLSVA |
| facilitated | Syndrome | | IFSVGGMIGSFSVGLFVNRFGRRNSMLMMNLLAFV |
| glucose | | | SAVLMGFSKLGKSFEMLILGRFIIGVYCGLTTGFV |
| transporter | | | PMYVGEVSPTALRGALGTLHQLGIVVGILIAQVEG |
| member 1 | | | LDSIMGNKDLWPLLLSIIFIPALLQCIVLPFCPES |
| (SLC2A1) | | | PRFLLINRNEENRAKSVLKKLRGTADVTHDLQEMK |
| | | | EESRQMMREKKVTILELFRSPAYRQPILIAVVLQL |
| | | | SQQLSGINAVFYYSTSIFEKAGVQQPVYATIGSGI |
| | | | VNTAFTVVSLFVVERAGRRTLHLIGLAGMAGCAIL |
| | | | MTIALALLEQLPWMSYLSIVAIFGFVAFFEVGPGP |
| | | | IPWFIVAELFSQGPRPAAIAVAGESNWTSNFIVGM |
| | | | CFQYVEQLCGPYVFIIFTVLLVLFFIFTYFKVPET |
| | | | KGRTFDEIASGFRQGGASQSDKTPEELFHPLGADS |
| | | | QV |
| Proline-rich | PRRT2 | 222 | MAASSSEISEMKGVEESPKVPGEGPGHSEAETGPP |
| transmembrane | Dyskinesia & | | QVLAGVPDQPEAPQPGPNTTAAPVDSGPKAGLAPE |
| protein 2 | Epilepsy; | | TTETPAGASETAQATDLSLSPGGESKANCSPEDPC |
| (PRRT2) | Episodic | | QETVSKPEVSKEATADQGSRLESAAPPEPAPEPAP |
| | kinesigenic | | QPDPRPDSQPTPKPALQPELPTQEDPTPEILSESV |
| | dyskinesia 1 | | GEKQENGAVVPLQAGDGEEGPAPEPHSPPSKKSPP |
| | | | ANGAPPRVLQQLVEEDRMRRAHSGHPGSPRGSLSR |
| | | | HPSSQLAGPGVEGGEGTQKPRDYIILAILSCFCPM |
| | | | WPVNIVAFAYAVMSRNSLQQGDVDGAQRLGRVAKL |
| | | | LSIVALVGGVLIIIASCVINLGVYK |
| Usherin | Usher | 223 | MNCPVLSLGSGFLFQVIEMLIFAYFASISLTESRG |
| (USH2A) | syndrome, type | | LFPRLENVGAFKKVSIVPTQAVCGLPDRSTFCHSS |
| Signal | 2A | | AAAESIQFCTQRFCIQDCPYRSSHPTYTALESAGL |
| Sequence | | | SSCITPDKNDLHPNAHSNSASFIFGNHKSCFSSPP |
| Underlined | | | SPKLMASFTLAVWLKPEQQGVMCVIEKTVDGQIVE |
| | | | KLTISEKETMFYYRTVNGLQPPIKVMTLGRILVKK |
| | | | WIHLSVQVHQTKISFFINGVEKDHTPENARTLSGS |
| | | | ITDFASGTVQIGQSLNGLEQFVGRMQDERLYQVAL |
| | | | TNREILEVESGDLLRLHAQSHCRCPGSHPRVHPLA |
| | | | QRYCIPNDAGDTADNRVSRLNPEAHPLSFVNDNDV |
| | | | GTSWVSNVFTNITQLNQGVTISVDLENGQYQVFYI |
| | | | IIQFFSPQPTEIRIQRKKENSLDWEDWQYFARNCG |
| | | | AFGMKNNGDLEKPDSVNCLQLSNFTPYSRGNVTFS |
| | | | ILTPGPNYRPGYNNFYNTPSLQEFVKATQIRFHFH |
| | | | GQYYTTETAVNLRHRYYAVDEITISGRCOCHGHAD |
| | | | NCDTTSQPYRCLCSQESFTEGLHCDRCLPLYNDKP |
| | | | FRQGDQVYAFNCKPCQCNSHSKSCHYNISVDPFPF |
| | | | EHFRGGGGVCDDCEHNTTGRNCELCKDYFFRQVGA |
| | | | DPSAIDVCKPCDCDTVGTRNGSILCDQIGGQCNCK |
| | | | RHVSGRQCNQCQNGFYNLQELDPDGCSPCNCNTSG |
| | | | TVDGDITCHQNSGQCKCKANVIGLRCDHCNFGEKF |
| | | | LRSFNDVGCEPCQCNLHGSVNKFCNPHSGQCECKK |
| | | | EAKGLQCDTCRENFYGLDVTNCKACDCDTAGSLPG |
| | | | TVCNAKTGQCICKPNVEGRQCNKCLEGNFYLRQNN |
| | | | SFLCLPCNCDKTGTINGSLLCNKSTGQCPCKLGVT |
| | | | GLRCNQCEPHRYNLTIDNFQHCQMCECDSLGTLPG |
| | | | TICDPISGQCLCVPNRQGRRCNQCQPGFYISPGNA |
| | | | TGCLPCSCHTTGAVNHICNSLTGQCVCQDASIAGO |
| | | | RCDQCKDHYFGFDPQTGRCQPCNCHLSGALNETCH |
| | | | LVTGQCFCKQFVTGSKCDACVPSASHLDVNNLLGC |
| | | | SKTPFQQPPPRGQVQSSSAINLSWSPPDSPNAHWL |
| | | | TYSLLRDGFEIYTTEDQYPYSIQYFLDTDLLPYTK |
| | | | YSYYIETTNVHGSTRSVAVTYKTKPGVPEGNLTLS |
| | | | YIIPIGSDSVTLTWTTLSNQSGPIEKYILSCAPLA |
| | | | GGQPCVSYEGHETSATIWNLVPFAKYDESVQACTS |
| | | | GGCLHSLPITVITAQAPPQRLSPPKMQKISSTELH |
| | | | VEWSPPAELNGIIIRYELYMRRLRSTKETTSEESR |
| | | | VFQSSGWLSPHSFVESANENALKPPQTMTTITGLE |
| | | | PYTKYEFRVLAVNMAGSVSSAWVSERTGESAPVEM |
| | | | IPPSVEPLSSYSLNISWEKPADNVTRGKVVGYDIN |
| | | | MLSEQSPQQSIPMAFSQLLHTAKSQELSYTVEGLK |
| | | | PYRIYEFTITLCNSVGCVTSASGAGQTLAAAPAQL |
| | | | RPPLVKGINSTTIHLRWFPPEELNGPSPIYQLERR |
| | | | ESSLPALMTTMMKGIRFIGNGYCKFPSSTHPVNTD |
| | | | FTGIKASFRTKVPEGLIVFAASPGNQEEYFALQLK |
| | | | KGRLYFLFDPQGSPVEVTTTNDHGKQYSDGKWHEI |
| | | | IAIRHQAFGQITLDGIYTGSSAILNGSTVIGDNTG |
| | | | VELGGLPRSYTILRKDPEIIQKGFVGCLKDVHEMK |
| | | | NYNPSAIWEPLDWQSSEEQINVYNSWEGCPASLNE |
| | | | GAQFLGAGFLELHPYMFHGGMNFEISEKFRTDQLN |
| | | | GLLLFVYNKDGPDFLAMELKSGILTFRLNTSLAFT |
| | | | QVDLLLGLSYCNGKWNKVIIKKEGSFISASVNGLM |
| | | | KHASESGDQPLVVNSPVYVGGIPQELLNSYQHLCL |
| | | | EQGFGGCMKDVKFTRGAVVNLASVSSGAVRVNLDG |
| | | | CLSTDSAVNCRGNDSILVYQGKEQSVYEGGLQPFT |
| | | | EYLYRVIASHEGGSVYSDWSRGRTTGAAPQSVPTP |
| | | | SRVRSLNGYSIEVTWDEPVVRGVIEKYILKAYSED |
| | | | STRPPRMPSASAEFVNTSNLTGILTGLLPFKNYAV |
| | | | TLTACTLAGCTESSHALNISTPQEAPQEVOPPVAK |
| | | | SLPSSLLLSWNPPKKANGIITQYCLYMDGRLIYSG |
| | | | SEENYIVTDLAVFTPHQFLLSACTHVGCTNSSWVL |
| | | | LYTAQLPPEHVDSPVLTVLDSRTIHIQWKQPRKIS |
| | | | GILERYVLYMSNHTHDFTIWSVIYNSTELFQDHML |
| | | | QYVLPGNKYLIKLGACTGGGCTVSEASEALTDEDI |
| | | | PEGVPAPKAHSYSPDSFNVSWTEPEYPNGVITSYG |
| | | | LYLDGILIHNSSELSYRAYGFAPWSLHSFRVQACT |
| | | | AKGCALGPLVENRTLEAPPEGTVNVFVKTQGSRKA |
| | | | HVRWEAPFRPNGLLTHSVLFTGIFYVDPVGNNYTL |
| | | | LNVTKVMYSGEETNLWVLIDGLVPFTNYTVQVNIS |
| | | | NSQGSLITDPITIAMPPGAPDGVLPPRLSSATPTS |
| | | | LQVVWSTPARNNAPGSPRYQLQMRSGDSTHGELEL |
| | | | FSNPSASLSYEVSDLQPYTEYMERLVASNGFGSAH |
| | | | SSWIPEMTAEDKPGPVVPPILLDVKSRMMLVTWQH |
| | | | PRKSNGVITHYNIYLHGRLYLRTPGNVTNCTVMHL |
| | | | HPYTAYKFQVEACTSKGCSLSPESQTVWTLPGAPE |
| | | | GIPSPELFSDTPTSVIISWQPPTHPNGLVENETIE |
| | | | RRVKGKEEVTTLVTLPRSHSMRFIDKTSALSPWTK |
| | | | YEYRVLMSTLHGGTNSSAWVEVTTRPSRPAGVQPP |
| | | | VVTVLEPDAVQVTWKPPLIQNGDILSYEIHMPDPH |
| | | | ITLTNVTSAVLSQKVTHLIPFTNYSVTIVACSGGN |
| | | | GYLGGCTESLPTYVTTHPTVPQNVGPLSVIPLSES |
| | | | YVVISWOPPSKPNGPNLRYELLRRKIQQPLASNPP |
| | | | EDLNRWHNIYSGTQWLYEDKGLSRFTTYEYMLFVH |
| | | | NSVGFTPSREVTVTTLAGLPERGANLTASVLNHTA |
| | | | IDVRWAKPTVQDLQGEVEYYTLFWSSATSNDSLKI |
| | | | LPDVNSHVIGHLKPNTEYWIFISVENGVHSINSAG |
| | | | LHATTCDGEPQGMLPPEVVIINSTAVRVIWTSPSN |
| | | | PNGVVTEYSIYVNNKLYKTGMNVPGSFILRDLSPF |
| | | | TIYDIQVEVCTIYACVKSNGTQITTVEDTPSDIPT |
| | | | PTIRGITSRSLQIDWVSPRKPNGIILGYDLLWKTW |
| | | | YPCAKTQKLVQDQSDELCKAVRCQKPESICGHICY |
| | | | SSEAKVCCNGVLYNPKPGHRCCEEKYIPFVLNSTG |
| | | | VCCGGRIQEAQPNHQCCSGYYARILPGEVCCPDEQ |
| | | | HNRVSVGIGDSCCGRMPYSTSGNQICCAGRLHDGH |
| | | | GQKCCGRQIVSNDLECCGGEEGVVYNRLPGMFCCG |
| | | | QDYVNMSDTICCSASSGESKAHIKKNDPVPVKCCE |
| | | | TELIPKSQKCCNGVGYNPLKYVCSDKISTGMMMKE |
| | | | TKECRILCPASMEATEHCGRCDENFTSHICTVIRG |
| | | | SHNSTGKASIEEMCSSAEETIHTGSVNTYSYTDVN |
| | | | LKPYMTYEYRISAWNSYGRGLSKAVRARTKEDVPQ |
| | | | GVSPPTWTKIDNLEDTIVLNWRKPIQSNGPIIYYI |
| | | | LLRNGIERFRGTSLSFSDKEGIQPFQEYSYQLKAC |
| | | | TVAGCATSSKVVAATTQGVPESILPPSITALSAVA |
| | | | LHLSWSVPEKSNGVIKEYQIRQVGKGLIHTDTTDR |
| | | | RQHTVTGLQPYTNYSFTLTACTSAGCTSSEPFLGQ |
| | | | TLQAAPEGVWVTPRHIIINSTTVELYWSLPEKPNG |
| | | | LVSQYQLSRNGNLLFLGGSEEQNFTDKNLEPNSRY |
| | | | TYKLEVKTGGGSSASDDYIVQTPMSTPEEIYPPYN |
| | | | ITVIGPYSIFVAWIPPGILIPEIPVEYNVLLNDGS |
| | | | VTPLAFSVGHHQSTLLENLTPFTQYEIRIQACONG |
| | | | SCGVSSRMFVKTPEAAPMDLNSPVLKALGSACIEI |
| | | | KWMPPEKPNGIIINYFIYRRPAGIEEESVLFVWSE |
| | | | GALEFMDEGDTLRPFTLYEYRVRACNSKGSVESLW |
| | | | SLTQTLEAPPQDEPAPWAQATSAHSVLLNWTKPES |
| | | | PNGIISHYRVVYQERPDDPTFNSPTVHAFTVKGTS |
| | | | HQAHLYGLEPFTTYRIGVVAANHAGEILSPWTLIQ |
| | | | TLESSPSGLRNFIVEQKENGRALLLQWSEPMRING |
| | | | VIKTYNIFSDGFLEYSGLNRQFLFRRLDPFTLYTL |
| | | | TLEACTRAGCAHSAPQPLWTDEAPPDSQLAPTVHS |
| | | | VKSTSVELSWSEPVNPNGKIIRYEVIRRCFEGKAW |
| | | | GNQTIQADEKIVFTEYNTERNTEMYNDTGLQPWTQ |
| | | | CEYKIYTWNSAGHTCSSWNVVRTLQAPPEGLSPPV |
| | | | ISYVSMNPQKLLISWIPPEQSNGIIQSYRLORNEM |
| | | | LYPFSFDPVTFNYTDEELLPFSTYSYALQACTSGG |
| | | | CSTSKPTSITTLEAAPSEVSPPDLWAVSATQMNVC |
| | | | WSPPTVQNGKITKYLVRYDNKESLAGQGLCLLVSH |
| | | | LQPYSQYNFSLVACTNGGCTASVSKSAWTMEALPE |
| | | | NMDSPTLQVTGSESIEITWKPPRNPNGQIRSYELR |
| | | | RDGTIVYTGLETRYRDFTLTPGVEYSYTVTASNSQ |
| | | | GGILSPLVKDRTSPSAPSGMEPPKLQARGPQEILV |
| | | | NWDPPVRTNGDIINYTLFIRELFERETKIIHINTT |
| | | | HNSFGMQSYIVNQLKPFHRYEIRIQACTTLGCASS |
| | | | DWTFIQTPEIAPLMQPPPHLEVQMAPGGFQPTVSL |
| | | | LWTGPLQPNGKVLYYELYRRQIATQPRKSNPVLIY |
| | | | NGSSTSFIDSELLPFTEYEYQVWAVNSAGKAPSSW |
| | | | TWCRTGPAPPEGLRAPTFHVISSTQAVVNISAPGK |
| | | | PNGIVSLYRLESSSAHGAETVLSEGMATQQTLHGL |
| | | | QAFTNYSIGVEACTCFNCCSKGPTAELRTHPAPPS |
| | | | GLSSPQIGTLASRTASFRWSPPMFPNGVIHSYELQ |
| | | | FHVACPPDSALPCTPSQIETKYTGLGQKASLGGLQ |
| | | | PYTTYKLRVVAHNEVGSTASEWISFTTQKELPQYR |
| | | | APFSVDSNLSVVCVNWSDTFLLNGQLKEYVLTDGG |
| | | | RRVYSGLDTTLYIPRTADKTFFFQVICTTDEGSVK |
| | | | TPLIQYDTSTGLGLVLTTPGKKKGSRSKSTEFYSE |
| | | | LWFIVLMAMLGLILLAIFLSLILQRKIHKEPYIRE |
| | | | RPPLVPLQKRMSPLNVYPPGENHMGLADTKIPRSG |
| | | | TPVSIRSNRSACVLRIPSQNQTSLTYSQGSLHRSV |
| | | | SQLMDIQDKKVLMDNSLWEAIMGHNSGLYVDEEDL |
| | | | MNAIKDESSVTKERTTFTDTHL |
| Protocadherin- | PCDH19 | 224 | MESLLLPVLLLLAILWTQAAALINLKYSVEEEQRA |
| 19 | Encephalopathy; | | GTVIANVAKDAREAGFALDPRQASAFRVVSNSAPH |
| (PCDH19) | Early Infantile | | LVDINPSSGLLVTKQKIDRDLLCRQSPKCIISLEV |
| Signal | Epileptic | | MSSSMEICVIKVEIKDLNDNAPSFPAAQIELEISE |
| Sequence | Encephalopathy | | AASPGTRIPLDSAYDPDSGSFGVQTYELTPNELFG |
| Underlined | 9 | | LEIKTRGDGSRFAELVVEKSLDRETQSHYSFRITA |
| | | | LDGGDPPRLGTVGLSIKVTDSNDNNPVFSESTYAV |
| | | | SVPENSPPNTPVIRLNASDPDEGINGQVVYSFYGY |
| | | | VNDRTRELFQIDPHSGLVTVTGALDYEEGHVYELD |
| | | | VQAKDLGPNSIPAHCKVTVSVLDTNDNPPVINLLS |
| | | | VNSELVEVSESAPPGYVIALVRVSDRDSGLNGRVQ |
| | | | CRLLGNVPFRLQEYESFSTILVDGRLDREQHDQYN |
| | | | LTIQARDGGVPMLQSAKSFTVLITDENDNHPHESK |
| | | | PYYQVIVOENNTPGAYLLSVSARDPDLGLNGSVSY |
| | | | QIVPSQVRDMPVFTYVSINPNSGDIYALRSENHEQ |
| | | | TKAFEFKVLAKDGGLPSLQSNATVRVIILDVNDNT |
| | | | PVITAPPLINGTAEVYIPRNSGIGYLVTVVKAEDY |
| | | | DEGENGRVTYDMTEGDRGFFEIDQVNGEVRTTRTF |
| | | | GESSKSSYELIVVAHDHGKTSLSASALVLIYLSPA |
| | | | LDAQESMGSVNLSLIFIIALGSIAGILFVTMIFVA |
| | | | IKCKRDNKEIRTYNCSNCLTITCLLGCFIKGQNSK |
| | | | CLHCISVSPISEEQDKKTEEKVSLRGKRIAEYSYG |
| | | | HQKKSSKKKKISKNDIRLVPRDVEETDKMNVVSCS |
| | | | SLTSSLNYFDYHQQTLPLGCRRSESTELNVENQNT |
| | | | RNTSANHIYHHSFNSQGPQQPDLIINGVPLPETEN |
| | | | YSFDSNYVNSRAHLIKSSSTFKDLEGNSLKDSGHE |
| | | | ESDQTDSEHDVQRSLYCDTAVNDVLNTSVTSMGSQ |
| | | | MPDHDQNEGFHCREECRILGHSDRCWMPRNPMPIR |
| | | | SKSPEHVRNIIALSIEATAADVEAYDDCGPTKRTF |
| | | | ATFGKDVSDHPAEERPTLKGKRTVDVTICSPKVNS |
| | | | VIREAGNGCEAISPVTSPLHLKSSLPTKPSVSYTI |
| | | | ALAPPARDLEQYVNNVNNGPTRPSEAEPRGADSEK |
| | | | VMHEVSPILKEGRNKESPGVKRLKDIVL |
| Tuberin | Tuberous | 225 | MAKPTSKDSGLKEKFKILLGLGTPRPNPRSAEGKQ |
| (TSC2) | sclerosis-2 | | TEFIITAEILRELSMECGLNNRIRMIGQICEVAKT |
| | | | KKFEEHAVEALWKAVADLLQPERPLEARHAVLALL |
| | | | KAIVQGQGERLGVLRALFFKVIKDYPSNEDLHERL |
| | | | EVFKALTDNGRHITYLEEELADFVLQWMDVGLSSE |
| | | | FLLVLVNLVKENSCYLDEYIARMVQMICLLCVRTA |
| | | | SSVDIEVSLQVLDAVVCYNCLPAESLPLFIVTLCR |
| | | | TINVKELCEPCWKLMRNLLGTHLGHSAIYNMCHLM |
| | | | EDRAYMEDAPLLRGAVFFVGMALWGAHRLYSLRNS |
| | | | PTSVLPSFYQAMACPNEVVSYEIVLSITRLIKKYR |
| | | | KELQVVAWDILLNIIERLLQQLQTLDSPELRTIVH |
| | | | DLLTTVEELCDQNEFHGSQERYFELVERCADQRPE |
| | | | SSLLNLISYRAQSIHPAKDGWIQNLQALMERFERS |
| | | | ESRGAVRIKVLDVLSFVLLINRQFYEEELINSVVI |
| | | | SQLSHIPEDKDHQVRKLATQLLVDLAEGCHTHHEN |
| | | | SLLDIIEKVMARSLSPPPELEERDVAAYSASLEDV |
| | | | KTAVLGLLVILQTKLYTLPASHATRVYEMLVSHIQ |
| | | | LHYKHSYTLPIASSIRLQAFDELLLLRADSLHRLG |
| | | | LPNKDGVVRFSPYCVCDYMEPERGSEKKTSGPLSP |
| | | | PTGPPGPAPAGPAVRLGSVPYSLLERVLLQCLKQE |
| | | | SDWKVLKLVLGRLPESLRYKVLIFTSPCSVDQLCS |
| | | | ALCSMLSGPKTLERLRGAPEGESRTDLHLAVVPVL |
| | | | TALISYHNYLDKTKQREMVYCLEQGLIHRCASQCV |
| | | | VALSICSVEMPDIIIKALPVLVVKLTHISATASMA |
| | | | VPLLEFLSTLARLPHLYRNFAAEQYASVFAISLPY |
| | | | TNPSKENQYIVCLAHHVIAMWFIRCRLPERKDEVP |
| | | | FITKGLRSNVLLSFDDTPEKDSFRARSTSLNERPK |
| | | | SLRIARPPKQGLNNSPPVKEFKESSAAEAFRCRSI |
| | | | SVSEHVVRSRIQTSLTSASLGSADENSVAQADDSL |
| | | | KNLHLELTETCLDMMARYVESNFTAVPKRSPVGEF |
| | | | LLAGGRTKTWLVGNKLVTVTTSVGTGTRSLLGLDS |
| | | | GELQSGPESSSSPGVHVRQTKEAPAKLESQAGQQV |
| | | | SRGARDRVRSMSGGHGLRVGALDVPASQFLGSATS |
| | | | PGPRTAPAAKPEKASAGTRVPVQEKTNLAAYVPLL |
| | | | TQGWAEILVRRPTGNTSWLMSLENPLSPFSSDINN |
| | | | MPLQELSNALMAAERFKEHRDTALYKSLSVPAAST |
| | | | AKPPPLPRSNTVASFSSLYQSSCQGQLHRSVSWAD |
| | | | SAVVMEEGSPGEVPVLVEPPGLEDVEAALGMDRRT |
| | | | DAYSRSSSVSSQEEKSLHAEELVGRGIPIERVVSS |
| | | | EGGRPSVDLSFQPSQPLSKSSSSPELQTLQDILGD |
| | | | PGDKADVGRLSPEVKARSQSGTLDGESAAWSASGE |
| | | | DSRGQPEGPLPSSSPRSPSGLRPRGYTISDSAPSR |
| | | | RGKRVERDALKSRATASNAEKVPGINPSFVFLQLY |
| | | | HSPFFGDESNKPILLPNESQSFERSVOLLDQIPSY |
| | | | DTHKIAVLYVGEGQSNSELAILSNEHGSYRYTEFL |
| | | | TGLGRLIELKDCQPDKVYLGGLDVCGEDGQFTYCW |
| | | | HDDIMQAVFHIATLMPTKDVDKHRCDKKRHLGNDE |
| | | | VSIVYNDSGEDFKLGTIKGQFNFVHVIVTPLDYEC |
| | | | NLVSLQCRKDMEGLVDTSVAKIVSDRNLPFVARQM |
| | | | ALHANMASQVHHSRSNPTDIYPSKWIARLRHIKRL |
| | | | RQRICEEAAYSNPSLPLVHPPSHSKAPAQTPAEPT |
| | | | PGYEVGQRKRLISSVEDFTEFV |
| Hamartin | Tuberous | 226 | MAQQANVGELLAMLDSPMLGVRDDVTAVEKENLNS |
| (TSC1) | sclerosis-1 | | DRGPMLVNTLVDYYLETSSQPALHILTTLQEPHDK |
| | | | HLLDRINEYVGKAATRLSILSLLGHVIRLQPSWKH |
| | | | KLSQAPLLPSLLKCLKMDTDVVVLTTGVLVLITML |
| | | | PMIPQSGKQHLLDFFDIFGRLSSWCLKKPGHVAEV |
| | | | YLVHLHASVYALFHRLYGMYPCNFVSFLRSHYSMK |
| | | | ENLETFEEVVKPMMEHVRIHPELVTGSKDHELDPR |
| | | | RWKRLETHDVVIECAKISLDPTEASYEDGYSVSHQ |
| | | | ISARFPHRSADVTTSPYADTQNSYGCATSTPYSTS |
| | | | RLMLLNMPGQLPQTLSSPSTRLITEPPQATLWSPS |
| | | | MVCGMTTPPTSPGNVPPDLSHPYSKVEGTTAGGKG |
| | | | TPLGTPATSPPPAPLCHSDDYVHISLPQATVTPPR |
| | | | KEERMDSARPCLHRQHHLLNDRGSEEPPGSKGSVT |
| | | | LSDLPGELGDLASEEDSIEKDKEEAAISRELSEIT |
| | | | TAEAEPVVPRGGFDSPFYRDSLPGSQRKTHSAASS |
| | | | SQGASVNPEPLHSSLDKLGPDTPKQAFTPIDLPCG |
| | | | SADESPAGDRECQTSLETSIFTPSPCKIPPPTRVG |
| | | | FGSGQPPPYDHLFEVALPKTAHHFVIRKTEELLKK |
| | | | AKGNTEEDGVPSTSPMEVLDRLIQQGADAHSKELN |
| | | | KLPLPSKSVDWTHEGGSPPSDEIRTLRDQLLLLHN |
| | | | QLLYERFKRQQHALRNRRLLRKVIKAAALEEHNAA |
| | | | MKDQLKLQEKDIQMWKVSLQKEQARYNQLQEQRDT |
| | | | MVTKLHSQIRQLQHDREEFYNQSQELQTKLEDCRN |
| | | | MIAELRIELKKANNKVCHTELLLSQVSQKLSNSES |
| | | | VQQQMEFLNROLLVLGEVNELYLEQLQNKHSDTTK |
| | | | EVEMMKAAYRKELEKNRSHVLQQTORLDTSQKRIL |
| | | | ELESHLAKKDHLLLEQKKYLEDVKLQARGQLQAAE |
| | | | SRYEAQKRITQVFELEILDLYGRLEKDGLLKKLEE |
| | | | EKAEAAEAAEERLDCCNDGCSDSMVGHNEEASGHN |
| | | | GETKTPRPSSARGSSGSRGGGGSSSSSSELSTPEK |
| | | | PPHQRAGPFSSRWETTMGEASASIPTTVGSLPSSK |
| | | | SFLGMKARELFRNKSESQCDEDGMTSSLSESLKTE |
| | | | LGKDLGVEAKIPLNLDGPHPSPPTPDSVGQLHIMD |
| | | | YNETHHEHS |
| Dystrophin | Becker | 227 | MLWWEEVEDCYEREDVQKKTFTKWVNAQFSKFGKQ |
| (DMD) | Muscular | | HIENLFSDLQDGRRLLDLLEGLTGQKLPKEKGSTR |
| | Dystrophy | | VHALNNVNKALRVLQNNNVDLVNIGSTDIVDGNHK |
| | | | LTLGLIWNIILHWQVKNVMKNIMAGLQQTNSEKIL |
| | | | LSWVRQSTRNYPQVNVINFTTSWSDGLALNALIHS |
| | | | HRPDLFDWNSVVCQQSATORLEHAFNIARYQLGIE |
| | | | KLLDPEDVDTTYPDKKSILMYITSLFQVLPQQVSI |
| | | | EAIQEVEMLPRPPKVTKEEHFQLHHQMHYSQQITV |
| | | | SLAQGYERTSSPKPRFKSYAYTQAAYVTTSDPTRS |
| | | | PFPSQHLEAPEDKSFGSSLMESEVNLDRYQTALEE |
| | | | VLSWLLSAEDTLQAQGEISNDVEVVKDQFHTHEGY |
| | | | MMDLTAHQGRVGNILQLGSKLIGTGKLSEDEETEV |
| | | | QEQMNLLNSRWECLRVASMEKQSNLHRVLMDLQNQ |
| | | | KLKELNDWLTKTEERTRKMEEEPLGPDLEDLKRQV |
| | | | QQHKVLQEDLEQEQVRVNSLTHMVVVVDESSGDHA |
| | | | TAALEEQLKVLGDRWANICRWTEDRWVLLQDILLK |
| | | | WQRLTEEQCLFSAWLSEKEDAVNKIHTTGFKDQNE |
| | | | MLSSLQKLAVLKADLEKKKQSMGKLYSLKQDLLST |
| | | | LKNKSVTQKTEAWLDNFARCWDNLVQKLEKSTAQI |
| | | | SQAVTTTQPSLTQTTVMETVTTVTTREQILVKHAQ |
| | | | EELPPPPPQKKRQITVDSEIRKRLDVDITELHSWI |
| | | | TRSEAVLQSPEFAIFRKEGNFSDLKEKVNAIEREK |
| | | | AEKFRKLQDASRSAQALVEQMVNEGVNADSIKQAS |
| | | | EQLNSRWIEFCOLLSERLNWLEYQNNIIAFYNQLQ |
| | | | QLEQMTTTAENWLKIQPTTPSEPTAIKSQLKICKD |
| | | | EVNRLSDLQPQIERLKIQSIALKEKGQGPMELDAD |
| | | | FVAFTNHFKQVESDVQAREKELQTIFDTLPPMRYQ |
| | | | ETMSAIRTWVQQSETKLSIPQLSVTDYEIMEQRLG |
| | | | ELQALQSSLQEQQSGLYYLSTTVKEMSKKAPSEIS |
| | | | RKYQSEFEEIEGRWKKLSSQLVEHCQKLEEQMNKL |
| | | | RKIQNHIQTLKKWMAEVDVELKEEWPALGDSEILK |
| | | | KQLKQCRLLVSDIQTIQPSLNSVNEGGQKIKNEAE |
| | | | PEFASRLETELKELNTQWDHMCQQVYARKEALKGG |
| | | | LEKTVSLQKDLSEMHEWMTQAEEEYLERDFEYKTP |
| | | | DELQKAVEEMKRAKEEAQQKEAKVKLLTESVNSVI |
| | | | AQAPPVAQEALKKELETLTTNYQWLCTRLNGKCKT |
| | | | LEEVWACWHELLSYLEKANKWLNEVEFKLKTTENI |
| | | | PGGAEEISEVLDSLENLMRHSEDNPNQIRILAQTL |
| | | | TDGGVMDELINEELETENSRWRELHEEAVRRQKLL |
| | | | EQSIQSAQETEKSLHLIQESLTFIDKQLAAYIADK |
| | | | VDAAQMPQEAQKIQSDLTSHEISLEEMKKHNQGKE |
| | | | AAQRVLSQIDVAQKKLQDVSMKERLFQKPANFEQR |
| | | | LQESKMILDEVKMHLPALETKSVEQEVVQSQLNHC |
| | | | VNLYKSLSEVKSEVEMVIKTGRQIVQKKQTENPKE |
| | | | LDERVTALKLHYNELGAKVTERKQQLEKCLKLSRK |
| | | | MRKEMNVLTEWLAATDMELTKRSAVEGMPSNLDSE |
| | | | VAWGKATQKEIEKQKVHLKSITEVGEALKTVLGKK |
| | | | ETLVEDKLSLLNSNWIAVTSRAEEWLNLLLEYQKH |
| | | | METFDQNVDHITKWIIQADTLLDESEKKKPQQKED |
| | | | VLKRLKAELNDIRPKVDSTRDQAANLMANRGDHCR |
| | | | KLVEPQISELNHRFAAISHRIKTGKASIPLKELEQ |
| | | | FNSDIQKLLEPLEAEIQQGVNLKEEDENKDMNEDN |
| | | | EGTVKELLQRGDNLQQRITDERKREEIKIKQQLLQ |
| | | | TKHNALKDLRSQRRKKALEISHOWYQYKRQADDLL |
| | | | KCLDDIEKKLASLPEPRDERKIKEIDRELQKKKEE |
| | | | LNAVRRQAEGLSEDGAAMAVEPTQIQLSKRWREIE |
| | | | SKFAQFRRLNFAQIHTVREETMMVMTEDMPLEISY |
| | | | VPSTYLTEITHVSQALLEVEQLLNAPDLCAKDFED |
| | | | LFKQEESLKNIKDSLQQSSGRIDIIHSKKTAALQS |
| | | | ATPVERVKLQEALSQLDFQWEKVNKMYKDRQGRED |
| | | | RSVEKWRRFHYDIKIFNQWLTEAEQFLRKTQIPEN |
| | | | WEHAKYKWYLKELQDGIGQRQTVVRTLNATGEEII |
| | | | QQSSKTDASILQEKLGSLNLRWQEVCKQLSDRKKR |
| | | | LEEQKNILSEFQRDLNEFVLWLEEADNIASIPLEP |
| | | | GKEQQLKEKLEQVKLLVEELPLRQGILKQLNETGG |
| | | | PVLVSAPISPEEQDKLENKLKQTNLQWIKVSRALP |
| | | | EKQGEIEAQIKDLGQLEKKLEDLEEQLNHLLLWLS |
| | | | PIRNQLEIYNQPNQEGPEDVKETEIAVQAKQPDVE |
| | | | EILSKGQHLYKEKPATQPVKRKLEDLSSEWKAVNR |
| | | | LLQELRAKQPDLAPGLTTIGASPTQTVTLVTQPVV |
| | | | TKETAISKLEMPSSLMLEVPALADENRAWTELTDW |
| | | | LSLLDQVIKSQRVMVGDLEDINEMIIKQKATMQDL |
| | | | EQRRPQLEELITAAQNLKNKTSNQEARTIITDRIE |
| | | | RIQNQWDEVQEHLQNRRQQLNEMLKDSTQWLEAKE |
| | | | EAEQVLGQARAKLESWKEGPYTVDAIQKKITETKQ |
| | | | LAKDLRQWQTNVDVANDLALKLLRDYSADDTRKVH |
| | | | MITENINASWRSIHKRVSEREAALEETHRLLQQFP |
| | | | LDLEKFLAWLTEAETTANVLQDATRKERLLEDSKG |
| | | | VKELMKQWQDLQGEIEAHTDVYHNLDENSQKILRS |
| | | | LEGSDDAVLLQRRLDNMNFKWSELRKKSLNIRSHL |
| | | | EASSDQWKRLHLSLQELLVWLQLKDDELSRQAPIG |
| | | | GDFPAVQKONDVHRAFKRELKTKEPVIMSTLETVR |
| | | | IFLTEQPLEGLEKLYQEPRELPPEERAQNVTRLLR |
| | | | KQAEEVNTEWEKLNLHSADWORKIDETLERLQELQ |
| | | | EATDELDLKLRQAEVIKGSWQPVGDLLIDSLQDHL |
| | | | EKVKALRGEIAPLKENVSHVNDLARQLTTLGIQLS |
| | | | PYNLSTLEDLNTRWKLLQVAVEDRVRQLHEAHRDE |
| | | | GPASQHELSTSVQGPWERAISPNKVPYYINHETQT |
| | | | TCWDHPKMTELYQSLADLNNVRESAYRTAMKLRRL |
| | | | QKALCLDLLSLSAACDALDQHNLKQNDQPMDILQI |
| | | | INCLTTIYDRLEQEHNNLVNVPLCVDMCLNWLLNV |
| | | | YDTGRTGRIRVLSEKTGIISLCKAHLEDKYRYLFK |
| | | | QVASSTGFCDQRRLGLLLHDSIQIPRQLGEVASFG |
| | | | GSNIEPSVRSCFQFANNKPEIEAALFLDWMRLEPQ |
| | | | SMVWLPVLHRVAAAETAKHQAKCNICKECPIIGER |
| | | | YRSLKHFNYDICQSCFFSGRVAKGHKMHYPMVEYC |
| | | | TPTTSGEDVRDFAKVLKNKERTKRYFAKHPRMGYL |
| | | | PVQTVLEGDNMETPVTLINFWPVDSAPASSPQLSH |
| | | | DDTHSRIEHYASRLAEMENSNGSYLNDSISPNESI |
| | | | DDEHLLIQHYCQSLNQDSPLSQPRSPAQILISLES |
| | | | EERGELERILADLEEENRNLQAEYDRLKQQHEHKG |
| | | | LSPLPSPPEMMPTSPQSPRDAELIAEAKLLRQHKG |
| | | | RLEARMQILEDHNKQLESQLHRLROLLEQPQAEAK |
| | | | VNGTTVSSPSTSLORSDSSQPMLLRVVGSQTSDSM |
| | | | GEEDLLSPPQDTSTGLEEVMEQLNNSFPSSRGRNT |
| | | | PGKPMREDTM |
| Glutamate | GRIN2B- | 228 | MKPRAECCSPKFWLVLAVLAVSGSRARSQKSPPSI |
| receptor | Related | | GIAVILVGTSDEVAIKDAHEKDDFHHLSVVPRVEL |
| ionotropic, | Disorder; | | VAMNETDPKSIITRICDLMSDRKIQGVVFADDTDQ |
| NMDA 2B | Epileptic | | EAIAQILDFISAQTLTPILGIHGGSSMIMADKDES |
| (GRIN2B) | encephalopathy, | | SMFFQFGPSIEQQASVMLNIMEEYDWYIFSIVTTY |
| Signal | early infantile, | | FPGYQDFVNKIRSTIENSFVGWELEEVLLLDMSLD |
| Sequence | 27 | | DGDSKIQNQLKKLQSPIILLYCTKEEATYIFEVAN |
| Underlined | | | SVGLTGYGYTWIVPSLVAGDTDTVPAEFPTGLISV |
| | | | SYDEWDYGLPARVRDGIAIITTAASDMLSEHSFIP |
| | | | EPKSSCYNTHEKRIYQSNMLNRYLINVTFEGRNLS |
| | | | FSEDGYQMHPKLVIILLNKERKWERVGKWKDKSLQ |
| | | | MKYYVWPRMCPETEEQEDDHLSIVTLEEAPFVIVE |
| | | | SVDPLSGTCMRNTVPCQKRIVTENKTDEEPGYIKK |
| | | | CCKGFCIDILKKISKSVKFTYDLYLVTNGKHGKKI |
| | | | NGTWNGMIGEVVMKRAYMAVGSLTINEERSEVVDE |
| | | | SVPFIETGISVMVSRSNGTVSPSAFLEPFSADVWV |
| | | | MMFVMLLIVSAVAVFVFEYFSPVGYNRCLADGREP |
| | | | GGPSFTIGKAIWLLWGLVENNSVPVQNPKGTTSKI |
| | | | MVSVWAFFAVIFLASYTANLAAFMIQEEYVDQVSG |
| | | | LSDKKFQRPNDESPPFRFGTVPNGSTERNIRNNYA |
| | | | EMHAYMGKFNQRGVDDALLSLKTGKLDAFIYDAAV |
| | | | LNYMAGRDEGCKLVTIGSGKVFASTGYGIAIQKDS |
| | | | GWKRQVDLAILQLEGDGEMEELEALWLTGICHNEK |
| | | | NEVMSSQLDIDNMAGVFYMLGAAMALSLITFICEH |
| | | | LFYWQFRHCFMGVCSGKPGMVFSISRGIYSCIHGV |
| | | | AIEERQSVMNSPTATMNNTHSNILRLLRTAKNMAN |
| | | | LSGVNGSPQSALDFIRRESSVYDISEHRRSFTHSD |
| | | | CKSYNNPPCEENLESDYISEVERTFGNLQLKDSNV |
| | | | YQDHYHHHHRPHSIGSASSIDGLYDCDNPPETTOS |
| | | | RSISKKPLDIGLPSSKHSQLSDLYGKESFKSDRYS |
| | | | GHDDLIRSDVSDISTHTVTYGNIEGNAAKRRKQQY |
| | | | KDSLKKRPASAKSRREFDEIELAYRRRPPRSPDHK |
| | | | RYFRDKEGLRDFYLDQFRTKENSPHWEHVDLTDIY |
| | | | KERSDDFKRDSVSGGGPCTNRSHIKHGTGDKHGVV |
| | | | SGVPAPWEKNLTNVEWEDRSGGNFCRSCPSKLHNY |
| | | | STTVTGQNSGRQACIRCEACKKAGNLYDISEDNSL |
| | | | QELDQPAAPVAVTSNASTTKYPQSPTNSKAQKKNR |
| | | | NKLRRQHSYDTFVDLQKEEAALAPRSVSLKDKGRF |
| | | | MDGSPYAHMFEMSAGESTFANNKSSVPTAGHHHHN |
| | | | NPGGGYMLSKSLYPDRVTQNPFIPTFGDDQCLLHG |
| | | | SKSYFFRQPTVAGASKARPDFRALVTNKPVVSALH |
| | | | GAVPARFQKDICIGNQSNPCVPNNKNPRAFNGSSN |
| | | | GHVYEKLSSIESDV |
| Cystic fibrosis | Cystic fibrosis | 229 | MQRSPLEKASVVSKLFFSWTRPILRKGYRORLELS |
| transmembrane | | | DIYQIPSVDSADNLSEKLEREWDRELASKKNPKLI |
| conductance | | | NALRRCFFWREMFYGIFLYLGEVTKAVQPLLLGRI |
| regulator | | | IASYDPDNKEERSIAIYLGIGLCLLFIVRTLLLHP |
| (CFTR) | | | AIFGLHHIGMQMRIAMESLIYKKTLKLSSRVLDKI |
| | | | SIGQLVSLLSNNLNKFDEGLALAHEVWIAPLQVAL |
| | | | LMGLIWELLQASAFCGLGFLIVLALFQAGLGRMMM |
| | | | KYRDQRAGKISERLVITSEMIENIQSVKAYCWEEA |
| | | | MEKMIENLRQTELKLTRKAAYVRYFNSSAFFFSGE |
| | | | FVVELSVLPYALIKGIILRKIFTTISFCIVLRMAV |
| | | | TRQFPWAVQTWYDSLGAINKIQDFLOKQEYKTLEY |
| | | | NLTTTEVVMENVTAFWEEGFGELFEKAKQNNNNRK |
| | | | TSNGDDSLFFSNFSLLGTPVLKDINFKIERGQLLA |
| | | | VAGSTGAGKTSLLMVIMGELEPSEGKIKHSGRISF |
| | | | CSQFSWIMPGTIKENIIFGVSYDEYRYRSVIKACQ |
| | | | LEEDISKFAEKDNIVLGEGGITLSGGQRARISLAR |
| | | | AVYKDADLYLLDSPFGYLDVLTEKEIFESCVCKLM |
| | | | ANKTRILVTSKMEHLKKADKILILHEGSSYFYGTE |
| | | | SELONLQPDFSSKLMGCDSFDQESAERRNSILTET |
| | | | LHRFSLEGDAPVSWTETKKQSFKQTGEFGEKRKNS |
| | | | ILNPINSIRKESIVQKTPLQMNGIEEDSDEPLERR |
| | | | LSLVPDSEQGEAILPRISVISTGPTLQARRRQSVL |
| | | | NLMTHSVNQGQNIHRKTTASTRKVSLAPQANLTEL |
| | | | DIYSRRLSQETGLEISEEINEEDLKECFEDDMESI |
| | | | PAVTTWNTYLRYITVHKSLIFVLIWCLVIFLAEVA |
| | | | ASLVVLWLLGNTPLQDKGNSTHSRNNSYAVIITST |
| | | | SSYYVFYIYVGVADTLLAMGFFRGLPLVHTLITVS |
| | | | KILHHKMLHSVLQAPMSTLNTLKAGGILNRFSKDI |
| | | | AILDDLLPLTIEDFIQLLLIVIGAIAVVAVLQPYI |
| | | | FVATVPVIVAFIMLRAYFLOTSQQLKOLESEGRSP |
| | | | IFTHLVTSLKGLWTLRAFGRQPYFETLFHKALNLH |
| | | | TANWFLYLSTLRWFQMRIEMIFVIFFIAVTFISIL |
| | | | TTGEGEGRVGIILTLAMNIMSTLQWAVNSSIDVDS |
| | | | LMRSVSRVFKFIDMPTEGKPTKSTKPYKNGQLSKV |
| | | | MIIENSHVKKDDIWPSGGQMTVKDLTAKYTEGGNA |
| | | | ILENISFSISPGQRVGLLGRTGSGKSTLLSAFLRL |
| | | | LNTEGEIQIDGVSWDSITLQQWRKAFGVIPQKVFI |
| | | | FSGTFRKNLDPYEQWSDQEIWKVADEVGLRSVIEQ |
| | | | FPGKLDFVLVDGGCVLSHGHKQLMCLARSVLSKAK |
| | | | ILLLDEPSAHLDPVTYQIIRRTLKQAFADCTVILC |
| | | | EHRIEAMLECQQFLVIEENKVRQYDSIQKLLNERS |
| | | | LFRQAISPSDRVKLEPHRNSSKCKSKPQIAALKEE |
| | | | TEEEVQDTRL |
| Sodium | Dravet | 230 | MEQTVLVPPGPDSENFFTRESLAAIERRIAEEKAK |
| channel protein | syndrome | | NPKPDKKDDDENGPKPNSDLEAGKNLPFIYGDIPP |
| type 1 subunit | | | EMVSEPLEDLDPYYINKKTFIVLNKGKAIFRESAT |
| alpha (SCN1A) | | | SALYILTPENPLRKIAIKILVHSLESMLIMCTILT |
| | | | NCVFMTMSNPPDWTKNVEYTFTGIYTFESLIKIIA |
| | | | RGFCLEDFTFLRDPWNWLDFTVITFAYVTEFVDLG |
| | | | NVSALRTFRVLRALKTISVIPGLKTIVGALIQSVK |
| | | | KLSDVMILTVFCLSVFALIGLQLEMGNLRNKCIQW |
| | | | PPTNASLEEHSIEKNITVNYNGTLINETVFEEDWK |
| | | | SYIQDSRYHYFLEGELDALLCGNSSDAGQCPEGYM |
| | | | CVKAGRNPNYGYTSFDTFSWAFLSLERLMTQDEWE |
| | | | NLYQLTLRAAGKTYMIFFVLVIFLGSFYLINLILA |
| | | | VVAMAYEEQNQATLEEAEQKEAEFQQMIEQLKKQQ |
| | | | EAAQQAATATASEHSREPSAAGRLSDSSSEASKLS |
| | | | SKSAKERRNRRKKRKQKEQSGGEEKDEDEFQKSES |
| | | | EDSIRRKGFRESIEGNRLTYEKRYSSPHQSLLSIR |
| | | | GSLFSPRRNSRTSLFSFRGRAKDVGSENDFADDEH |
| | | | STFEDNESRRDSLFVPRRHGERRNSNLSQTSRSSR |
| | | | MLAVFPANGKMHSTVDCNGVVSLVGGPSVPTSPVG |
| | | | QLLPEVIIDKPATDDNGTTTETEMRKRRSSSFHVS |
| | | | MDFLEDPSQRQRAMSIASILTNTVEELEESROKCP |
| | | | PCWYKFSNIFLIWDCSPYWLKVKHVVNLVVMDPFV |
| | | | DLAITICIVLNTLFMAMEHYPMTDHENNVLTVGNL |
| | | | VFTGIFTAEMELKIIAMDPYYYFQEGWNIEDGFIV |
| | | | TLSLVELGLANVEGLSVLRSERLLRVEKLAKSWPT |
| | | | LNMLIKIIGNSVGALGNLTLVLAIIVFIFAVVGMQ |
| | | | LFGKSYKDCVCKIASDCQLPRWHMNDFFHSFLIVE |
| | | | RVLCGEWIETMWDCMEVAGQAMCLTVFMMVMVIGN |
| | | | LVVLNLFLALLLSSFSADNLAATDDDNEMNNLQIA |
| | | | VDRMHKGVAYVKRKIYEFIQQSFIRKQKILDEIKP |
| | | | LDDLNNKKDSCMSNHTAEIGKDLDYLKDVNGTTSG |
| | | | IGTGSSVEKYIIDESDYMSFINNPSLTVTVPIAVG |
| | | | ESDFENLNTEDESSESDLEESKEKLNESSSSSEGS |
| | | | TVDIGAPVEEQPVVEPEETLEPEACFTEGCVQRFK |
| | | | CCQINVEEGRGKQWWNLRRTCFRIVEHNWFETFIV |
| | | | FMILLSSGALAFEDIYIDQRKTIKTMLEYADKVET |
| | | | YIFILEMLLKWVAYGYQTYFTNAWCWLDFLIVDVS |
| | | | LVSLTANALGYSELGAIKSLRTLRALRPLRALSRE |
| | | | EGMRVVVNALLGAIPSIMNVLLVCLIFWLIFSIMG |
| | | | VNLFAGKFYHCINTTTGDREDIEDVNNHTDCLKLI |
| | | | ERNETARWKNVKVNFDNVGFGYLSLLQVATFKGWM |
| | | | DIMYAAVDSRNVELQPKYEESLYMYLYFVIFIIFG |
| | | | SFFTLNLFIGVIIDNFNQQKKKFGGQDIEMTEEQK |
| | | | KYYNAMKKLGSKKPQKPIPRPGNKFQGMVFDFVTR |
| | | | QVFDISIMILICLNMVTMMVETDDQSEYVTTILSR |
| | | | INLVFIVLFTGECVLKLISLRHYYFTIGWNIFDFV |
| | | | VVILSIVGMFLAELIEKYFVSPTLERVIRLARIGR |
| | | | ILRLIKGAKGIRTLLFALMMSLPALENIGLLLFLV |
| | | | MFIYAIFGMSNFAYVKREVGIDDMENFETEGNSMI |
| | | | CLFQITTSAGWDGLLAPILNSKPPDCDPNKVNPGS |
| | | | SVKGDCGNPSVGIFFFVSYIIISELVVVNMYIAVI |
| | | | LENESVATEESAEPLSEDDFEMFYEVWEKEDPDAT |
| | | | QFMEFEKLSQFAAALEPPLNLPQPNKLQLIAMDLP |
| | | | MVSGDRIHCLDILFAFTKRVLGESGEMDALRIQME |
| | | | ERFMASNPSKVSYQPITTTLKRKQEEVSAVIIQRA |
| | | | YRRHLLKRTVKQASFTYNKNKIKGGANLLIKEDMI |
| | | | IDRINENSITEKTDLTMSTAACPPSYDRVTKPIVE |
| | | | KHEQEGKDEKAKGK |
| Copper- | Wilson disease | 231 | MPEQERQITAREGASRKILSKLSLPTRAWEPAMKK |
| transporting | | | SFAFDNVGYEGGLDGLGPSSQVATSTVRILGMTCQ |
| ATPase 2 | | | SCVKSIEDRISNLKGIISMKVSLEQGSATVKYVPS |
| (ATP7B) | | | VVCLQQVCHQIGDMGFEASIAEGKAASWPSRSLPA |
| | | | QEAVVKLRVEGMTCQSCVSSIEGKVRKLQGVVRVK |
| | | | VSLSNQEAVITYQPYLIQPEDLRDHVNDMGFEAAI |
| | | | KSKVAPLSLGPIDIERLQSTNPKRPLSSANQNENN |
| | | | SETLGHQGSHVVTLQLRIDGMHCKSCVLNIEENIG |
| | | | QLLGVQSIQVSLENKTAQVKYDPSCTSPVALQRAI |
| | | | EALPPGNFKVSLPDGAEGSGTDHRSSSSHSPGSPP |
| | | | RNQVQGTCSTTLIAIAGMTCASCVHSIEGMISQLE |
| | | | GVQQISVSLAEGTATVLYNPSVISPEELRAAIEDM |
| | | | GFEASVVSESCSTNPLGNHSAGNSMVQTTDGTPTS |
| | | | VQEVAPHTGRLPANHAPDILAKSPQSTRAVAPQKC |
| | | | FLQIKGMTCASCVSNIERNLQKEAGVLSVLVALMA |
| | | | GKAEIKYDPEVIQPLEIAQFIQDLGFEAAVMEDYA |
| | | | GSDGNIELTITGMTCASCVHNIESKLTRINGITYA |
| | | | SVALATSKALVKEDPEIIGPRDIIKIIEEIGFHAS |
| | | | LAQRNPNAHHLDHKMEIKQWKKSFLCSLVFGIPVM |
| | | | ALMIYMLIPSNEPHQSMVLDHNIIPGLSILNLIFF |
| | | | ILCTFVQLLGGWYFYVQAYKSLRHRSANMDVLIVL |
| | | | ATSIAYVYSLVILVVAVAEKAERSPVTFEDTPPML |
| | | | FVFIALGRWLEHLAKSKTSEALAKLMSLQATEATV |
| | | | VTLGEDNLIIREEQVPMELVQRGDIVKVVPGGKEP |
| | | | VDGKVLEGNTMADESLITGEAMPVTKKPGSTVIAG |
| | | | SINAHGSVLIKATHVGNDTTLAQIVKLVEEAQMSK |
| | | | APIQQLADRESGYFVPFIIIMSTLTLVVWIVIGFI |
| | | | DFGVVQRYFPNPNKHISQTEVIIRFAFQTSITVLC |
| | | | IACPCSLGLATPTAVMVGTGVAAQNGILIKGGKPL |
| | | | EMAHKIKTVMFDKTGTITHGVPRVMRVLLLGDVAT |
| | | | LPLRKVLAVVGTAEASSEHPLGVAVTKYCKEELGT |
| | | | ETLGYCTDFQAVPGCGIGCKVSNVEGILAHSERPL |
| | | | SAPASHLNEAGSLPAEKDAVPQTFSVLIGNREWLR |
| | | | RNGLTISSDVSDAMTDHEMKGQTAILVAIDGVLCG |
| | | | MIAIADAVKQEAALAVHTLQSMGVDVVLITGDNRK |
| | | | TARAIATQVGINKVFAEVLPSHKVAKVQELONKGK |
| | | | KVAMVGDGVNDSPALAQADMGVAIGTGTDVAIEAA |
| | | | DVVLIRNDLLDVVASIHLSKRTVRRIRINLVLALI |
| | | | YNLVGIPIAAGVEMPIGIVLQPWMGSAAMAASSVS |
| | | | VVLSSLQLKCYKKPDLERYEAQAHGHMKPLTASQV |
| | | | SVHIGMDDRWRDSPRATPWDQVSYVSQVSLSSLTS |
| | | | DKPSRHSAAADDDGDKWSLLLNGRDEEQYI |
| Potassium | KCNQ2-Related | 232 | MVQKSRNGGVYPGPSGEKKLKVGFVGLDPGAPDST |
| voltage-gated | Disorders | | RDGALLIAGSEAPKRGSILSKPRAGGAGAGKPPKR |
| channel | (Epileptic | | NAFYRKLONFLYNVLERPRGWAFIYHAYVELLVES |
| subfamily KQT | Encephalopathy) | | CLVLSVESTIKEYEKSSEGALYILEIVTIVVEGVE |
| member 2 | | | YFVRIWAAGCCCRYRGWRGRLKFARKPFCVIDIMV |
| (KCNQ2) | | | LIASIAVLAAGSQGNVFATSALRSLRELQILRMIR |
| | | | MDRRGGTWKLLGSVVYAHSKELVTAWYIGELCLIL |
| | | | ASFLVYLAEKGENDHEDTYADALWWGLITLTTIGY |
| | | | GDKYPQTWNGRLLAATFTLIGVSFFALPAGILGSG |
| | | | FALKVQEQHRQKHFEKRRNPAAGLIQSAWRFYATN |
| | | | LSRTDLHSTWQYYERTVTVPMYSSQTQTYGASRLI |
| | | | PPLNQLELLRNLKSKSGLAFRKDPPPEPSPSKGSP |
| | | | CRGPLCGCCPGRSSQKVSLKDRVESSPRGVAAKGK |
| | | | GSPQAQTVRRSPSADQSLEDSPSKVPKSWSFGDRS |
| | | | RARQAFRIKGAASRONSEEASLPGEDIVDDKSCPC |
| | | | EFVTEDLTPGLKVSIRAVCVMRFLVSKRKFKESLR |
| | | | PYDVMDVIEQYSAGHLDMLSRIKSLQSRVDQIVGR |
| | | | GPAITDKDRTKGPAEAELPEDPSMMGRLGKVEKQV |
| | | | LSMEKKLDELVNIYMQRMGIPPTETEAYFGAKEPE |
| | | | PAPPYHSPEDSREHVDRHGCIVKIVRSSSSTGQKN |
| | | | FSAPPAAPPVQCPPSTSWQPQSHPRQGHGTSPVGD |
| | | | HGSLVRIPPPPAHERSLSAYGGGNRASMEFLRQED |
| | | | TPGCRPPEGNLRDSDTSISIPSVDHEELERSFSGF |
| | | | SISQSKENLDALNSCYAAVAPCAKVRPYIAEGESD |
| | | | TDSDLCTPCGPPPRSATGEGPFGDVGWAGPRK |
| Sodium | SCN2A-Related | 233 | MAQSVLVPPGPDSFRFFTRESLAAIEQRIAEEKAK |
| channel protein | Disorders; | | RPKQERKDEDDENGPKPNSDLEAGKSLPFIYGDIP |
| type 2 subunit | Epileptic | | PEMVSVPLEDLDPYYINKKTFIVLNKGKAISRESA |
| alpha (SCN2A) | encephalopathy, | | TPALYILTPENPIRKLAIKILVHSLENMLIMCTIL |
| | early infantile, | | TNCVFMTMSNPPDWTKNVEYTFTGIYTFESLIKIL |
| | 11 | | ARGFCLEDETFLRDPWNWLDFTVITFAYVTEFVDL |
| | | | GNVSALRTERVLRALKTISVIPGLKTIVGALIQSV |
| | | | KKLSDVMILTVFCLSVFALIGLQLEMGNLRNKCLQ |
| | | | WPPDNSSFEINITSFENNSLDGNGTTENRTVSIEN |
| | | | WDEYIEDKSHFYFLEGQNDALLCGNSSDAGQCPEG |
| | | | YICVKAGRNPNYGYTSFDTESWAFLSLERLMTQDE |
| | | | WENLYQLTLRAAGKTYMIFFVLVIFLGSFYLINLI |
| | | | LAVVAMAYEEQNQATLEEAEQKEAEFQQMLEQLKK |
| | | | QQEEAQAAAAAASAESRDFSGAGGIGVFSESSSVA |
| | | | SKLSSKSEKELKNRRKKKKQKEQSGEEEKNDRVRK |
| | | | SESEDSIRRKGFRESLEGSRLTYEKRFSSPHQSLL |
| | | | SIRGSLFSPRRNSRASLESFRGRAKDIGSENDFAD |
| | | | DEHSTFEDNDSRRDSLFVPHRHGERRHSNVSQASR |
| | | | ASRVLPILPMNGKMHSAVDCNGVVSLVGGPSTLTS |
| | | | AGQLLPEGTTTETEIRKRRSSSYHVSMDLLEDPTS |
| | | | RQRAMSIASILTNTMEELEESRQKCPPCWYKFANM |
| | | | CLIWDCCKPWLKVKHLVNLVVMDPFVDLAITICIV |
| | | | LNTLFMAMEHYPMTEQFSSVLSVGNLVETGIFTAE |
| | | | MFLKIIAMDPYYYFQEGWNIFDGFIVSLSLMELGL |
| | | | ANVEGLSVLRSFRLLRVFKLAKSWPTLNMLIKIIG |
| | | | NSVGALGNLTLVLAIIVFIFAVVGMQLFGKSYKEC |
| | | | VCKISNDCELPRWHMHDFFHSFLIVERVLCGEWIE |
| | | | TMWDCMEVAGQTMCLTVFMMVMVIGNLVVLNLFLA |
| | | | LLLSSESSDNLAATDDDNEMNNLQIAVGRMQKGID |
| | | | FVKRKIREFIQKAFVRKQKALDEIKPLEDLNNKKD |
| | | | SCISNHTTIEIGKDLNYLKDGNGTTSGIGSSVEKY |
| | | | VVDESDYMSFINNPSLTVTVPIAVGESDFENLNTE |
| | | | EFSSESDMEESKEKLNATSSSEGSTVDIGAPAEGE |
| | | | QPEVEPEESLEPEACFTEDCVRKFKCCQISIEEGK |
| | | | GKLWWNLRKTCYKIVEHNWFETFIVEMILLSSGAL |
| | | | AFEDIYIEQRKTIKTMLEYADKVFTYIFILEMLLK |
| | | | WVAYGFQVYFTNAWCWLDFLIVDVSLVSLTANALG |
| | | | YSELGAIKSLRTLRALRPLRALSRFEGMRVVVNAL |
| | | | LGAIPSIMNVLLVCLIFWLIFSIMGVNLFAGKFYH |
| | | | CINYTTGEMFDVSVVNNYSECKALIESNQTARWKN |
| | | | VKVNFDNVGLGYLSLLQVATFKGWMDIMYAAVDSR |
| | | | NVELQPKYEDNLYMYLYFVIFIIFGSFFTLNLFIG |
| | | | VIIDNENQQKKKFGGQDIEMTEEQKKYYNAMKKLG |
| | | | SKKPQKPIPRPANKFQGMVFDFVTKQVEDISIMIL |
| | | | ICLNMVTMMVETDDQSQEMTNILYWINLVFIVLET |
| | | | GECVLKLISLRYYYFTIGWNIFDFVVVILSIVGME |
| | | | LAELIEKYFVSPTLERVIRLARIGRILRLIKGAKG |
| | | | IRTLLFALMMSLPALFNIGLLLFLVMFIYAIFGMS |
| | | | NFAYVKREVGIDDMENFETEGNSMICLFQITTSAG |
| | | | WDGLLAPILNSGPPDCDPDKDHPGSSVKGDCGNPS |
| | | | VGIFFFVSYIIISELVVVNMYIAVILENFSVATEE |
| | | | SAEPLSEDDFEMFYEVWEKFDPDATQFIEFAKLSD |
| | | | FADALDPPLLIAKPNKVQLIAMDLPMVSGDRIHCL |
| | | | DILFAFTKRVLGESGEMDALRIQMEERFMASNPSK |
| | | | VSYEPITTTLKRKQEEVSAIIIQRAYRRYLLKQKV |
| | | | KKVSSIYKKDKGKECDGTPIKEDTLIDKLNENSTP |
| | | | EKTDMTPSTTSPPSYDSVTKPEKEKFEKDKSEKED |
| | | | KGKDIRESKK |
| Voltage- | CACNA1A- | 234 | MARFGDEMPARYGGGGSGAAAGVVVGSGGGRGAGG |
| dependent P/Q- | Related | | SRQGGQPGAQRMYKQSMAQRARTMALYNPIPVRON |
| type calcium | Disorders; | | CLTVNRSLFLESEDNVVRKYAKKITEWPPFEYMIL |
| channel subunit | Episodic ataxia, | | ATIIANCIVLALEQHLPDDDKTPMSERLDDTEPYF |
| alpha-1A | type 2 | | IGIFCFEAGIKIIALGFAFHKGSYLRNGWNVMDEV |
| (CACNA1A) | | | VVLTGILATVGTEFDLRTLRAVRVLRPLKLVSGIP |
| | | | SLQVVLKSIMKAMIPLLQIGLLLFFAILIFAIIGL |
| | | | EFYMGKFHTTCFEEGTDDIQGESPAPCGTEEPART |
| | | | CPNGTKCOPYWEGPNNGITQFDNILFAVLTVFQCI |
| | | | TMEGWTDLLYNSNDASGNTWNWLYFIPLIIIGSFF |
| | | | MLNLVLGVLSGEFAKERERVENRRAFLKLRRQQQI |
| | | | ERELNGYMEWISKAEEVILAEDETDGEQRHPEDAL |
| | | | RRTTIKKSKTDLLNPEEAEDQLADIASVGSPFARA |
| | | | SIKSAKLENSTFFHKKERRMRFYIRRMVKTQAFYW |
| | | | TVLSLVALNTLCVAIVHYNQPEWLSDFLYYAEFIF |
| | | | LGLFMSEMFIKMYGLGTRPYFHSSENCEDCGVIIG |
| | | | SIFEVIWAVIKPGTSFGISVLRALRLLRIFKVTKY |
| | | | WASLRNLVVSLLNSMKSIISLLELLFLFIVVFALL |
| | | | GMQLFGGQFNFDEGTPPTNEDTFPAAIMTVFQILT |
| | | | GEDWNEVMYDGIKSQGGVQGGMVESIYFIVLTLFG |
| | | | NYTLLNVELAIAVDNLANAQELTKDEQEEEEAANQ |
| | | | KLALQKAKEVAEVSPLSAANMSIAVKEQQKNOKPA |
| | | | KSVWEQRTSEMRKONLLASREALYNEMDPDERWKA |
| | | | AYTRHLRPDMKTHLDRPLVVDPQENRNNNTNKSRA |
| | | | AEPTVDORLGQQRAEDELRKQARYHDRARDPSGSA |
| | | | GLDARRPWAGSQEAELSREGPYGRESDHHAREGSL |
| | | | EQPGFWEGEAERGKAGDPHRRHVHRQGGSRESRSG |
| | | | SPRTGADGEHRRHRAHRRPGEEGPEDKAERRARHR |
| | | | EGSRPARGGEGEGEGPDGGERRRRHRHGAPATYEG |
| | | | DARREDKERRHRRRKENQGSGVPVSGPNLSTTRPI |
| | | | QQDLGRQDPPLAEDIDNMKNNKLATAESAAPHGSL |
| | | | GHAGLPQSPAKMGNSTDPGPMLAIPAMATNPQNAA |
| | | | SRRTPNNPGNPSNPGPPKTPENSLIVINPSGTQTN |
| | | | SAKTARKPDHTTVDIPPACPPPLNHTVVQVNKNAN |
| | | | PDPLPKKEEEKKEEEEDDRGEDGPKPMPPYSSMFI |
| | | | LSTTNPLRRLCHYILNLRYFEMCILMVIAMSSIAL |
| | | | AAEDPVQPNAPRNNVLRYFDYVFTGVFTFEMVIKM |
| | | | IDLGLVLHQGAYFRDLWNILDFIVVSGALVAFAFT |
| | | | GNSKGKDINTIKSLRVLRVLRPLKTIKRLPKLKAV |
| | | | FDCVVNSLKNVFNILIVYMLFMFIFAVVAVQLEKG |
| | | | KFFHCTDESKEFEKDCRGKYLLYEKNEVKARDREW |
| | | | KKYEFHYDNVLWALLTLFTVSTGEGWPQVLKHSVD |
| | | | ATFENQGPSPGYRMEMSIFYVVYFVVFPFFFVNIF |
| | | | VALIIITFQEQGDKMMEEYSLEKNERACIDFAISA |
| | | | KPLTRHMPQNKQSFQYRMWQFVVSPPFEYTIMAMI |
| | | | ALNTIVLMMKFYGASVAYENALRVENIVETSLESL |
| | | | ECVLKVMAFGILNYFRDAWNIFDFVTVLGSITDIL |
| | | | VTEFGNNFINLSFLRLFRAARLIKLLRQGYTIRIL |
| | | | LWTFVQSFKALPYVCLLIAMLFFIYAIIGMQVEGN |
| | | | IGIDVEDEDSDEDEFQITEHNNERTFFQALMLLER |
| | | | SATGEAWHNIMLSCLSGKPCDKNSGILTRECGNEF |
| | | | AYFYFVSFIFLCSFLMLNLFVAVIMDNFEYLTRDS |
| | | | SILGPHHLDEYVRVWAEYDPAAWGRMPYLDMYQML |
| | | | RHMSPPLGLGKKCPARVAYKRLLRMDLPVADDNTV |
| | | | HFNSTLMALIRTALDIKIAKGGADKQQMDAELRKE |
| | | | MMAIWPNLSQKTLDLLVTPHKSTDLTVGKIYAAMM |
| | | | IMEYYRQSKAKKLQAMREEQDRTPLMFQRMEPPSP |
| | | | TQEGGPGQNALPSTQLDPGGALMAHESGLKESPSW |
| | | | VTQRAQEMFQKTGTWSPEQGPPTDMPNSQPNSQSV |
| | | | EMREMGRDGYSDSEHYLPMEGQGRAASMPRLPAEN |
| | | | QRRRGRPRGNNLSTISDTSPMKRSASVLGPKARRL |
| | | | DDYSLERVPPEENQRHHQRRRDRSHRASERSLGRY |
| | | | TDVDTGLGTDLSMTTQSGDLPSKERDQERGRPKDR |
| | | | KHRQHHHHHHHHHHPPPPDKDRYAQERPDHGRARA |
| | | | RDQRWSRSPSEGREHMAHRQGSSSVSGSPAPSTSG |
| | | | TSTPRRGRRQLPQTPSTPRPHVSYSPVIRKAGGSG |
| | | | PPQQQQQQQQQQQQQAVARPGRAATSGPRRYPGPT |
| | | | AEPLAGDRPPTGGHSSGRSPRMERRVPGPARSESP |
| | | | RACRHGGARWPASGPHVSEGPPGPRHHGYYRGSDY |
| | | | DEADGPGSGGGEEAMAGAYDAPPPVRHASSGATGR |
| | | | SPRTPRASGPACASPSRHGRRLPNGYYPAHGLARP |
| | | | RGPGSRKGLHEPYSESDDDWC |
| Sodium | SCN8A-Related | 235 | MAARLLAPPGPDSFKPFTPESLANIERRIAESKLK |
| channel protein | Disorders; | | KPPKADGSHREDDEDSKPKPNSDLEAGKSLPFIYG |
| type 8 subunit | Epileptic | | DIPQGLVAVPLEDFDPYYLTQKTFVVLNRGKTLER |
| alpha (SCN8A) | encephalopathy, | | FSATPALYILSPENLIRRIAIKILIHSVFSMIIMC |
| | early infantile, | | TILTNCVEMTFSNPPDWSKNVEYTFTGIYTFESLV |
| | 13 | | KIIARGFCIDGFTFLRDPWNWLDESVIMMAYITEF |
| | | | VNLGNVSALRTFRVLRALKTISVIPGLKTIVGALI |
| | | | QSVKKLSDVMILTVFCLSVFALIGLQLEMGNLRNK |
| | | | CVVWPINFNESYLENGTKGEDWEEYINNKTNFYTV |
| | | | PGMLEPLLCGNSSDAGQCPEGYQCMKAGRNPNYGY |
| | | | TSFDTESWAFLALFRLMTQDYWENLYQLTLRAAGK |
| | | | TYMIFFVLVIFVGSFYLVNLILAVVAMAYEEQNQA |
| | | | TLEEAEQKEAEFKAMLEQLKKQQEEAQAAAMATSA |
| | | | GTVSEDAIEEEGEEGGGSPRSSSEISKLSSKSAKE |
| | | | RRNRRKKRKQKELSEGEEKGDPEKVEKSESEDGMR |
| | | | RKAFRLPDNRIGRKESIMNQSLLSIPGSPELSRHN |
| | | | SKSSIFSFRGPGRERDPGSENEFADDEHSTVEESE |
| | | | GRRDSLFIPIRARERRSSYSGYSGYSQGSRSSRIF |
| | | | PSLRRSVKRNSTVDCNGVVSLIGGPGSHIGGRLLP |
| | | | EATTEVEIKKKGPGSLLVSMDQLASYGRKDRINSI |
| | | | MSVVTNTLVEELEESQRKCPPCWYKFANTFLIWEC |
| | | | HPYWIKLKEIVNLIVMDPFVDLAITICIVLNTLFM |
| | | | AMEHHPMTPQFEHVLAVGNLVETGIFTAEMELKLI |
| | | | AMDPYYYFQEGWNIFDGFIVSLSLMELSLADVEGL |
| | | | SVLRSFRLLRVFKLAKSWPTLNMLIKIIGNSVGAL |
| | | | GNLTLVLAIIVFIFAVVGMQLFGKSYKECVCKINQ |
| | | | DCELPRWHMHDFFHSFLIVERVLCGEWIETMWDCM |
| | | | EVAGQAMCLIVFMMVMVIGNLVVLNLFLALLLSSE |
| | | | SADNLAATDDDGEMNNLQISVIRIKKGVAWTKLKV |
| | | | HAFMQAHFKOREADEVKPLDELYEKKANCIANHTG |
| | | | ADIHRNGDFQKNGNGTTSGIGSSVEKYIIDEDHMS |
| | | | FINNPNLTVRVPIAVGESDFENLNTEDVSSESDPE |
| | | | GSKDKLDDTSSSEGSTIDIKPEVEEVPVEQPEEYL |
| | | | DPDACFTEGCVQRFKCCQVNIEEGLGKSWWILRKT |
| | | | CFLIVEHNWFETFIIFMILLSSGALAFEDIYIEQR |
| | | | KTIRTILEYADKVETYIFILEMLLKWTAYGFVKFF |
| | | | TNAWCWLDELIVAVSLVSLIANALGYSELGAIKSL |
| | | | RTLRALRPLRALSRFEGMRVVVNALVGAIPSIMNV |
| | | | LLVCLIFWLIFSIMGVNLFAGKYHYCFNETSEIRE |
| | | | EIEDVNNKTECEKLMEGNNTEIRWKNVKINEDNVG |
| | | | AGYLALLQVATFKGWMDIMYAAVDSRKPDEQPKYE |
| | | | DNIYMYIYFVIFIIFGSFFTLNLFIGVIIDNENQQ |
| | | | KKKFGGQDIFMTEEQKKYYNAMKKLGSKKPQKPIP |
| | | | RPLNKIQGIVFDFVTQQAFDIVIMMLICLNMVTMM |
| | | | VETDTQSKQMENILYWINLVFVIFFTCECVLKMFA |
| | | | LRHYYFTIGWNIFDFVVVILSIVGMFLADIIEKYF |
| | | | VSPTLERVIRLARIGRILRLIKGAKGIRTLLFALM |
| | | | MSLPALFNIGLLLFLVMFIFSIFGMSNFAYVKHEA |
| | | | GIDDMENFETEGNSMICLFQITTSAGWDGLLLPIL |
| | | | NRPPDCSLDKEHPGSGFKGDCGNPSVGIFFFVSYI |
| | | | IISFLIVVNMYIAIILENFSVATEESADPLSEDDE |
| | | | ETFYEIWEKFDPDATQFIEYCKLADFADALEHPLR |
| | | | VPKPNTIELIAMDLPMVSGDRIHCLDILFAFTKRV |
| | | | LGDSGELDILRQQMEERFVASNPSKVSYEPITTTL |
| | | | RRKQEEVSAVVLQRAYRGHLARRGFICKKTTSNKL |
| | | | ENGGTHREKKESTPSTASLPSYDSVTKPEKEKQQR |
| | | | AEEGRRERAKRQKEVRESKC |
| Glutamate | GRIN2A- | 236 | MGRVGYWTLLVLPALLVWRGPAPSAAAEKGPPALN |
| receptor | Related | | IAVMLGHSHDVTERELRTLWGPEQAAGLPLDVNVV |
| ionotropic, | Disorder; | | ALLMNRTDPKSLITHVCDLMSGARIHGLVFGDDTD |
| NMDA 2A | Epilepsy, focal, | | QEAVAQMLDFISSHTFVPILGIHGGASMIMADKDP |
| (GRIN2A) | with speech | | TSTFFQFGASIQQQATVMLKIMQDYDWHVESLVTT |
| Signal | disorder and | | IFPGYREFISFVKTTVDNSFVGWDMQNVITLDTSF |
| Sequence | with or without | | EDAKTQVQLKKIHSSVILLYCSKDEAVLILSEARS |
| Underlined | mental | | LGLTGYDFFWIVPSLVSGNTELIPKEFPSGLISVS |
| | retardation | | YDDWDYSLEARVRDGIGILTTAASSMLEKESYIPE |
| | | | AKASCYGQMERPEVPMHTLHPFMVNVTWDGKDLSF |
| | | | TEEGYQVHPRLVVIVLNKDREWEKVGKWENHTLSL |
| | | | RHAVWPRYKSFSDCEPDDNHLSIVTLEEAPFVIVE |
| | | | DIDPLTETCVRNTVPCRKFVKINNSTNEGMNVKKC |
| | | | CKGFCIDILKKLSRTVKFTYDLYLVTNGKHGKKVN |
| | | | NVWNGMIGEVVYQRAVMAVGSLTINEERSEVVDES |
| | | | VPFVETGISVMVSRSNGTVSPSAFLEPESASVWVM |
| | | | MFVMLLIVSAIAVFVFEYFSPVGYNRNLAKGKAPH |
| | | | GPSFTIGKAIWLLWGLVENNSVPVQNPKGTTSKIM |
| | | | VSVWAFFAVIFLASYTANLAAFMIQEEFVDQVTGL |
| | | | SDKKFORPHDYSPPFRFGTVPNGSTERNIRNNYPY |
| | | | MHQYMTKFNQKGVEDALVSLKTGKLDAFIYDAAVL |
| | | | NYKAGRDEGCKLVTIGSGYIFATTGYGIALQKGSP |
| | | | WKRQIDLALLQFVGDGEMEELETLWLTGICHNEKN |
| | | | EVMSSQLDIDNMAGVFYMLAAAMALSLITFIWEHL |
| | | | FYWKLRFCFTGVCSDRPGLLESISRGIYSCIHGVH |
| | | | IEEKKKSPDFNLTGSQSNMLKLLRSAKNISSMSNM |
| | | | NSSRMDSPKRAADFIQRGSLIMDMVSDKGNLMYSD |
| | | | NRSFQGKESIFGDNMNELQTFVANRQKDNLNNYVE |
| | | | QGQHPLTLNESNPNTVEVAVSTESKANSRPRQLWK |
| | | | KSVDSIRQDSLSQNPVSQRDEATAENRTHSLKSPR |
| | | | YLPEEMAHSDISETSNRATCHREPDNSKNHKTKDN |
| | | | FKRSVASKYPKDCSEVERTYLKTKSSSPRDKIYTI |
| | | | DGEKEPGFHLDPPQFVENVTLPENVDEPDPYQDPS |
| | | | ENFRKGDSTLPMNRNPLHNEEGLSNNDQYKLYSKH |
| | | | FTLKDKGSPHSETSERYRQNSTHCRSCLSNMPTYS |
| | | | GHFTMRSPFKCDACLRMGNLYDIDEDQMLQETGNP |
| | | | ATGEQVYQQDWAQNNALQLQKNKLRISRQHSYDNI |
| | | | VDKPRELDLSRPSRSISLKDRERLLEGNFYGSLES |
| | | | VPSSKLSGKKSSLFPQGLEDSKRSKSLLPDHTSDN |
| | | | PFLHSHRDDQRLVIGRCPSDPYKHSLPSQAVNDSY |
| | | | LRSSLRSTASYCSRDSRGHNDVYISEHVMPYAANK |
| | | | NNMYSTPRVLNSCSNRRVYKKMPSIESDV |
| Sodium- and | SLC6A1- | 237 | MATNGSKVADGQISTEVSEAPVANDKPKTLVVKVQ |
| chloride- | Related | | KKAADLPDRDTWKGREDFLMSCVGYAIGLGNVWRF |
| dependent | Disorder; | | PYLCGKNGGGAFLIPYFLTLIFAGVPLELLECSLG |
| GABA | Myoclonic- | | QYTSIGGLGVWKLAPMFKGVGLAAAVLSFWLNIYY |
| transporter 1 | atonic epilepsy | | IVIISWAIYYLYNSFTTTLPWKQCDNPWNTDRCES |
| (SLC6A1) | | | NYSMVNTTNMTSAVVEFWERNMHQMTDGLDKPGQI |
| | | | RWPLAITLAIAWILVYFCIWKGVGWTGKVVYFSAT |
| | | | YPYIMLIILFFRGVTLPGAKEGILFYITPNERKLS |
| | | | DSEVWLDAATQIFFSYGLGLGSLIALGSYNSFHNN |
| | | | VYRDSIIVCCINSCTSMFAGFVIFSIVGEMAHVTK |
| | | | RSIADVAASGPGLAFLAYPEAVTQLPISPLWAILF |
| | | | FSMLLMLGIDSQFCTVEGFITALVDEYPRLLRNRR |
| | | | ELFIAAVCIISYLIGLSNITQGGIYVEKLFDYYSA |
| | | | SGMSLLFLVFFECVSISWFYGVNRFYDNIQEMVGS |
| | | | RPCIWWKLCWSFFTPIIVAGVFIFSAVQMTPLTMG |
| | | | NYVFPKWGQGVGWLMALSSMVLIPGYMAYMELTLK |
| | | | GSLKQRIQVMVQPSEDIVRPENGPEQPQAGSSTSK |
| | | | EAYI |
| Sodium/ | Alternating | 238 | MGRGAGREYSPAATTAENGGGKKKQKEKELDELKK |
| potassium- | hemiplegia of | | EVAMDDHKLSLDELGRKYQVDLSKGLTNORAQDVL |
| transporting | childhood | | ARDGPNALTPPPTTPEWVKFCRQLEGGESILLWIG |
| ATPase | | | AILCFLAYGIQAAMEDEPSNDNLYLGVVLAAVVIV |
| subunit alpha-2 | | | TGCFSYYQEAKSSKIMDSFKNMVPQQALVIREGEK |
| (ATP1A2) | | | MQINAEEVVVGDLVEVKGGDRVPADLRIISSHGCK |
| Signal | | | VDNSSLTGESEPQTRSPEFTHENPLETRNICFEST |
| Sequence | | | NCVEGTARGIVIATGDRTVMGRIATLASGLEVGRT |
| Underlined | | | PIAMEIEHFIQLITGVAVELGVSFFVLSLILGYSW |
| | | | LEAVIFLIGIIVANVPEGLLATVTVCLTLTAKRMA |
| | | | RKNCLVKNLEAVETLGSTSTICSDKTGTLTQNRMT |
| | | | VAHMWFDNQIHEADTTEDQSGATFDKRSPTWTALS |
| | | | RIAGLCNRAVFKAGQENISVSKRDTAGDASESALL |
| | | | KCIELSCGSVRKMRDRNPKVAEIPENSTNKYQLSI |
| | | | HEREDSPQSHVLVMKGAPERILDRCSTILVQGKEI |
| | | | PLDKEMQDAFQNAYMELGGLGERVLGFCQLNLPSG |
| | | | KFPRGFKFDTDELNFPTEKLCFVGLMSMIDPPRAA |
| | | | VPDAVGKCRSAGIKVIMVTGDHPITAKAIAKGVGI |
| | | | ISEGNETVEDIAARLNIPMSQVNPREAKACVVHGS |
| | | | DLKDMTSEQLDEILKNHTEIVFARTSPQQKLIIVE |
| | | | GCQRQGAIVAVTGDGVNDSPALKKADIGIAMGISG |
| | | | SDVSKQAADMILLDDNFASIVTGVEEGRLIEDNLK |
| | | | KSIAYTLTSNIPEITPELLFIIANIPLPLGTVTIL |
| | | | CIDLGTDMVPAISLAYEAAESDIMKRQPRNSQTDK |
| | | | LVNERLISMAYGQIGMIQALGGFFTYFVILAENGE |
| | | | LPSRLLGIRLDWDDRTMNDLEDSYGQEWTYEQRKV |
| | | | VEFTCHTAFFASIVVVQWADLIICKTRRNSVFQQG |
| | | | MKNKILIFGLLEETALAAFLSYCPGMGVALRMYPL |
| | | | KVTWWFCAFPYSLLIFIYDEVRKLILRRYPGGWVE |
| | | | KETYY |
| Sodium/ | Alternating | 239 | MGDKKDDKDSPKKNKGKERRDLDDLKKEVAMTEHK |
| potassium- | hemiplegia of | | MSVEEVCRKYNTDCVQGLTHSKAQEILARDGPNAL |
| transporting | childhood 2 | | TPPPTTPEWVKFCRQLEGGESILLWIGAILCFLAY |
| ATPase | | | GIQAGTEDDPSGDNLYLGIVLAAVVIITGCESYYQ |
| subunit alpha-3 | | | EAKSSKIMESFKNMVPQQALVIREGEKMQVNAEEV |
| (ATP1A3) | | | VVGDLVEIKGGDRVPADLRIISAHGCKVDNSSLTG |
| | | | ESEPQTRSPDCTHDNPLETRNITFFSTNCVEGTAR |
| | | | GVVVATGDRTVMGRIATLASGLEVGKTPIAIEIEH |
| | | | FIQLITGVAVELGVSFFILSLILGYTWLEAVIFLI |
| | | | GIIVANVPEGLLATVTVCLTLTAKRMARKNCLVKN |
| | | | LEAVETLGSTSTICSDKTGTLTQNRMTVAHMWEDN |
| | | | QIHEADTTEDQSGTSFDKSSHTWVALSHIAGLCNR |
| | | | AVFKGGQDNIPVLKRDVAGDASESALLKCIELSSG |
| | | | SVKLMRERNKKVAEIPENSTNKYQLSIHETEDPND |
| | | | NRYLLVMKGAPERILDRCSTILLQGKEQPLDEEMK |
| | | | EAFQNAYLELGGLGERVLGFCHYYLPEEQFPKGFA |
| | | | FDCDDVNFTTDNLCFVGLMSMIDPPRAAVPDAVGK |
| | | | CRSAGIKVIMVTGDHPITAKAIAKGVGIISEGNET |
| | | | VEDIAARLNIPVSQVNPRDAKACVIHGTDLKDETS |
| | | | EQIDEILQNHTEIVFARTSPQQKLIIVEGCQRQGA |
| | | | IVAVTGDGVNDSPALKKADIGVAMGIAGSDVSKQA |
| | | | ADMILLDDNFASIVTGVEEGRLIFDNLKKSIAYTL |
| | | | TSNIPEITPELLFIMANIPLPLGTITILCIDLGTD |
| | | | MVPAISLAYEAAESDIMKRQPRNPRTDKLVNERLI |
| | | | SMAYGQIGMIQALGGFFSYFVILAENGELPGNLVG |
| | | | IRLNWDDRTVNDLEDSYGQQWTYEQRKVVEFTCHT |
| | | | AFFVSIVVVQWADLIICKTRRNSVFQQGMKNKILI |
| | | | FGLFEETALAAFLSYCPGMDVALRMYPLKPSWWFC |
| | | | AFPYSFLIFVYDEIRKLILRRNPGGWVEKETYY |
| Sodium | SCN9A | 240 | MAMLPPPGPQSFVHFTKQSLALIEQRIAERKSKEP |
| channel protein | Epilepsy; | | KEEKKDDDEEAPKPSSDLEAGKQLPFIYGDIPPGM |
| type 9 subunit | Epilepsy, | | VSEPLEDLDPYYADKKTFIVLNKGKTIFRENATPA |
| alpha | generalized, | | LYMLSPFSPLRRISIKILVHSLFSMLIMCTILTNC |
| (SCN9A) | with febrile | | IFMTMNNPPDWTKNVEYTFTGIYTFESLVKILARG |
| | seizures plus, | | FCVGEFTFLRDPWNWLDFVVIVFAYLTEFVNLGNV |
| | type 7 | | SALRTFRVLRALKTISVIPGLKTIVGALIQSVKKL |
| | | | SDVMILTVFCLSVFALIGLQLFMGNLKHKCERNSL |
| | | | ENNETLESIMNTLESEEDERKYFYYLEGSKDALLC |
| | | | GFSTDSGQCPEGYTCVKIGRNPDYGYTSEDTESWA |
| | | | FLALFRLMTQDYWENLYQQTLRAAGKTYMIFFVVV |
| | | | IFLGSFYLINLILAVVAMAYEEQNQANIEEAKQKE |
| | | | LEFQQMLDRLKKEQEEAEAIAAAAAEYTSIRRSRI |
| | | | MGLSESSSETSKLSSKSAKERRNRRKKKNQKKLSS |
| | | | GEEKGDAEKLSKSESEDSIRRKSFHLGVEGHRRAH |
| | | | EKRLSTPNQSPLSIRGSLFSARRSSRTSLFSFKGR |
| | | | GRDIGSETEFADDEHSIFGDNESRRGSLFVPHRPQ |
| | | | ERRSSNISQASRSPPMLPVNGKMHSAVDCNGVVSL |
| | | | VDGRSALMLPNGQLLPEVIIDKATSDDSGTTNQIH |
| | | | KKRRCSSYLLSEDMLNDPNLRQRAMSRASILTNTV |
| | | | EELEESRQKCPPWWYRFAHKFLIWNCSPYWIKFKK |
| | | | CIYFIVMDPFVDLAITICIVLNTLFMAMEHHPMTE |
| | | | EFKNVLAIGNLVFTGIFAAEMVLKLIAMDPYEYFQ |
| | | | VGWNIFDSLIVTLSLVELFLADVEGLSVLRSERLL |
| | | | RVFKLAKSWPTLNMLIKIIGNSVGALGNLTLVLAI |
| | | | IVFIFAVVGMQLFGKSYKECVCKINDDCTLPRWHM |
| | | | NDFFHSFLIVERVLCGEWIETMWDCMEVAGQAMCL |
| | | | IVYMMVMVIGNLVVLNLFLALLLSSESSDNLTAIE |
| | | | EDPDANNLQIAVTRIKKGINYVKQTLREFILKAFS |
| | | | KKPKISREIRQAEDLNTKKENYISNHTLAEMSKGH |
| | | | NFLKEKDKISGFGSSVDKHLMEDSDGQSFIHNPSL |
| | | | TVTVPIAPGESDLENMNAEELSSDSDSEYSKVRLN |
| | | | RSSSSECSTVDNPLPGEGEEAEAEPMNSDEPEACE |
| | | | TDGCVWRESCCQVNIESGKGKIWWNIRKTCYKIVE |
| | | | HSWFESFIVLMILLSSGALAFEDIYIERKKTIKII |
| | | | LEYADKIFTYIFILEMLLKWIAYGYKTYFTNAWCW |
| | | | LDFLIVDVSLVTLVANTLGYSDLGPIKSLRTLRAL |
| | | | RPLRALSRFEGMRVVVNALIGAIPSIMNVLLVCLI |
| | | | FWLIFSIMGVNLFAGKFYECINTTDGSREPASQVP |
| | | | NRSECFALMNVSQNVRWKNLKVNFDNVGLGYLSLL |
| | | | QVATFKGWTIIMYAAVDSVNVDKQPKYEYSLYMYI |
| | | | YFVVFIIFGSFFTLNLFIGVIIDNENQQKKKLGGQ |
| | | | DIFMTEEQKKYYNAMKKLGSKKPQKPIPRPGNKIQ |
| | | | GCIFDLVTNQAFDISIMVLICLNMVTMMVEKEGQS |
| | | | QHMTEVLYWINVVFIILFTGECVLKLISLRHYYFT |
| | | | VGWNIFDFVVVIISIVGMFLADLIETYFVSPTLER |
| | | | VIRLARIGRILRLVKGAKGIRTLLFALMMSLPALF |
| | | | NIGLLLFLVMFIYAIFGMSNFAYVKKEDGINDMEN |
| | | | FETFGNSMICLFQITTSAGWDGLLAPILNSKPPDC |
| | | | DPKKVHPGSSVEGDCGNPSVGIFYFVSYIIISELV |
| | | | VVNMYIAVILENFSVATEESTEPLSEDDFEMFYEV |
| | | | WEKFDPDATQFIEFSKLSDFAAALDPPLLIAKPNK |
| | | | VQLIAMDLPMVSGDRIHCLDILFAFTKRVLGESGE |
| | | | MDSLRSQMEERFMSANPSKVSYEPITTTLKRKQED |
| | | | VSATVIQRAYRRYRLRQNVKNISSIYIKDGDRDDD |
| | | | LLNKKDMAFDNVNENSSPEKTDATSSTTSPPSYDS |
| | | | VTKPDKEKYEQDRTEKEDKGKDSKESKK |
| Gamma- | GABRB3 | 241 | MWGLAGGRLFGIFSAPVLVAVVCCAQSVNDPGNMS |
| aminobutyric | Associated | | FVKETVDKLLKGYDIRLRPDFGGPPVCVGMNIDIA |
| acid receptor | Epilepsy | | SIDMVSEVNMDYTLTMYFQQYWRDKRLAYSGIPLN |
| subunit beta-3 | | | LTLDNRVADQLWVPDTYFLNDKKSFVHGVTVKNRM |
| (GABRB3) | | | IRLHPDGTVLYGLRITTTAACMMDLRRYPLDEQNC |
| Signal | | | TLEIESYGYTTDDIEFYWRGGDKAVTGVERIELPQ |
| Sequence | | | FSIVEHRLVSRNVVFATGAYPRLSLSERLKRNIGY |
| Underlined | | | FILQTYMPSILITILSWVSFWINYDASAARVALGI |
| | | | TTVLTMTTINTHLRETLPKIPYVKAIDMYLMGCFV |
| | | | FVFLALLEYAFVNYIFFGRGPQRQKKLAEKTAKAK |
| | | | NDRSKSESNRVDAHGNILLTSLEVHNEMNEVSGGI |
| | | | GDTRNSAISFDNSGIQYRKQSMPREGHGRELGDRS |
| | | | LPHKKTHLRRRSSQLKIKIPDLTDVNAIDRWSRIV |
| | | | FPFTFSLENLVYWLYYVN |
| Potassium | Heterotetramer- | 242 | MGLKARRAAGAAGGGGDGGGGGGGAANPAGGDAAA |
| voltage-gated | izes with KCNQ2 | | AGDEERKVGLAPGDVEQVTLALGAGADKDGTLLLE |
| channel | KCNQ2-Related | | GGGRDEGQRRTPQGIGLLAKTPLSRPVKRNNAKYR |
| subfamily KQT | Disorders | | RIQTLIYDALERPRGWALLYHALVFLIVLGCLILA |
| member 3 | (Epileptic | | VLTTFKEYETVSGDWLLLLETFAIFIFGAEFALRI |
| (KCNQ3) | Encephalopathy) | | WAAGCCCRYKGWRGRLKFARKPLCMLDIFVLIASV |
| | | | PVVAVGNQGNVLATSLRSLRFLQILRMLRMDRRGG |
| | | | TWKLLGSAICAHSKELITAWYIGELTLILSSELVY |
| | | | LVEKDVPEVDAQGEEMKEEFETYADALWWGLITLA |
| | | | TIGYGDKTPKTWEGRLIAATFSLIGVSFFALPAGI |
| | | | LGSGLALKVQEQHRQKHFEKRRKPAAELIQAAWRY |
| | | | YATNPNRIDLVATWRFYESVVSFPFFRKEQLEAAS |
| | | | SQKLGLLDRVRLSNPRGSNTKGKLFTPLNVDAIEE |
| | | | SPSKEPKPVGLNNKERFRTAFRMKAYAFWQSSEDA |
| | | | GTGDPMAEDRGYGNDEPIEDMIPTLKAAIRAVRIL |
| | | | QFRLYKKKFKETLRPYDVKDVIEQYSAGHLDMLSR |
| | | | IKYLQTRIDMIFTPGPPSTPKHKKSQKGSAFTFPS |
| | | | QQSPRNEPYVARPSTSEIEDQSMMGKFVKVERQVQ |
| | | | DMGKKLDFLVDMHMQHMERLQVQVTEYYPTKGTSS |
| | | | PAEAEKKEDNRYSDLKTIICNYSETGPPEPPYSFH |
| | | | QVTIDKVSPYGFFAHDPVNLPRGGPSSGKVQATPP |
| | | | SSATTYVERPTVLPILTLLDSRVSCHSQADLQGPY |
| | | | SDRISPRQRRSITRDSDTPLSLMSVNHEELERSPS |
| | | | GFSISQDRDDYVFGPNGGSSWMREKRYLAEGETDT |
| | | | DTDPFTPSGSMPLSSTGDGISDSVWTPSNKPI |
| Rhodopsin | Autosomal | 243 | MNGTEGPNFYVPFSNATGVVRSPFEYPQYYLAEPW |
| (RHO) | Dominant RP | | QFSMLAAYMFLLIVLGFPINFLTLYVTVQHKKLRT |
| | | | PLNYILLNLAVADLFMVLGGFTSTLYTSLHGYFVE |
| | | | GPTGCNLEGFFATLGGEIALWSLVVLAIERYVVVC |
| | | | KPMSNERFGENHAIMGVAFTWVMALACAAPPLAGW |
| | | | SRYIPEGLQCSCGIDYYTLKPEVNNESFVIYMFVV |
| | | | HFTIPMIIIFFCYGQLVFTVKEAAAQQQESATTQK |
| | | | AEKEVTRMVIIMVIAFLICWVPYASVAFYIFTHQG |
| | | | SNEGPIEMTIPAFFAKSAAIYNPVIYIMMNKQFRN |
| | | | CMLTTICCGKNPLGDDEASATVSKTETSQVAPA |
| Protein jagged- | Alagille | 244 | MRSPRTRGRSGRPLSLLLALLCALRAKVCGASGQF |
| 1 (JAG1) | syndrome 1 | | ELEILSMQNVNGELQNGNCCGGARNPGDRKCTRDE |
| Signal Peptide | | | CDTYFKVCLKEYQSRVTAGGPCSFGSGSTPVIGGN |
| Underlined | | | TFNLKASRGNDRNRIVLPESFAWPRSYTLLVEAWD |
| | | | SSNDTVQPDSIIEKASHSGMINPSRQWQTLKQNTG |
| | | | VAHFEYQIRVTCDDYYYGFGCNKFCRPRDDFFGHY |
| | | | ACDQNGNKTCMEGWMGPECNRAICRQGCSPKHGSC |
| | | | KLPGDCRCQYGWQGLYCDKCIPHPGCVHGICNEPW |
| | | | QCLCETNWGGQLCDKDLNYCGTHQPCLNGGTCSNT |
| | | | GPDKYQCSCPEGYSGPNCEIAEHACLSDPCHNRGS |
| | | | CKETSLGFECECSPGWTGPTCSTNIDDCSPNNCSH |
| | | | GGTCQDLVNGFKCVCPPQWTGKTCQLDANECEAKP |
| | | | CVNAKSCKNLIASYYCDCLPGWMGQNCDININDCL |
| | | | GQCQNDASCRDLVNGYRCICPPGYAGDHCERDIDE |
| | | | CASNPCLNGGHCQNEINRFQCLCPTGFSGNLCQLD |
| | | | IDYCEPNPCQNGAQCYNRASDYFCKCPEDYEGKNC |
| | | | SHLKDHCRTTPCEVIDSCTVAMASNDTPEGVRYIS |
| | | | SNVCGPHGKCKSQSGGKFTCDCNKGFTGTYCHENI |
| | | | NDCESNPCRNGGTCIDGVNSYKCICSDGWEGAYCE |
| | | | TNINDCSQNPCHNGGTCRDLVNDFYCDCKNGWKGK |
| | | | TCHSRDSQCDEATCNNGGTCYDEGDAFKCMCPGGW |
| | | | EGTTCNIARNSSCLPNPCHNGGTCVVNGESFTCVC |
| | | | KEGWEGPICAQNTNDCSPHPCYNSGTCVDGDNWYR |
| | | | CECAPGFAGPDCRININECQSSPCAFGATCVDEIN |
| | | | GYRCVCPPGHSGAKCQEVSGRPCITMGSVIPDGAK |
| | | | WDDDCNTCQCLNGRIACSKVWCGPRPCLLHKGHSE |
| | | | CPSGQSCIPILDDQCFVHPCTGVGECRSSSLQPVK |
| | | | TKCTSDSYYQDNCANITFTENKEMMSPGLTTEHIC |
| | | | SELRNLNILKNVSAEYSIYIACEPSPSANNEIHVA |
| | | | ISAEDIRDDGNPIKEITDKIIDLVSKRDGNSSLIA |
| | | | AVAEVRVQRRPLKNRTDFLVPLLSSVLTVAWICCL |
| | | | VTAFYWCLRKRRKPGSHTHSASEDNTTNNVREQLN |
| | | | QIKNPIEKHGANTVPIKDYENKNSKMSKIRTHNSE |
| | | | VEEDDMDKHQQKARFAKQPAYTLVDREEKPPNGTP |
| | | | TKHPNWTNKQDNRDLESAQSLNRMEYIV |
| Inositol 1,4,5- | Gillespie | 245 | MSDKMSSFLHIGDICSLYAEGSTNGFISTLGLVDD |
| trisphosphate | Syndrome | | RCVVQPETGDLNNPPKKERDCLFKLCPMNRYSAQK |
| receptor type 1 | | | QFWKAAKPGANSTTDAVLLNKLHHAADLEKKQNET |
| (ITPR1) | | | ENRKLLGTVIQYGNVIQLLHLKSNKYLTVNKRLPA |
| | | | LLEKNAMRVTLDEAGNEGSWFYIQPFYKLRSIGDS |
| | | | VVIGDKVVLNPVNAGQPLHASSHQLVDNPGCNEVN |
| | | | SVNCNTSWKIVLFMKWSDNKDDILKGGDVVRLFHA |
| | | | EQEKFLTCDEHRKKQHVFLRTTGRQSATSATSSKA |
| | | | LWEVEVVQHDPCRGGAGYWNSLFRFKHLATGHYLA |
| | | | AEVDPDFEEECLEFQPSVDPDQDASRSRLRNAQEK |
| | | | MVYSLVSVPEGNDISSIFELDPTTLRGGDSLVPRN |
| | | | SYVRLRHLCTNTWVHSTNIPIDKEEEKPVMLKIGT |
| | | | SPVKEDKEAFAIVPVSPAEVRDLDFANDASKVLGS |
| | | | IAGKLEKGTITQNERRSVTKLLEDLVYFVTGGINS |
| | | | GQDVLEVVFSKPNRERQKLMREQNILKQIFKLLQA |
| | | | PFTDCGDGPMLRLEELGDQRHAPFRHICRLCYRVL |
| | | | RHSQQDYRKNQEYIAKQFGFMQKQIGYDVLAEDTI |
| | | | TALLHNNRKLLEKHITAAEIDTEVSLVRKNREPRE |
| | | | LDYLSDLCVSMNKSIPVTQELICKAVLNPTNADIL |
| | | | IETKLVLSRFEFEGVSSTGENALEAGEDEEEVWLE |
| | | | WRDSNKEIRSKSVRELAQDAKEGQKEDRDVLSYYR |
| | | | YQLNLFARMCLDRQYLAINEISGOLDVDLILRCMS |
| | | | DENLPYDLRASFCRLMLHMHVDRDPQEQVTPVKYA |
| | | | RLWSEIPSEIAIDDYDSSGASKDEIKERFAQTMEF |
| | | | VEEYLRDVVCQRFPFSDKEKNKLTFEVVNLARNLI |
| | | | YFGFYNFSDLLRLTKILLAILDCVHVTTIFPISKM |
| | | | AKGEENKGNNDVEKLKSSNVMRSIHGVGELMTQVV |
| | | | LRGGGFLPMTPMAAAPEGNVKQAEPEKEDIMVMDT |
| | | | KLKIIEILQFILNVRLDYRISCLLCIFKREFDESN |
| | | | SQTSETSSGNSSQEGPSNVPGALDFEHIEEQAEGI |
| | | | FGGSEENTPLDLDDHGGRTFLRVLLHLTMHDYPPL |
| | | | VSGALQLLFRHFSQRQEVLQAFKQVQLLVTSQDVD |
| | | | NYKQIKQDLDQLRSIVEKSELWVYKGQGPDETMDG |
| | | | ASGENEHKKTEEGNNKPQKHESTSSYNYRVVKEIL |
| | | | IRLSKLCVQESASVRKSRKQQQRLLRNMGAHAVVL |
| | | | ELLQIPYEKAEDTKMQEIMRLAHEFLONFCAGNQQ |
| | | | NQALLHKHINLFLNPGILEAVTMQHIFMNNFQLCS |
| | | | EINERVVQHFVHCIETHGRNVQYIKFLQTIVKAEG |
| | | | KFIKKCQDMVMAELVNSGEDVLVFYNDRASFQTLI |
| | | | QMMRSERDRMDENSPLMYHIHLVELLAVCTEGKNV |
| | | | YTEIKCNSLLPLDDIVRVVTHEDCIPEVKIAYINE |
| | | | LNHCYVDTEVEMKEIYTSNHMWKLFENFLVDICRA |
| | | | CNNTSDRKHADSILEKYVTEIVMSIVTTFFSSPES |
| | | | DQSTTLQTRQPVFVQLLQGVFRVYHCNWLMPSQKA |
| | | | SVESCIRVLSDVAKSRAIAIPVDLDSQVNNLELKS |
| | | | HSIVQKTAMNWRLSARNAARRDSVLAASRDYRNII |
| | | | ERLQDIVSALEDRLRPLVQAELSVLVDVLHRPELL |
| | | | FPENTDARRKCESGGFICKLIKHTKQLLEENEEKL |
| | | | CIKVLQTLREMMTKDRGYGEKLISIDELDNAELPP |
| | | | APDSENATEELEPSPPLRQLEDHKRGEALRQVLVN |
| | | | RYYGNVRPSGRRESLTSFGNGPLSAGGPGKPGGGG |
| | | | GGSGSSSMSRGEMSLAEVQCHLDKEGASNLVIDLI |
| | | | MNASSDRVFHESILLAIALLEGGNTTIQHSFFCRL |
| | | | TEDKKSEKFFKVFYDRMKVAQQEIKATVTVNTSDL |
| | | | GNKKKDDEVDRDAPSRKKAKEPTTQITEEVRDQLL |
| | | | EASAATRKAFTTFRREADPDDHYQPGEGTQATADK |
| | | | AKDDLEMSAVITIMQPILRFLQLLCENHNRDLQNE |
| | | | LRCQNNKTNYNLVCETLQFLDCICGSTTGGLGLLG |
| | | | LYINEKNVALINQTLESLTEYCQGPCHENONCIAT |
| | | | HESNGIDIITALILNDINPLGKKRMDLVLELKNNA |
| | | | SKLLLAIMESRHDSENAERILYNMRPKELVEVIKK |
| | | | AYMQGEVEFEDGENGEDGAASPRNVGHNIYILAHQ |
| | | | LARHNKELQSMLKPGGQVDGDEALEFYAKHTAQIE |
| | | | IVRLDRTMEQIVFPVPSICEFLTKESKLRIYYTTE |
| | | | RDEQGSKINDFFLRSEDLENEMNWQKKLRAQPVLY |
| | | | WCARNMSFWSSISENLAVLMNLLVAFFYPFKGVRG |
| | | | GTLEPHWSGLLWTAMLISLAIVIALPKPHGIRALI |
| | | | ASTILRLIFSVGLQPTLELLGAFNVCNKIIFLMSE |
| | | | VGNCGTFTRGYRAMVLDVEFLYHLLYLVICAMGLE |
| | | | VHEFFYSLLLEDLVYREETLLNVIKSVTRNGRSII |
| | | | LTAVLALILVYLFSIVGYLFFKDDFILEVDRLPNE |
| | | | TAVPETGESLASEFLESDVCRVESGENCSSPAPRE |
| | | | ELVPAEETEQDKEHTCETLLMCIVTVLSHGLRSGG |
| | | | GVGDVLRKPSKEEPLFAARVIYDLLFFFMVIIIVL |
| | | | NLIFGVIIDTFADLRSEKQKKEEILKTTCFICGLE |
| | | | RDKFDNKTVTFEEHIKEEHNMWHYLCFIVLVKVKD |
| | | | STEYTGPESYVAEMIKERNLDWFPRMRAMSLVSSD |
| | | | SEGEQNELRNLQEKLESTMKLVTNLSGQLSELKDQ |
| | | | MTEQRKQKQRIGLLGHPPHMNVNPQQPA |
| Sugar | — | 294 | MEAGGFLDSLIYGACVVFTLGMFSAGLSDLRHMRM |
| transporter | | | TRSVDNVQFLPFLTTEVNNLGWLSYGALKGDGILI |
| SWEET1 | | | VVNTVGAALQTLYILAYLHYCPRKRVVLLQTATLL |
| (SLC50A1) | | | GVLLLGYGYFWLLVPNPEARLQQLGLFCSVETISM |
| | | | YLSPLADLAKVIQTKSTQCLSYPLTIATLLTSASW |
| | | | CLYGFRLRDPYIMVSNFPGIVTSFIRFWLFWKYPQ |
| | | | EQDRNYWLLQT |
| Transmembrane | — | 295 | MELEAMSRYTSPVNPAVFPHLTVVLLAIGMFFTAW |
| protein 258 | | | FFVYEVTSTKYTRDIYKELLISLVASLEMGFGVLE |
| (TMEM258) | | | LLLWVGIYV |
| Follicle- | Ovarian | 296 | MALLLVSLLAFLSLGSGCHHRICHCSNRVFLCQES |
| stimulating | dysgenesis 1 | | KVTEIPSDLPRNAIELRFVLTKLRVIQKGAFSGFG |
| hormone | (ODG1) | | DLEKIEISQNDVLEVIEADVESNLPKLHEIRIEKA |
| receptor | | | NNLLYINPEAFQNLPNLQYLLISNTGIKHLPDVHK |
| (FSHR) | | | IHSLQKVLLDIQDNINIHTIERNSFVGLSFESVIL |
| Signal | | | WLNKNGIQEIHNCAFNGTQLDELNLSDNNNLEELP |
| Sequence | | | NDVFHGASGPVILDISRTRIHSLPSYGLENLKKLR |
| Underlined | | | ARSTYNLKKLPTLEKLVALMEASLTYPSHCCAFAN |
| | | | WRRQISELHPICNKSILRQEVDYMTQARGORSSLA |
| | | | EDNESSYSRGFDMTYTEFDYDLCNEVVDVTCSPKP |
| | | | DAFNPCEDIMGYNILRVLIWFISILAITGNIIVLV |
| | | | ILTTSQYKLTVPRELMCNLAFADLCIGIYLLLIAS |
| | | | VDIHTKSQYHNYAIDWQTGAGCDAAGFFTVFASEL |
| | | | SVYTLTAITLERWHTITHAMQLDCKVQLRHAASVM |
| | | | VMGWIFAFAAALFPIFGISSYMKVSICLPMDIDSP |
| | | | LSQLYVMSLLVLNVLAFVVICGCYIHIYLTVRNPN |
| | | | IVSSSSDTRIAKRMAMLIFTDELCMAPISFFAISA |
| | | | SLKVPLITVSKAKILLVLFHPINSCANPFLYAIFT |
| | | | KNFRRDFFILLSKCGCYEMQAQIYRTETSSTVHNT |
| | | | HPRNGHCSSAPRVTNGSTYILVPLSHLAQN |

### 5.3.3 Orientation and Linkers

In some embodiments, the effector domain is N-terminal of the targeting domain in the fusion protein. In some embodiments, the targeting domain is N-terminal of the effector domain in the fusion protein. In some embodiments, the effector domain is operably connected (directly or indirectly) to the C terminus of the targeting domain. In some embodiments, the effector domain is operably connected (directly or indirectly) to the N terminus of the targeting domain. In some embodiments, the effector domain is directly operably connected to the C terminus of the targeting domain. In some embodiments, the effector domain is directly operably connected to the N terminus of the targeting domain.

In some embodiments, the effector domain is indirectly operably connected to the C terminus of the targeting domain. In some embodiments, the effector domain is indirectly operably connected to the N terminus of the targeting domain. One or more amino acid sequences comprising e.g., a linker, or encoding one or more polypeptides may be positioned between the effector moiety and the targeting moiety. In some embodiments, the effector domain is indirectly operably connected to the C terminus of the targeting domain through a peptide linker. In some embodiments, the effector domain is indirectly operably connected to the N terminus of the targeting domain through a peptide linker.

Each component of the fusion protein described herein can be directly linked to the other to indirectly linked to the other via a peptide linker.

Any suitable peptide linker known in the art can be used that enables the effector domain and the targeting domain to bind their respective antigens. In some embodiments, the linker is one or any combination of a cleavable linker, a non-cleavable linker, a peptide linker, a flexible linker, a rigid linker, a helical linker, or a non-helical linker. In some embodiments, the linker is a peptide linker. In some embodiments, the linker is a peptide linker that comprises glycine or serine, or both glycine and serine amino acid residues. In some embodiments, the peptide linker comprises from about 1-20, 1-15, 1-10, 1-5, 5-20, 5-15, 5-10, or 15-20 amino acids. In some embodiments, the peptide linker comprises from or from about 2-25, 5-25, 10-25, 15-25, 20-25, 2-20, 5-20, 10-20, 15-20, 2-15, 5-15, 10-15, 2-10, or 5-10 amino acids. In some embodiments, the linker is a peptide linker that consists of glycine or serine, or both glycine and serine amino acid residues. In some embodiments, the peptide linker consists of from or from about 2-25, 5-25, 10-25, 15-25, 20-25, 2-20, 5-20, 10-20, 15-20, 2-15, 5-15, 10-15, 2-10, or 5-10 amino acids. In some embodiments, the peptide linker comprises at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker is at least 11 amino acids in length. In some embodiments, the linker is at least 15 amino acids in length. In some embodiments, the linker is 10, 11, 12, 13, 14, 15, 16, 17, 18, 19, 20, 21, 22, 23, 24, 25, 26, 27, 28, 29, or 30 amino acid residues in length.

In some embodiments, the linker is a glycine/serine linker, e.g., a peptide linker substantially consisting of the amino acids glycine and serine. In some embodiments, the linker is a glycine/serine/proline linker, e.g., a peptide linker substantially consisting of the amino acids glycine, serine, and proline.

In some embodiments, the amino acid sequence of the linker comprises the amino acid sequence of any one of SEQ ID NOS: 297-424, or the amino acid sequence of any one of SEQ ID NOS: 297-424 comprising 1, 2, or 3 amino acid modifications (e.g., a substitution, deletion, or addition). In some embodiments, the amino acid sequence of the linker consists of the amino acid sequence of any one of SEQ ID NOS: 297-424, or the amino acid sequence of any one of SEQ ID NOS: 297-424 comprising 1, 2, or 3 amino acid modifications (e.g., a substitution, deletion, or addition).

In some embodiments, the amino acid sequence of the linker comprises the amino acid sequence of any one of SEQ ID NOS: 297-306, or the amino acid sequence of any one of SEQ ID NOS: 297-288 comprising 1, 2, or 3 amino acid modifications (e.g., a substitution, deletion, or addition). In some embodiments, the amino acid sequence of the linker consists of the amino acid sequence of any one of SEQ ID NOS: 297-306, or the amino acid sequence of any one of SEQ ID NOS: 297-306 comprising 1, 2, or 3 amino acid modifications (e.g., a substitution, deletion, or addition).

The amino acid sequence of exemplary linkers for use in any one or more of the fusion proteins described herein is provided in Table 3 below.

TABLE 3
Amino Acid Sequence of Exemplary Linkers

| | SEQ |
|---|---|
| Amino Acid Sequence | ID NO |
| GGGGSGGGGSGGGGSGGGGSGGGGS | 297 |
| GGGGSGGGGSGGGGSGGGGS | 298 |
| GGGGSGGGGSGGGGS | 299 |
| GGGGSGGGGS | 300 |
| GGGGS | 301 |
| SGGGGSGGGGSGGGGS | 302 |
| SGGGGSGGGGSGGGG | 303 |
| SGGGGSGGGG | 304 |
| SGGGG | 305 |
| GGSGG | 306 |
| AHFKISGEKRPSTDPGKKAKNPKKKKKKDP | 307 |
| AHRAKKMSKTHA | 308 |
| ASPEYVNLPINGNG | 309 |
| CTKRPRW | 310 |
| DKAKRVSRNKSEKKRR | 311 |
| EELRLKEELLKGIYA | 312 |
| EEQLRRRKNSRLNNTG | 313 |
| EVLKVIRTGKRKKKAWKRMVTKVC | 314 |
| HHHHHHHHHHHHQPH | 315 |
| HKKKHPDASVNFSEFSK | 316 |
| HKRTKKNLS | 317 |
| IINGRKLKLKKSRRRSSQTSNNSFTSRRS | 318 |
| KAEQERRK | 319 |
| KEKRKRREELFIEQKKRK | 320 |
| KKGKDEWFSRGKKP | 321 |
| KKGPSVQKRKKTNLS | 322 |
| KKKTVINDLLHYKKEK | 323 |
| KKNGGKGKNKPSAKIKK | 324 |
| KKPKWDDFKKKKK | 325 |
| KKRKKDNLS | 326 |
| KKRRKRRRK | 327 |
| KKRRRRARK | 328 |
| KKSKRGR | 329 |
| KKSRKRGS | 330 |
| KKSTALSRELGKIMRRR | 331 |
| KKSYQDPEIIAHSRPRK | 332 |
| KKTGKNRKLKSKRVKTR | 333 |
| KKVSIAGQSGKLWRWKR | 334 |
| KKYENVVIKRSPRKRGRPRK | 335 |
| KNKKRK | 336 |
| KPKKKR | 337 |
| KRAMKDDSHGNSTSPKRRK | 338 |
| KRANSNLVAAYEKAKKK | 339 |
| KRASEDTTSGSPPKKSSAGPKR | 340 |
| KRFKRRWMVRKMKTKK | 341 |
| KRGLNSSFETSPKKVK | 342 |
| KRGNSSIGPNDLSKRKQRKK | 343 |
| KRIHSVSLSQSQIDPSKKVKRAK | 344 |
| KRKGKLKNKGSKRKK | 345 |
| KRRRRRRREKRKR | 346 |
| KRSNDRTYSPEEEKQRRA | 347 |
| KRTVATNGDASGAHRAKKMSK | 348 |
| KRVYNKGEDEQEHLPKGKKR | 349 |
| KSGKAPRRRAVSMDNSNK | 350 |
| KVNFLDMSLDDIIIYKELE | 351 |
| KVQHRIAKKTTRRRR | 352 |
| LSPSLSPL | 353 |
| MDSLLMNRRKFLYQFKNVRWAKGRRETYLC | 354 |
| MPQNEYIELHRKRYGYRLDYHEKKRKKESREAHERSKKAKK | 355 |
| MIGLKAKLYHK | |
| MVQLRPRASR | 356 |
| NNKLLAKRRKGGASPKDDPMDDIK | 357 |
| NYKRPMDGTYGPPAKRHEGE | 358 |
| PDTKRAKLDSSETTMVKKK | 359 |
| PEKRTKI | 360 |
| PGGRGKKK | 361 |
| PGKMDKGEHRQERRDRPY | 362 |
| PKKGDKYDKTD | 363 |
| PKKKSRK | 364 |
| PKKNKPE | 365 |
| PKKRAKV | 366 |
| PKPKKLKVE | 367 |
| PKRGRGR | 368 |
| PKRRLVDDA | 369 |
| PKRRRTY | 370 |
| PLEKRR | 371 |
| PLRKAKR | 372 |
| PPAKRKCIF | 373 |
| PPARRRRL | 374 |
| PPKKKRKV | 375 |
| PPNKRMKVKH | 376 |
| PPRIYPQLPSAPT | 377 |
| PQRSPFPKSSVKR | 378 |
| PRPRKVPR | 379 |
| PRRRVQRKR | 380 |
| PRRVRLK | 381 |
| PSRKRPR | 382 |
| PSSKKRKV | 383 |
| PTKKRVK | 384 |
| QRPGPYDRP | 385 |
| RGKGGKGLGKGGAKRHRK | 386 |
| RKAGKGGGGHKTTKKRSAKDEKVP | 387 |
| RKIKLKRAK | 388 |
| RKIKRKRAK | 389 |
| RKKEAPGPREELRSRGR | 390 |
| RKKRKGK | 391 |
| RKKRRQRRR | 392 |
| RKKSIPLSIKNLKRKHKRKKNKITR | 393 |
| RKLVKPKNTKMKTKLRTNPY | 394 |
| RKRLILSDKGQLDWKK | 395 |
| RKRLKSK | 396 |
| RKRRVRDNM | 397 |
| RKRSPKDKKEKDLDGAGKRRKT | 398 |
| RKRTPRVDGQTGENDMNKRRRK | 399 |
| RLPVRRRRRR | 400 |
| RLRFRKPKSK | 401 |
| RQQRKR | 402 |
| RRDLNSSFETSPKKVK | 403 |
| RRDRAKLR | 404 |
| RRGDGRRR | 405 |
| RRGRKRKAEKQ | 406 |
| RRKKRR | 407 |
| RRKRSKSEDMDSVESKRRR | 408 |
| RRKRSR | 409 |
| RRPKGKTLQKRKPK | 410 |
| RRRGFERFGPDNMGRKRK | 411 |
| RRRGKNKVAAQNCRK | 412 |
| RRRKRRNLS | 413 |
| RRRQKQKGGASRRR | 414 |
| RRRREGPRARRRR | 415 |
| RRTIRLKLVYDKCDRSCKIQKKNRNKCQYCRFHKCLSVGMS | 416 |
| HNAIREGRMPRSEKAKLKAE | |
| RRVPQRKEVSRCRKCRK | 417 |
| RVGGRRQAVECIEDLLNEPGQPLDLSCKRPRP | 418 |
| RVVKLRIAP | 419 |
| RVVRRR | 420 |
| SKRKTKISRKTR | 421 |
| SYVKTVPNRTRTYIKL | 422 |
| TGKNEAKKRKIA | 423 |
| TLSPASSPSSVSCPVIPASTDESPGSALNI | 424 |

### 5.3.3.1 Conditional Constructs

Also described herein are constructs that comprise a targeting domain (e.g., a VHH, (VHH)₂) bound to an effector domain (e.g., an effector domain that comprises a catalytic domain of an deubiquitinase, or an effector domain that comprises a deubiquitinase). In some embodiments, the association of the targeting domain and the effector domain is mediated by binding of a first agent (e.g., a small molecule, protein, or peptide) attached to the targeting domain and a second agent (e.g., a small, molecule, protein, or peptide) attached to the effector domain. For example, in one embodiment, the targeting domain may be attached to a first agent that specifically binds to a second agent that is attached to the effector domain. In some embodiments, specific binding of the first agent to the second agent is mediated by addition of a third agent (e.g., a small molecule).

For example, a conditional construct includes an KBP/FRB-based dimerization switch, e.g., as described in US20170081411 (the entire contents of which are incorporated by reference herein), can be utilized herein. FKBP12 (FKBP or FK506 binding protein) is an abundant cytoplasmic protein that serves as the initial intracellular target for the natural product immunosuppressive drug, rapamycin. Rapamycin binds to FKBP and to the large PI3K homolog FRAP (RAFT, mTOR), thereby acting to dimerize these molecules. In some embodiments, an FKBP/FRAP based switch, also referred to herein as an FKBP/FRB based switch, can utilize a heterodimerization molecule, e.g., rapamycin or a rapamycin analog. FRB is a 93 amino acid portion of FRAP, that is sufficient for binding the FKBP-rapamycin complex (Chen, J., Zheng, X. F., Brown, E. J. & Schreiber, S. L. (1995) Identification of an 11-kDa FKBP12-rapamycin-binding domain within the 289-kDa FKBP12-rapamycin-associated protein and characterization of a critical serine residue. Proc Natl Acad Sci USA 92: 4947-51), the entire contents of which is incorporated by reference herein. For example, the targeting domain can be attached to FKBP and the effector domain attached to FRB. Thereby, the association of the targeting domain and the effector domain is mediated by rapamycin and only takes place in the presence of rapamycin.

Exemplary conditional activation systems that can be used here include, but are not limited to those described in US20170081411; Lajoie M J, et al. Designed protein logic to target cells with precise combinations of surface antigens. Science. 2020 Sep. 25; 369(6511):1637-1643. doi: 10.1126/science.aba6527. Epub 2020 Aug. 20. PMID: 32820060; Farrants H, et al. Chemogenetic Control of Nanobodies. Nat Methods. 2020 March; 17(3):279-282. doi: 10.1038/s41592-020-0746-7. Epub 2020 Feb. 17. PMID: 32066961; and US20170081411, the entire contents of each of which is incorporated by reference herein for all purposes.

### 5.3.4 Exemplary Fusion Proteins

Exemplary fusion proteins of the present disclosure include, but are not limited to, those described below. In one embodiment, the fusion protein comprises an effector domain comprising a catalytic domain of a cysteine protease deubiquitinase, or a functional fragment or functional variant thereof; and a targeting domain comprising a targeting moiety that specifically binds a membrane protein, wherein the membrane protein is GRIN2B, CFTR, SCN1A, ATP7B, KCNQ2, SCN2A, CACNA1A, SLC2A1, SCN8A, PRRT2, GRIN2A, SLC6A1, USH2A, ATP1A2, ATP1A3, SCN9A, PCDH19, GABRB3, TSC2, TSC1, KCNQ3, DMD, RHO, JAG1, ITPR1, SLC50A1, TMEM258, or FSHR.

In one embodiment, the fusion protein comprises an effector domain comprising a catalytic domain of a metalloprotease deubiquitinase, or a functional fragment or functional variant thereof; and a targeting domain comprising a targeting moiety that specifically binds a membrane protein, wherein the membrane protein is GRIN2B, CFTR, SCN1A, ATP7B, KCNQ2, SCN2A, CACNA1A, SLC2A1, SCN8A, PRRT2, GRIN2A, SLC6A1, USH2A, ATP1A2, ATP1A3, SCN9A, PCDH19, GABRB3, TSC2, TSC1, KCNQ3, DMD, RHO, JAG1, ITPR1, SLC50A1, TMEM258, or FSHR.

In one embodiment, the fusion protein comprises an effector domain comprising a catalytic domain of a deubiquitinase, or a functional fragment or functional variant thereof, wherein the deubiquitinase is a ubiquitin-specific protease (USP), a ubiquitin C-terminal hydrolase (UCH), a Machado-Josephin domain protease (MJD), an ovarian tumour protease (OTU), a MINDY protease, or a ZUFSP protease; and a targeting domain comprising a targeting moiety that specifically binds a membrane protein, wherein the membrane protein is GRIN2B, CFTR, SCN1A, ATP7B, KCNQ2, SCN2A, CACNA1A, SLC2A1, SCN8A, PRRT2, GRIN2A, SLC6A1, USH2A, ATP1A2, ATP1A3, SCN9A, PCDH19, GABRB3, TSC2, TSC1, KCNQ3, DMD, RHO, JAG1, ITPR1, SLC50A1, TMEM258, or FSHR1.

In one embodiment, the fusion protein comprises an effector domain comprising a catalytic domain of a deubiquitinase, or a functional fragment or functional variant thereof, wherein the deubiquitinase is selected from the group consisting of USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, USP46, BAP1, UCHL1, UCHL3, UCHL5, ATXN3 ATXN3L, OTUB1, OTUB2 MINDY1, MINDY2, MINDY3, MINDY4, or ZUP1; and a targeting domain comprising a targeting moiety that specifically binds a membrane protein, wherein the membrane protein is GRIN2B, CFTR, SCN1A, ATP7B, KCNQ2, SCN2A, CACNA1A, SLC2A1, SCN8A, PRRT2, GRIN2A, SLC6A1, USH2A, ATP1A2, ATP1A3, SCN9A, PCDH19, GABRB3, TSC2, TSC1, KCNQ3, DMD, RHO, JAG1, ITPR1, SLC50A1, TMEM258, or FSHR.

In one embodiment, the fusion protein comprises an effector domain comprising a catalytic domain of a deubiquitinase, or a functional fragment or functional variant thereof, wherein the deubiquitinase is described in Table 1; and a targeting domain comprising a targeting moiety that specifically binds a membrane protein, wherein the membrane protein is GRIN2B, CFTR, SCN1A, ATP7B, KCNQ2, SCN2A, CACNA1A, SLC2A1, SCN8A, PRRT2, GRIN2A, SLC6A1, USH2A, ATP1A2, ATP1A3, SCN9A, PCDH19, GABRB3, TSC2, TSC1, KCNQ3, DMD, RHO, JAG1, ITPR1, SLC50A1, TMEM258, or FSHR.

In one embodiment, the fusion protein comprises an effector domain comprising a catalytic domain of a deubiquitinase, or a functional fragment or functional variant thereof, wherein the catalytic domain is described in Table 1; and a targeting domain comprising a targeting moiety that specifically binds a membrane protein, wherein the membrane protein is GRIN2B, CFTR, SCN1A, ATP7B, KCNQ2, SCN2A, CACNA1A, SLC2A1, SCN8A, PRRT2, GRIN2A, SLC6A1, USH2A, ATP1A2, ATP1A3, SCN9A, PCDH19, GABRB3, TSC2, TSC1, KCNQ3, DMD, RHO, JAG1, ITPR1, SLC50A1, TMEM258, or FSHR.

In one embodiment, the fusion protein comprises an effector domain comprising a catalytic domain of a deubiquitinase, or a functional fragment or functional variant thereof, wherein the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 1-112; and a targeting domain comprising a targeting moiety that specifically binds a membrane protein, wherein the membrane protein is GRIN2B, CFTR, SCN1A, ATP7B, KCNQ2, SCN2A, CACNA1A, SLC2A1, SCN8A, PRRT2, GRIN2A, SLC6A1, USH2A, ATP1A2, ATP1A3, SCN9A, PCDH19, GABRB3, TSC2, TSC1, KCNQ3, DMD, RHO, JAG1, ITPR1, SLC50A1, TMEM258, or FSHR.

In one embodiment, the fusion protein comprises an effector domain comprising a catalytic domain of a deubiquitinase, or a functional fragment or functional variant thereof, wherein the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 113-220 or 293; and a targeting domain comprising a targeting moiety that specifically binds a membrane protein, wherein the membrane protein is GRIN2B, CFTR, SCN1A, ATP7B, KCNQ2, SCN2A, CACNA1A, SLC2A1, SCN8A, PRRT2, GRIN2A, SLC6A1, USH2A, ATP1A2, ATP1A3, SCN9A, PCDH19, GABRB3, TSC2, TSC1, KCNQ3, DMD, RHO, JAG1, ITPR1, SLC50A1, TMEM258, or FSHR.

In one embodiment, the fusion protein comprises an effector domain comprising a catalytic domain of a deubiquitinase, or a functional fragment or functional variant thereof, wherein the deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 1-112; and a targeting domain comprising a targeting moiety that specifically binds a membrane protein, wherein the membrane protein comprises an amino acid sequence at least 90%, 91%, 92%, 93%, 94%, 95%, 96%, 97%, 98%, 99%, or 100% identical to any one of SEQ ID NOS: 221-245 or 294-296.

In one embodiment, the fusion protein comprises an effector domain comprising a catalytic domain of a deubiquitinase, or a functional fragment or functional variant thereof, wherein the catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOS: 113-220 or 293; and a targeting domain comprising a targeting moiety that specifically binds a membrane protein, wherein the membrane protein comprises an amino acid sequence at least 90%, 91%, 92%, 93%, 94%, 95%, 96%, 97%, 98%, 99%, or 100% identical to any one of SEQ ID NOS: 221-245 or 294-296.

### 5.3.4.1 Additional Exemplary Embodiments

Additional exemplary embodiments of fusion proteins described herein are provided below, which should not be construed as limiting.

Embodiment 1. A fusion protein comprising: (a) an effector moiety comprising a functional fragment of a human deubiquitinase that is capable of mediating deubiquitination, wherein the human deubiquitinase comprises an amino acid sequence at least 80%, 81%, 82%, 83%, 84%, 85%, 86%, 87%, 88%, 89%, 90%, 95%, 96%, 97%, 98%, 99%, or 100% identical to the amino acid sequence of any one of SEQ ID NOS: 1-112, and a targeting moiety comprising a VHH, (VHH)₂, or scFv that specifically binds to a membrane protein.

Embodiment 2. A fusion protein comprising an effector moiety comprising a functional fragment of a human deubiquitinase that is capable of mediating deubiquitination that comprises an amino acid sequence at least 80%, 81%, 82%, 83%, 84%, 85%, 86%, 87%, 88%, 89%, 90%, 95%, 96%, 97%, 98%, 99%, or 100% identical to the amino acid sequence of any one of SEQ ID NOS: 113-220 or 293, and a targeting moiety comprising a VHH, (VHH)₂, or scFv that specifically binds to a membrane protein.

Embodiment 3. A fusion protein comprising an effector moiety comprising a functional fragment of a human deubiquitinase that is capable of mediating deubiquitination that comprises an amino acid sequence at least 80%, 81%, 82%, 83%, 84%, 85%, 86%, 87%, 88%, 89%, 90%, 95%, 96%, 97%, 98%, 99%, or 100% identical to the amino acid sequence of SEQ ID NO: 293, and a targeting moiety comprising a VHH, (VHH)₂, or scFv that specifically binds to a membrane protein.

Embodiment 4. The fusion protein of any one of Embodiments 1-3, wherein said targeting moiety is a VHH or (VHH)₂.

Embodiment 5. The fusion protein of any one of Embodiments 1-4, wherein said membrane protein is GRIN2B, CFTR, SCN1A, ATP7B, KCNQ2, SCN2A, CACNA1A, SLC2A1, SCN8A, PRRT2, GRIN2A, SLC6A1, USH2A, ATP1A2, ATP1A3, SCN9A, PCDH19, GABRB3, TSC2, TSC1, KCNQ3, DMD, RHO, JAG1, ITPR1, SLC50A1, TMEM258, or FSHR.

Embodiment 6. The fusion protein of any one of Embodiments 1-3, wherein the membrane protein is SCN1A, GRIN2B, SLC50A1, TMEM258, or FSHR.

### 5.3.5 Methods of Making Fusion Proteins

Fusion proteins described herein can be made by any conventional technique known in the art, for example, recombinant techniques or chemical synthesis (e.g., solid phase peptide synthesis). In some embodiments, the fusion protein is made through recombinant expression in a cell (e.g., a eukaryotic cell, e.g., a mammalian cell). Briefly, the fusion protein can be made by synthesizing the DNA encoding the fusion protein and cloning the DNA into any suitable expression vector. Numerous cloning vectors are known to those of skill in the art, and the selection of an appropriate cloning vector is a matter of choice. The gene can be placed under the control of a promoter, ribosome binding site (for bacterial expression) and, optionally, an operator and/or one or more enhancer elements, so that the DNA sequence encoding the fusion protein is transcribed into RNA in the host cell transformed by a vector containing this expression construction. The coding sequence may or may not contain a signal peptide or leader sequence. Heterologous leader sequences can be added to the coding sequence that causes the secretion of the expressed polypeptide from the host organism. Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences. The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

The expression vector may then be used to transform an appropriate host cell. A number of mammalian cell lines are known in the art and include immortalized cell lines available from the American Type Culture Collection (ATCC), such as, but not limited to, Chinese hamster ovary (CHO) cells, CHO-suspension cells (CHO-S), HeLa cells, HEK293, baby hamster kidney (BHK) cells, monkey kidney cells (COS), VERO, HepG2, MadinDarby bovine kidney (MDBK) cells, NOS, U2OS, A549, HT1080, CAD, P19, NIH3T3, L929, N2a, MCF-7, Y79, SO-Rb50, DUKX-X11, and J558L.

Depending on the expression system and host selected, the fusion protein is produced by growing host cells transformed by an expression vector described above under conditions whereby the fusion protein is expressed. The fusion protein is then isolated from the host cells and purified. If the expression system secretes the fusion protein into growth media, the fusion protein can be purified directly from the media. If the fusion protein is not secreted, it is isolated from cell lysates. The selection of the appropriate growth conditions and recovery methods are within the skill of the art. Once purified, the amino acid sequences of the fusion proteins can be determined, i.e., by repetitive cycles of Edman degradation, followed by amino acid analysis by HPLC. Other methods of amino acid sequencing are also known in the art. Once purified, the functionality of the fusion protein can be assessed, e.g., as described herein, e.g., utilizing a bifunctional ELISA.

As described above, functionality of the fusion protein can be tested by any method known in the art. Each functionality can be measured in a separate assay. For example, binding of the targeting domain to the target protein can be measure using an enzyme linked immunosorbent assay (ELISA). Catalytic activity of the effector domain can be measured using any standard deubiquitinase activity assay known in the art. For example, BioVision Deubiquitinase Activity Assay Kit (Fluorometric) Catalog #K485-100 according to the manufacturer's instructions. The deubiquitinase activity of a fusion protein described herein can be measured for example by using a fluorescent deubiquitinase substrate to detect deubiquitinase activity upon cleavage of the fluorescent substrate. The deubiquitinase activity can also be measured according to the materials and methods set forth in the Examples provided herein.

### 5.4 Nucleic Acids, Host Cells, Vectors, and Viral Particles

In one aspect, provided herein are nucleic acid molecules encoding a fusion protein described herein. In some embodiments, the nucleic acid molecule is a DNA molecule. In some embodiments, the nucleic acid molecule is an RNA molecule. In some embodiments, the nucleic acid molecule contains at least one modified nucleic acid (e.g., that increases stability of the nucleic acid molecule), e.g., phosphorothioate, N6-methyladenosine (m6A), N6,2′-O-dimethyladenosine (m6Am), 8-oxo-7,8-dihydroguanosine (8-oxoG), pseudouridine (Ψ), 5-methylcytidine (m5C), and N4-acetylcytidine (ac4C).

In one aspect, provided herein is a host cell (or population of host cells) comprising a nucleic acid encoding a fusion protein described herein. In some embodiments, the nucleic acid is incorporated into the genome of the host cell. In some embodiments, the nucleic acid is not incorporated into the genome of the host cell. In some embodiments, the nucleic acid is present in the cell episomally. In some embodiments, the host cell is a human cell. In some embodiments, the host cell is a mammalian cell. In some embodiments, the host cell is a mouse, rat, hamster, guinea pig, cat, dog, or human cell. In some embodiments, the host cell is modified in vitro, ex vivo, or in vivo.

The nucleic acid can be introduced into the host cell by any suitable method known in the art (e.g., as described herein). For example, a viral delivery system (e.g., a retrovirus, an adenovirus, an adeno associated virus, a herpes virus, a lentivirus, a pox virus, a vaccinia virus, a vesicular stomatitis virus, a polio virus, a Newcastle's Disease virus, an Epstein-Barr virus, an influenza virus, a reoviruses, a myxoma virus, a maraba virus, a rhabdovirus, or a coxsackie virus delivery system) can be utilized to deliver a nucleic acid (e.g., DNA or RNA molecule) encoding the fusion protein for expression with the host cell. In some embodiments, the nucleic acid encoding the fusion protein is present episomally within the host cell. In some embodiments, the nucleic acid encoding the fusion protein is incorporated into the genome of the host cell. In some embodiments, the virus replication competent. In some embodiments, the virus is replication deficient.

In some embodiments, a nucleic acid (DNA or RNA) is delivered to the host cell using a non-viral vector (e.g., a plasmid) encoding the fusion protein. In some embodiments, the nucleic acid encoding the fusion protein is present episomally within the host cell. In some embodiments, the nucleic acid encoding the fusion protein is incorporated into the genome of the host cell. Exemplary non-viral transfection methods known in the art include, but are not limited to, direct delivery of DNA such as by ex vivo transfection, by injection (e.g., microinjection), electroporation, liposome mediated transfection, receptor-mediated transfection, microprojectile bombardment, by agitation with silicon carbide fibers Through the application of techniques such as these cells may be stably or transiently transfected with a nucleic acid encoding a fusion protein described herein to express the encoded fusion protein.

In one aspect, provided herein are vectors comprising a nucleic acid encoding a fusion protein described herein (e.g., a nucleic acid described herein). In some embodiments, the vector is a viral vector. Exemplary viral vectors include, but are not limited to, retroviral vectors, adenoviral vectors, adeno associated viral vectors, herpes viral vectors, lentiviral vectors, pox viral vectors, vaccinia viral vectors, vesicular stomatitis viral vectors, polio viral vectors, Newcastle's Disease viral vectors, Epstein-Barr viral vectors, influenza viral vectors, reovirus vectors, myxoma viral vectors, maraba viral vectors, rhabdoviral vectors, and coxsackie viral vectors. In some embodiments, the vector is a non-viral vector. In some embodiments, the non-viral vector is a plasmid.

In one aspect, provided herein is a viral particle (or population of viral particles) that comprise a nucleic acid encoding a fusion protein described herein (e.g., a nucleic acid described herein). In some embodiments, the viral particle is an RNA virus. In some embodiments, the viral particle is a DNA virus. In some embodiments, the viral particle comprises a double stranded genome. In some embodiments, the viral particle comprises a single stranded genome. Exemplary viral particles include, but are not limited to, a retrovirus, an adenovirus, an adeno associated virus, a herpes virus, a lentivirus, a pox virus, a vaccinia virus, a vesicular stomatitis virus, a polio virus, a Newcastle's Disease virus, an Epstein-Barr virus, an influenza virus, a reoviruses, a myxoma virus, a maraba virus, a rhabdovirus, or a coxsackie.

### 5.5 Pharmaceutical Compositions

In one aspect, provided herein are pharmaceutical compositions comprising 1) a fusion protein described herein, a nucleic acid encoding a fusion protein described herein, a vector comprising a nucleic acid encoding a fusion protein described herein, or a viral particle comprising a nucleic acid encoding a fusion protein described herein; and 2) at least one pharmaceutically acceptable carrier, excipient, stabilizer buffer, diluent, surfactant, preservative and/or adjuvant, etc (see, e.g., Remington's Pharmaceutical Sciences (1990) Mack Publishing Co., Easton, PA). A person of ordinary skill in the art can select suitable excipient for inclusion in the pharmaceutical composition. For example, the formulation of the pharmaceutical composition may differ based on the route of administration (e.g., intravenous, subcutaneous, etc.), and/or the active molecule contained within the pharmaceutical composition (e.g., a viral particle, a non-viral vector, a nucleic acid not contained within a vector).

Acceptable carriers, excipients, or stabilizers are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

In one embodiment, the present disclosure provides a pharmaceutical composition comprising a fusion protein described herein for use as a medicament. In another embodiment, the disclosure provides a pharmaceutical composition for use in a method for the treatment of cancer. In some embodiments, pharmaceutical compositions comprise a fusion protein disclosed herein, and optionally one or more additional prophylactic or therapeutic agents, in a pharmaceutically acceptable carrier.

A pharmaceutical composition may be formulated for any route of administration to a subject. Specific examples of routes of administration include parenteral administration (e.g., intravenous, subcutaneous, intramuscular). In some embodiments, the pharmaceutical composition is formulated for intravenous administration. In some embodiments, the pharmaceutical composition is formulated for subcutaneous administration. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins.

In some embodiments, the pharmaceutical composition is formulated for intravenous administration. Suitable carriers for intravenous administration include physiological saline or phosphate buffered saline (PBS), or solutions containing thickening or solubilizing agents, such as glucose, polyethylene glycol, or polypropylene glycol or mixtures thereof.

The compositions to be used for in vivo administration can be sterile. This is readily accomplished by filtration through, e.g., sterile filtration membranes.

Pharmaceutically acceptable carriers used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; or sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.6 Methods of Therapeutic Use

In one aspect, provided herein are methods of treating a disease in a subject by administering to the subject having the disease a fusion protein described herein, a nucleic acid encoding a fusion protein described herein, a vector comprising a nucleic acid encoding a fusion protein described herein, or a viral particle comprising a nucleic acid encoding a fusion protein described herein.

The fusion protein can be delivered to host cells via any method known in the art. For example, a viral delivery system (e.g., a retrovirus, an adenovirus, an adeno associated virus, a herpes virus, a lentivirus, a pox virus, a vaccinia virus, a vesicular stomatitis virus, a polio virus, a Newcastle's Disease virus, an Epstein-Barr virus, an influenza virus, a reoviruses, a myxoma virus, a maraba virus, a rhabdovirus, an enadenotucirev or a coxsackie) can be utilized to deliver a nucleic acid (e.g., DNA or RNA molecule) encoding the fusion protein for expression within a population of cells of a subject. In some embodiments, the nucleic acid encoding the fusion protein is present episomally within the population of cells of the subject. In some embodiments, the nucleic acid encoding the fusion protein is incorporated into the genome of the population of cells of the subject. In some embodiments, the virus is replication competent. In some embodiments, the virus is replication deficient.

In some embodiments, the fusion protein is administered to the subject. In some embodiments, a nucleic acid (DNA or RNA) is administered to the subject. In some embodiments, the nucleic acid (DNA or RNA) is complexed within a carrier (e.g., a nanoparticle, a liposome, a microsphere). In some embodiments, a nucleic acid (DNA or RNA) within a non-viral vector (e.g., a plasmid) encoding the fusion protein is administered to the subject.

### 5.6.1 Administration

The fusion protein can be delivered to host cells via any method known in the art. For example, a viral delivery system (e.g., a retrovirus, an adenovirus, an adeno associated virus, a herpes virus, a lentivirus, a pox virus, a vaccinia virus, a vesicular stomatitis virus, a polio virus, a Newcastle's Disease virus, an Epstein-Barr virus, an influenza virus, a reoviruses, a myxoma virus, a maraba virus, a rhabdovirus, an enadenotucirev or a coxsackie) can be utilized to deliver a nucleic acid (e.g., DNA or RNA molecule) encoding the fusion protein for expression within a population of cells of a subject. In some embodiments, the nucleic acid encoding the fusion protein is present episomally within the population of cells of the subject. In some embodiments, the nucleic acid encoding the fusion protein is incorporated into the genome of the population of cells of the subject. In some embodiments, the virus is replication competent. In some embodiments, the virus is replication deficient.

In some embodiments, the fusion protein is administered to the subject. In some embodiments, a nucleic acid (DNA or RNA) is administered to the subject. In some embodiments, the nucleic acid (DNA or RNA) is complexed within a carrier (e.g., a nanoparticle, a liposome, a microsphere). In some embodiments, a nucleic acid (DNA or RNA) within a non-viral vector (e.g., a plasmid) encoding the fusion protein is administered to the subject.

In some embodiment, the fusion protein is administered parenterally. In some embodiments, the fusion protein is administered via intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural or intrasternal injection or infusion. In some embodiments, the fusion protein is intravenously administered. In some embodiments, the fusion protein is subcutaneously administered. In some embodiments, the fusion protein is administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

In some embodiments, the methods disclosed herein are used in place of standard of care therapies. In certain embodiments, a standard of care therapy is used in combination with any method disclosed herein. In some embodiments, the methods disclosed herein are used after standard of care therapy has failed. In some embodiments, the fusion protein is co-administered, administered prior to, or administered after, an additional therapeutic agent. In some embodiments, the disease is a genetic disease.

### 5.6.2 Exemplary Genetic Diseases

In some embodiments, the disease is a genetic disease. In some embodiments, the genetic disease is associated with decreased expression of a functional target membrane protein. In some embodiments, the genetic disease is associated with decreased stability of a functional target membrane protein. In some embodiments, the genetic disease is associated with increased ubiquitination of a target membrane protein. In some embodiments, the genetic disease is associated with increased ubiquitination and degradation of a target membrane protein. In some embodiments, the genetic disease is a haploinsufficiency disease.

In some embodiments, the disease is an epileptic encephalopathy. In some embodiments, the epileptic encephalopathy is an early infantile epileptic encephalopathy. In some embodiments, the early infantile epileptic encephalopathy is selected from the group consisting of early infantile epileptic encephalopathy type 9, early infantile epileptic encephalopathy type 11, early infantile epileptic encephalopathy type 13, and early infantile epileptic encephalopathy type 27. In some embodiments, the disease is an episodic ataxia. In some embodiments, the disease is episodic ataxia type 2. In some embodiments, the disease is an episodic kinesigenic dyskinesia. In some embodiments, the disease is episodic kinesigenic dyskinesia type 1. In some embodiments, the disease is epilepsy. In some embodiments, the epilepsy is focal, with speech disorder and with or without mental retardation; myoclonic-atonic epilepsy; epilepsy type 7; or GABRB3 associated epilepsy. In some embodiments, the disease is tuberous sclerosis. In some embodiments, the disease is tuberous sclerosis type 1 or tuberous sclerosis type 2. In some embodiments, the disease is KCNQ2 encephalopathy.

In some embodiments, the disease is selected from the group consisting of early a GRIN2B-Related Disorder, a SCN2A-Related Disorder, a SCN8A-Related Disorder, SLC6A1-Related Disorder, a PRRT2 Dyskinesia & Epilepsy, a GRIN2A-Related Disorder, a CACNA1A-Related Disorder, a SCN9A Epilepsy, a PCDH19 Encephalopathy, KCNQ2 encephalopathy, infantile epileptic encephalopathy type 9, early infantile epileptic encephalopathy type 11, early infantile epileptic encephalopathy type 13, early infantile epileptic encephalopathy type 27, cystic fibrosis, Dravet syndrome, Wilson disease, episodic ataxia type 2; GLUT1 deficiency syndrome, episodic kinesigenic dyskinesia 1, epilepsy (e.g., focal, with speech disorder and with or without mental retardation), myoclonic-atonic epilepsy, Usher syndrome type 2A, alternating hemiplegia of childhood, alternating hemiplegia of childhood type 2, epilepsy type 7, GABRB3 associated epilepsy; tuberous sclerosis type 2; tuberous sclerosis type 1, KCNQ2-Related Disorders (Epileptic Encephalopathy), Becker Muscular Dystrophy, Autosomal Dominant RP, Alagille syndrome 1, Gillespie Syndrome, and Ovarian dysgenesis 1 (ODG1).

In some embodiments, the target membrane protein is GRIN2B, and the disease is a GRIN2B related disorder (e.g., an epileptic encephalopathy). In some embodiments, the target membrane protein is GRIN2B, and the disease is an early infantile epileptic encephalopathy. In some embodiments, the target membrane protein is GRIN2B, and the disease is early infantile epileptic encephalopathy type 27. In some embodiments, the target membrane protein is CFTR, and the disease is cystic fibrosis. In some embodiments, the target membrane protein is SCN1A, and the disease is Dravet syndrome. In some embodiments, the target membrane protein is ATP7B, and the disease is Wilson disease. In some embodiments, the target membrane protein is CACNA1A, and the disease is a CACA1A related disorder. In some embodiments, the target membrane protein is CACNA1A, and the disease is episodic ataxia type 2. In some embodiments, the target membrane protein is KCNQ2, and the disease is an KCNQ2 encephalopathy. In some embodiments, the target membrane protein is KCNQ2, and the disease is an epileptic encephalopathy. In some embodiments, the target membrane protein is SCN2A, and the disease is a SCN2A related disorder (e.g., an epileptic encephalopathy). In some embodiments, the target membrane protein is SCN2A, and the disease is early infantile epileptic encephalopathy type 11. In some embodiments, the target membrane protein is SLC2A1, and the disease is GLUT1 deficiency syndrome. In some embodiments, the target membrane protein is SCN8A, and the disease is a SCN8A related disorder (e.g., an epileptic encephalopathy). In some embodiments, the target membrane protein is SCN8A, and the disease is an epileptic encephalopathy. In some embodiments, the target membrane protein is SCN8A, and the disease is early infantile epileptic encephalopathy type 13. In some embodiments, the target membrane protein is PRRT2, and the disease is a PRRPT2 dyskinesia and/or epilepsy. In some embodiments, the target membrane protein is PRRT2, and the disease is an episodic kinesigenic dyskinesia type. In some embodiments, the target membrane protein is PRRT2, and the disease is episodic kinesigenic dyskinesia type 1. In some embodiments, the target membrane protein is GRIN2A, and the disease is a GRIN2A related disorder. In some embodiments, the target membrane protein is GRIN2A, and the disease is epilepsy. In some embodiments, the target membrane protein is GRIN2A, and the disease is focal epilepsy. In some embodiments, the target membrane protein is GRIN2A, and the disease is focal epilepsy with speech disorder and with or without mental retardation. In some embodiments, the target membrane protein is SLC6A1, and the disease is a SLC6A1 related disorder. In some embodiments, the target membrane protein is SLC6A1, and the disease is epilepsy. In some embodiments, the target membrane protein is SLC6A1, and the disease is myoclonic-atonic epilepsy. In some embodiments, the target membrane protein is USH2A, and the disease is Usher syndrome. In some embodiments, the target membrane protein is USH2A, and the disease is Usher syndrome type 2A. In some embodiments, the target membrane protein is ATP1A2, and the disease is alternating hemiplegia of childhood. In some embodiments, the target membrane protein is ATP1A2, and the disease is alternating hemiplegia of childhood type 1. In some embodiments, the target membrane protein is ATP1A3, and the disease is alternating hemiplegia of childhood. In some embodiments, the target membrane protein is ATP1A3, and the disease is alternating hemiplegia of childhood type 2. In some embodiments, the target membrane protein is SCN9A, and the disease an SCN9A epilepsy. In some embodiments, the target membrane protein is SCN9A1, and the disease an SCN9A epilepsy. In some embodiments, the target membrane protein is SCN9A1, and the disease is epilepsy. In some embodiments, the target membrane protein is SCN9A1, and the disease is epilepsy type 7. In some embodiments, the target membrane protein is PCDH19, and the disease is PCDH19 encephalopathy. In some embodiments, the target membrane protein is PCDH19, and the disease is an early infantile epileptic encephalopathy. In some embodiments, the target membrane protein is PCDH19, and the disease is early infantile epileptic encephalopathy type 9. In some embodiments, the target membrane protein is GABRB3, and the disease is epilepsy. In some embodiments, the target membrane protein is GABRB3, and the disease is GABRB3 associated epilepsy. In some embodiments, the target membrane protein is TSC2, and the disease is tuberous sclerosis. In some embodiments, the target membrane protein is TSC2, and the disease is tuberous sclerosis type 2. In some embodiments, the target membrane protein is TSC2, and the disease is tuberous sclerosis type 1. In some embodiments, the target membrane protein is TSC1, and the disease is tuberous sclerosis. In some embodiments, the target membrane protein is TSC1, and the disease is tuberous sclerosis type 1. In some embodiments, the target membrane protein is TSC1, and the disease is tuberous sclerosis type 2. In some embodiments, the target membrane protein is KCNQ3, and the disease is KCNQ2-Related Disorders (Epileptic Encephalopathy). In some embodiments, the target membrane protein is DMD, and the disease is Becker Muscular Dystrophy. In some embodiments, the target membrane protein is RHO, and the disease is Autosomal Dominant RP. In some embodiments, the target membrane protein is JAG1, and the disease is Alagille syndrome 1. In some embodiments, the target membrane protein is ITPR1, and the disease is Gillespie Syndrome. In some embodiments, the target membrane protein is FSHR, and the disease is ovarian dysgenesis 1 (ODG1).

### 5.7 Kits

In one aspect, provided herein are kits comprising a fusion protein described herein, a nucleic acid encoding a fusion protein described herein, a vector comprising a nucleic acid encoding a fusion protein described herein, or a viral particle comprising a nucleic acid encoding a fusion protein described herein, for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a disease (e.g., a genetic disease), the kit comprising: (a) a dosage of a fusion protein, a nucleic acid encoding a fusion protein described herein, a vector comprising a nucleic acid encoding a fusion protein described herein, or a viral particle comprising a nucleic acid encoding a fusion described herein; and (b) instructions for using the fusion protein in any of the therapy methods disclosed herein.

### 6. EXAMPLES

The present invention is further illustrated by the following examples which should not be construed as further limiting.

### 6.1 Example 1. Generation of Targeted Engineered Deubiquitinases

This example provides general experimental methods of using fluorescent tagged target proteins together with fluorophore tagged engineered deubiquitinases (enDUBs) to demonstrate up-regulation of expression in the context of an enDUB. For illustrative purposes the constructs disclosed below will be synthesized in a suitable vector for mammalian expression. Generally, the target protein will be expressed with a C-terminal YFP followed by a P2A cleavage signal and an mCherry protein as a second reporter (Target protein-YFP-P2A-mCherry). This construct will be co-transfected in the presence of a trifunctional fusion protein comprising of a CFP protein followed by a P2A signal and a nanobody specifically binding to YPF followed by the engineered DUB (CFP-P2A-Anti-YFPnanobody-enDUB). In applications for drug treatment the targeting nanobodies (or other specific binders) will be directed to the wild type (or disease-causing mutant) protein in the cell to be upregulated while the enDUB is fused to a binding protein directed to the target protein. Target protein binding moieties could be any antibody or antibody fragments, nanobodies, or any other non-antibody scaffold such as fibronectins, anticalins, ankyrin repeats or natural binding proteins interacting specifically with the target protein to be upregulated. The amino acid sequence of the components of the test fusion proteins is provided in Table 4 below.

TABLE 4
Amino Acid Sequence of Components of test fusion proteins

| Description | SEQ | |
|---|---|---|
| Target Proteins | ID NO | Amino Acid Sequence |
| Beta 2 | 246 | MGQPGNGSAFLLAPNGSHAPDHDVTQERDEVWVVGMGIVMSLIVLA |
| adrenergic | | IVFGNVLVITAIAKFERLQTVTNYFITSLACADLVMGLAVVPFGAA |
| receptor human | | HILMKMWTFGNFWCEFWTSIDVLCVTASIETLCVIAVDRYFAITSP |
| | | FKYQSLLTKNKARVIILMVWIVSGLTSFLPIQMHWYRATHQEAINC |
| | | YANETCCDFFTNQAYAIASSIVSFYVPLVIMVFVYSRVFQEAKRQL |
| | | QKIDKSEGRFHVQNLSQVEQDGRTGHGLRRSSKFCLKEHKALKTLG |
| | | IIMGTFTLCWLPFFIVNIVHVIQDNLIRKEVYILLNWIGYVNSGEN |
| | | PLIYCRSPDFRIAFQELLCLRRSSLKAYGNGYSSNGNTGEQSGYHV |
| | | EQEKENKLLCEDLPGTEDFVGHQGTVPSDNIDSQGRNCSTNDSLL |
| Kappa-type | 247 | MDSPIQIFRGEPGPTCAPSACLPPNSSAWFPGWAEPDSNGSAGSED |
| opioid receptor | | AQLEPAHISPAIPVIITAVYSVVFVVGLVGNSLVMFVIIRYTKMKT |
| human | | ATNIYIFNLALADALVTTTMPFQSTVYLMNSWPFGDVLCKIVISID |
| | | YYNMFTSIFTLTMMSVDRYIAVCHPVKALDERTPLKAKIINICIWL |
| | | LSSSVGISAIVLGGTKVREDVDVIECSLQFPDDDYSWWDLEMKICV |
| | | FIFAFVIPVLIIIVCYTLMILRLKSVRLLSGSREKDRNLRRITRLV |
| | | LVVVAVFVVCWTPIHIFILVEALGSTSHSTAALSSYYFCIALGYTN |
| | | SSLNPILYAFLDENFKRCFRDFCFPLKMRMERQSTSRVRNTVQDPA |
| | | YLRDIDGMNKPV |
| Muscarinic | 248 | MNNSTNSSNNSLALTSPYKTFEVVFIVLVAGSLSLVTIIGNILVMV |
| acetylcholine | | SIKVNRHLQTVNNYFLESLACADLIIGVESMNLYTLYTVIGYWPLG |
| receptor M2 | | PVVCDLWLALDYVVSNASVMNLLIISEDRYFCVTKPLTYPVKRTTK |
| human | | MAGMMIAAAWVLSFILWAPAILFWQFIVGVRTVEDGECYIQFFSNA |
| | | AVTFGTAIAAFYLPVIIMTVLYWHISRASKSRIKKDKKEPVANQDP |
| | | VSPSLVQGRIVKPNNNNMPSSDDGLEHNKIQNGKAPRDPVTENCVQ |
| | | GEEKESSNDSTSVSAVASNMRDDEITQDENTVSTSLGHSKDENSKQ |
| | | TCIRIGTKTPKSDSCTPTNTTVEVVGSSGQNGDEKQNIVARKIVKM |
| | | TKQPAKKKPPPSREKKVTRTILAILLAFIITWAPYNVMVLINTFCA |
| | | PCIPNTVWTIGYWLCYINSTINPACYALCNATFKKTFKHLLMCHYK |
| | | NIGATR |

Fluorescent Proteins

| | | |
|---|---|---|
| YFP | 249 | VSKGEELFTGVVPILVELDGDVNGHKFSVSGEGEGDATYGKLTLKF |
| | | ICTTGKLPVPWPTLVTTFGYGLQCFARYPDHMKQHDFFKSAMPEGY |
| | | VQERTIFFKDDGNYKTRAEVKFEGDTLVNRIELKGIDFKEDGNILG |
| | | HKLEYNYNSHNVYIMADKQKNGIKVNFKIRHNIEDGSVQLADHYQQ |
| | | NTPIGDGPVLLPDNHYLSYQSALSKDPNEKRDHMVLLEFVTAAGIT |
| | | LGMDELYK |
| mCherry | 250 | MVSKGEEDNMAIIKEFMRFKVHMEGSVNGHEFEIEGEGEGRPYEGT |
| | | QTAKLKVTKGGPLPFAWDILSPQFMYGSKAYVKHPADIPDYLKLSF |
| | | PEGFKWERVMNFEDGGVVTVTQDSSLQDGEFIYKVKLRGTNFPSDG |
| | | PVMQKKTMGWEASSERMYPEDGALKGEIKQRLKLKDGGHYDAEVKT |
| | | TYKAKKPVQLPGAYNVNIKLDITSHNEDYTIVEQYERAEGRHSTGG |
| | | MDELYK |
| CFP | 251 | MVSKGEELFTGVVPILVELDGDVNGHKESVSGEGEGDATYGKLTLK |
| | | FICTTGKLPVPWPTLVTTLTWGVQCFSRYPDHMKQHDFFKSAMPEG |
| | | YVQERTIFFKDDGNYKTRAEVKFEGDTLVNRIELKGIDFKEDGNIL |
| | | GHKLEYNYISHNVYITADKQKNGIKANFKIRHNIEDGSVQLADHYQ |
| | | QNTPIGDGPVLLPDNHYLSTQSALSKDPNEKRDHMVLLEFVTAAGI |
| | | TLGMDELYK |

A2 Peptides

| | | |
|---|---|---|
| P2A | 252 | GSGATNFSLLKQAGDVEENPGP |
| T2A | 253 | GSGEGRGSLLTCGDVEENPGP |
| E2A | 254 | GSGQCTNYALLKLAGDVESNPGP |

Target Binders

| | | |
|---|---|---|
| YFP targeting | 255 | WVAGMSSAGDRSSYEDSVKGRFTISRDDARNTVYLQMNSLKPEDTA |
| nanobody | | QVQLVESGGALVQPGGSLRLSCAASGFPVNRYSMRWYRQAPGKERE |
| | | VYYCNVNVGFEYWGQGTQVTVSS |
| Beta 2 | 256 | QVQLQESGGGLVQAGGSLRLSCAASGSIFALNIMGWYRQAPGKQRE |
| adrenergic | | LVAAIHSGGTTNYANSVKGRFTISRDNAANTVYLQMNSLKPEDTAV |
| receptor human | | YYCNVKDFGAIIYDYDYWGQGTQVTVSS |
| binder | | |
| (monobody) | | |
| Kappa-type | 257 | MAQVQLVESGGGLVRPGGSLRLSCVDSERTSYPMGWERRAPGKERE |
| opioid receptor | | FVASITWSGIDPTYADSVADRETTSRDVANNTLYLQMNSLKHEDTA |
| human binder | | VYYCAARAPVGQSSSPYDYDYWGQGTQVTVSSHHHHHHEPEA |
| (monobody) | | |
| Muscarinic | 258 | GPGSQVQLQESGGGLVQAGDSLRLSCAASGFDEDNFDDYAIGWFRQ |
| acetylcholine | | APGQEREGVSCIDPSDGSTIYADSAKGRFTISSDNAENTVYLQMNS |
| receptor M2 | | LKPEDTAVYVCSAWTLFHSDEYWGQGTQVTVSS |
| human | | |
| (monobody) | | |

EnDUBS

| | | |
|---|---|---|
| Cezanne | 259 | PPSFSEGSGGSRTPEKGFSDREPTRPPRPILQRQDDIVQEKRLSRG |
| | | ISHASSSIVSLARSHVSSNGGGGGSNEHPLEMPICAFQLPDLTVYN |
| | | EDERSFIERDLIEQSMLVALEQAGRLNWWVSVDPTSQRLLPLATTG |
| | | DGNCLLHAASLGMWGFHDRDLMLRKALYALMEKGVEKEALKRRWRW |
| | | QQTQQNKESGLVYTEDEWQKEWNELIKLASSEPRMHLGTNGANCGG |
| | | VESSEEPVYESLEEFHVFVLAHVLRRPIVVVADTMLRDSGGEAFAP |
| | | IPFGGIYLPLEVPASQCHRSPLVLAYDQAHFSALVSMEQKENTKEQ |
| | | AVIPLTDSEYKLLPLHFAVDPGKGWEWGKDDSDNVRLASVILSLEV |
| | | KLHLLHSYMNVKWIPLSSDAQAPLAQ |
| OTUD1 | 260 | DEKLALYLAEVEKQDKYLRQRNKYRFHIIPDGNCLYRAVSKTVYGD |
| | | QSLHRELREQTVHYIADHLDHFSPLIEGDVGEFIIAAAQDGAWAGY |
| | | PELLAMGQMLNVNIHLTTGGRLESPTVSTMIHYLGPEDSLRPSIWL |
| | | SWLSNGHYDAVEDHSYPNPEYDNWCKQTQVQRKRDEELAKSMAISL |
| | | SKMYIEQNACS |
| TRABID | 261 | LEVDFKKLKQIKNRMKKTDWLFLNACVGVVEGDLAAIEAYKSSGGD |
| | | IARQLTADEVRLLNRPSAFDVGYTLVHLAIRFQRQDMLAILLTEVS |
| | | QQAAKCIPAMVCPELTEQIRREIAASLHQRKGDFACYFLTDLVTFT |
| | | LPADIEDLPPTVQEKLFDEVLDRDVQKELEEESPIINWSLELATRL |
| | | DSRLYALWNRTAGDCLLDSVLQATWGIYDKDSVLRKALHDSLHDCS |
| | | HWFYTRWKDWESWYSQSFGLHESLREEQWQEDWAFILSLASQPGAS |
| | | LEQTHIFVLAHILRRPIIVYGVKYYKSFRGETLGYTRFQGVYLPLL |
| | | WEQSFCWKSPIALGYTRGHFSALVAMENDGYGNRGAGANLNTDDDV |
| | | TITFLPLVDSERKLLHVHELSAQELGNEEQQEKLLREWLDCCVTEG |
| | | GVLVAMQKSSRRRNHPLVTQMVEKWLDRYRQIRPCTSLS |
| USP21 | 262 | SDDKMAHHTLLLGSGHVGLRNLGNTCELNAVLQCLSSTRPLRDFCL |
| | | RRDFRQEVPGGGRAQELTEAFADVIGALWHPDSCEAVNPTRFRAVE |
| | | QKYVPSFSGYSQQDAQEFLKLLMERLHLEINRRGRRAPPILANGPV |
| | | PSPPRRGGALLEEPELSDDDRANLMWKRYLEREDSKIVDLFVGQLK |
| | | SCLKCQACGYRSTTFEVFCDLSLPIPKKGFAGGKVSLRDCFNLFTK |
| | | EEELESENAPVCDRCRQKTRSTKKLTVQRFPRILVLHLNRESASRG |
| | | SIKKSSVGVDFPLQRLSLGDFASDKAGSPVYQLYALCNHSGSVHYG |
| | | HYTALCRCQTGWHVYNDSRVSPVSENQVASSEGYVLFYQLMQEPPR |
| | | CL |
| OTUD4 | 263 | ATPMDAYLRKLGLYRKLVAKDGSCLFRAVAEQVLHSQSRHVEVRMA |
| | | CIHYLRENREKFEAFIEGSFEEYLKRLENPQEWVGQVEISALSLMY |
| | | RKDFIIYREPNVSPSQVTENNFPEKVLLCFSNGNHYDIVYPIKYKE |
| | | SSAMCQSLLYELLYEKVEKTDVSKIVMELDTLEVADE |
| Human USP3 | 264 | MECPHLSSSVCIAPDSAKFPNGSPSSWCCSVCRSNKSPWVCLTCSS |
| (full length) | | VHCGRYVNGHAKKHYEDAQVPLTNHKKSEKQDKVQHTVCMDCSSYS |
| nuclear located | | TYCYRCDDFVVNDTKLGLVQKVREHLQNLENSAFTADRHKKRKLLE |
| | | NSTLNSKLLKVNGSTTAICATGLRNLGNTCEMNAILQSLSNIEQFC |
| | | CYFKELPAVELRNGKTAGRRTYHTRSQGDNNVSLVEEFRKTLCALW |
| | | QGSQTAFSPESLFYVVWKIMPNERGYQQQDAHEFMRYLLDHLHLEL |
| | | QGGFNGVSRSAILQENSTLSASNKCCINGASTVVTAIFGGILQNEV |
| | | NCLICGTESRKFDPELDLSLDIPSQFRSKRSKNQENGPVCSLRDCL |
| | | RSFTDLEELDETELYMCHKCKKKQKSTKKFWIQKLPKVLCLHLKRF |
| | | HWTAYLRNKVDTYVEFPLRGLDMKCYLLEPENSGPESCLYDLAAVV |
| | | VHHGSGVGSGHYTAYATHEGRWFHENDSTVTLTDEETVVKAKAYIL |
| | | FYVEHQAKAGSDKL |

The amino acid sequence of the test fusion proteins is provided in Table 5 below.

TABLE 5
Amino acid sequence of exemplary test fusion proteins

| Description | SEQ ID NO | Amino Acid Sequence |
|---|---|---|
| Beta 2 | 265 | MGQPGNGSAFLLAPNGSHAPDHDVTQERDEVWVVGMGIVMSLIVLA |
| adrenergic | | IVFGNVLVITAIAKFERLQTVTNYFITSLACADLVMGLAVVPFGAA |
| receptor human | | HILMKMWTFGNFWCEFWTSIDVLCVTASIETLCVIAVDRYFAITSP |
| Target-YFP- | | FKYQSLLTKNKARVIILMVWIVSGLTSELPIQMHWYRATHQEAINC |
| P2A-mCherrry | | YANETCCDFFTNQAYAIASSIVSFYVPLVIMVFVYSRVFQEAKRQL |
| | | QKIDKSEGRFHVQNLSQVEQDGRTGHGLRRSSKFCLKEHKALKTLG |
| | | IIMGTFTLCWLPFFIVNIVHVIQDNLIRKEVYILLNWIGYVNSGEN |
| | | PLIYCRSPDFRIAFQELLCLRRSSLKAYGNGYSSNGNTGEQSGYHV |
| | | EQEKENKLLCEDLPGTEDFVGHQGTVPSDNIDSQGRNCSTNDSLLV |
| | | SKGEELFTGVVPILVELDGDVNGHKESVSGEGEGDATYGKLTLKFI |
| | | CTTGKLPVPWPTLVTTFGYGLQCFARYPDHMKQHDFFKSAMPEGYV |
| | | QERTIFFKDDGNYKTRAEVKFEGDTLVNRIELKGIDFKEDGNILGH |
| | | KLEYNYNSHNVYIMADKQKNGIKVNFKIRHNIEDGSVQLADHYQQN |
| | | TPIGDGPVLLPDNHYLSYQSALSKDPNEKRDHMVLLEFVTAAGITL |
| | | GMDELYKGSGATNFSLLKQAGDVEENPGPMVSKGEEDNMAIIKEFM |
| | | RFKVHMEGSVNGHEFEIEGEGEGRPYEGTQTAKLKVTKGGPLPFAW |
| | | DILSPQFMYGSKAYVKHPADIPDYLKLSFPEGEKWERVMNFEDGGV |
| | | VTVTQDSSLQDGEFIYKVKLRGTNFPSDGPVMQKKTMGWEASSERM |
| | | YPEDGALKGEIKQRLKLKDGGHYDAEVKTTYKAKKPVQLPGAYNVN |
| | | IKLDITSHNEDYTIVEQYERAEGRHSTGGMDELYK |
| Human kappa- | 266 | MDSPIQIFRGEPGPTCAPSACLPPNSSAWFPGWAEPDSNGSAGSED |
| type opioid- | | AQLEPAHISPAIPVIITAVYSVVFVVGLVGNSLVMFVIIRYTKMKT |
| receptor Target | | ATNIYIFNLALADALVTTTMPFQSTVYLMNSWPFGDVLCKIVISID |
| YFP-P2A- | | YYNMFTSIFTLTMMSVDRYIAVCHPVKALDERTPLKAKIINICIWL |
| mCherrry | | LSSSVGISAIVLGGTKVREDVDVIECSLQFPDDDYSWWDLEMKICV |
| | | FIFAFVIPVLIIIVCYTLMILRLKSVRLLSGSREKDRNLRRITRLV |
| | | LVVVAVFVVCWTPIHIFILVEALGSTSHSTAALSSYYFCIALGYTN |
| | | SSLNPILYAFLDENFKRCFRDFCFPLKMRMERQSTSRVRNTVQDPA |
| | | YLRDIDGMNKPVVSKGEELFTGVVPILVELDGDVNGHKESVSGEGE |
| | | GDATYGKLTLKFICTTGKLPVPWPTLVTTFGYGLQCFARYPDHMKQ |
| | | HDFFKSAMPEGYVQERTIFFKDDGNYKTRAEVKFEGDTLVNRIELK |
| | | GIDFKEDGNILGHKLEYNYNSHNVYIMADKQKNGIKVNFKIRHNIE |
| | | DGSVQLADHYQQNTPIGDGPVLLPDNHYLSYQSALSKDPNEKRDHM |
| | | VLLEFVTAAGITLGMDELYKGSGATNFSLLKQAGDVEENPGPMVSK |
| | | GEEDNMAIIKEFMRFKVHMEGSVNGHEFEIEGEGEGRPYEGTQTAK |
| | | LKVTKGGPLPFAWDILSPQFMYGSKAYVKHPADIPDYLKLSFPEGE |
| | | KWERVMNFEDGGVVTVTQDSSLQDGEFIYKVKLRGTNFPSDGPVMQ |
| | | KKTMGWEASSERMYPEDGALKGEIKQRLKLKDGGHYDAEVKTTYKA |
| | | KKPVQLPGAYNVNIKLDITSHNEDYTIVEQYERAEGRHSTGGMDEL |
| | | YK |
| Muscarinic | 267 | MNNSTNSSNNSLALTSPYKTFEVVFIVLVAGSLSLVTIIGNILVMV |
| acetylcholine | | SIKVNRHLQTVNNYFLESLACADLIIGVESMNLYTLYTVIGYWPLG |
| receptor M2 | | PVVCDLWLALDYVVSNASVMNLLIISEDRYFCVTKPLTYPVKRTTK |
| Target-YFP- | | MAGMMIAAAWVLSFILWAPAILFWQFIVGVRTVEDGECYIQFFSNA |
| P2A-mCherrry | | AVTFGTAIAAFYLPVIIMTVLYWHISRASKSRIKKDKKEPVANQDP |
| | | VSPSLVQGRIVKPNNNNMPSSDDGLEHNKIQNGKAPRDPVTENCVQ |
| | | GEEKESSNDSTSVSAVASNMRDDEITQDENTVSTSLGHSKDENSKQ |
| | | TCIRIGTKTPKSDSCTPTNTTVEVVGSSGQNGDEKQNIVARKIVKM |
| | | TKQPAKKKPPPSREKKVTRTILAILLAFIITWAPYNVMVLINTFCA |
| | | PCIPNTVWTIGYWLCYINSTINPACYALCNATFKKTFKHLLMCHYK |
| | | NIGATRVSKGEELFTGVVPILVELDGDVNGHKESVSGEGEGDATYG |
| | | KLTLKFICTTGKLPVPWPTLVTTFGYGLQCFARYPDHMKQHDFFKS |
| | | AMPEGYVQERTIFFKDDGNYKTRAEVKFEGDTLVNRIELKGIDFKE |
| | | DGNILGHKLEYNYNSHNVYIMADKQKNGIKVNFKIRHNIEDGSVQL |
| | | ADHYQQNTPIGDGPVLLPDNHYLSYQSALSKDPNEKRDHMVLLEFV |
| | | TAAGITLGMDELYKGSGATNFSLLKQAGDVEENPGPMVSKGEEDNM |
| | | AIIKEFMRFKVHMEGSVNGHEFEIEGEGEGRPYEGTQTAKLKVTKG |
| | | GPLPFAWDILSPQFMYGSKAYVKHPADIPDYLKLSFPEGEKWERVM |
| | | NEEDGGVVTVTQDSSLQDGEFIYKVKLRGTNFPSDGPVMQKKTMGW |
| | | EASSERMYPEDGALKGEIKQRLKLKDGGHYDAEVKTTYKAKKPVQL |
| | | PGAYNVNIKLDITSHNEDYTIVEQYERAEGRHSTGGMDELYK |
| CFP-P2A- | 268 | MVSKGEELFTGVVPILVELDGDVNGHKFSVSGEGEGDATYGKLTLK |
| Cezanne enDUB | | FICTTGKLPVPWPTLVTTLTWGVQCFSRYPDHMKQHDEFKSAMPEG |
| | | YVQERTIFFKDDGNYKTRAEVKFEGDTLVNRIELKGIDFKEDGNIL |
| | | GHKLEYNYISHNVYITADKQKNGIKANFKIRHNIEDGSVQLADHYQ |
| | | QNTPIGDGPVLLPDNHYLSTQSALSKDPNEKRDHMVLLEFVTAAGI |
| | | TLGMDELYKGSGATNESLLKQAGDVEENPGPPPSFSEGSGGSRTPE |
| | | KGFSDREPTRPPRPILQRQDDIVQEKRLSRGISHASSSIVSLARSH |
| | | VSSNGGGGGSNEHPLEMPICAFQLPDLTVYNEDERSFIERDLIEQS |
| | | MLVALEQAGRLNWWVSVDPTSQRLLPLATTGDGNCLLHAASLGMWG |
| | | FHDRDLMLRKALYALMEKGVEKEALKRRWRWQQTQQNKESGLVYTE |
| | | DEWQKEWNELIKLASSEPRMHLGTNGANCGGVESSEEPVYESLEEF |
| | | HVFVLAHVLRRPIVVVADTMLRDSGGEAFAPIPEGGIYLPLEVPAS |
| | | QCHRSPLVLAYDQAHFSALVSMEQKENTKEQAVIPLTDSEYKLLPL |
| | | HFAVDPGKGWEWGKDDSDNVRLASVILSLEVKLHLLHSYMNVKWIP |
| | | LSSDAQAPLAQ |
| CFP-P2A- | 269 | MVSKGEELFTGVVPILVELDGDVNGHKFSVSGEGEGDATYGKLTLK |
| OTUD1 enDUB | | FICTTGKLPVPWPTLVTTLTWGVQCFSRYPDHMKQHDFFKSAMPEG |
| | | YVQERTIFFKDDGNYKTRAEVKFEGDTLVNRIELKGIDFKEDGNIL |
| | | GHKLEYNYISHNVYITADKQKNGIKANFKIRHNIEDGSVQLADHYQ |
| | | QNTPIGDGPVLLPDNHYLSTQSALSKDPNEKRDHMVLLEFVTAAGI |
| | | TLGMDELYKGSGATNESLLKQAGDVEENPGPDEKLALYLAEVEKQD |
| | | KYLRQRNKYRFHIIPDGNCLYRAVSKTVYGDQSLHRELREQTVHYI |
| | | ADHLDHFSPLIEGDVGEFIIAAAQDGAWAGYPELLAMGQMLNVNIH |
| | | LTTGGRLESPTVSTMIHYLGPEDSLRPSIWLSWLSNGHYDAVEDHS |
| | | YPNPEYDNWCKQTQVQRKRDEELAKSMAISLSKMYIEQNACS |
| CFP-P2A- | 270 | MVSKGEELFTGVVPILVELDGDVNGHKFSVSGEGEGDATYGKLTLK |
| TRABID | | FICTTGKLPVPWPTLVTTLTWGVQCFSRYPDHMKQHDFFKSAMPEG |
| enDUB | | YVQERTIFFKDDGNYKTRAEVKFEGDTLVNRIELKGIDFKEDGNIL |
| | | GHKLEYNYISHNVYITADKQKNGIKANFKIRHNIEDGSVQLADHYQ |
| | | QNTPIGDGPVLLPDNHYLSTQSALSKDPNEKRDHMVLLEFVTAAGI |
| | | TLGMDELYKGSGATNFSLLKQAGDVEENPGPLEVDEKKLKQIKNRM |
| | | KKTDWLFLNACVGVVEGDLAAIEAYKSSGGDIARQLTADEVRLLNR |
| | | PSAFDVGYTLVHLAIRFQRQDMLAILLTEVSQQAAKCIPAMVCPEL |
| | | TEQIRREIAASLHQRKGDFACYFLTDLVTFTLPADIEDLPPTVQEK |
| | | LFDEVLDRDVQKELEEESPIINWSLELATRLDSRLYALWNRTAGDC |
| | | LLDSVLQATWGIYDKDSVLRKALHDSLHDCSHWFYTRWKDWESWYS |
| | | QSFGLHESLREEQWQEDWAFILSLASQPGASLEQTHIFVLAHILRR |
| | | PIIVYGVKYYKSFRGETLGYTRFQGVYLPLLWEQSFCWKSPIALGY |
| | | TRGHFSALVAMENDGYGNRGAGANLNTDDDVTITFLPLVDSERKLL |
| | | HVHELSAQELGNEEQQEKLLREWLDCCVTEGGVLVAMQKSSRRRNH |
| | | PLVTQMVEKWLDRYRQIRPCTSLS |
| CFP-P2A- | 271 | MVSKGEELFTGVVPILVELDGDVNGHKFSVSGEGEGDATYGKLTLK |
| USP21 enDUB | | FICTTGKLPVPWPTLVTTLTWGVQCESRYPDHMKQHDFFKSAMPEG |
| | | YVQERTIFFKDDGNYKTRAEVKFEGDTLVNRIELKGIDFKEDGNIL |
| | | GHKLEYNYISHNVYITADKQKNGIKANFKIRHNIEDGSVQLADHYQ |
| | | QNTPIGDGPVLLPDNHYLSTQSALSKDPNEKRDHMVLLEFVTAAGI |
| | | TLGMDELYKGSGATNFSLLKQAGDVEENPGPSDDKMAHHTLLLGSG |
| | | HVGLRNLGNTCFLNAVLQCLSSTRPLRDFCLRRDFRQEVPGGGRAQ |
| | | ELTEAFADVIGALWHPDSCEAVNPTRFRAVFQKYVPSFSGYSQQDA |
| | | QEFLKLLMERLHLEINRRGRRAPPILANGPVPSPPRRGGALLEEPE |
| | | LSDDDRANLMWKRYLEREDSKIVDLFVGQLKSCLKCQACGYRSTTE |
| | | EVFCDLSLPIPKKGFAGGKVSLRDCFNLFTKEEELESENAPVCDRC |
| | | RQKTRSTKKLTVQRFPRILVLHLNRFSASRGSIKKSSVGVDFPLQR |
| | | LSLGDFASDKAGSPVYQLYALCNHSGSVHYGHYTALCRCQTGWHVY |
| | | NDSRVSPVSENQVASSEGYVLFYQLMQEPPRCL |
| CFP-P2A- | 272 | MVSKGEELFTGVVPILVELDGDVNGHKFSVSGEGEGDATYGKLTLK |
| OTUD4 enDUB | | FICTTGKLPVPWPTLVTTLTWGVQCFSRYPDHMKQHDFFKSAMPEG |
| | | YVQERTIFFKDDGNYKTRAEVKFEGDTLVNRIELKGIDFKEDGNIL |
| | | GHKLEYNYISHNVYITADKQKNGIKANFKIRHNIEDGSVQLADHYQ |
| | | QNTPIGDGPVLLPDNHYLSTQSALSKDPNEKRDHMVLLEFVTAAGI |
| | | TLGMDELYKGSGATNFSLLKQAGDVEENPGPATPMDAYLRKLGLYR |
| | | KLVAKDGSCLFRAVAEQVLHSQSRHVEVRMACIHYLRENREKFEAF |
| | | IEGSFEEYLKRLENPQEWVGQVEISALSLMYRKDFIIYREPNVSPS |
| | | QVTENNFPEKVLLCESNGNHYDIVYPIKYKESSAMCQSLLYELLYE |
| | | KVFKTDVSKIVMELDTLEVADE |
| CFP-P2A-a- | 273 | MVSKGEELFTGVVPILVELDGDVNGHKFSVSGEGEGDATYGKLTLK |
| YFPnanobody- | | FICTTGKLPVPWPTLVTTLTWGVQCFSRYPDHMKQHDFFKSAMPEG |
| Cezanne enDUB | | YVQERTIFFKDDGNYKTRAEVKFEGDTLVNRIELKGIDFKEDGNIL |
| | | GHKLEYNYISHNVYITADKQKNGIKANFKIRHNIEDGSVQLADHYQ |
| | | QNTPIGDGPVLLPDNHYLSTQSALSKDPNEKRDHMVLLEFVTAAGI |
| | | TLGMDELYKGSGATNFSLLKQAGDVEENPGPQVQLVESGGALVQPG |
| | | GSLRLSCAASGFPVNRYSMRWYRQAPGKEREWVAGMSSAGDRSSYE |
| | | DSVKGRFTISRDDARNTVYLQMNSLKPEDTAVYYCNVNVGFEYWGQ |
| | | GTQVTVSSPPSFSEGSGGSRTPEKGESDREPTRPPRPILQRQDDIV |
| | | QEKRLSRGISHASSSIVSLARSHVSSNGGGGGSNEHPLEMPICAFQ |
| | | LPDLTVYNEDERSFIERDLIEQSMLVALEQAGRLNWWVSVDPTSQR |
| | | LLPLATTGDGNCLLHAASLGMWGFHDRDLMLRKALYALMEKGVEKE |
| | | ALKRRWRWQQTQQNKESGLVYTEDEWQKEWNELIKLASSEPRMHLG |
| | | TNGANCGGVESSEEPVYESLEEFHVFVLAHVLRRPIVVVADTMLRD |
| | | SGGEAFAPIPEGGIYLPLEVPASQCHRSPLVLAYDQAHFSALVSME |
| | | QKENTKEQAVIPLTDSEYKLLPLHFAVDPGKGWEWGKDDSDNVRLA |
| | | SVILSLEVKLHLLHSYMNVKWIPLSSDAQAPLAQ |
| CFP-P2A-a- | 274 | MVSKGEELFTGVVPILVELDGDVNGHKFSVSGEGEGDATYGKLTLK |
| YFPnanobody- | | FICTTGKLPVPWPTLVTTLTWGVQCFSRYPDHMKQHDFFKSAMPEG |
| OTUD1 enDUB | | YVQERTIFFKDDGNYKTRAEVKFEGDTLVNRIELKGIDFKEDGNIL |
| | | GHKLEYNYISHNVYITADKQKNGIKANFKIRHNIEDGSVQLADHYQ |
| | | QNTPIGDGPVLLPDNHYLSTQSALSKDPNEKRDHMVLLEFVTAAGI |
| | | TLGMDELYKGSGATNFSLLKQAGDVEENPGPQVQLVESGGALVQPG |
| | | GSLRLSCAASGFPVNRYSMRWYRQAPGKEREWVAGMSSAGDRSSYE |
| | | DSVKGRFTISRDDARNTVYLQMNSLKPEDTAVYYCNVNVGFEYWGQ |
| | | GTQVTVSSDEKLALYLAEVEKQDKYLRQRNKYRFHIIPDGNCLYRA |
| | | VSKTVYGDQSLHRELREQTVHYIADHLDHESPLIEGDVGEFIIAAA |
| | | QDGAWAGYPELLAMGQMLNVNIHLTTGGRLESPTVSTMIHYLGPED |
| | | SLRPSIWLSWLSNGHYDAVEDHSYPNPEYDNWCKQTQVQRKRDEEL |
| | | AKSMAISLSKMYIEQNACS |
| CFP-P2A-a- | 275 | MVSKGEELFTGVVPILVELDGDVNGHKFSVSGEGEGDATYGKLTLK |
| YFPnanobody- | | FICTTGKLPVPWPTLVTTLTWGVQCFSRYPDHMKQHDFFKSAMPEG |
| TRABID | | YVQERTIFFKDDGNYKTRAEVKFEGDTLVNRIELKGIDFKEDGNIL |
| enDUB | | GHKLEYNYISHNVYITADKQKNGIKANFKIRHNIEDGSVQLADHYQ |
| | | QNTPIGDGPVLLPDNHYLSTQSALSKDPNEKRDHMVLLEFVTAAGI |
| | | TLGMDELYKGSGATNFSLLKQAGDVEENPGPQVQLVESGGALVQPG |
| | | GSLRLSCAASGFPVNRYSMRWYRQAPGKEREWVAGMSSAGDRSSYE |
| | | DSVKGRFTISRDDARNTVYLQMNSLKPEDTAVYYCNVNVGFEYWGQ |
| | | GTQVTVSSLEVDFKKLKQIKNRMKKTDWLFLNACVGVVEGDLAAIE |
| | | AYKSSGGDIARQLTADEVRLLNRPSAFDVGYTLVHLAIRFQRQDML |
| | | AILLTEVSQQAAKCIPAMVCPELTEQIRREIAASLHQRKGDFACYF |
| | | LTDLVTFTLPADIEDLPPTVQEKLEDEVLDRDVQKELEEESPIINW |
| | | SLELATRLDSRLYALWNRTAGDCLLDSVLQATWGIYDKDSVLRKAL |
| | | HDSLHDCSHWFYTRWKDWESWYSQSFGLHESLREEQWQEDWAFILS |
| | | LASQPGASLEQTHIFVLAHILRRPIIVYGVKYYKSFRGETLGYTRE |
| | | QGVYLPLLWEQSFCWKSPIALGYTRGHFSALVAMENDGYGNRGAGA |
| | | NLNTDDDVTITELPLVDSERKLLHVHELSAQELGNEEQQEKLLREW |
| | | LDCCVTEGGVLVAMQKSSRRRNHPLVTQMVEKWLDRYRQIRPCTSL |
| CFP-P2A-a- | 276 | MVSKGEELFTGVVPILVELDGDVNGHKESVSGEGEGDATYGKLTLK |
| YFPnanobody- | | FICTTGKLPVPWPTLVTTLTWGVQCFSRYPDHMKQHDFFKSAMPEG |
| USP21 enDUB | | YVQERTIFFKDDGNYKTRAEVKFEGDTLVNRIELKGIDFKEDGNIL |
| | | GHKLEYNYISHNVYITADKQKNGIKANFKIRHNIEDGSVQLADHYQ |
| | | QNTPIGDGPVLLPDNHYLSTQSALSKDPNEKRDHMVLLEFVTAAGI |
| | | TLGMDELYKGSGATNFSLLKQAGDVEENPGPQVQLVESGGALVQPG |
| | | GSLRLSCAASGFPVNRYSMRWYRQAPGKEREWVAGMSSAGDRSSYE |
| | | DSVKGRFTISRDDARNTVYLQMNSLKPEDTAVYYCNVNVGFEYWGQ |
| | | GTQVTVSSSDDKMAHHTLLLGSGHVGLRNLGNTCFLNAVLQCLSST |
| | | RPLRDFCLRRDFRQEVPGGGRAQELTEAFADVIGALWHPDSCEAVN |
| | | PTRFRAVFQKYVPSFSGYSQQDAQEFLKLLMERLHLEINRRGRRAP |
| | | PILANGPVPSPPRRGGALLEEPELSDDDRANLMWKRYLEREDSKIV |
| | | DLFVGQLKSCLKCQACGYRSTTFEVECDLSLPIPKKGFAGGKVSLR |
| | | DCFNLFTKEEELESENAPVCDRCRQKTRSTKKLTVQRFPRILVLHL |
| | | NRFSASRGSIKKSSVGVDFPLQRLSLGDFASDKAGSPVYQLYALCN |
| | | HSGSVHYGHYTALCRCQTGWHVYNDSRVSPVSENQVASSEGYVLFY |
| | | QLMQEPPRCL |
| CFP-P2A-a- | 277 | MVSKGEELFTGVVPILVELDGDVNGHKFSVSGEGEGDATYGKLTLK |
| YFPnanobody- | | FICTTGKLPVPWPTLVTTLTWGVQCESRYPDHMKQHDFFKSAMPEG |
| OTUD4 enDUB | | YVQERTIFFKDDGNYKTRAEVKFEGDTLVNRIELKGIDFKEDGNIL |
| | | GHKLEYNYISHNVYITADKQKNGIKANFKIRHNIEDGSVQLADHYQ |
| | | QNTPIGDGPVLLPDNHYLSTQSALSKDPNEKRDHMVLLEFVTAAGI |
| | | TLGMDELYKGSGATNFSLLKQAGDVEENPGPQVQLVESGGALVQPG |
| | | GSLRLSCAASGFPVNRYSMRWYRQAPGKEREWVAGMSSAGDRSSYE |
| | | DSVKGRFTISRDDARNTVYLQMNSLKPEDTAVYYCNVNVGFEYWGQ |
| | | GTQVTVSSATPMDAYLRKLGLYRKLVAKDGSCLFRAVAEQVLHSQS |
| | | RHVEVRMACIHYLRENREKFEAFIEGSFEEYLKRLENPQEWVGQVE |
| | | ISALSLMYRKDFIIYREPNVSPSQVTENNFPEKVLLCESNGNHYDI |
| | | VYPIKYKESSAMCQSLLYELLYEKVFKTDVSKIVMELDTLEVADE |
| CFP-P2A-anti- | 278 | MVSKGEELFTGVVPILVELDGDVNGHKESVSGEGEGDATYGKLTLK |
| beta 2 | | FICTTGKLPVPWPTLVTTLTWGVQCFSRYPDHMKQHDFFKSAMPEG |
| adrenergic | | YVQERTIFFKDDGNYKTRAEVKFEGDTLVNRIELKGIDFKEDGNIL |
| receptor | | GHKLEYNYISHNVYITADKQKNGIKANFKIRHNIEDGSVQLADHYQ |
| targeting binder- | | QNTPIGDGPVLLPDNHYLSTQSALSKDPNEKRDHMVLLEFVTAAGI |
| Cezanne enDUB | | TLGMDELYKGSGATNESLLKQAGDVEENPGPQVQLQESGGGLVQAG |
| | | GSLRLSCAASGSIFALNIMGWYRQAPGKQRELVAAIHSGGTTNYAN |
| | | SVKGRFTISRDNAANTVYLQMNSLKPEDTAVYYCNVKDEGAIIYDY |
| | | DYWGQGTQVTVSSPPSFSEGSGGSRTPEKGESDREPTRPPRPILQR |
| | | QDDIVQEKRLSRGISHASSSIVSLARSHVSSNGGGGGSNEHPLEMP |
| | | ICAFQLPDLTVYNEDERSFIERDLIEQSMLVALEQAGRLNWWVSVD |
| | | PTSQRLLPLATTGDGNCLLHAASLGMWGFHDRDLMLRKALYALMEK |
| | | GVEKEALKRRWRWQQTQQNKESGLVYTEDEWQKEWNELIKLASSEP |
| | | RMHLGTNGANCGGVESSEEPVYESLEEFHVEVLAHVLRRPIVVVAD |
| | | TMLRDSGGEAFAPIPFGGIYLPLEVPASQCHRSPLVLAYDQAHFSA |
| | | LVSMEQKENTKEQAVIPLTDSEYKLLPLHFAVDPGKGWEWGKDDSD |
| | | NVRLASVILSLEVKLHLLHSYMNVKWIPLSSDAQAPLAQ |
| CFP-P2A-anti- | 279 | MVSKGEELFTGVVPILVELDGDVNGHKESVSGEGEGDATYGKLTLK |
| beta 2 | | FICTTGKLPVPWPTLVTTLTWGVQCESRYPDHMKQHDFFKSAMPEG |
| adrenergic | | YVQERTIFFKDDGNYKTRAEVKFEGDTLVNRIELKGIDFKEDGNIL |
| receptor | | GHKLEYNYISHNVYITADKQKNGIKANFKIRHNIEDGSVQLADHYQ |
| targeting binder- | | QNTPIGDGPVLLPDNHYLSTQSALSKDPNEKRDHMVLLEFVTAAGI |
| OTUD1 enDUB | | TLGMDELYKGSGATNFSLLKQAGDVEENPGPQVQLQESGGGLVQAG |
| | | GSLRLSCAASGSIFALNIMGWYRQAPGKQRELVAAIHSGGTTNYAN |
| | | SVKGRFTISRDNAANTVYLQMNSLKPEDTAVYYCNVKDEGAIIYDY |
| | | DYWGQGTQVTVSSDEKLALYLAEVEKQDKYLRQRNKYRFHIIPDGN |
| | | CLYRAVSKTVYGDQSLHRELREQTVHYIADHLDHESPLIEGDVGEF |
| | | IIAAAQDGAWAGYPELLAMGQMLNVNIHLTTGGRLESPTVSTMIHY |
| | | LGPEDSLRPSIWLSWLSNGHYDAVEDHSYPNPEYDNWCKQTQVQRK |
| | | RDEELAKSMAISLSKMYIEQNACS |
| CFP-P2A-anti- | 280 | MVSKGEELFTGVVPILVELDGDVNGHKESVSGEGEGDATYGKLTLK |
| beta 2 | | FICTTGKLPVPWPTLVTTLTWGVQCFSRYPDHMKQHDFFKSAMPEG |
| adrenergic | | YVQERTIFFKDDGNYKTRAEVKFEGDTLVNRIELKGIDFKEDGNIL |
| receptor | | GHKLEYNYISHNVYITADKQKNGIKANFKIRHNIEDGSVQLADHYQ |
| targeting binder- | | QNTPIGDGPVLLPDNHYLSTQSALSKDPNEKRDHMVLLEFVTAAGI |
| TRABID | | TLGMDELYKGSGATNFSLLKQAGDVEENPGPQVQLQESGGGLVQAG |
| enDUB | | GSLRLSCAASGSIFALNIMGWYRQAPGKQRELVAAIHSGGTTNYAN |
| | | SVKGRFTISRDNAANTVYLQMNSLKPEDTAVYYCNVKDEGAIIYDY |
| | | DYWGQGTQVTVSSLEVDFKKLKQIKNRMKKTDWLFLNACVGVVEGD |
| | | LAAIEAYKSSGGDIARQLTADEVRLLNRPSAFDVGYTLVHLAIRFQ |
| | | RQDMLAILLTEVSQQAAKCIPAMVCPELTEQIRREIAASLHQRKGD |
| | | FACYFLTDLVTFTLPADIEDLPPTVQEKLEDEVLDRDVQKELEEES |
| | | PIINWSLELATRLDSRLYALWNRTAGDCLLDSVLQATWGIYDKDSV |
| | | LRKALHDSLHDCSHWFYTRWKDWESWYSQSFGLHESLREEQWQEDW |
| | | AFILSLASQPGASLEQTHIFVLAHILRRPIIVYGVKYYKSERGETL |
| | | GYTRFQGVYLPLLWEQSFCWKSPIALGYTRGHFSALVAMENDGYGN |
| | | RGAGANLNTDDDVTITFLPLVDSERKLLHVHELSAQELGNEEQQEK |
| | | LLREWLDCCVTEGGVLVAMQKSSRRRNHPLVTQMVEKWLDRYRQIR |
| | | PCTSLS |
| CFP-P2A-anti- | 281 | MVSKGEELFTGVVPILVELDGDVNGHKFSVSGEGEGDATYGKLTLK |
| beta 2 | | FICTTGKLPVPWPTLVTTLTWGVQCFSRYPDHMKQHDFFKSAMPEG |
| adrenergic | | YVQERTIFFKDDGNYKTRAEVKFEGDTLVNRIELKGIDFKEDGNIL |
| receptor | | GHKLEYNYISHNVYITADKQKNGIKANFKIRHNIEDGSVQLADHYQ |
| targeting binder- | | QNTPIGDGPVLLPDNHYLSTQSALSKDPNEKRDHMVLLEFVTAAGI |
| USP21 enDUB | | TLGMDELYKGSGATNE |
| | | QVQLQESGGGLVQAGGSLRLSCAASGSIFALNIMGWYRQAPGKQRE |
| | | LVAAIHSGGTTNYANSVKGRFTISRDNAANTVYLQMNSLKPEDTAV |
| | | YYCNVKDFGAIIYDYDYWGQGTQVTVSSSDDKMAHHTLLLGSGHVG |
| | | LRNLGNTCFLNAVLQCLSSTRPLRDFCLRRDERQEVPGGGRAQELT |
| | | EAFADVIGALWHPDSCEAVNPTRFRAVFQKYVPSFSGYSQQDAQEF |
| | | LKLLMERLHLEINRRGRRAPPILANGPVPSPPRRGGALLEEPELSD |
| | | DDRANLMWKRYLEREDSKIVDLFVGQLKSCLKCQACGYRSTTFEVE |
| | | CDLSLPIPKKGFAGGKVSLRDCENLFTKEEELESENAPVCDRCRQK |
| | | TRSTKKLTVQRFPRILVLHLNRESASRGSIKKSSVGVDFPLQRLSL |
| | | GDFASDKAGSPVYQLYALCNHSGSVHYGHYTALCRCQTGWHVYNDS |
| | | RVSPVSENQVASSEGYVLFYQLMQEPPRCL |
| CFP-P2A-anti- | 282 | MVSKGEELFTGVVPILVELDGDVNGHKESVSGEGEGDATYGKLTLK |
| beta 2 | | FICTTGKLPVPWPTLVTTLTWGVQCFSRYPDHMKQHDFFKSAMPEG |
| adrenergic | | YVQERTIFFKDDGNYKTRAEVKFEGDTLVNRIELKGIDFKEDGNIL |
| receptor | | GHKLEYNYISHNVYITADKQKNGIKANFKIRHNIEDGSVQLADHYQ |
| targeting binder- | | QNTPIGDGPVLLPDNHYLSTQSALSKDPNEKRDHMVLLEFVTAAGI |
| OTUD4 enDUB | | TLGMDELYKGSGATNFSLLKQAGDVEENPGPQVQLQESGGGLVQAG |
| | | GSLRLSCAASGSIFALNIMGWYRQAPGKQRELVAAIHSGGTTNYAN |
| | | SVKGRFTISRDNAANTVYLQMNSLKPEDTAVYYCNVKDEGAIIYDY |
| | | DYWGQGTQVTVSSATPMDAYLRKLGLYRKLVAKDGSCLFRAVAEQV |
| | | LHSQSRHVEVRMACIHYLRENREKFEAFIEGSFEEYLKRLENPQEW |
| | | VGQVEISALSLMYRKDFIIYREPNVSPSQVTENNFPEKVLLCESNG |
| | | NHYDIVYPIKYKESSAMCQSLLYELLYEKVFKTDVSKIVMELDTLE |
| | | VADE |
| CFP-P2A-anti- | 283 | MVSKGEELFTGVVPILVELDGDVNGHKFSVSGEGEGDATYGKLTLK |
| Kappa-type | | FICTTGKLPVPWPTLVTTLTWGVQCFSRYPDHMKQHDFFKSAMPEG |
| opioid receptor | | YVQERTIFFKDDGNYKTRAEVKFEGDTLVNRIELKGIDFKEDGNIL |
| targeting binder- | | GHKLEYNYISHNVYITADKQKNGIKANFKIRHNIEDGSVQLADHYQ |
| Cezanne enDUB | | QNTPIGDGPVLLPDNHYLSTQSALSKDPNEKRDHMVLLEFVTAAGI |
| | | TLGMDELYKGSGATNFSLLKQAGDVEENPGPMAQVQLVESGGGLVR |
| | | PGGSLRLSCVDSERTSYPMGWFRRAPGKEREFVASITWSGIDPTYA |
| | | DSVADRETTSRDVANNTLYLQMNSLKHEDTAVYYCAARAPVGQSSS |
| | | PYDYDYWGQGTQVTVSSHHHHHHEPEAPPSFSEGSGGSRTPEKGES |
| | | DREPTRPPRPILQRQDDIVQEKRLSRGISHASSSIVSLARSHVSSN |
| | | GGGGGSNEHPLEMPICAFQLPDLTVYNEDERSFIERDLIEQSMLVA |
| | | LEQAGRLNWWVSVDPTSQRLLPLATTGDGNCLLHAASLGMWGFHDR |
| | | DLMLRKALYALMEKGVEKEALKRRWRWQQTQQNKESGLVYTEDEWQ |
| | | KEWNELIKLASSEPRMHLGTNGANCGGVESSEEPVYESLEEFHVEV |
| | | LAHVLRRPIVVVADTMLRDSGGEAFAPIPEGGIYLPLEVPASQCHR |
| | | SPLVLAYDQAHFSALVSMEQKENTKEQAVIPLTDSEYKLLPLHFAV |
| | | DPGKGWEWGKDDSDNVRLASVILSLEVKLHLLHSYMNVKWIPLSSD |
| | | AQAPLAQ |
| CFP-P2A-anti- | 284 | MVSKGEELFTGVVPILVELDGDVNGHKFSVSGEGEGDATYGKLTLK |
| Kappa-type | | FICTTGKLPVPWPTLVTTLTWGVQCFSRYPDHMKQHDFFKSAMPEG |
| opioid receptor | | YVQERTIFFKDDGNYKTRAEVKFEGDTLVNRIELKGIDFKEDGNIL |
| targeting binder- | | GHKLEYNYISHNVYITADKQKNGIKANFKIRHNIEDGSVQLADHYQ |
| OTUD1 enDUB | | QNTPIGDGPVLLPDNHYLSTQSALSKDPNEKRDHMVLLEFVTAAGI |
| | | TLGMDELYKGSGATNESLLKQAGDVEENPGPMAQVQLVESGGGLVR |
| | | PGGSLRLSCVDSERTSYPMGWFRRAPGKEREFVASITWSGIDPTYA |
| | | DSVADRETTSRDVANNTLYLQMNSLKHEDTAVYYCAARAPVGQSSS |
| | | PYDYDYWGQGTQVTVSSHHHHHHEPEADEKLALYLAEVEKQDKYLR |
| | | QRNKYRFHIIPDGNCLYRAVSKTVYGDQSLHRELREQTVHYIADHL |
| | | DHFSPLIEGDVGEFIIAAAQDGAWAGYPELLAMGQMLNVNIHLTTG |
| | | GRLESPTVSTMIHYLGPEDSLRPSIWLSWLSNGHYDAVEDHSYPNP |
| | | EYDNWCKQTQVQRKRDEELAKSMAISLSKMYIEQNACS |
| CFP-P2A-anti- | 285 | MVSKGEELFTGVVPILVELDGDVNGHKFSVSGEGEGDATYGKLTLK |
| Kappa-type | | FICTTGKLPVPWPTLVTTLTWGVQCFSRYPDHMKQHDFFKSAMPEG |
| opioid receptor | | YVQERTIFFKDDGNYKTRAEVKFEGDTLVNRIELKGIDFKEDGNIL |
| targeting binder- | | GHKLEYNYISHNVYITADKQKNGIKANFKIRHNIEDGSVQLADHYQ |
| TRABID | | QNTPIGDGPVLLPDNHYLSTQSALSKDPNEKRDHMVLLEFVTAAGI |
| enDUB | | TLGMDELYKGSGATNFSLLKQAGDVEENPGPMAQVQLVESGGGLVR |
| | | PGGSLRLSCVDSERTSYPMGWFRRAPGKEREFVASITWSGIDPTYA |
| | | DSVADRFTTSRDVANNTLYLQMNSLKHEDTAVYYCAARAPVGQSSS |
| | | PYDYDYWGQGTQVTVSSHHHHHHEPEALEVDFKKLKQIKNRMKKTD |
| | | WLFLNACVGVVEGDLAAIEAYKSSGGDIARQLTADEVRLLNRPSAF |
| | | DVGYTLVHLAIRFQRQDMLAILLTEVSQQAAKCIPAMVCPELTEQI |
| | | RREIAASLHQRKGDFACYFLTDLVTFTLPADIEDLPPTVQEKLEDE |
| | | VLDRDVQKELEEESPIINWSLELATRLDSRLYALWNRTAGDCLLDS |
| | | VLQATWGIYDKDSVLRKALHDSLHDCSHWFYTRWKDWESWYSQSFG |
| | | LHFSLREEQWQEDWAFILSLASQPGASLEQTHIFVLAHILRRPIIV |
| | | YGVKYYKSFRGETLGYTRFQGVYLPLLWEQSFCWKSPIALGYTRGH |
| | | FSALVAMENDGYGNRGAGANLNTDDDVTITFLPLVDSERKLLHVHE |
| | | LSAQELGNEEQQEKLLREWLDCCVTEGGVLVAMQKSSRRRNHPLVT |
| | | QMVEKWLDRYRQIRPCTSLS |
| CFP-P2A-anti- | 286 | MVSKGEELFTGVVPILVELDGDVNGHKFSVSGEGEGDATYGKLTLK |
| Kappa-type | | FICTTGKLPVPWPTLVTTLTWGVQCFSRYPDHMKQHDFFKSAMPEG |
| opioid receptor | | YVQERTIFFKDDGNYKTRAEVKFEGDTLVNRIELKGIDFKEDGNIL |
| targeting binder- | | GHKLEYNYISHNVYITADKQKNGIKANFKIRHNIEDGSVQLADHYQ |
| USP21 enDUB | | QNTPIGDGPVLLPDNHYLSTQSALSKDPNEKRDHMVLLEFVTAAGI |
| | | TLGMDELYKGSGATNFSLLKQAGDVEENPGPMAQVQLVESGGGLVR |
| | | PGGSLRLSCVDSERTSYPMGWFRRAPGKEREFVASITWSGIDPTYA |
| | | DSVADRETTSRDVANNTLYLQMNSLKHEDTAVYYCAARAPVGQSSS |
| | | PYDYDYWGQGTQVTVSSHHHHHHEPEASDDKMAHHTLLLGSGHVGL |
| | | RNLGNTCFLNAVLQCLSSTRPLRDFCLRRDERQEVPGGGRAQELTE |
| | | AFADVIGALWHPDSCEAVNPTRFRAVFQKYVPSFSGYSQQDAQEFL |
| | | KLLMERLHLEINRRGRRAPPILANGPVPSPPRRGGALLEEPELSDD |
| | | DRANLMWKRYLEREDSKIVDLFVGQLKSCLKCQACGYRSTTFEVFC |
| | | DLSLPIPKKGFAGGKVSLRDCENLFTKEEELESENAPVCDRCRQKT |
| | | RSTKKLTVQRFPRILVLHLNRESASRGSIKKSSVGVDFPLQRLSLG |
| | | DFASDKAGSPVYQLYALCNHSGSVHYGHYTALCRCQTGWHVYNDSR |
| | | VSPVSENQVASSEGYVLFYQLMQEPPRCL |
| CFP-P2A-anti- | 287 | MVSKGEELFTGVVPILVELDGDVNGHKFSVSGEGEGDATYGKLTLK |
| Kappa-type | | FICTTGKLPVPWPTLVTTLTWGVQCFSRYPDHMKQHDFFKSAMPEG |
| opioid receptor | | YVQERTIFFKDDGNYKTRAEVKFEGDTLVNRIELKGIDFKEDGNIL |
| targeting binder- | | GHKLEYNYISHNVYITADKQKNGIKANFKIRHNIEDGSVQLADHYQ |
| OTUD4 enDUB | | QNTPIGDGPVLLPDNHYLSTQSALSKDPNEKRDHMVLLEFVTAAGI |
| | | TLGMDELYKGSGATNFSLLKQAGDVEENPGPMAQVQLVESGGGLVR |
| | | PGGSLRLSCVDSERTSYPMGWERRAPGKEREFVASITWSGIDPTYA |
| | | DSVADRETTSRDVANNTLYLQMNSLKHEDTAVYYCAARAPVGQSSS |
| | | PYDYDYWGQGTQVTVSSHHHHHHEPEAATPMDAYLRKLGLYRKLVA |
| | | KDGSCLFRAVAEQVLHSQSRHVEVRMACIHYLRENREKFEAFIEGS |
| | | FEEYLKRLENPQEWVGQVEISALSLMYRKDFIIYREPNVSPSQVTE |
| | | NNFPEKVLLCESNGNHYDIVYPIKYKESSAMCQSLLYELLYEKVEK |
| | | TDVSKIVMELDTLEVADE |
| CFP-P2A-anti- | 288 | MVSKGEELFTGVVPILVELDGDVNGHKFSVSGEGEGDATYGKLTLK |
| Muscarinic | | FICTTGKLPVPWPTLVTTLTWGVQCFSRYPDHMKQHDFFKSAMPEG |
| acetylcholine | | YVQERTIFFKDDGNYKTRAEVKFEGDTLVNRIELKGIDFKEDGNIL |
| receptor M2 | | GHKLEYNYISHNVYITADKQKNGIKANFKIRHNIEDGSVQLADHYQ |
| targeting binder- | | QNTPIGDGPVLLPDNHYLSTQSALSKDPNEKRDHMVLLEFVTAAGI |
| Cezanne enDUB | | TLGMDELYKGSGATNFSLLKQAGDVEENPGPGPGSQVQLQESGGGL |
| | | VQAGDSLRLSCAASGEDEDNEDDYAIGWFRQAPGQEREGVSCIDPS |
| | | DGSTIYADSAKGRFTISSDNAENTVYLQMNSLKPEDTAVYVCSAWT |
| | | LFHSDEYWGQGTQVTVSSPPSFSEGSGGSRTPEKGESDREPTRPPR |
| | | PILQRQDDIVQEKRLSRGISHASSSIVSLARSHVSSNGGGGGSNEH |
| | | PLEMPICAFQLPDLTVYNEDERSFIERDLIEQSMLVALEQAGRLNW |
| | | WVSVDPTSQRLLPLATTGDGNCLLHAASLGMWGFHDRDLMLRKALY |
| | | ALMEKGVEKEALKRRWRWQQTQQNKESGLVYTEDEWQKEWNELIKL |
| | | ASSEPRMHLGTNGANCGGVESSEEPVYESLEEFHVFVLAHVLRRPI |
| | | VVVADTMLRDSGGEAFAPIPEGGIYLPLEVPASQCHRSPLVLAYDQ |
| | | AHFSALVSMEQKENTKEQAVIPLTDSEYKLLPLHFAVDPGKGWEWG |
| | | KDDSDNVRLASVILSLEVKLHLLHSYMNVKWIPLSSDAQAPLAQ |
| CFP-P2A-anti- | 289 | MVSKGEELFTGVVPILVELDGDVNGHKFSVSGEGEGDATYGKLTLK |
| Muscarinic | | FICTTGKLPVPWPTLVTTLTWGVQCFSRYPDHMKQHDFFKSAMPEG |
| acetylcholine | | YVQERTIFFKDDGNYKTRAEVKFEGDTLVNRIELKGIDFKEDGNIL |
| receptor M2 | | GHKLEYNYISHNVYITADKQKNGIKANFKIRHNIEDGSVQLADHYQ |
| targeting binder- | | QNTPIGDGPVLLPDNHYLSTQSALSKDPNEKRDHMVLLEFVTAAGI |
| OTUD1 enDUB | | TLGMDELYKGSGATNFSLLKQAGDVEENPGPGPGSQVQLQESGGGL |
| | | VQAGDSLRLSCAASGEDEDNEDDYAIGWFRQAPGQEREGVSCIDPS |
| | | DGSTIYADSAKGRFTISSDNAENTVYLQMNSLKPEDTAVYVCSAWT |
| | | LFHSDEYWGQGTQVTVSSDEKLALYLAEVEKQDKYLRQRNKYRFHI |
| | | IPDGNCLYRAVSKTVYGDQSLHRELREQTVHYIADHLDHESPLIEG |
| | | DVGEFIIAAAQDGAWAGYPELLAMGQMLNVNIHLTTGGRLESPTVS |
| | | TMIHYLGPEDSLRPSIWLSWLSNGHYDAVEDHSYPNPEYDNWCKQT |
| | | QVQRKRDEELAKSMAISLSKMYIEQNACS |
| CFP-P2A-anti- | 290 | MVSKGEELFTGVVPILVELDGDVNGHKFSVSGEGEGDATYGKLTLK |
| Muscarinic | | FICTTGKLPVPWPTLVTTLTWGVQCFSRYPDHMKQHDFFKSAMPEG |
| acetylcholine | | YVQERTIFFKDDGNYKTRAEVKFEGDTLVNRIELKGIDFKEDGNIL |
| receptor M2 | | GHKLEYNYISHNVYITADKQKNGIKANFKIRHNIEDGSVQLADHYQ |
| targeting binder- | | QNTPIGDGPVLLPDNHYLSTQSALSKDPNEKRDHMVLLEFVTAAGI |
| TRABID | | TLGMDELYKGSGATNFSLLKQAGDVEENPGPGPGSQVQLQESGGGL |
| enDUB | | VQAGDSLRLSCAASGEDEDNEDDYAIGWFRQAPGQEREGVSCIDPS |
| | | DGSTIYADSAKGRFTISSDNAENTVYLQMNSLKPEDTAVYVCSAWT |
| | | LFHSDEYWGQGTQVTVSSLEVDFKKLKQIKNRMKKTDWLFLNACVG |
| | | VVEGDLAAIEAYKSSGGDIARQLTADEVRLLNRPSAFDVGYTLVHL |
| | | AIRFQRQDMLAILLTEVSQQAAKCIPAMVCPELTEQIRREIAASLH |
| | | QRKGDFACYFLTDLVTFTLPADIEDLPPTVQEKLEDEVLDRDVQKE |
| | | LEEESPIINWSLELATRLDSRLYALWNRTAGDCLLDSVLQATWGIY |
| | | DKDSVLRKALHDSLHDCSHWFYTRWKDWESWYSQSFGLHESLREEQ |
| | | WQEDWAFILSLASQPGASLEQTHIFVLAHILRRPIIVYGVKYYKSE |
| | | RGETLGYTRFQGVYLPLLWEQSFCWKSPIALGYTRGHFSALVAMEN |
| | | DGYGNRGAGANLNTDDDVTITFLPLVDSERKLLHVHELSAQELGNE |
| | | EQQEKLLREWLDCCVTEGGVLVAMQKSSRRRNHPLVTQMVEKWLDR |
| | | YRQIRPCTSLS |
| CFP-P2A-anti- | 291 | MVSKGEELFTGVVPILVELDGDVNGHKFSVSGEGEGDATYGKLTLK |
| Muscarinic | | FICTTGKLPVPWPTLVTTLTWGVQCFSRYPDHMKQHDEFKSAMPEG |
| acetylcholine | | YVQERTIFFKDDGNYKTRAEVKFEGDTLVNRIELKGIDFKEDGNIL |
| receptor M2 | | GHKLEYNYISHNVYITADKQKNGIKANFKIRHNIEDGSVQLADHYQ |
| targeting binder- | | QNTPIGDGPVLLPDNHYLSTQSALSKDPNEKRDHMVLLEFVTAAGI |
| USP21 enDUB | | TLGMDELYKGSGATNFSLLKQAGDVEENPGPGPGSQVQLQESGGGL |
| | | VQAGDSLRLSCAASGEDEDNEDDYAIGWFRQAPGQEREGVSCIDPS |
| | | DGSTIYADSAKGRFTISSDNAENTVYLQMNSLKPEDTAVYVCSAWT |
| | | LFHSDEYWGQGTQVTVSSSDDKMAHHTLLLGSGHVGLRNLGNTCEL |
| | | NAVLQCLSSTRPLRDFCLRRDFRQEVPGGGRAQELTEAFADVIGAL |
| | | WHPDSCEAVNPTRFRAVFQKYVPSFSGYSQQDAQEFLKLLMERLHL |
| | | EINRRGRRAPPILANGPVPSPPRRGGALLEEPELSDDDRANLMWKR |
| | | YLEREDSKIVDLFVGQLKSCLKCQACGYRSTTFEVFCDLSLPIPKK |
| | | GFAGGKVSLRDCENLFTKEEELESENAPVCDRCRQKTRSTKKLTVQ |
| | | RFPRILVLHLNRESASRGSIKKSSVGVDFPLQRLSLGDFASDKAGS |
| | | PVYQLYALCNHSGSVHYGHYTALCRCQTGWHVYNDSRVSPVSENQV |
| | | ASSEGYVLFYQLMQEPPRCL |
| CFP-P2A-anti- | 292 | MVSKGEELFTGVVPILVELDGDVNGHKFSVSGEGEGDATYGKLTLK |
| Muscarinic | | FICTTGKLPVPWPTLVTTLTWGVQCFSRYPDHMKQHDFFKSAMPEG |
| acetylcholine | | YVQERTIFFKDDGNYKTRAEVKFEGDTLVNRIELKGIDFKEDGNIL |
| receptor M2 | | GHKLEYNYISHNVYITADKQKNGIKANFKIRHNIEDGSVQLADHYQ |
| targeting binder- | | QNTPIGDGPVLLPDNHYLSTQSALSKDPNEKRDHMVLLEFVTAAGI |
| OTUD4 enDUB | | TLGMDELYKGSGATNFSLLKQAGDVEENPGPGPGSQVQLQESGGGL |
| | | VQAGDSLRLSCAASGEDEDNEDDYAIGWFRQAPGQEREGVSCIDPS |
| | | DGSTIYADSAKGRFTISSDNAENTVYLQMNSLKPEDTAVYVCSAWT |
| | | LFHSDEYWGQGTQVTVSSATPMDAYLRKLGLYRKLVAKDGSCLFRA |
| | | VAEQVLHSQSRHVEVRMACIHYLRENREKFEAFIEGSFEEYLKRLE |
| | | NPQEWVGQVEISALSLMYRKDFIIYREPNVSPSQVTENNFPEKVLL |
| | | CFSNGNHYDIVYPIKYKESSAMCQSLLYELLYEKVEKTDVSKIVME |
| | | LDTLEVADE |

### 6.2 Example 2. Testing of Targeted Engineered Deubiquitinases

To demonstrate upregulation of a target protein in the context of a specific targeting enDUB the following experiments will be performed.

Schematic constructs used:
Control experiment using non-targeting enDUB fusion
   Target-YFP-P2A-mCherrry
   CFP-P2A-enDUB (nontargeting control enDUB)
Test constructs for up-regulation:
   Target-YFP-P2A-mCherry
   CFP-P2A-a-YFPnanobody-enDUB
Or specific targeting enDUB fusion composed of
   CFP-P2A-anti-targeting binder-enDUB

Co-transfection of both plasmids carrying the YFP tagged target protein together with the enDUB fused to a target binding protein into HEK cells will be performed. A control construct carrying the enDUB in the absence of the targeting binder will also be co-transfected together with the labeled target protein. After 24-48 hours the transfected cells will be analyzed by FACS or upregulation over the control. The mCherry signal on the target protein will be used to normalize for transfection efficiency while the CFP signal will be used to normalize for the transfection efficiency of the enDUB constructs. The YFP fused to the target protein is the read-out for target gene expression and will be plotted vs the signal in the control transfection. Relative increase in the YFP fluorescence over control will demonstrate upregulation in the presence of the enDUB.

### 6.3 Example 3. Screening Assay for Testing Fusion Proteins

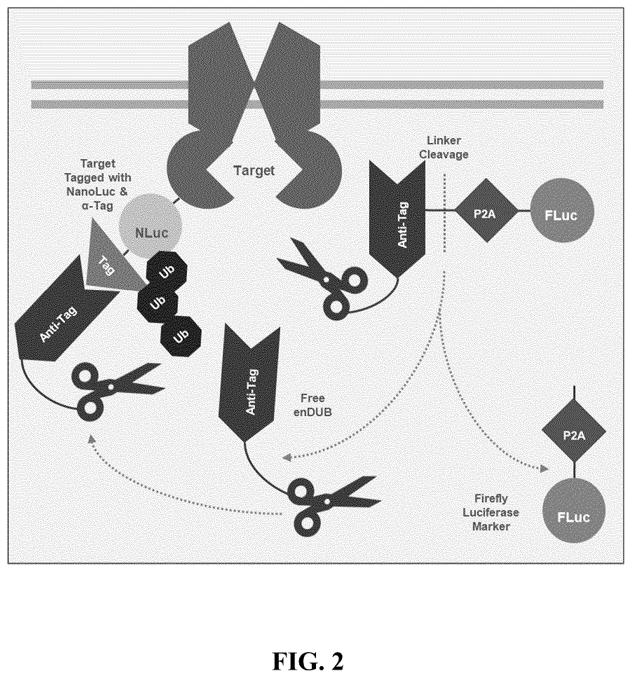

The following example describes an assay to analyze the ability of a targeted engineered deubiquitinase (enDub) (e.g., an enDub described herein) to increase expression of a target protein. Generally, the assay involves tagging the target protein with a fluorescent tag (e.g., NanoLuciferase (NLuc)) and an alfa-tag (α-Tag); and tagging a fusion protein of the enDub and an anti-alfa Tag nanobody with a different fluorescent tag (e.g., Firefly Luciferase (FLuc)) through a cleavable linker. The use of two different fluorescent tags enables normalization of the signal to compensate for variation in transfection/expression, as the second fluorescent tag is rapidly cleaved from the enDub-anti-alfa tag fusion protein inside the cell through cleavage of the cleavable linker. FIG. 2 provides a general schematic of the cellular aspects of the assay. The protocol, including materials and methods is described below.

CHO-K1 cells were digested with 0.25% (w/v) Trypsin-EDTA, at 37° C., for 5 min. Complete medium was added for the CHO-K1 cell cultures to stop the digestion. The CHO-K1 cells were centrifuges at 800 rpm for 5 minutes. After centrifugation, the supernatant was discarded and the CHO-K1 cells were resuspend in 2 mL culture medium and counted. 10\hat{ }6 CHO-K1 cells were electroporated under 440V with 0.5 ug of a plasmid encoding the target protein tagged with NLuc and alfa-tag, and 1 ug of a plasmid encoding a) enDub-anti-alfa tag nanobody-FLuc fusion protein (experimental), b) the enDub (control), or the anti-alfa tag nanobody (control). 5E+4 cells/well were placed in in 24 well plates and cultured for 24 h, at 37° C., 5% CO₂. The cells were digested with 0.25% (w/v) Trypsin-EDTA, at 37° C. for 5 min. Complete medium was added to the culture to stop the digestion and the cells were counted for use in NanoGlo® Dual Luciferase® Assay (Promega), which enables detection of FLuc and NLuc® in a single sample. The NanoGlo® Dual Luciferase® Assay was carried out according to manufacturer's instructions (Promega, Nano-Glo® Dual-Luciferase® Reporter Assay Technical Manual #TM426). Briefly, 1E+4 cells/well were placed in 96 well black plates and cultured for 24 h, at 37° C., 5% CO₂. The plates were removed from the incubator and allowed to equilibrate to room temperature. The samples were modified as needed to have a starting volume of 80 μl per well. All sample wells were injected with 80 μl of ONE-Glo™ EX Reagent and incubated for 3 minutes. The firefly luminescence was read in all sample wells using a 1-second integration time. All sample wells were injected with 80 μl of NanoDLR™ Stop & Glo® Reagent; and incubated for 5 minutes. The NanoLuc® luminescence of all sample wells was read using a 1-second integration time. The dispensing lines were cleaned according to manufacturer's instructions (Nano-Glo® Dual-Luciferase® Reporter Assay Technical Manual #TM426.) and the data analyzed.

The amino acid sequence of the components of the fusion proteins used in the assay are detailed in Table 6 below.

TABLE 6
Amino acid sequence of components of test fusion proteins

| Description | | SEQ ID NO | Amino Acid Sequence |
|---|---|---|---|
| Fluorescent Protein | NanoLuc | 425 | VFTLEDFVGDWRQTAGYNLDQVLEQGGVSSLFQ |
| | | | NLGVSVTPIQRIVLSGENGLKIDIHVIIPYEGL |
| | | | SGDQMGQIEKIFKVVYPVDDHHFKVILHYGTLV |
| | | | IDGVTPNMIDYFGRPYEGIAVFDGKKITVTGTL |
| | | | WNGNKIIDERLINPDGSLLFRVTINGVTGWRLC |
| | | | ERILA |
| | Firefly | 426 | MEDAKNIKKGPAPFYPLEDGTAGEQLHKAMKRY |
| | Luciferase | | ALVPGTIAFTDAHIEVDITYAEYFEMSVRLAEA |
| | | | MKRYGLNTNHRIVVCSENSLQFFMPVLGALFIG |
| | | | VAVAPANDIYNERELLNSMGISQPTVVFVSKKG |
| | | | LQKILNVQKKLPIIQKIIIMDSKTDYQGFQSMY |
| | | | TFVTSHLPPGENEYDFVPESEDRDKTIALIMNS |
| | | | SGSTGLPKGVALPHRTACVRESHARDPIFGNQI |
| | | | IPDTAILSVVPFHHGFGMFTTLGYLICGFRVVL |
| | | | MYRFEEELFLRSLQDYKIQSALLVPTLESFFAK |
| | | | STLIDKYDLSNLHEIASGGAPLSKEVGEAVAKR |
| | | | FHLPGIRQGYGLTETTSAILITPEGDDKPGAVG |
| | | | KVVPFFEAKVVDLDTGKTLGVNQRGELCVRGPM |
| | | | IMSGYVNNPEATNALIDKDGWLHSGDIAYWDED |
| | | | EHFFIVDRLKSLIKYKGYQVAPAELESILLQHP |
| | | | NIFDAGVAGLPDDDAGELPAAVVVLEHGKTMTE |
| | | | KEIVDYVASQVTTAKKLRGGVVFVDEVPKGLTG |
| | | | KLDARKIREILIKAKKGGKIAVTRLK |

| | | |
|---|---|---|
| Alfa Tag | 427 | PSRLEEELRRRLTEP |
| P2A | 428 | GSGATNFSLLKQAGDVEENPGP |
| Cezanne (Exemplary Catalytic | 429 | PPSFSEGSGGSRTPEKGFSDREPTRPPRPILQR |
| Domain) | | QDDIVQEKRLSRGISHASSSIVSLARSHVSSNG |
| | | GGGGSNEHPLEMPICAFQLPDLTVYNEDERSFI |
| | | ERDLIEQSMLVALEQAGRLNWWVSVDPTSQRLL |
| | | PLATTGDGNCLLHAASLGMWGFHDRDLMLRKAL |
| | | YALMEKGVEKEALKRRWRWQQTQQNKESGLVYT |
| | | EDEWQKEWNELIKLASSEPRMHLGTNGANCGGV |
| | | ESSEEPVYESLEEFHVFVLAHVLRRPIVVVADT |
| | | MLRDSGGEAFAPIPFGGIYLPLEVPASQCHRSP |
| | | LVLAYDQAHFSALVSMEQKENTKEQAVIPLTDS |
| | | EYKLLPLHFAVDPGKGWEWGKDDSDNVRLASVI |
| | | LSLEVKLHLLHSYMNVKWIPLSSDAQAPLAQ |

The amino acid sequence of exemplary target fusion proteins comprising a target protein, NLuc, and the alfa tag are detailed in Table 7 below.

TABLE 7
Amino Acid Sequence of exemplary Target Protein-NLuc-Alfa Tag Fusion Proteins

| Test Protein | SEQ ID NO | Amino Acid Sequence |
|---|---|---|
| GRIN2A-nanoluc- | 430 | MGRVGYWTLLVLPALLVWRGPAPSAAAEKGPPALNIAVMLGHSHD |
| alfa-tag-fusion | | VTERELRTLWGPEQAAGLPLDVNVVALLMNRTDPKSLITHVCDLM |
| | | SGARIHGLVFGDDTDQEAVAQMLDFISSHTFVPILGIHGGASMIM |
| | | ADKDPTSTFFQFGASIQQQATVMLKIMQDYDWHVESLVTTIFPGY |
| | | REFISFVKTTVDNSFVGWDMQNVITLDTSFEDAKTQVQLKKIHSS |
| | | VILLYCSKDEAVLILSEARSLGLTGYDFFWIVPSLVSGNTELIPK |
| | | EFPSGLISVSYDDWDYSLEARVRDGIGILTTAASSMLEKFSYIPE |
| | | AKASCYGQMERPEVPMHTLHPFMVNVTWDGKDLSFTEEGYQVHPR |
| | | LVVIVLNKDREWEKVGKWENHTLSLRHAVWPRYKSFSDCEPDDNH |
| | | LSIVTLEEAPFVIVEDIDPLTETCVRNTVPCRKFVKINNSTNEGM |
| | | NVKKCCKGFCIDILKKLSRTVKFTYDLYLVTNGKHGKKVNNVWNG |
| | | MIGEVVYQRAVMAVGSLTINEERSEVVDESVPFVETGISVMVSRS |
| | | NGTVSPSAFLEPFSASVWVMMFVMLLIVSAIAVFVFEYESPVGYN |
| | | RNLAKGKAPHGPSFTIGKAIWLLWGLVENNSVPVQNPKGTTSKIM |
| | | VSVWAFFAVIFLASYTANLAAFMIQEEFVDQVTGLSDKKFQRPHD |
| | | YSPPFRFGTVPNGSTERNIRNNYPYMHQYMTKENQKGVEDALVSL |
| | | KTGKLDAFIYDAAVLNYKAGRDEGCKLVTIGSGYIFATTGYGIAL |
| | | QKGSPWKRQIDLALLQFVGDGEMEELETLWLTGICHNEKNEVMSS |
| | | QLDIDNMAGVFYMLAAAMALSLITFIWEHLFYWKLRFCFTGVCSD |
| | | RPGLLESISRGIYSCIHGVHIEEKKKSPDENLTGSQSNMLKLLRS |
| | | AKNISSMSNMNSSRMDSPKRAADFIQRGSLIMDMVSDKGNLMYSD |
| | | NRSFQGKESIFGDNMNELQTFVANRQKDNLNNYVFQGQHPLTLNE |
| | | SNPNTVEVAVSTESKANSRPRQLWKKSVDSIRQDSLSQNPVSQRD |
| | | EATAENRTHSLKSPRYLPEEMAHSDISETSNRATCHREPDNSKNH |
| | | KTKDNFKRSVASKYPKDCSEVERTYLKTKSSSPRDKIYTIDGEKE |
| | | PGFHLDPPQFVENVTLPENVDFPDPYQDPSENFRKGDSTLPMNRN |
| | | PLHNEEGLSNNDQYKLYSKHFTLKDKGSPHSETSERYRQNSTHCR |
| | | SCLSNMPTYSGHFTMRSPFKCDACLRMGNLYDIDEDQMLQETGNP |
| | | ATGEQVYQQDWAQNNALQLQKNKLRISRQHSYDNIVDKPRELDLS |
| | | RPSRSISLKDRERLLEGNFYGSLFSVPSSKLSGKKSSLFPQGLED |
| | | SKRSKSLLPDHTSDNPFLHSHRDDQRLVIGRCPSDPYKHSLPSQA |
| | | VNDSYLRSSLRSTASYCSRDSRGHNDVYISEHVMPYAANKNNMYS |
| | | TPRVLNSCSNRRVYKKMPSIESDVKVPVFTLEDFVGDWRQTAGYN |
| | | LDQVLEQGGVSSLFQNLGVSVTPIQRIVLSGENGLKIDIHVIIPY |
| | | EGLSGDQMGQIEKIFKVVYPVDDHHFKVILHYGTLVIDGVTPNMI |
| | | DYFGRPYEGIAVEDGKKITVTGTLWNGNKIIDERLINPDGSLLER |
| | | VTINGVTGWRLCERILAGGGGSPSRLEEELRRRLTEP |
| SLC2A1-nanoluc- | 431 | MEPSSKKLTGRLMLAVGGAVLGSLQFGYNTGVINAPQKVIEEFYN |
| alfa-tag-fusion | | QTWVHRYGESILPTTLTTLWSLSVAIFSVGGMIGSFSVGLFVNRE |
| | | GRRNSMLMMNLLAFVSAVLMGFSKLGKSFEMLILGRFIIGVYCGL |
| | | TTGFVPMYVGEVSPTALRGALGTLHQLGIVVGILIAQVFGLDSIM |
| | | GNKDLWPLLLSIIFIPALLQCIVLPFCPESPRELLINRNEENRAK |
| | | SVLKKLRGTADVTHDLQEMKEESRQMMREKKVTILELFRSPAYRQ |
| | | PILIAVVLQLSQQLSGINAVFYYSTSIFEKAGVQQPVYATIGSGI |
| | | VNTAFTVVSLFVVERAGRRTLHLIGLAGMAGCAILMTIALALLEQ |
| | | LPWMSYLSIVAIFGFVAFFEVGPGPIPWFIVAELFSQGPRPAAIA |
| | | VAGFSNWTSNFIVGMCFQYVEQLCGPYVFIIFTVLLVLFFIFTYF |
| | | KVPETKGRTFDEIASGFRQGGASQSDKTPEELFHPLGADSQVKVP |
| | | VFTLEDFVGDWRQTAGYNLDQVLEQGGVSSLFQNLGVSVTPIQRI |
| | | VLSGENGLKIDIHVIIPYEGLSGDQMGQIEKIFKVVYPVDDHHFK |
| | | VILHYGTLVIDGVTPNMIDYFGRPYEGIAVEDGKKITVTGTLWNG |
| | | NKIIDERLINPDGSLLFRVTINGVTGWRLCERILAGGGGSPSRLE |
| | | EELRRRLTEP |
| CACNA1A- | 432 | MARFGDEMPARYGGGGSGAAAGVVVGSGGGRGAGGSRQGGQPGAQ |
| nanoluc-alfa-tag- | | RMYKQSMAQRARTMALYNPIPVRQNCLTVNRSLFLESEDNVVRKY |
| fusion | | AKKITEWPPFEYMILATIIANCIVLALEQHLPDDDKTPMSERLDD |
| | | TEPYFIGIFCFEAGIKIIALGFAFHKGSYLRNGWNVMDFVVVLTG |
| | | ILATVGTEFDLRTLRAVRVLRPLKLVSGIPSLQVVLKSIMKAMIP |
| | | LLQIGLLLFFAILIFAIIGLEFYMGKFHTTCFEEGTDDIQGESPA |
| | | PCGTEEPARTCPNGTKCQPYWEGPNNGITQFDNILFAVLTVFQCI |
| | | TMEGWTDLLYNSNDASGNTWNWLYFIPLIIIGSFFMLNLVLGVLS |
| | | GEFAKERERVENRRAFLKLRRQQQIERELNGYMEWISKAEEVILA |
| | | EDETDGEQRHPFDALRRTTIKKSKTDLLNPEEAEDQLADIASVGS |
| | | PFARASIKSAKLENSTFFHKKERRMRFYIRRMVKTQAFYWTVLSL |
| | | VALNTLCVAIVHYNQPEWLSDFLYYAEFIFLGLFMSEMFIKMYGL |
| | | GTRPYFHSSENCFDCGVIIGSIFEVIWAVIKPGTSFGISVLRALR |
| | | LLRIFKVTKYWASLRNLVVSLLNSMKSIISLLFLLFLFIVVFALL |
| | | GMQLFGGQFNFDEGTPPTNEDTFPAAIMTVFQILTGEDWNEVMYD |
| | | GIKSQGGVQGGMVFSIYFIVLTLFGNYTLLNVELAIAVDNLANAQ |
| | | ELTKDEQEEEEAANQKLALQKAKEVAEVSPLSAANMSIAVKEQQK |
| | | NQKPAKSVWEQRTSEMRKQNLLASREALYNEMDPDERWKAAYTRH |
| | | LRPDMKTHLDRPLVVDPQENRNNNTNKSRAAEPTVDQRLGQQRAE |
| | | DELRKQARYHDRARDPSGSAGLDARRPWAGSQEAELSREGPYGRE |
| | | SDHHAREGSLEQPGFWEGEAERGKAGDPHRRHVHRQGGSRESRSG |
| | | SPRTGADGEHRRHRAHRRPGEEGPEDKAERRARHREGSRPARGGE |
| | | GEGEGPDGGERRRRHRHGAPATYEGDARREDKERRHRRRKENQGS |
| | | GVPVSGPNLSTTRPIQQDLGRQDPPLAEDIDNMKNNKLATAESAA |
| | | PHGSLGHAGLPQSPAKMGNSTDPGPMLAIPAMATNPQNAASRRTP |
| | | NNPGNPSNPGPPKTPENSLIVTNPSGTQTNSAKTARKPDHTTVDI |
| | | PPACPPPLNHTVVQVNKNANPDPLPKKEEEKKEEEEDDRGEDGPK |
| | | PMPPYSSMFILSTTNPLRRLCHYILNLRYFEMCILMVIAMSSIAL |
| | | AAEDPVQPNAPRNNVLRYFDYVFTGVFTFEMVIKMIDLGLVLHQG |
| | | AYFRDLWNILDFIVVSGALVAFAFTGNSKGKDINTIKSLRVLRVL |
| | | RPLKTIKRLPKLKAVFDCVVNSLKNVENILIVYMLEMFIFAVVAV |
| | | QLFKGKFFHCTDESKEFEKDCRGKYLLYEKNEVKARDREWKKYEF |
| | | HYDNVLWALLTLFTVSTGEGWPQVLKHSVDATFENQGPSPGYRME |
| | | MSIFYVVYFVVFPFFFVNIFVALIIITFQEQGDKMMEEYSLEKNE |
| | | RACIDFAISAKPLTRHMPQNKQSFQYRMWQFVVSPPFEYTIMAMI |
| | | ALNTIVLMMKFYGASVAYENALRVENIVFTSLESLECVLKVMAFG |
| | | ILNYFRDAWNIFDFVTVLGSITDILVTEFGNNFINLSFLRLFRAA |
| | | RLIKLLRQGYTIRILLWTFVQSFKALPYVCLLIAMLFFIYAIIGM |
| | | QVFGNIGIDVEDEDSDEDEFQITEHNNERTFFQALMLLERSATGE |
| | | AWHNIMLSCLSGKPCDKNSGILTRECGNEFAYFYFVSFIFLCSEL |
| | | MLNLFVAVIMDNFEYLTRDSSILGPHHLDEYVRVWAEYDPAAWGR |
| | | MPYLDMYQMLRHMSPPLGLGKKCPARVAYKRLLRMDLPVADDNTV |
| | | HFNSTLMALIRTALDIKIAKGGADKQQMDAELRKEMMAIWPNLSQ |
| | | KTLDLLVTPHKSTDLTVGKIYAAMMIMEYYRQSKAKKLQAMREEQ |
| | | DRTPLMFQRMEPPSPTQEGGPGQNALPSTQLDPGGALMAHESGLK |
| | | ESPSWVTQRAQEMFQKTGTWSPEQGPPTDMPNSQPNSQSVEMREM |
| | | GRDGYSDSEHYLPMEGQGRAASMPRLPAENQRRRGRPRGNNLSTI |
| | | SDTSPMKRSASVLGPKARRLDDYSLERVPPEENQRHHQRRRDRSH |
| | | RASERSLGRYTDVDTGLGTDLSMTTQSGDLPSKERDQERGRPKDR |
| | | KHRQHHHHHHHHHHPPPPDKDRYAQERPDHGRARARDQRWSRSPS |
| | | EGREHMAHRQGSSSVSGSPAPSTSGTSTPRRGRRQLPQTPSTPRP |
| | | HVSYSPVIRKAGGSGPPQQQQQQQQQQQQQAVARPGRAATSGPRR |
| | | YPGPTAEPLAGDRPPTGGHSSGRSPRMERRVPGPARSESPRACRH |
| | | GGARWPASGPHVSEGPPGPRHHGYYRGSDYDEADGPGSGGGEEAM |
| | | AGAYDAPPPVRHASSGATGRSPRTPRASGPACASPSRHGRRLPNG |
| | | YYPAHGLARPRGPGSRKGLHEPYSESDDDWCKVPVFTLEDFVGDW |
| | | RQTAGYNLDQVLEQGGVSSLFQNLGVSVTPIQRIVLSGENGLKID |
| | | IHVIIPYEGLSGDQMGQIEKIFKVVYPVDDHHFKVILHYGTLVID |
| | | GVTPNMIDYFGRPYEGIAVEDGKKITVTGTLWNGNKIIDERLINP |
| | | DGSLLFRVTINGVTGWRLCERILAGGGGSPSRLEEELRRRLTEP |
| GABRB3-nanoluc- | 433 | MWGLAGGRLFGIFSAPVLVAVVCCAQSVNDPGNMSFVKETVDKLL |
| alfa-tag-fusion | | KGYDIRLRPDFGGPPVCVGMNIDIASIDMVSEVNMDYTLTMYFQQ |
| | | YWRDKRLAYSGIPLNLTLDNRVADQLWVPDTYFLNDKKSFVHGVT |
| | | VKNRMIRLHPDGTVLYGLRITTTAACMMDLRRYPLDEQNCTLEIE |
| | | SYGYTTDDIEFYWRGGDKAVTGVERIELPQFSIVEHRLVSRNVVE |
| | | ATGAYPRLSLSFRLKRNIGYFILQTYMPSILITILSWVSFWINYD |
| | | ASAARVALGITTVLTMTTINTHLRETLPKIPYVKAIDMYLMGCFV |
| | | FVFLALLEYAFVNYIFFGRGPQRQKKLAEKTAKAKNDRSKSESNR |
| | | VDAHGNILLTSLEVHNEMNEVSGGIGDTRNSAISEDNSGIQYRKQ |
| | | SMPREGHGRELGDRSLPHKKTHLRRRSSQLKIKIPDLTDVNAIDR |
| | | WSRIVFPFTFSLENLVYWLYYVNKVPVFTLEDFVGDWRQTAGYNL |
| | | DQVLEQGGVSSLFQNLGVSVTPIQRIVLSGENGLKIDIHVIIPYE |
| | | GLSGDQMGQIEKIFKVVYPVDDHHFKVILHYGTLVIDGVTPNMID |
| | | YFGRPYEGIAVEDGKKITVTGTLWNGNKIIDERLINPDGSLLERV |
| | | TINGVTGWRLCERILAGGGGSPSRLEEELRRRLTEP |
| SLC6A1-nanoluc- | 434 | MATNGSKVADGQISTEVSEAPVANDKPKTLVVKVQKKAADLPDRD |
| alfa-tag-fusion | | TWKGRFDFLMSCVGYAIGLGNVWRFPYLCGKNGGGAFLIPYFLTL |
| | | IFAGVPLFLLECSLGQYTSIGGLGVWKLAPMFKGVGLAAAVLSFW |
| | | LNIYYIVIISWAIYYLYNSFTTTLPWKQCDNPWNTDRCESNYSMV |
| | | NTTNMTSAVVEFWERNMHQMTDGLDKPGQIRWPLAITLAIAWILV |
| | | YFCIWKGVGWTGKVVYFSATYPYIMLIILFFRGVTLPGAKEGILF |
| | | YITPNFRKLSDSEVWLDAATQIFFSYGLGLGSLIALGSYNSFHNN |
| | | VYRDSIIVCCINSCTSMFAGFVIFSIVGFMAHVTKRSIADVAASG |
| | | PGLAFLAYPEAVTQLPISPLWAILFFSMLLMLGIDSQFCTVEGFI |
| | | TALVDEYPRLLRNRRELFIAAVCIISYLIGLSNITQGGIYVFKLE |
| | | DYYSASGMSLLFLVFFECVSISWFYGVNRFYDNIQEMVGSRPCIW |
| | | WKLCWSFFTPIIVAGVFIFSAVQMTPLTMGNYVFPKWGQGVGWLM |
| | | ALSSMVLIPGYMAYMFLTLKGSLKQRIQVMVQPSEDIVRPENGPE |
| | | QPQAGSSTSKEAYIKVPVFTLEDFVGDWRQTAGYNLDQVLEQGGV |
| | | SSLFQNLGVSVTPIQRIVLSGENGLKIDIHVIIPYEGLSGDQMGQ |
| | | IEKIFKVVYPVDDHHFKVILHYGTLVIDGVTPNMIDYEGRPYEGI |
| | | AVFDGKKITVTGTLWNGNKIIDERLINPDGSLLFRVTINGVTGWR |
| | | LCERILAGGGGSPSRLEEELRRRLTEP |
| KCNQ2-nanoluc- | 435 | MVQKSRNGGVYPGPSGEKKLKVGFVGLDPGAPDSTRDGALLIAGS |
| alfa-tag-fusion | | EAPKRGSILSKPRAGGAGAGKPPKRNAFYRKLQNFLYNVLERPRG |
| | | WAFIYHAYVELLVESCLVLSVESTIKEYEKSSEGALYILEIVTIV |
| | | VFGVEYFVRIWAAGCCCRYRGWRGRLKFARKPFCVIDIMVLIASI |
| | | AVLAAGSQGNVFATSALRSLRFLQILRMIRMDRRGGTWKLLGSVV |
| | | YAHSKELVTAWYIGELCLILASELVYLAEKGENDHEDTYADALWW |
| | | GLITLTTIGYGDKYPQTWNGRLLAATFTLIGVSFFALPAGILGSG |
| | | FALKVQEQHRQKHFEKRRNPAAGLIQSAWRFYATNLSRTDLHSTW |
| | | QYYERTVTVPMYSSQTQTYGASRLIPPLNQLELLRNLKSKSGLAF |
| | | RKDPPPEPSPSKGSPCRGPLCGCCPGRSSQKVSLKDRVESSPRGV |
| | | AAKGKGSPQAQTVRRSPSADQSLEDSPSKVPKSWSEGDRSRARQA |
| | | FRIKGAASRQNSEEASLPGEDIVDDKSCPCEFVTEDLTPGLKVSI |
| | | RAVCVMRFLVSKRKFKESLRPYDVMDVIEQYSAGHLDMLSRIKSL |
| | | QSRVDQIVGRGPAITDKDRTKGPAEAELPEDPSMMGRLGKVEKQV |
| | | LSMEKKLDELVNIYMQRMGIPPTETEAYFGAKEPEPAPPYHSPED |
| | | SREHVDRHGCIVKIVRSSSSTGQKNFSAPPAAPPVQCPPSTSWQP |
| | | QSHPRQGHGTSPVGDHGSLVRIPPPPAHERSLSAYGGGNRASMEF |
| | | LRQEDTPGCRPPEGNLRDSDTSISIPSVDHEELERSESGFSISQS |
| | | KENLDALNSCYAAVAPCAKVRPYIAEGESDTDSDLCTPCGPPPRS |
| | | ATGEGPFGDVGWAGPRKKVPVFTLEDFVGDWRQTAGYNLDQVLEQ |
| | | GGVSSLFQNLGVSVTPIQRIVLSGENGLKIDIHVIIPYEGLSGDQ |
| | | MGQIEKIFKVVYPVDDHHFKVILHYGTLVIDGVTPNMIDYFGRPY |
| | | EGIAVFDGKKITVTGTLWNGNKIIDERLINPDGSLLERVTINGVT |
| | | GWRLCERILAGGGGSPSRLEEELRRRLTEP |
| SCN8A-nanoluc- | 436 | MAARLLAPPGPDSFKPFTPESLANIERRIAESKLKKPPKADGSHR |
| alfa-tag-fusion | | EDDEDSKPKPNSDLEAGKSLPFIYGDIPQGLVAVPLEDEDPYYLT |
| | | QKTFVVLNRGKTLFRFSATPALYILSPENLIRRIAIKILIHSVES |
| | | MIIMCTILTNCVEMTFSNPPDWSKNVEYTFTGIYTFESLVKIIAR |
| | | GFCIDGFTFLRDPWNWLDESVIMMAYITEFVNLGNVSALRTERVL |
| | | RALKTISVIPGLKTIVGALIQSVKKLSDVMILTVFCLSVFALIGL |
| | | QLFMGNLRNKCVVWPINENESYLENGTKGEDWEEYINNKTNFYTV |
| | | PGMLEPLLCGNSSDAGQCPEGYQCMKAGRNPNYGYTSFDTFSWAF |
| | | LALFRLMTQDYWENLYQLTLRAAGKTYMIFFVLVIFVGSFYLVNL |
| | | ILAVVAMAYEEQNQATLEEAEQKEAEFKAMLEQLKKQQEEAQAAA |
| | | MATSAGTVSEDAIEEEGEEGGGSPRSSSEISKLSSKSAKERRNRR |
| | | KKRKQKELSEGEEKGDPEKVEKSESEDGMRRKAFRLPDNRIGRKE |
| | | SIMNQSLLSIPGSPFLSRHNSKSSIFSFRGPGRERDPGSENEFAD |
| | | DEHSTVEESEGRRDSLFIPIRARERRSSYSGYSGYSQGSRSSRIF |
| | | PSLRRSVKRNSTVDCNGVVSLIGGPGSHIGGRLLPEATTEVEIKK |
| | | KGPGSLLVSMDQLASYGRKDRINSIMSVVTNTLVEELEESQRKCP |
| | | PCWYKFANTFLIWECHPYWIKLKEIVNLIVMDPFVDLAITICIVL |
| | | NTLFMAMEHHPMTPQFEHVLAVGNLVFTGIFTAEMELKLIAMDPY |
| | | YYFQEGWNIFDGFIVSLSLMELSLADVEGLSVLRSERLLRVEKLA |
| | | KSWPTLNMLIKIIGNSVGALGNLTLVLAIIVFIFAVVGMQLFGKS |
| | | YKECVCKINQDCELPRWHMHDFFHSFLIVERVLCGEWIETMWDCM |
| | | EVAGQAMCLIVEMMVMVIGNLVVLNLFLALLLSSFSADNLAATDD |
| | | DGEMNNLQISVIRIKKGVAWTKLKVHAFMQAHFKQREADEVKPLD |
| | | ELYEKKANCIANHTGADIHRNGDFQKNGNGTTSGIGSSVEKYIID |
| | | EDHMSFINNPNLTVRVPIAVGESDFENLNTEDVSSESDPEGSKDK |
| | | LDDTSSSEGSTIDIKPEVEEVPVEQPEEYLDPDACFTEGCVQRFK |
| | | CCQVNIEEGLGKSWWILRKTCFLIVEHNWFETFIIFMILLSSGAL |
| | | AFEDIYIEQRKTIRTILEYADKVETYIFILEMLLKWTAYGFVKFF |
| | | TNAWCWLDELIVAVSLVSLIANALGYSELGAIKSLRTLRALRPLR |
| | | ALSRFEGMRVVVNALVGAIPSIMNVLLVCLIFWLIFSIMGVNLFA |
| | | GKYHYCFNETSEIRFEIEDVNNKTECEKLMEGNNTEIRWKNVKIN |
| | | FDNVGAGYLALLQVATFKGWMDIMYAAVDSRKPDEQPKYEDNIYM |
| | | YIYFVIFIIFGSFFTLNLFIGVIIDNENQQKKKFGGQDIFMTEEQ |
| | | KKYYNAMKKLGSKKPQKPIPRPLNKIQGIVFDFVTQQAFDIVIMM |
| | | LICLNMVTMMVETDTQSKQMENILYWINLVFVIFFTCECVLKMFA |
| | | LRHYYFTIGWNIFDFVVVILSIVGMFLADIIEKYFVSPTLERVIR |
| | | LARIGRILRLIKGAKGIRTLLFALMMSLPALFNIGLLLFLVMFIF |
| | | SIFGMSNFAYVKHEAGIDDMENFETFGNSMICLFQITTSAGWDGL |
| | | LLPILNRPPDCSLDKEHPGSGFKGDCGNPSVGIFFFVSYIIISFL |
| | | IVVNMYIAIILENFSVATEESADPLSEDDFETFYEIWEKFDPDAT |
| | | QFIEYCKLADFADALEHPLRVPKPNTIELIAMDLPMVSGDRIHCL |
| | | DILFAFTKRVLGDSGELDILRQQMEERFVASNPSKVSYEPITTTL |
| | | RRKQEEVSAVVLQRAYRGHLARRGFICKKTTSNKLENGGTHREKK |
| | | ESTPSTASLPSYDSVTKPEKEKQQRAEEGRRERAKRQKEVRESKC |
| | | KVPVFTLEDFVGDWRQTAGYNLDQVLEQGGVSSLFQNLGVSVTPI |
| | | QRIVLSGENGLKIDIHVIIPYEGLSGDQMGQIEKIFKVVYPVDDH |
| | | HFKVILHYGTLVIDGVTPNMIDYFGRPYEGIAVEDGKKITVTGTL |
| | | WNGNKIIDERLINPDGSLLFRVTINGVTGWRLCERILAGGGGSPS |
| | | RLEEELRRRLTEP |
| SCN2A-nanoluc- | 437 | MAQSVLVPPGPDSFRFFTRESLAAIEQRIAEEKAKRPKQERKDED |
| alfa-tag-fusion | | DENGPKPNSDLEAGKSLPFIYGDIPPEMVSVPLEDLDPYYINKKT |
| | | FIVLNKGKAISRFSATPALYILTPENPIRKLAIKILVHSLENMLI |
| | | MCTILTNCVFMTMSNPPDWTKNVEYTFTGIYTFESLIKILARGFC |
| | | LEDFTFLRDPWNWLDFTVITFAYVTEFVDLGNVSALRTERVLRAL |
| | | KTISVIPGLKTIVGALIQSVKKLSDVMILTVECLSVFALIGLQLE |
| | | MGNLRNKCLQWPPDNSSFEINITSFENNSLDGNGTTFNRTVSIEN |
| | | WDEYIEDKSHFYFLEGQNDALLCGNSSDAGQCPEGYICVKAGRNP |
| | | NYGYTSFDTFSWAFLSLERLMTQDFWENLYQLTLRAAGKTYMIFF |
| | | VLVIFLGSFYLINLILAVVAMAYEEQNQATLEEAEQKEAEFQQML |
| | | EQLKKQQEEAQAAAAAASAESRDESGAGGIGVFSESSSVASKLSS |
| | | KSEKELKNRRKKKKQKEQSGEEEKNDRVRKSESEDSIRRKGFRES |
| | | LEGSRLTYEKRFSSPHQSLLSIRGSLFSPRRNSRASLESFRGRAK |
| | | DIGSENDFADDEHSTFEDNDSRRDSLFVPHRHGERRHSNVSQASR |
| | | ASRVLPILPMNGKMHSAVDCNGVVSLVGGPSTLTSAGQLLPEGTT |
| | | TETEIRKRRSSSYHVSMDLLEDPTSRQRAMSIASILTNTMEELEE |
| | | SRQKCPPCWYKFANMCLIWDCCKPWLKVKHLVNLVVMDPFVDLAI |
| | | TICIVLNTLFMAMEHYPMTEQFSSVLSVGNLVETGIFTAEMELKI |
| | | IAMDPYYYFQEGWNIFDGFIVSLSLMELGLANVEGLSVLRSERLL |
| | | RVFKLAKSWPTLNMLIKIIGNSVGALGNLTLVLAIIVFIFAVVGM |
| | | QLFGKSYKECVCKISNDCELPRWHMHDFFHSFLIVERVLCGEWIE |
| | | TMWDCMEVAGQTMCLTVFMMVMVIGNLVVLNLFLALLLSSFSSDN |
| | | LAATDDDNEMNNLQIAVGRMQKGIDFVKRKIREFIQKAFVRKQKA |
| | | LDEIKPLEDLNNKKDSCISNHTTIEIGKDLNYLKDGNGTTSGIGS |
| | | SVEKYVVDESDYMSFINNPSLTVTVPIAVGESDFENLNTEEFSSE |
| | | SDMEESKEKLNATSSSEGSTVDIGAPAEGEQPEVEPEESLEPEAC |
| | | FTEDCVRKFKCCQISIEEGKGKLWWNLRKTCYKIVEHNWFETFIV |
| | | FMILLSSGALAFEDIYIEQRKTIKTMLEYADKVETYIFILEMLLK |
| | | WVAYGFQVYFTNAWCWLDFLIVDVSLVSLTANALGYSELGAIKSL |
| | | RTLRALRPLRALSRFEGMRVVVNALLGAIPSIMNVLLVCLIFWLI |
| | | FSIMGVNLFAGKFYHCINYTTGEMFDVSVVNNYSECKALIESNQT |
| | | ARWKNVKVNFDNVGLGYLSLLQVATFKGWMDIMYAAVDSRNVELQ |
| | | PKYEDNLYMYLYFVIFIIFGSFFTLNLFIGVIIDNFNQQKKKFGG |
| | | QDIFMTEEQKKYYNAMKKLGSKKPQKPIPRPANKFQGMVEDFVTK |
| | | QVFDISIMILICLNMVTMMVETDDQSQEMTNILYWINLVFIVLFT |
| | | GECVLKLISLRYYYFTIGWNIFDFVVVILSIVGMFLAELIEKYFV |
| | | SPTLERVIRLARIGRILRLIKGAKGIRTLLFALMMSLPALENIGL |
| | | LLFLVMFIYAIFGMSNFAYVKREVGIDDMENFETEGNSMICLFQI |
| | | TTSAGWDGLLAPILNSGPPDCDPDKDHPGSSVKGDCGNPSVGIFF |
| | | FVSYIIISFLVVVNMYIAVILENFSVATEESAEPLSEDDFEMFYE |
| | | VWEKFDPDATQFIEFAKLSDFADALDPPLLIAKPNKVQLIAMDLP |
| | | MVSGDRIHCLDILFAFTKRVLGESGEMDALRIQMEEREMASNPSK |
| | | VSYEPITTTLKRKQEEVSAIIIQRAYRRYLLKQKVKKVSSIYKKD |
| | | KGKECDGTPIKEDTLIDKLNENSTPEKTDMTPSTTSPPSYDSVTK |
| | | PEKEKFEKDKSEKEDKGKDIRESKKKVPVFTLEDFVGDWRQTAGY |
| | | NLDQVLEQGGVSSLFQNLGVSVTPIQRIVLSGENGLKIDIHVIIP |
| | | YEGLSGDQMGQIEKIFKVVYPVDDHHFKVILHYGTLVIDGVTPNM |
| | | IDYFGRPYEGIAVEDGKKITVTGTLWNGNKIIDERLINPDGSLLF |
| | | RVTINGVTGWRLCERILAGGGGSPSRLEEELRRRLTEP |
| Scn1a-nanoluc- | 438 | MEQTVLVPPGPDSENFFTRESLAAIERRIAEEKAKNPKPDKKDDD |
| alfa-tag-fusion | | ENGPKPNSDLEAGKNLPFIYGDIPPEMVSEPLEDLDPYYINKKTE |
| | | IVLNKGKAIFRESATSALYILTPFNPLRKIAIKILVHSLESMLIM |
| | | CTILTNCVEMTMSNPPDWTKNVEYTFTGIYTFESLIKIIARGECL |
| | | EDFTELRDPWNWLDFTVITFAYVTEFVDLGNVSALRTERVLRALK |
| | | TISVIPGLKTIVGALIQSVKKLSDVMILTVFCLSVFALIGLQLEM |
| | | GNLRNKCIQWPPTNASLEEHSIEKNITVNYNGTLINETVFEEDWK |
| | | SYIQDSRYHYFLEGELDALLCGNSSDAGQCPEGYMCVKAGRNPNY |
| | | GYTSFDTESWAFLSLERLMTQDFWENLYQLTLRAAGKTYMIFFVL |
| | | VIFLGSFYLINLILAVVAMAYEEQNQATLEEAEQKEAEFQQMIEQ |
| | | LKKQQEAAQQAATATASEHSREPSAAGRLSDSSSEASKLSSKSAK |
| | | ERRNRRKKRKQKEQSGGEEKDEDEFQKSESEDSIRRKGFRESIEG |
| | | NRLTYEKRYSSPHQSLLSIRGSLFSPRRNSRTSLESERGRAKDVG |
| | | SENDFADDEHSTFEDNESRRDSLFVPRRHGERRNSNLSQTSRSSR |
| | | MLAVFPANGKMHSTVDCNGVVSLVGGPSVPTSPVGQLLPEVIIDK |
| | | PATDDNGTTTETEMRKRRSSSFHVSMDFLEDPSQRQRAMSIASIL |
| | | TNTVEELEESRQKCPPCWYKESNIFLIWDCSPYWLKVKHVVNLVV |
| | | MDPFVDLAITICIVLNTLFMAMEHYPMTDHENNVLTVGNLVETGI |
| | | FTAEMELKIIAMDPYYYFQEGWNIFDGFIVTLSLVELGLANVEGL |
| | | SVLRSFRLLRVFKLAKSWPTLNMLIKIIGNSVGALGNLTLVLAII |
| | | VFIFAVVGMQLFGKSYKDCVCKIASDCQLPRWHMNDFFHSFLIVE |
| | | RVLCGEWIETMWDCMEVAGQAMCLTVFMMVMVIGNLVVLNLFLAL |
| | | LLSSFSADNLAATDDDNEMNNLQIAVDRMHKGVAYVKRKIYEFIQ |
| | | QSFIRKQKILDEIKPLDDLNNKKDSCMSNHTAEIGKDLDYLKDVN |
| | | GTTSGIGTGSSVEKYIIDESDYMSFINNPSLTVTVPIAVGESDFE |
| | | NLNTEDESSESDLEESKEKLNESSSSSEGSTVDIGAPVEEQPVVE |
| | | PEETLEPEACFTEGCVQRFKCCQINVEEGRGKQWWNLRRTCFRIV |
| | | EHNWFETFIVEMILLSSGALAFEDIYIDQRKTIKTMLEYADKVET |
| | | YIFILEMLLKWVAYGYQTYFTNAWCWLDFLIVDVSLVSLTANALG |
| | | YSELGAIKSLRTLRALRPLRALSRFEGMRVVVNALLGAIPSIMNV |
| | | LLVCLIFWLIFSIMGVNLFAGKFYHCINTTTGDREDIEDVNNHTD |
| | | CLKLIERNETARWKNVKVNFDNVGFGYLSLLQVATFKGWMDIMYA |
| | | AVDSRNVELQPKYEESLYMYLYFVIFIIFGSFFTLNLFIGVIIDN |
| | | FNQQKKKFGGQDIFMTEEQKKYYNAMKKLGSKKPQKPIPRPGNKF |
| | | QGMVFDFVTRQVEDISIMILICLNMVTMMVETDDQSEYVTTILSR |
| | | INLVFIVLFTGECVLKLISLRHYYFTIGWNIFDFVVVILSIVGME |
| | | LAELIEKYFVSPTLFRVIRLARIGRILRLIKGAKGIRTLLFALMM |
| | | SLPALFNIGLLLFLVMFIYAIFGMSNFAYVKREVGIDDMENFETE |
| | | GNSMICLFQITTSAGWDGLLAPILNSKPPDCDPNKVNPGSSVKGD |
| | | CGNPSVGIFFFVSYIIISELVVVNMYIAVILENESVATEESAEPL |
| | | SEDDFEMFYEVWEKFDPDATQFMEFEKLSQFAAALEPPLNLPQPN |
| | | KLQLIAMDLPMVSGDRIHCLDILFAFTKRVLGESGEMDALRIQME |
| | | ERFMASNPSKVSYQPITTTLKRKQEEVSAVIIQRAYRRHLLKRTV |
| | | KQASFTYNKNKIKGGANLLIKEDMIIDRINENSITEKTDLTMSTA |
| | | ACPPSYDRVTKPIVEKHEQEGKDEKAKGKKVPVFTLEDFVGDWRQ |
| | | TAGYNLDQVLEQGGVSSLFQNLGVSVTPIQRIVLSGENGLKIDIH |
| | | VIIPYEGLSGDQMGQIEKIFKVVYPVDDHHFKVILHYGTLVIDGV |
| | | TPNMIDYFGRPYEGIAVEDGKKITVTGTLWNGNKIIDERLINPDG |
| | | SLLFRVTINGVTGWRLCERILAGGGGSPSRLEEELRRRLTEP |
| Grin2b-nanoluc- | 439 | MKPRAECCSPKFWLVLAVLAVSGSRARSQKSPPSIGIAVILVGTS |
| alfa-tag-fusion | | DEVAIKDAHEKDDFHHLSVVPRVELVAMNETDPKSIITRICDLMS |
| | | DRKIQGVVFADDTDQEAIAQILDFISAQTLTPILGIHGGSSMIMA |
| | | DKDESSMFFQFGPSIEQQASVMLNIMEEYDWYIFSIVTTYFPGYQ |
| | | DFVNKIRSTIENSFVGWELEEVLLLDMSLDDGDSKIQNQLKKLQS |
| | | PIILLYCTKEEATYIFEVANSVGLTGYGYTWIVPSLVAGDTDTVP |
| | | AEFPTGLISVSYDEWDYGLPARVRDGIAIITTAASDMLSEHSFIP |
| | | EPKSSCYNTHEKRIYQSNMLNRYLINVTFEGRNLSFSEDGYQMHP |
| | | KLVIILLNKERKWERVGKWKDKSLQMKYYVWPRMCPETEEQEDDH |
| | | LSIVTLEEAPFVIVESVDPLSGTCMRNTVPCQKRIVTENKTDEEP |
| | | GYIKKCCKGFCIDILKKISKSVKFTYDLYLVTNGKHGKKINGTWN |
| | | GMIGEVVMKRAYMAVGSLTINEERSEVVDFSVPFIETGISVMVSR |
| | | SNGTVSPSAFLEPFSADVWVMMFVMLLIVSAVAVFVFEYESPVGY |
| | | NRCLADGREPGGPSFTIGKAIWLLWGLVENNSVPVQNPKGTTSKI |
| | | MVSVWAFFAVIFLASYTANLAAFMIQEEYVDQVSGLSDKKFQRPN |
| | | DFSPPFRFGTVPNGSTERNIRNNYAEMHAYMGKFNQRGVDDALLS |
| | | LKTGKLDAFIYDAAVLNYMAGRDEGCKLVTIGSGKVFASTGYGIA |
| | | IQKDSGWKRQVDLAILQLFGDGEMEELEALWLTGICHNEKNEVMS |
| | | SQLDIDNMAGVFYMLGAAMALSLITFICEHLFYWQFRHCFMGVCS |
| | | GKPGMVESISRGIYSCIHGVAIEERQSVMNSPTATMNNTHSNILR |
| | | LLRTAKNMANLSGVNGSPQSALDFIRRESSVYDISEHRRSFTHSD |
| | | CKSYNNPPCEENLESDYISEVERTFGNLQLKDSNVYQDHYHHHHR |
| | | PHSIGSASSIDGLYDCDNPPFTTQSRSISKKPLDIGLPSSKHSQL |
| | | SDLYGKESFKSDRYSGHDDLIRSDVSDISTHTVTYGNIEGNAAKR |
| | | RKQQYKDSLKKRPASAKSRREFDEIELAYRRRPPRSPDHKRYFRD |
| | | KEGLRDFYLDQFRTKENSPHWEHVDLTDIYKERSDDFKRDSVSGG |
| | | GPCTNRSHIKHGTGDKHGVVSGVPAPWEKNLTNVEWEDRSGGNFC |
| | | RSCPSKLHNYSTTVTGQNSGRQACIRCEACKKAGNLYDISEDNSL |
| | | QELDQPAAPVAVTSNASTTKYPQSPTNSKAQKKNRNKLRRQHSYD |
| | | TFVDLQKEEAALAPRSVSLKDKGREMDGSPYAHMFEMSAGESTFA |
| | | NNKSSVPTAGHHHHNNPGGGYMLSKSLYPDRVTQNPFIPTFGDDQ |
| | | CLLHGSKSYFFRQPTVAGASKARPDFRALVTNKPVVSALHGAVPA |
| | | RFQKDICIGNQSNPCVPNNKNPRAFNGSSNGHVYEKLSSIESDVK |
| | | VPVFTLEDFVGDWRQTAGYNLDQVLEQGGVSSLFQNLGVSVTPIQ |
| | | RIVLSGENGLKIDIHVIIPYEGLSGDQMGQIEKIFKVVYPVDDHH |
| | | FKVILHYGTLVIDGVTPNMIDYFGRPYEGIAVEDGKKITVTGTLW |
| | | NGNKIIDERLINPDGSLLFRVTINGVTGWRLCERILAGGGGSPSR |
| | | LEEELRRRLTEP |
| SLC50A1-nanoluc- | 440 | MEAGGFLDSLIYGACVVFTLGMESAGLSDLRHMRMTRSVDNVQFL |
| alfa-tag-fusion | | PFLTTEVKKVPVFTLEDFVGDWRQTAGYNLDQVLEQGGVSSLFQN |
| | | LGVSVTPIQRIVLSGENGLKIDIHVIIPYEGLSGDQMGQIEKIFK |
| | | VVYPVDDHHFKVILHYGTLVIDGVTPNMIDYFGRPYEGIAVEDGK |
| | | KITVTGTLWNGNKIIDERLINPDGSLLFRVTINGVTGWRLCERIL |
| | | AGGGGSPSRLEEELRRRLTEP |
| TMEM258- | 441 | MELEAMSRYTSPVNPAVFPHLTVVLLAIGMFFTAWFFVYPFTEQP |
| nanoluc-alfa-tag- | | EDQHKVPVFTLEDFVGDWRQTAGYNLDQVLEQGGVSSLFQNLGVS |
| fusion | | VTPIQRIVLSGENGLKIDIHVIIPYEGLSGDQMGQIEKIFKVVYP |
| | | VDDHHFKVILHYGTLVIDGVTPNMIDYFGRPYEGIAVEDGKKITV |
| | | TGTLWNGNKIIDERLINPDGSLLFRVTINGVTGWRLCERILAGGG |
| | | GSPSRLEEELRRRLTEP |
| FSHR-nanoluc- | 442 | MALLLVSLLAFLSLGSGCHHRICHCSNRVFLCQESKVTEIPSDLP |
| alfa-tag-fusion | | RNAIELKVPVFTLEDFVGDWRQTAGYNLDQVLEQGGVSSLFQNLG |
| | | VSVTPIQRIVLSGENGLKIDIHVIIPYEGLSGDQMGQIEKIFKVV |
| | | YPVDDHHFKVILHYGTLVIDGVTPNMIDYFGRPYEGIAVEDGKKI |
| | | TVTGTLWNGNKIIDERLINPDGSLLFRVTINGVTGWRLCERILAG |
| | | GGGSPSRLEEELRRRLTEP |
| KCNQ1-nanoluc- | 446 | MAAASSPPRAERKRWGWGRLPGARRGSAGLAKKCPFSLELAEGGP |
| alfa-tag-fusion | | AGGALYAPIAPGAPGPAPPASPAAPAAPPVASDLGPRPPVSLDPR |
| | | VSIYSTRRPVLARTHVQGRVYNFLERPTGWKCFVYHFAVELIVLV |
| | | CLIFSVLSTIEQYAWRYYESSLEPYPDALATGTLEWMEIVLVVFF |
| | | GTEYVVRLWSAGCRSKYVGLWGRLRFARKPISIIDLIVVVASMVV |
| | | LCVGSKGQVFATSAIRGIRFLQILRMLHVDRQGGTWRLLGSVVFI |
| | | HRQELITTLYIGELGLIFSSYFVYLAEKDAVNESGRVEFGSYADA |
| | | LWWGVVTVTTIGYGDKVPQTWVGKTIASCFSVFAISFFALPAGIL |
| | | GSGFALKVQQKQRQKHENRQIPAAASLIQTAWRCYAAENPDSSTW |
| | | KIYIRKAPRSHTLLSPSPKPKKSVVVKKKKFKLDKDNGVTPGEKM |
| | | LTVPHITCDPPEERRLDHFSVDGYDSSVRKSPTLLEVSMPHEMRT |
| | | NSFAEDLDLEGETLLTPITHISQLREHHRATIKVIRRMQYFVAKK |
| | | KFQQARKPYDVRDVIEQYSQGHLNLMVRIKELQRRLDQSIGKPSL |
| | | FISVSEKSKDRGSNTIGARLNRVEDKVTQLDQRLALITDMLHQLL |
| | | SLHGGSTPGSGGPPREGGAHITQPCGSGGSVDPELELPSNTLPTY |
| | | EQLTVPRRGPDEGSKVPVFTLEDFVGDWRQTAGYNLDQVLEQGGV |
| | | SSLFQNLGVSVTPIQRIVLSGENGLKIDIHVIIPYEGLSGDQMGQ |
| | | IEKIFKVVYPVDDHHFKVILHYGTLVIDGVTPNMIDYFGRPYEGI |
| | | AVFDGKKITVTGTLWNGNKIIDERLINPDGSLLFRVTINGVTGWR |
| | | LCERILAGGGGSPSRLEEELRRRLTEP |

The amino acid sequence of exemplary fusion proteins comprising a control or a targeted engineered deubiquitinase are detailed in Table 8 below.

TABLE 8
Amino Acid Sequence of exemplary enDub Control and Screening Fusion Proteins

| Description | SEQ ID NO | Amino Acid Sequence |
|---|---|---|
| FireflyLuciferase- | 443 | MEDAKNIKKGPAPFYPLEDGTAGEQLHKAMKRYALVPGTIAFTDA |
| P2A-nano | | HIEVDITYAEYFEMSVRLAEAMKRYGLNTNHRIVVCSENSLQFEM |
| (Control) | | PVLGALFIGVAVAPANDIYNERELLNSMGISQPTVVFVSKKGLQK |
| | | ILNVQKKLPIIQKIIIMDSKTDYQGFQSMYTFVTSHLPPGENEYD |
| | | FVPESFDRDKTIALIMNSSGSTGLPKGVALPHRTACVRESHARDP |
| | | IFGNQIIPDTAILSVVPFHHGFGMFTTLGYLICGFRVVLMYRFEE |
| | | ELFLRSLQDYKIQSALLVPTLESFFAKSTLIDKYDLSNLHEIASG |
| | | GAPLSKEVGEAVAKRFHLPGIRQGYGLTETTSAILITPEGDDKPG |
| | | AVGKVVPFFEAKVVDLDTGKTLGVNQRGELCVRGPMIMSGYVNNP |
| | | EATNALIDKDGWLHSGDIAYWDEDEHFFIVDRLKSLIKYKGYQVA |
| | | PAELESILLQHPNIFDAGVAGLPDDDAGELPAAVVVLEHGKTMTE |
| | | KEIVDYVASQVTTAKKLRGGVVFVDEVPKGLTGKLDARKIREILI |
| | | KAKKGGKIAVTRLKGSGATNFSLLKQAGDVEENPGPRSGTGSSGE |
| | | VQLQESGGGLVQPGGSLRLSCTASGVTISALNAMAMGWYRQAPGE |
| | | RRVMVAAVSERGNAMYRESVQGRFTVTRDFTNKMVSLQMDNLKPE |
| | | DTAVYYCHVLEDRVDSFHDYWGQGTQVTVSS |
| FireflyLuciferase- | 444 | MEDAKNIKKGPAPFYPLEDGTAGEQLHKAMKRYALVPGTIAFTDA |
| P2A-Cezanne | | HIEVDITYAEYFEMSVRLAEAMKRYGLNTNHRIVVCSENSLQFEM |
| (Control) | | PVLGALFIGVAVAPANDIYNERELLNSMGISQPTVVFVSKKGLQK |
| | | ILNVQKKLPIIQKIIIMDSKTDYQGFQSMYTFVTSHLPPGENEYD |
| | | FVPESEDRDKTIALIMNSSGSTGLPKGVALPHRTACVRESHARDP |
| | | IFGNQIIPDTAILSVVPFHHGFGMFTTLGYLICGERVVLMYRFEE |
| | | ELFLRSLQDYKIQSALLVPTLESFFAKSTLIDKYDLSNLHEIASG |
| | | GAPLSKEVGEAVAKRFHLPGIRQGYGLTETTSAILITPEGDDKPG |
| | | AVGKVVPFFEAKVVDLDTGKTLGVNQRGELCVRGPMIMSGYVNNP |
| | | EATNALIDKDGWLHSGDIAYWDEDEHFFIVDRLKSLIKYKGYQVA |
| | | PAELESILLQHPNIFDAGVAGLPDDDAGELPAAVVVLEHGKTMTE |
| | | KEIVDYVASQVTTAKKLRGGVVFVDEVPKGLTGKLDARKIREILI |
| | | KAKKGGKIAVTRLKGSGATNFSLLKQAGDVEENPGPRSGTGSPPS |
| | | FSEGSGGSRTPEKGFSDREPTRPPRPILQRQDDIVQEKRLSRGIS |
| | | HASSSIVSLARSHVSSNGGGGGSNEHPLEMPICAFQLPDLTVYNE |
| | | DERSFIERDLIEQSMLVALEQAGRLNWWVSVDPTSQRLLPLATTG |
| | | DGNCLLHAASLGMWGFHDRDLMLRKALYALMEKGVEKEALKRRWR |
| | | WQQTQQNKESGLVYTEDEWQKEWNELIKLASSEPRMHLGTNGANC |
| | | GGVESSEEPVYESLEEFHVFVLAHVLRRPIVVVADTMLRDSGGEA |
| | | FAPIPFGGIYLPLEVPASQCHRSPLVLAYDQAHFSALVSMEQKEN |
| | | TKEQAVIPLTDSEYKLLPLHFAVDPGKGWEWGKDDSDNVRLASVI |
| | | LSLEVKLHLLHSYMNVKWIPLSSDAQAPLAQ |
| FireflyLuciferase- | 445 | MEDAKNIKKGPAPFYPLEDGTAGEQLHKAMKRYALVPGTIAFTDA |
| P2A- | | HIEVDITYAEYFEMSVRLAEAMKRYGLNTNHRIVVCSENSLQFEM |
| a_alfatag_nano- | | PVLGALFIGVAVAPANDIYNERELLNSMGISQPTVVFVSKKGLQK |
| Cezanne | | ILNVQKKLPIIQKIIIMDSKTDYQGFQSMYTFVTSHLPPGENEYD |
| | | FVPESEDRDKTIALIMNSSGSTGLPKGVALPHRTACVRESHARDP |
| | | IFGNQIIPDTAILSVVPFHHGFGMFTTLGYLICGFRVVLMYRFEE |
| | | ELFLRSLQDYKIQSALLVPTLFSFFAKSTLIDKYDLSNLHEIASG |
| | | GAPLSKEVGEAVAKRFHLPGIRQGYGLTETTSAILITPEGDDKPG |
| | | AVGKVVPFFEAKVVDLDTGKTLGVNQRGELCVRGPMIMSGYVNNP |
| | | EATNALIDKDGWLHSGDIAYWDEDEHFFIVDRLKSLIKYKGYQVA |
| | | PAELESILLQHPNIFDAGVAGLPDDDAGELPAAVVVLEHGKTMTE |
| | | KEIVDYVASQVTTAKKLRGGVVFVDEVPKGLTGKLDARKIREILI |
| | | KAKKGGKIAVTRLKGSGATNFSLLKQAGDVEENPGPRSGTGSSGE |
| | | VQLQESGGGLVQPGGSLRLSCTASGVTISALNAMAMGWYRQAPGE |
| | | RRVMVAAVSERGNAMYRESVQGRFTVTRDFTNKMVSLQMDNLKPE |
| | | DTAVYYCHVLEDRVDSFHDYWGQGTQVTVSSGAPGSGPPSESEGS |
| | | GGSRTPEKGFSDREPTRPPRPILQRQDDIVQEKRLSRGISHASSS |
| | | IVSLARSHVSSNGGGGGSNEHPLEMPICAFQLPDLTVYNEDERSE |
| | | IERDLIEQSMLVALEQAGRLNWWVSVDPTSQRLLPLATTGDGNCL |
| | | LHAASLGMWGFHDRDLMLRKALYALMEKGVEKEALKRRWRWQQTQ |
| | | QNKESGLVYTEDEWQKEWNELIKLASSEPRMHLGTNGANCGGVES |
| | | SEEPVYESLEEFHVEVLAHVLRRPIVVVADTMLRDSGGEAFAPIP |
| | | FGGIYLPLEVPASQCHRSPLVLAYDQAHFSALVSMEQKENTKEQA |
| | | VIPLTDSEYKLLPLHFAVDPGKGWEWGKDDSDNVRLASVILSLEV |
| | | KLHLLHSYMNVKWIPLSSDAQAPLAQ |

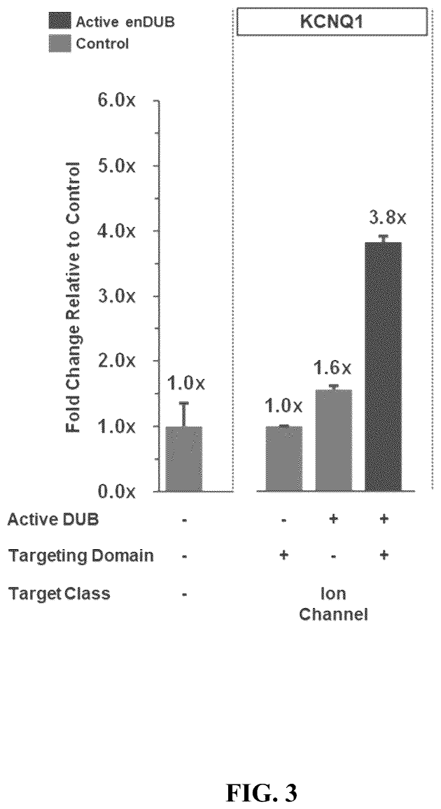

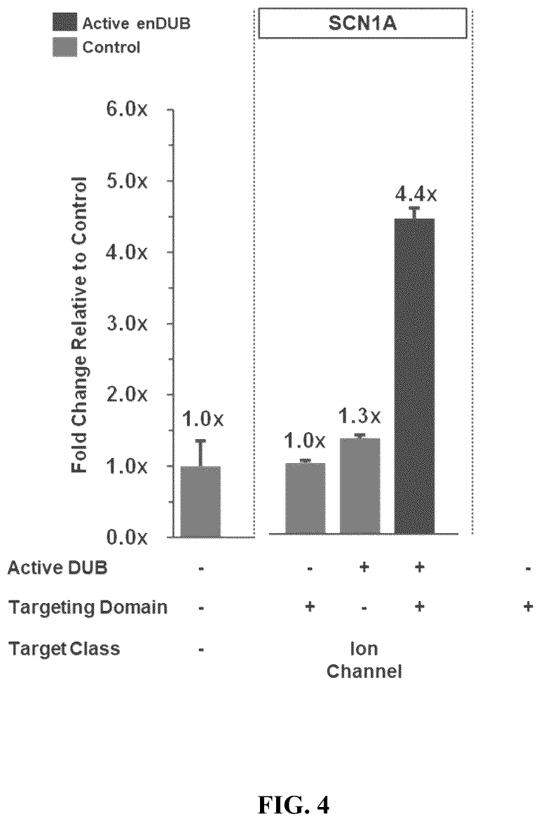

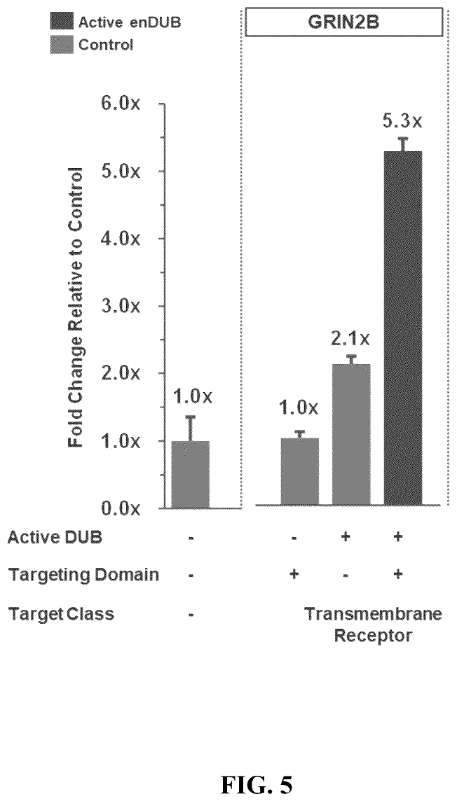

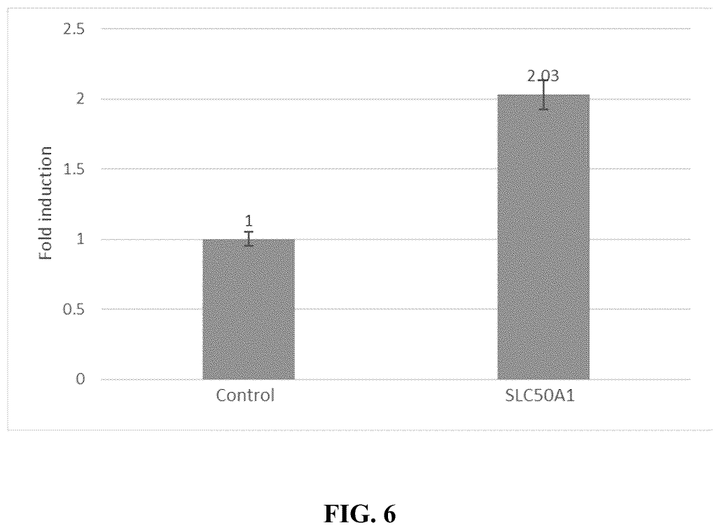

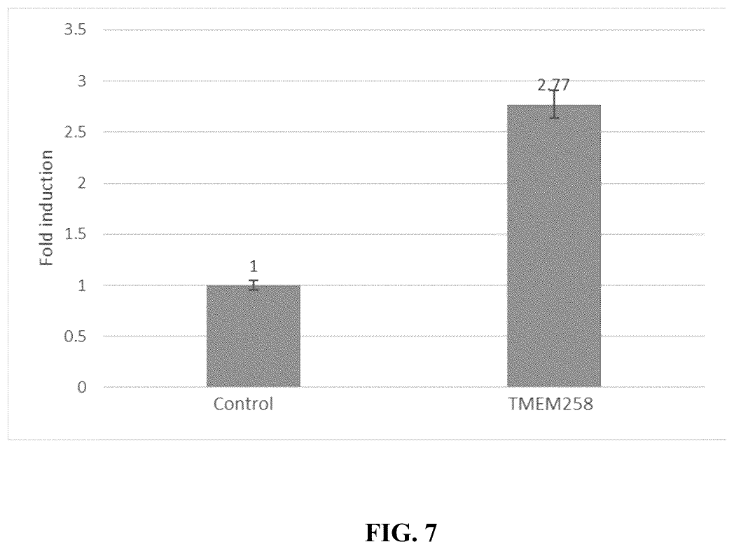

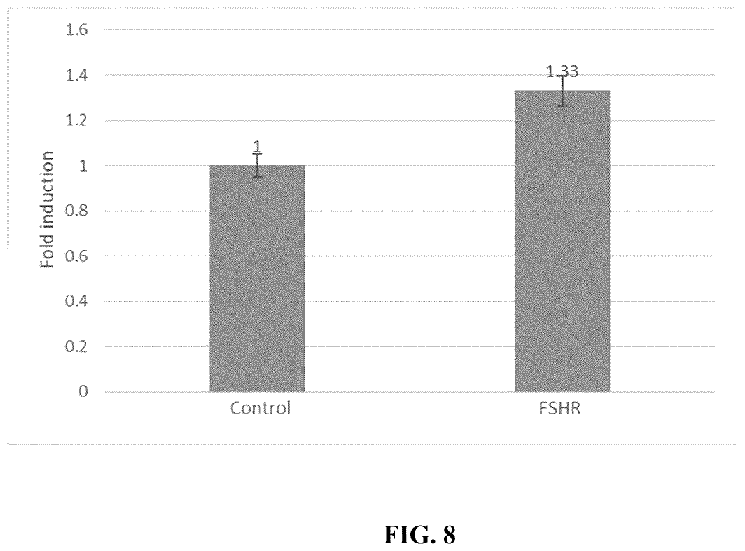

The assay was conducted with utilizing the tagged proteins and targeted enDubs described above in Tables 7 and 8. The results of the KCNQ1 targeting are shown in FIG. 3, showing a 3.8-fold increase in KCNQ1 protein expression. The results of the SCN1A targeting are shown in FIG. 4, showing a 4.4-fold increase in SCN1A protein expression. The results of the GRIN2B targeting are shown in FIG. 5, showing a 5.3-fold increase in GRIN2B protein expression. The results of the SLC50A1 targeting are shown in FIG. 6, showing a 2.03-fold increase in SLC50A1 protein expression. The results of the TREM258 targeting are shown in FIG. 7, showing a 2.77-fold increase in TREM258 protein expression. The results of the FSHR targeting are shown in FIG. 8, showing a 1.33-fold increase in FSHR protein expression. The control used for the SLC50A1, TREM258, and FSHR experiments is the engineered deubiquitinase without the nanobody targeting the alfa-tag. Normalization of transduction efficiency was performed using the firefly luciferase signal as the reference and the ratio between NLuc signal divided by firefly luciferase signal plotted on the y axes.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes. Other embodiments are within the following claims.

What is claimed is:

1. A fusion protein comprising:
a. an effector domain comprising a catalytic domain of a deubiquitinase, or a functional fragment or functional variant thereof; and
b. a targeting domain comprising a targeting moiety that specifically binds a membrane protein that is not an ion channel.

2. The fusion protein of claim 1, wherein said deubiquitinase is a cysteine protease or a metalloprotease.

3. The fusion protein of claim 2, wherein said deubiquitinase is a cysteine protease.

4. The fusion protein of claim 3, wherein said cysteine protease is a ubiquitin-specific protease (USP), a ubiquitin C-terminal hydrolase (UCH), a Machado-Josephin domain protease (MJD), an ovarian tumour protease (OTU), a MINDY protease, or a ZUFSP protease.

5. The fusion protein of claim 4, wherein said cysteine protease is a USP.

6. The fusion protein of claim 5, wherein said USP is USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, or USP46.

7. The fusion protein of claim 4, wherein said cysteine protease is a UCH.

8. The fusion protein of claim 7, wherein said UCH is BAP1, UCHL1, UCHL3, or UCHL5.

9. The fusion protein of claim 4, wherein said cysteine protease is a MJD.

10. The fusion protein of claim 9, wherein said MJD is ATXN3 or ATXN3L.

11. The fusion protein of claim 4, wherein said cysteine protease is a OTU.

12. The fusion protein of claim 11, wherein said OTU is OTUB1 or OTUB2.

13. The fusion protein of claim 4, wherein said cysteine protease is a MINDY.

14. The fusion protein of claim 13, wherein said MINDY is MINDY1, MINDY2, MINDY3, or MINDY4.

15. The fusion protein of claim 4, wherein said cysteine protease is a ZUFSP.

16. The fusion protein of claim 15, wherein said ZUFSP is ZUP1.

17. The fusion protein of claim 2, wherein said deubiquitinase is a metalloprotease.

18. The fusion protein of claim 17, wherein said metalloprotease is a Jab1/Mov34/Mpr1 Pad1 N-terminal+(MPN+) (JAMM) domain protease.

19. The fusion protein of any one of the preceding claims, wherein said deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-112.

20. The fusion protein of any one of the preceding claims, wherein said catalytic domain comprises a catalytic domain derived from a deubiquitinase at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-112.

21. The fusion protein of any one of the preceding claims, wherein said catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 113-220 or 293.

22. The fusion protein of any one of the preceding claims, wherein said catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 293.

23. The fusion protein of any one of the preceding claims, wherein said moiety that specifically binds a membrane protein comprises an antibody, or functional fragment or functional variant thereof.

24. The fusion protein of claim 23, wherein said antibody, or functional fragment or functional variant thereof, comprises a full-length antibody, a single chain variable fragment (scFv), a scFv2, a scFv-Fc, a Fab, a Fab′, a F(ab′)2, a F(v), a VHH, or a (VHH)₂.

25. The fusion protein of claim 23, wherein said antibody, or functional fragment or functional variant thereof, comprises a VHH or a (VHH)₂.

26. The fusion protein of any one of the preceding claims, wherein the membrane protein is selected from the group consisting of solute carrier family 2, facilitated glucose transporter member 1 (SLC2A1), proline-rich transmembrane protein 2 (PRRT2), usherin (USH2A), protocadherin-19 (PCDH19), tuberin (TSC2), hamartin (TSC1), dystrophin (DMD), Rhodopsin (RHO), protein jagged-1 (JAG1), inositol 1,4,5-trisphosphate receptor type 1 (ITPR1), sugar transporter SWEET1 (SLC50A1), transmembrane protein 258 (TMEM258), or follicle stimulating hormone receptor (FSHR).

27. The fusion protein of any one of the preceding claims, wherein the membrane protein comprises an amino acid sequence at least 90%, 91%, 92%, 93%, 94%, 95%, 96%, 97%, 98%, 99%, or 100% identical to the amino acid sequence of any one of SEQ ID NOS: 221-227 or 243-245.

28. The fusion protein of any one of the preceding claims, wherein said effector domain is directly operably connected to said targeting domain.

29. The fusion protein of any one of claims 1-28, wherein said effector domain is indirectly operably connected to said targeting domain.

30. The fusion protein of claim 28, wherein said effector domain is indirectly operably connected to said targeting domain via a peptide linker.

31. The fusion protein of claim 29, wherein said effector domain is indirectly operably connected to said targeting domain via a peptide linker of sufficient length such that said effector domain and said targeting domain can simultaneous bind the respective target proteins.

32. The fusion protein of claim 30 or 31, wherein said peptide linker comprises the amino acid sequence of any one of SEQ ID NOS: 297-424, or the amino acid sequence of any one of SEQ ID NOS: 297-424 comprising 1, 2, or 3 amino acid modifications.

33. The fusion protein of claim 32, wherein said peptide linker comprises the amino acid sequence of any one of SEQ ID NOS: 297-306, or the amino acid sequence of any one of SEQ ID NOS: 297-306 comprising 1, 2, or 3 amino acid modifications.

34. The fusion protein of any one of the preceding claims, wherein said effector domain is operably connected either directly or indirectly to the C terminus of said targeting domain.

35. The fusion protein of any one of claims 1-33, wherein said effector moiety is operably connected either directly or indirectly to the N terminus of said targeting domain.

36. A fusion protein comprising:
a. an effector domain comprising a catalytic domain of a deubiquitinase, or a functional fragment or functional variant thereof; and
b. a targeting domain comprising a targeting moiety that specifically binds a membrane protein selected from the group consisting of glutamate receptor ionotropic NMDA 2B (GRIN2B), cystic fibrosis transmembrane conductance regulator (CFTR), sodium channel protein type 1 subunit alpha (SCN1A), copper-transporting ATPase 2 (ATP7B), potassium voltage-gated channel subfamily KQT member 2 (KCNQ2), sodium channel protein type 2 subunit alpha (SCN2A), voltage-dependent P/Q-type calcium channel subunit alpha-1A (CACNA1A), sodium channel protein type 8 subunit alpha (SCN8A), glutamate receptor ionotropic, NMDA 2A (GRIN2A), sodium- and chloride-dependent GABA transporter 1 (SLC6A1), sodium/potassium-transporting ATPase subunit alpha-2 (ATP1A2), sodium/potassium-transporting ATPase subunit alpha-3 (ATP1A3), sodium channel protein type 9 subunit alpha (SCN9A), gamma-aminobutyric acid receptor subunit beta-3 (GABRB3), and potassium voltage-gated channel subfamily KQT member 3 (KCNQ3).

37. The fusion protein of claim 36, wherein said moiety that specifically binds a membrane protein comprises an antibody, or functional fragment or functional variant thereof.

38. The fusion protein of claim 37, wherein said antibody, or functional fragment or functional variant thereof, comprises a full-length antibody, a single chain variable fragment (scFv), a scFv2, a scFv-Fc, a Fab, a Fab′, a F(ab′)2, a F(v), a VHH, or a (VHH)₂.

39. The fusion protein of claim 38, wherein said antibody, or functional fragment or functional variant thereof, comprises a VHH or a (VHH)₂.

40. The fusion protein of any one of claims 36-39, wherein the membrane protein comprises an amino acid sequence at least 90%, 91%, 92%, 93%, 94%, 95%, 96%, 97%, 98%, 99%, or 100% identical to the amino acid sequence of any one of SEQ ID NOS: 228-245.

41. The fusion protein of any one of claims 36-40, wherein said deubiquitinase is a cysteine protease or a metalloprotease.

42. The fusion protein of claim 41, wherein said deubiquitinase is a cysteine protease.

43. The fusion protein of claim 41, wherein said cysteine protease is a ubiquitin-specific protease (USP), a ubiquitin C-terminal hydrolase (UCH), a Machado-Josephin domain protease (MJD), an ovarian tumour protease (OTU), a MINDY protease, or a ZUFSP protease.

44. The fusion protein of claim 43, wherein said cysteine protease is a USP.

45. The fusion protein of claim 44, wherein said USP is USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, or USP46.

46. The fusion protein of claim 43, wherein said cysteine protease is a UCH.

47. The fusion protein of claim 46, wherein said UCH is BAP1, UCHL1, UCHL3, or UCHL5.

48. The fusion protein of claim 43, wherein said cysteine protease is a MJD.

49. The fusion protein of claim 48, wherein said MJD is ATXN3 or ATXN3L.

50. The fusion protein of claim 43, wherein said cysteine protease is a OTU.

51. The fusion protein of claim 50, wherein said OTU is OTUB1 or OTUB2.

52. The fusion protein of claim 43, wherein said cysteine protease is a MINDY.

53. The fusion protein of claim 52, wherein said MINDY is MINDY1, MINDY2, MINDY3, or MINDY4.

54. The fusion protein of claim 43, wherein said cysteine protease is a ZUFSP.

55. The fusion protein of claim 54, wherein said ZUFSP is ZUP1.

56. The fusion protein of claim 41, wherein said deubiquitinase is a metalloprotease.

57. The fusion protein of claim 56, wherein said metalloprotease is a Jab1/Mov34/Mpr1 Pad1 N-terminal+(MPN+) (JAMM) domain protease.

58. The fusion protein of any one of claims 36-57, wherein said deubiquitinase comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-112.

59. The fusion protein of any one of claims 36-58, wherein said catalytic domain comprises a catalytic domain derived from a deubiquitinase at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 1-112.

60. The fusion protein of any one of claims 36-59, wherein said catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 113-220 or 293.

61. The fusion protein of any one of claims 36-60, wherein said catalytic domain comprises an amino acid sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 293.

62. The fusion protein of any one of claims 36-61, wherein said effector domain is directly operably connected to said targeting domain.

63. The fusion protein of any one of claims 36-62, wherein said effector domain is indirectly operably connected to said targeting domain.

64. The fusion protein of claim 63, wherein said effector domain is indirectly operably connected to said targeting domain via a peptide linker.

65. The fusion protein of claim 64, wherein said effector domain is indirectly operably connected to said targeting domain via a peptide linker of sufficient length such that said effector domain and said targeting domain can simultaneous bind the respective target proteins.

66. The fusion protein of claim 64 or 65, wherein said peptide linker comprises the amino acid sequence of any one of SEQ ID NOS: 297-424, or the amino acid sequence of any one of SEQ ID NOS: 297-424 comprising 1, 2, or 3 amino acid modifications.

67. The fusion protein of claim 66, wherein said peptide linker comprises the amino acid sequence of any one of SEQ ID NOS: 297-306, or the amino acid sequence of any one of SEQ ID NOS: 297-306 comprising 1, 2, or 3 amino acid modifications.

68. The fusion protein of any one of claims 36-67, wherein said effector domain is operably connected either directly or indirectly to the C terminus of said targeting domain.

69. The fusion protein of any one of claims 36-68, wherein said effector moiety is operably connected either directly or indirectly to the N terminus of said targeting domain.

70. A nucleic acid molecule encoding the fusion protein of any one of claims 1-69.

71. The nucleic acid molecule of claim 70, wherein the nucleic acid molecule is a DNA molecule.

72. The nucleic acid molecule of claim 70, wherein the nucleic acid molecule is an RNA molecule.

73. A vector comprising the nucleic acid molecule of any one of claims 70-72.

74. The vector of claim 73, wherein the vector is a plasmid or a viral vector.

75. A viral particle comprising the nucleic acid of any one of claims 70-72.

76. An in vitro cell or population of cells comprising the fusion protein of any one of claims 1-69, the nucleic acid molecule of any one of claims 70-72, or the vector of any one of claims 73-74.

77. A pharmaceutical composition comprising the fusion protein of any one of claims 1-69, the nucleic acid molecule of any one of claims 70-72, the vector of any one of claims 73-74, or the viral particle of claim 75, and an excipient.

78. A method of making the fusion protein of any one of claims 1-69, comprising
a. introducing into an in vitro cell or population of cells the nucleic acid molecule of any one of claims 70-72, the vector of any one of claims 73-74, the viral particle of claim 75;
b. culturing the cell or population of cells in a culture medium under conditions suitable for expression of the fusion protein,
c. isolating the fusion protein from the culture medium, and
d. optionally purifying the fusion protein.

79. A method of treating or preventing a disease in a subject comprising administering the fusion protein of any one of claims 1-69, the nucleic acid of any one of claims 70-72, the vector of any one of claims 73-74, the viral particle of claim 75, or the pharmaceutical composition of claim 77, to a subject in need thereof.

80. The method of claim 79, wherein the subject is human.

81. The method of any one of claims 79-80, wherein the disease is associated with decreased expression of a functional version of the membrane protein relative to a non-diseased control.

82. The method of any one of claims 79-81, wherein the disease is associated with decreased stability of a functional version of the membrane protein relative to a non-diseased control.

83. The method of any one of claims 79-82, wherein the disease is associated with increased ubiquitination and degradation of the membrane protein relative to a non-diseased control.

84. The method of any one of claims 79-83, wherein the disease is a genetic disease.

85. The method of claim 84, wherein the genetic disease is a haploinsufficiency disease.

86. The method of any one of claims 79-85, wherein the disease is a GRIN2B-Related Disorder, a SCN2A-Related Disorder, a SCN8A-Related Disorder, SLC6A1-Related Disorder, a PRRT2 Dyskinesia & Epilepsy, a GRIN2A-Related Disorder, a CACNA1A-Related Disorder, a SCN9A Epilepsy, a PCDH19 Encephalopathy, GLUT1 deficiency syndrome, episodic kinesigenic dyskinesia 1, Usher syndrome type 2A, early infantile epileptic encephalopathy type 9, tuberous sclerosis type 2; tuberous sclerosis type 1, a KCNQ2-Related Disorder (e.g., epileptic encephalopathy), Becker Muscular Dystrophy, autosomal Dominant RP, or Alagille syndrome 1, Gillespie Syndrome.

87. The method of any one of claims 79-86, wherein the disease is early infantile epileptic encephalopathy type 11, early infantile epileptic encephalopathy type 13, early infantile epileptic encephalopathy type 27, cystic fibrosis, Dravet syndrome, Wilson disease, episodic ataxia type 2; epilepsy (e.g., focal, with speech disorder and with or without mental retardation), myoclonic-atonic epilepsy, alternating hemiplegia of childhood, alternating hemiplegia of childhood type 2, epilepsy type 7, GABRB3 associated epilepsy, or a KCNQ2-Related Disorder (e.g., epileptic encephalopathy).

88. The method of any one of claims 79-87, wherein the disease is a GRIN2B-Related Disorder, a SCN2A-Related Disorder, a SCN8A-Related Disorder, SLC6A1-Related Disorder, a PRRT2 Dyskinesia & Epilepsy, a GRIN2A-Related Disorder, a CACNA1A-Related Disorder, a SCN9A Epilepsy, a PCDH19 Encephalopathy, early infantile epileptic encephalopathy type 9, early infantile epileptic encephalopathy type 11, early infantile epileptic encephalopathy type 13, early infantile epileptic encephalopathy type 27, cystic fibrosis, Dravet syndrome, Wilson disease, episodic ataxia type 2; GLUT1 deficiency syndrome, episodic kinesigenic dyskinesia 1, epilepsy (e.g., focal, with speech disorder and with or without mental retardation), KCNQ2 encephalopathy, myoclonic-atonic epilepsy, Usher syndrome type 2A, alternating hemiplegia of childhood, alternating hemiplegia of childhood type 2, epilepsy type 7, GABRB3 associated epilepsy; tuberous sclerosis type 2; tuberous sclerosis type 1, Becker Muscular Dystrophy, autosomal Dominant RP, Alagille syndrome 1, or Gillespie Syndrome.

89. Said method of any one of claims 79-88, wherein
a. said target membrane protein is GRIN2B, and said disease is a GRIN2B related disorder (e.g., an epileptic encephalopathy);
b. said target membrane protein is GRIN2B, and said disease is an early infantile epileptic encephalopathy;
c. said target membrane protein is GRIN2B, and said disease is early infantile epileptic encephalopathy type 27;
d. said target membrane protein is CFTR, and said disease is cystic fibrosis;
e. said target membrane protein is SCN1A, and said disease is Dravet syndrome;
f. said target membrane protein is ATP7B, and said disease is Wilson disease;
g. said target membrane protein is CACNA1A, and said disease is a CACA1A related disorder;
h. said target membrane protein is CACNA1A, and said disease is episodic ataxia type 2;
i. said target membrane protein is KCNQ2, and said disease is an KCNQ2 encephalopathy;
j. said target membrane protein is KCNQ2, and said disease is an epileptic encephalopathy;
k. said target membrane protein is SCN2A, and said disease is a SCN2A related disorder (e.g., an epileptic encephalopathy);
l. said target membrane protein is SCN2A, and said disease is early infantile epileptic encephalopathy type 11;
m. said target membrane protein is SLC2A1, and said disease is GLUT1 deficiency syndrome;
n. said target membrane protein is SCN8A, and said disease is a SCN8A related disorder (e.g., an epileptic encephalopathy);
o. said target membrane protein is SCN8A, and said disease is an epileptic encephalopathy;
p. said target membrane protein is SCN8A, and said disease is early infantile epileptic encephalopathy type 13;
q. said target membrane protein is PRRT2, and said disease is a PRRPT2 dyskinesia and/or epilepsy;
r. said target membrane protein is PRRT2, and said disease is an episodic kinesigenic dyskinesia type;
s. said target membrane protein is PRRT2, and said disease is episodic kinesigenic dyskinesia type 1;
t. said target membrane protein is GRIN2A, and said disease is a GRIN2A related disorder;
u. said target membrane protein is GRIN2A, and said disease is epilepsy;
v. said target membrane protein is GRIN2A, and said disease is focal epilepsy;
w. said target membrane protein is GRIN2A, and said disease is focal epilepsy with speech disorder and with or without mental retardation;
x. said target membrane protein is SLC6A1, and said disease is a SLC6A1 related disorder;
y. said target membrane protein is SLC6A1, and said disease is epilepsy;
z. said target membrane protein is SLC6A1, and said disease is myoclonic-atonic epilepsy;
aa. said target membrane protein is USH2A, and said disease is Usher syndrome;
bb. said target membrane protein is USH2A, and said disease is Usher syndrome type 2A;
cc. said target membrane protein is ATP1A2, and said disease is alternating hemiplegia of childhood;
dd. said target membrane protein is ATP1A2, and said disease is alternating hemiplegia of childhood type 1;
ee. said target membrane protein is ATP1A3, and said disease is alternating hemiplegia of childhood;
ff. said target membrane protein is ATP1A3, and said disease is alternating hemiplegia of childhood type 2;
gg. said target membrane protein is SCN9A, and said disease an SCN9A epilepsy;
hh. said target membrane protein is SCN9A1, and said disease an SCN9A epilepsy;
ii. said target membrane protein is SCN9A1, and said disease is epilepsy;
jj. said target membrane protein is SCN9A1, and said disease is epilepsy type 7;
kk. said target membrane protein is PCDH19, and said disease is PCDH19 encephalopathy;
ll. said target membrane protein is PCDH19, and said disease is an early infantile epileptic encephalopathy;
mm. said target membrane protein is PCDH19, and said disease is early infantile epileptic encephalopathy type 9;
nn. said target membrane protein is GABRB3, and said disease is epilepsy;
oo. said target membrane protein is GABRB3, and said disease is GABRB3 associated epilepsy;
pp. said target membrane protein is TSC2, and said disease is tuberous sclerosis;
qq. said target membrane protein is TSC2, and said disease is tuberous sclerosis type 2;
rr. said target membrane protein is TSC2, and said disease is tuberous sclerosis type 1;
ss. said target membrane protein is TSC1, and said disease is tuberous sclerosis;
tt. said target membrane protein is TSC1, and said disease is tuberous sclerosis type 1;
uu. said target membrane protein is TSC1, and said disease is tuberous sclerosis type 2;
vv. said target membrane protein is KCNQ3, and said disease is KCNQ2-Related Disorders (Epileptic Encephalopathy);
ww. said target membrane protein is DMD, and said disease is Becker Muscular Dystrophy;
xx. said target membrane protein is RHO, and said disease is Autosomal Dominant RP;
yy. said target membrane protein is JAG1, and said disease is Alagille syndrome 1;
zz. said target membrane protein is ITPR1, and said disease is Gillespie Syndrome; or
aaa. said target membrane protein is FSHR, and said disease is ovarian dysgenesis 1 (ODG1).

90. The method of any one of claims 79-89, wherein the fusion protein is administered at a therapeutically effective dose.

91. The method of any one of claims 79-90, wherein the fusion protein is administered systematically or locally.

92. The method of any one of claims 79-91, wherein the fusion protein is administered intravenously, subcutaneously, or intramuscularly.

93. The fusion protein of any one of claims 1-69, the polynucleotide of claim 70, the DNA of claim 71, the RNA of claim 72, the vector of any one of claims 73-74, the viral particle of claim 75, or the pharmaceutical composition of claim 77 for use as a medicament.

94. The fusion protein of any one of claims 1-69, the polynucleotide of claim 70, the DNA of claim 71, the RNA of claim 72, the vector of any one of claims 73-74, the viral particle of claim 75, or the pharmaceutical composition of claim 77 for use in treating or inhibiting a genetic disorder.
